(19) United States
(12) Patent Application Publication
(10) Pub. No.: US 2026/0347353 A1
(43) Pub. Date: Nov. 5, 2026

(54) INDOLIZINE DERIVATIVES FOR TREATING CANCER

(71) Applicant: Antares Therapeutics, Inc., Boston, MA (US)

(72) Inventors: Benjamin C. Milgram, Weston, MA (US); Jack Anthony Henderson, Whitman, MA (US); Angel Guzman-Perez, Belmont, MA (US); David St. Jean, Jr., Natick, MA (US); Heidi Koldsoe, Boston, MA (US); Robert Hicklin, Longmeadow, MA (US)

(21) Appl. No.: 19/120,060

(22) PCT Filed: Oct. 20, 2023
(86) PCT No.: PCT/US2023/077433

§ 371 Date: Apr. 10, 2025

Related U.S. Application Data
(60) Provisional application No. 63/418,102, filed on Oct. 21, 2022.

(51) Int. Cl. C07D 471/04; A61K 31/437

(52) U.S. Cl. CPC C07D 471/04; A61K 31/437

(57) ABSTRACT

This disclosure provides compounds of Formula (I), and pharmaceutically acceptable salts thereof, that restore p53 function. These compounds are useful, e.g., for treating a disease in which decreased p53 function contributes to the pathology and/or symptoms and/or progression of the disease (e.g., cancer) in a subject (e.g., a human).

### CROSS REFERENCE TO RELATED APPLICATIONS

This application claims the benefit of U.S. Provisional Application Ser. No. 63/418,102, filed on Oct. 21, 2022; which is incorporated herein by reference in its entirety.

### SEQUENCE LISTING

This application contains a Sequence Listing that has been submitted electronically as an XML file named 50006-0109WO1_ST26_SL.XML.” The XML file, created on Oct. 19, 2023, is 2,257 bytes in size. The material in the XML file is hereby incorporated by reference in its entirety.

### TECHNICAL FIELD

This disclosure provides compounds of Formula (I), and pharmaceutically acceptable salts thereof, that restore p53 function. These compounds are useful, e.g., for treating a disease in which decreased p53 function contributes to the pathology and/or symptoms and/or progression of the disease (e.g., cancer) in a subject (e.g., a human).

### BACKGROUND

The tumor suppressor p53, encoded by the TP53 gene, is a transcription factor that regulates the expression of genes required for DNA repair, cell cycle arrest, senescence, and apoptosis, and p53 plays a critical role in mediating each of these processes (Alvarado-Ortiz et al., Frontiers in Cell and Developmental Biology (2021) 8, Article 607670; Vousden et al., Cell (2009) 137, 413-431; Bieging et al., Nat. Rev. Cancer (2014) 14, 359-370). TP53 is altered in over 50% of all human cancers, making it the most frequently mutated gene among oncogenes and tumor suppressor genes (Hainaut et al., Adv Cancer Res (2000) 77, 81-137; Joerger et al., Cold Spring Harb. Perspect. Biol. (2010) 2(6), Article a000919). Mutations in TP53 result in loss of its normal function, rendering cells incapable of responding to a variety of cellular stresses such as DNA damage or oncogene activation, making them susceptible to tumorigenesis (Joerger et al., Oncogene (2007) 26, 2226-2242). The great majority of TP53 mutations are missense mutations, located within or proximal to its DNA-binding domain (Baugh et al., Cell Death & Differentiation (2018) 25, 154-160). Mutations leading to p53 loss of function can be categorized into two main types: (1) DNA contact mutations, where the mutant protein loses its ability to bind DNA; (2) structural mutations, which destabilize the p53 protein (Brosh et al., Nat. Rev. Cancer (2009) 9, 701-713; Hollstein et al., Science (1991) 253, 49-53). Both classes of mutations prevent p53-driven transcriptional activation, thus abrogating p53-mediated tumor suppression (Zhu et al., Frontiers in Oncology (2020) 10, Article 595187).

Reactivation of the mutant p53 protein emerges as an attractive approach to treat TP53 mutant cancers (Degtjarik et al., Nature Communications (2021) 12, Article 7057; Bykov et al., FEBS Letters (2014) 588, 2622-2627). Theoretically, mutant p53 reactivation will restore its tumor suppressive functions, stimulating p53-dependent arrest or apoptosis and resulting in efficient elimination of tumor cells (Selivanova et al., Oncogene (2007) 26, 2243-2254). The p53^{Y220C} mutation occurs in ~1% of human cancers; ~100,000 new cancer cases per year worldwide (Joerger et al., Annu. Rev. Biochem. (2016) 85, 375-404; Bouaoun et al., Hum. Mutat. (2016) 37, 865-876). Stabilization of the mutant protein may restore and/or maintain the functional conformation of the protein (Baud et al., Eur J Med Chem. (2018) 25, 101-114; Rauf et al., Protein J (2013) 32, 68-74). In some instances, such as the Y200C mutation, there is a small molecule binding pocket far away from the binding interface between p53 and DNA, such that small molecule engagement at this pocket will not disrupt DNA binding (Bauer et al., Future Med. Chem. (2019) 11, 2491-2504).

### SUMMARY

Some embodiments provide a compound of Formula (I):

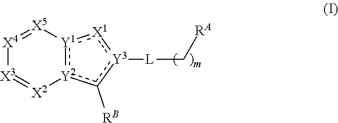

or a pharmaceutically acceptable salt thereof, wherein:
X¹ is CR¹ or N;
R¹ is hydrogen, halogen, cyano, —OR⁴, —NR⁴R⁵, —C(═O)R⁴, —OC(═O)R⁴, —C(═O)OR⁴, —C(═O)NR⁴R⁵, —SR⁴, —S(═O)R⁴, —S(═O)R⁴, —NR⁴C(═O)R⁵, —R⁵C(═O)R⁵, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
   each of X², X³, X⁴, and X⁵ are CH, N, CR² or CR³, wherein two or more of X², X³, X⁴, and X⁵ are independently CH, CR², or CR³;
each of Y¹, Y², and Y³ are C or N, wherein one of Y¹, Y², and Y³ is N;
   R^{A} is hydrogen, —OR⁶, —NR⁶R⁷, —C(═O)R⁶, —R⁶C(═O)R⁷, —OC(═O)R⁶, —OC(═O)NR⁶, —C(═O)OR⁶, —NR⁶C(═O)OR⁷, —C(═O)NR⁶R⁷, —SR⁶, —S(═O)R⁶, —S(O₂)R⁶, —S(O₂)NR⁶, —NR⁶S(O₂)R⁷, —NR⁶C(═O)R⁷, —NR⁶C(═O)NR⁷, —SiR⁶R⁷R′, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
R^{B} is halogen, cyano, hydroxyl, —NR⁸R⁹, —OR, —C(═O)NR⁸R⁹, —C(═O)R⁹, —C(═O)OR⁸, —NR⁸C(═O)OR⁹, —OC(═O)R⁸, —OC(═O)NR⁸, —C(═O)NR⁸R⁹, —NR⁸C(═O)R⁹, —NR⁸C(═O)NR⁹, —SR⁸, —S(═O)R⁸, —S(O₂)R⁸, —S(O₂)NR⁸, —NR⁸S(O₂)R⁹, —R⁸C(═O)R⁹, —NR⁸C(═O)R⁹, —NR⁸C(═O)NR⁹, optionally substituted C1-C6 alkyl, C1-C6 haloalkyl, optionally substituted C2-C6 alkenyl, optionally substituted C2-C6 alkynyl, optionally substituted C3-C10 cycloalkyl, optionally substituted phenyl, optionally substituted 3-12 membered heterocyclyl, or optionally substituted 5-10 membered heteroaryl;
each R² is

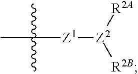

Z¹ is a bond, —C═O—, —S(O₂)-optionally substituted C1-C6 alkylene, optionally substituted C2-C6 alkenylene, optionally substituted C2-C6 alkynylene, or an optionally substituted C3-C4 cycloalkylene;
Z² is CR^{2C}, N, O, or a bond; wherein when Z² is O, R^{2B} is absent, and when Z¹ is a bond and Z² is a bond, R^{2B} is absent and R^{2A} is directly connected to Formula (I) via Z¹;
R^{2A} and R^{2B} are independently hydrogen, —C(═O)R¹⁰, —C(═O)OR¹⁰, —C(═O)NR¹⁰R¹¹, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl; or
R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl; or Z² is O and R^{2B} is absent
R^{2C} is hydrogen, halogen, or C1-C6 alkyl;
each R³ is independently halogen, cyano, —NR¹²R¹³, —OR¹², —C(═O)NR¹²R¹³, —C(═O)R², —C(═O)OR², —OC(═O)R², —NR²(C═O)NR¹²R¹³, —SR¹², —S(═O)R¹², —S(O₂)R¹², —S(O₂)NR¹², R¹³, —NR¹²S(O₂)NR¹³R¹⁴, —R¹²C(═O)R¹³, —NR¹²C(═O)R¹³, optionally substituted C1-C6 alkyl, optionally substituted C2-C6 alkenyl, optionally substituted C2-C6 alkynyl, optionally substituted C3-C6 cycloalkyl, optionally substituted phenyl, optionally substituted 4-6 membered heterocyclyl, or optionally substituted 5-6 membered heteroaryl;
L is an optionally substituted 4-6 membered heterocyclylene or an optionally substituted 5-6 membered heteroarylene;
m is 0, 1, or 2; and
each R⁴, R⁵, R⁶, R⁷, R⁸, R⁹, R¹⁰, R¹¹, R¹², R¹³, and R¹⁴ are independently hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

Also provided herein is a pharmaceutical composition comprising a compound of Formula (I), or a pharmaceutically acceptable salt thereof, and one or more pharmaceutically acceptable excipients.

Provided herein is a method for treating cancer in a subject in need thereof, the method comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, the method comprising (a) determining that the cancer is associated with a dysregulation of a TP53 gene, a p53 protein, or activity of any of the same; and (b) administering to the subject a therapeutically effective amount of a compound of Formula (I), or a pharmaceutically acceptable salt thereof, or a pharmaceutical composition as provided herein.

Provided herein is a method of treating a p53-associated cancer in a subject, the method comprising administering to a subject identified or diagnosed as having a p53-associated cancer a therapeutically effective amount of a compound of Formula (I), or a pharmaceutically acceptable salt thereof, or a pharmaceutical composition as provided herein.

This disclosure also provides a method of treating a p53-associated cancer in a subject, the method comprising: determining that the cancer in the subject is a p53-associated cancer; and administering to the subject a therapeutically effective amount of a compound of Formula (I), or a pharmaceutically acceptable salt thereof, or a pharmaceutical composition as provided herein.

Further provided herein is a method of treating a p53-associated cancer in a subject, the method comprising administering to a subject identified or diagnosed as having a p53-associated cancer a therapeutically effective amount of a compound of Formula (I), or a pharmaceutically acceptable salt thereof, or a pharmaceutical composition as provided herein.

This disclosure also provides a method of treating a p53-associated cancer in a subject, the method comprising: determining that the cancer in the subject is a p53-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of a 7P53 gene, a p53 protein, or activity of any of the same.

This disclosure also provides a method for restoring p53 function in a mammalian cell, the method comprising contacting the mammalian cell with an effective amount of a compound of Formula (I), or a pharmaceutically acceptable salt thereof.

Other embodiments include those described in the Detailed Description and/or in the claims.

### ADDITIONAL DEFINITIONS

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to +10% of the stated number or numerical range.

The term “acceptable” with respect to a formulation, composition or ingredient, as used herein, means having no persistent detrimental effect on the general health of the subject being treated.

The phrase “therapeutically effective amount” means an amount of compound that, when administered to a subject in need of such treatment, is sufficient to (i) treat a p53 protein-associated cancer, (ii) attenuate, ameliorate, or eliminate one or more symptoms of the particular cancer or (iii) delay the onset of one or more symptoms of the particular cancer, described herein.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

As used herein, the “subject” refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the cancer to be treated.

As used herein, terms “treat” or “treatment” refer to therapeutic or palliative measures. Beneficial or desired clinical results include, but are not limited to, alleviation, in whole or in part, of symptoms associated with a cancer, diminishment of the extent of the cancer, stabilized (i.e., not worsening) state of disease, delay or slowing of cancer progression, amelioration or palliation of the disease state (e.g., one or more symptoms of the cancer), and remission (whether partial or total), whether detectable or undetectable. “Treatment” can also mean prolonging survival as compared to expected survival if not receiving treatment.

Whenever a group is described as being “optionally substituted” that group may be unsubstituted or substituted with one or more of the indicated substituents. Likewise, when a group is described as being “substituted” the substituent(s) may be selected from one or more the indicated substituents. If no substituents are indicated, it is meant that the indicated “optionally substituted” or “substituted” group may be substituted with one or more individually and independently selected group(s) that are stable and chemically acceptable for the group being substituted. Non-limiting examples of optional substituents are halogen, cyano, hydroxyl, nitro, nitroso, azido, sulfhydryl, acyl, alkyl, hydroxyalkyl, aminoalkyl, alkoxyamino, haloalkyl, alkenyl, haloalkenyl, alkynyl, haloalkynyl, alkoxy, hydroxyalkoxy, alkoxyalkoxy, alkenoxy, alkynoxy, haloalkoxy, haloalkenoxy, haloalkynoxy, cycloalkyl, halocycloalkyl, cycloalkoxy, aryl, aryloxy, heteroaryl, heteroaryloxy, heterocyclyl, heterocyclyloxy, aralkyl, cycloalkylalkyl, heteroaralkyl, alkoxyalkyl, heterocyclylalkyl, thiocarbonyl, O-carbamyl, N-carbamyl, O-thiocarbamyl, N-thiocarbamyl, alkoxycarbonyl, C-amido, N-amido, S-sulfonamido, N-sulfonamido, sulfenyl, halosulfenyl, sulfonyl, sulfinyl, sulfoximino, sulfonimidamido, phosphine oxide, C-carboxy, O-carboxy, arylalkoxy, cycloalkylalkoxy, carboxaldehyde, iminyl, trihalomethanesulfonyl, trihalomethanesulfonamido, phosphityl, phosphonityl, phosphorothioityl, phophoamidityl, phosphonamidityl, phosphinityl, phosphinyl, phosphonothioityl, phosphorodiamidityl, phosphinamidityl, phosphorodithioityl, phosphonodiamidityl, phosphorotriamidityl, phosphatyl, phosphinatyl, phosphonatyl, phosphoroamidatyl, phosphorodiamidatyl, phosphonodiamidatyl, phosphonamidatyl, phosphinamidatyl, phosphorotriamidatyl, phosphorothiatyl, dithiophosphinatyl, phosphorodithioatyl, phosphonothioatyl, thiophosphatyl, thiophosphinatyl, phosphorodithiatyl, thiophosphonatyl, phosphorofluoridatyl, bisphosphonatyl, triphosphatyl, pyrophosphatyl, tetraphosphatyl, and ureido.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “sulfhydryl” refers to a —SH radical.

The term “cyano” refers to a —CN radical.

The term “azido” refers to a —N₃ radical.

The term “nitro” refers to a —NO₂ radical.

The term “nitroso” refers to a —N═O radical.

The term “alkyl” refers to a saturated acyclic hydrocarbon radical that may be a straight chain or branched chain, containing the indicated number of carbon atoms. For example, C1-C10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “acyl” refers to a —C(═O)alkyl radical (e.g., acetyl), or a —C(═O)alkenyl radical (e.g., —C(═O)—CH═CH₂), or —C(═O)alkynyl radical

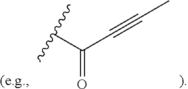 [SMILES: *C(=O)C#CC]

Acyl groups can be substituted with cyano or with 1-3 independently selected halogens.

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated or partially unsaturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, S, P, B, and Si and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of

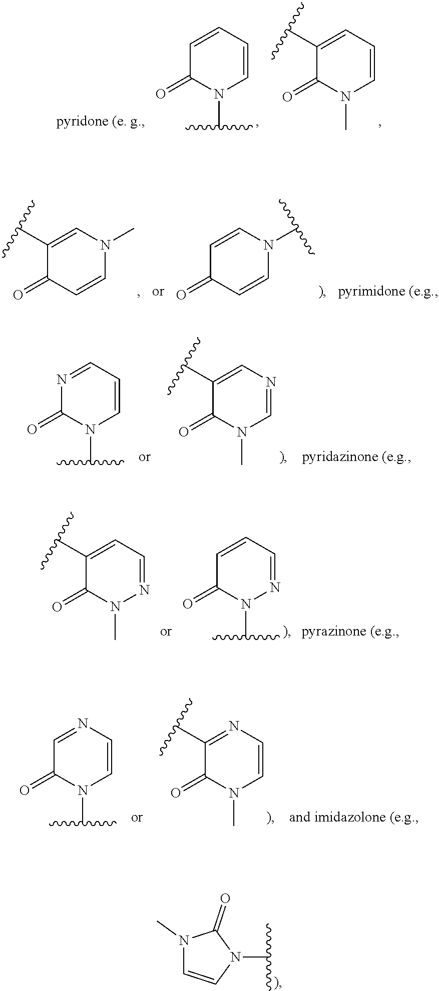

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, P, S, B, or Si (e.g., carbon atoms and 1-3, 1-6, or 1-9 heteroatoms of N, O, P, S, B, or Si if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by 1-2 substituents. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, oxaphosphinanyl oxide, azaphosphinanyl oxide, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom).

Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

The term “haloalkyl” refers to an alkyl, in which one or more hydrogen atoms is/are replaced with an independently selected halogen.

The term “halocycloalkyl” refers to a cycloalkyl, in which one or more hydrogen atoms is/are replaced with an independently selected halogen.

The term “hydroxyalkyl” refers to an alkyl, in which one or more hydrogen atoms is/are replaced with hydroxyl.

The term “haloalkenyl” refers to an alkenyl, in which one or more hydrogen atoms is/are replaced with an independently selected halogen.

The term “haloalkynyl” refers to an alkynyl, in which one or more hydrogen atoms is/are replaced with an independently selected halogen.

The term “alkoxy” refers to an —O-alkyl radical (e.g., —OCH₃).

The term “alkoxyalkyl” refers to an alkyl, in which one or two hydrogen atoms is/are replaced with an independently selected alkoxy (e.g., methoxyethyl).

The term “hydroxyalkoxy” refers to an alkoxy group, in which one or two hydrogen atoms is/are replaced with hydroxy.

The term “alkoxyalkoxy” refers to an alkoxy group, in which one or two hydrogen atoms is/are replaced with an independently selected alkoxy.

The term “alkoxyamino” refers to an —O-amino radical (e.g., —OCH₂CH₂N(CH₃)₂).

The term “haloalkoxy” refers to an —O-haloalkyl radical (e.g., —OCF₃).

The term “alkenoxy” refers to an —O-alkenyl radical (e.g., —O-allyl).

The term “haloalkenoxy” refers to an —O-haloalkenyl radical.

The term “alkynoxy” refers to an —O-alkynyl radical (e.g., —O-propargyl).

The term “haloalkynoxy” refers to an —O-haloalkynyl radical.

The term “cycloalkoxy” refers to an —O-cycloalkyl radical (e.g., —O-cyclopropyl).

The term “aryloxy” refers to an —O-aryl radical (e.g., phenoxy).

The term “heteroaryloxy” refers to an —O-heteroaryl radical (e.g., pyridinoxy).

The term “heterocyclyloxy” refers to an —O-heterocyclyl radical (e.g., —O-pyrrolidinyl or —O-oxetanyl).

The term “aralkyl” refer to an aryl group connected, as a substituent, via an alkyl group (e.g., benzyl).

The term “cycloalkylalkyl” refers to a cycloalkyl group connected, as a substituent, via an alkyl group (e.g., ethylcyclobutyl).

The term “heteroaralkyl” refers to a heteroaryl group connected, as a substituent, via an alkyl group (e.g., methylpyrimidinyl).

The term “heterocyclylalkyl” refers to a heterocyclyl group connected, as a substituent, via an alkyl group (e.g., methyloxetanyl).

The term “aralkoxy” refers to an aryl group connected, as a substituent, via an alkoxy group (e.g., benzyloxy).

The term “cycloalkylalkoxy” refers to a cycloalkyl connected, as a substituent, via an alkoxy group (e.g., methoxycyclopropyl).

The term “aminoalkyl” refers to an amino group connected, as a substituent, via an alkyl group (e.g., methyl(dimethylamino)).

A “sulfenyl” group refers to an “—SR” group in which R can be hydrogen, alkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “halosulfenyl” group refers to a sulfenyl, in which one or more hydrogen atoms is/are replaced with an independently selected halogen (e.g., —S(CF₃) or —S(CHF₂)).

A “sulfinyl” group refers to an “—S(═O)” group in which R can be the same as defined with respect to sulfenyl.

A “sulfonyl” group refers to an “SO₂R” group in which R can be the same as defined with respect to sulfenyl.

A “sulfoximine” group refers to an —S(═O)(═NR)R′, where R is hydrogen, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl; and where R′ alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “sulfonimidamido” group refers to an —S(═O)(═NR)NR′R″ where R, R′, and R″ are independently hydrogen, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl; and where R′ alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-carboxy” group refers to a “RC(═O)O—” group in which R can be hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The terms “ester” and “C-carboxy” refer to a “—C(═O)OR” group in which R can be the same as defined with respect to O-carboxy.

A “thiocarbonyl” group refers to a “—C(═S)R” group in which R can be the same as defined with respect to O-carboxy.

A “trihalomethanesulfonyl” group refers to an “X₃CSO₂—” group wherein each X is a halogen.

A “trihalomethanesulfonamido” group refers to an “X₃CS(O)₂N(R′)—” group wherein each X is a halogen, and R′ is hydrogen, alkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “S-sulfonamido” group refers to a “—SO₂N(RR′)” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-sulfonamido” group refers to a “RSO₂N(R′)—” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-carbamyl” group refers to a “—OC(═O)N(RR′)” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-carbamyl” group refers to an “ROC(═O)N(R′)—” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “O-thiocarbamyl” group refers to a “—OC(═S)N(RR′)” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-thiocarbamyl” group refers to an “ROC(═S)N(R′)—” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

A “C-amido” group refers to a “—C(═O)N(RR′)” group in which R and R′ are independently hydrogen, alkyl, alkoxy, alkoxyalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

An “N-amido” group refers to a “RC(═O)N(R′)” group in which R and R′ are independently hydrogen, alkyl, alkoxy, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The terms “ureido” or “urea” refer to an —NR(C═O)NR′R″″ group, in which R, R′, and R″ are independently hydrogen, hydroxyl, alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The term “carboxaldehyde” refers to a —C(═O)H radical.

The term “imine” or “imino” refers to a —N═R radical, in which R is hydrogen, hydroxyl, alkyl, haloalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

The term “amino” refers to a —NRR′ radical, where R and R′ are independently hydrogen, alkyl, haloalkyl, hydroxyalkyl, alkenyl, alkynyl, cycloalkyl, cycloalkenyl, aryl, heteroaryl, heterocyclyl, aralkyl heteroaralkyl, heterocyclylalkyl, or cycloalkylalkyl. In some instances, an amino group is —NH₂, a mono-alkyl amine (R is hydrogen and R′ is alkyl) or a dialkylamine (R and R′ are independently selected alkyl).

The term “phosphine oxide” refers to a —P(═O)RR′ radical, where R and R′ are independently alkyl, haloalkyl, hydroxyalkyl, aminoalkyl, alkenyl, alkynyl, cycloalkyl, cycloalkenyl, aryl, heteroaryl, heterocyclyl, aralkyl heteroaralkyl, heterocyclylalkyl, or cycloalkylalkyl.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

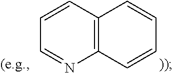

(ii) a single ring atom (spiro-fused ring systems)

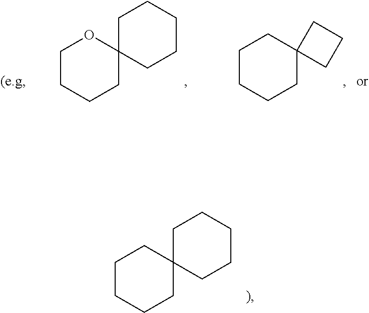 [SMILES: C1CCC2(CC1)CCCCO2 ; C1CCC2(CC1)CCC2 ; C1CCC2(CC1)CCCCC2]

or (iii) a contiguous array of ring atoms (bridged ring systems having all bridge lengths >0)

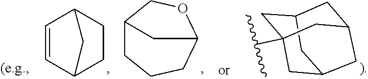

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

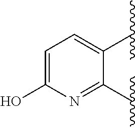 [SMILES: *c1ccc(O)nc1*]

encompasses the tautomeric form containing the moiety:

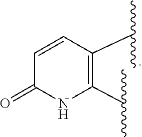 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### DETAILED DESCRIPTION

This disclosure provides compounds of Formula (I), and pharmaceutically acceptable salts thereof, that restore p53 function. These compounds are useful, e.g., for treating a disease in which decreased p53 function contributes to the pathology and/or symptoms and/or progression of the disease (e.g., cancer) in a subject (e.g., a human).

### Formulae (I) Compounds

Some embodiments provide a compound of Formula (I):

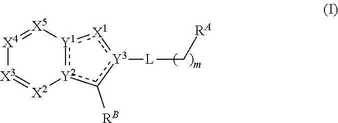

or a pharmaceutically acceptable salt thereof, wherein:
X¹ is CR¹ or N;
R¹ is hydrogen, halogen, cyano, —OR⁴, —NR⁴R⁵, —C(═O)R⁴, —OC(═O)R⁴, —C(═O)OR⁴, C(═O)NR⁴R⁵, SR⁴, S(═O)R⁴, S(O₂)R⁴, —NR⁴C(═O)R⁵, R⁴C(—O)R⁵, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
each of X², X³, X⁴, and X⁵ are CH, N, CR² or CR³, wherein two or more of X², X³, X⁴, and X⁵ are independently CH, CR², or CR³;
each of Y¹, Y², and Y³ are C or N, wherein one of Y¹, Y², and Y³ is N;
R^{A} is hydrogen, —OR⁶, —NR⁶R⁷, —C(═O)R⁶, —R⁶C(═O)R⁷, —OC(═O)R⁶, —OC(═O)NR⁶, —C(═O)OR⁶, —NR⁶C(═O)OR⁷, —C(═O)NR⁶R⁷, —SR⁶, —S(═O)R⁶, —S(O₂)R⁶, —S(O₂)NR⁶, —NR⁶S(O₂)R⁷, —NR⁶C(═O)R⁷, —NR⁶C(═O)NR⁷, —SiR⁶R⁷R′, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
R^{B} is halogen, cyano, hydroxyl, —NR⁸R⁹, —OR⁸, —C(═O)NR⁸R⁹, —C(═O)R⁸, —C(═O)OR⁸, —NR⁸C(═O)OR⁹, —OC(═O)R⁸, —OC(═O)NR⁸, —C(═O)NR⁸R⁹, —NR⁸C(═O)R⁹, —NR⁸C(═O)NR⁹, —SR⁸, —S(═O)R⁸, —S(O₂)R⁸, —S(O₂)NR⁸, —NR⁸S(O₂)R⁹, —R⁸C(═O)R⁹, —NR⁸C(═O)R⁹, —NR⁸C(═O)NR⁹, optionally substituted C1-C6 alkyl, C1-C6 haloalkyl, optionally substituted C2-C6 alkenyl, optionally substituted C2-C6 alkynyl, optionally substituted C3-C10 cycloalkyl, optionally substituted phenyl, optionally substituted 3-12 membered heterocyclyl, or optionally substituted 5-10 membered heteroaryl;
each R² is

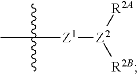

Z¹ is a bond, —C═O—, —S(O₂)-optionally substituted C1-C6 alkylene, optionally substituted C2-C6 alkenylene, optionally substituted C2-C6 alkynylene, or an optionally substituted C3-C4 cycloalkylene;
Z² is CR^{2C}, N, O, or a bond; wherein when Z² is 0, R^{2B} is absent, and when Z¹ is a bond and Z² is a bond, R^{2B} is absent and R^{2A} is directly connected to Formula (I) via Z¹;
R^{2A} and R^{2B} are independently hydrogen, —C(═O)R¹⁰, —C(═O)OR¹⁰, —C(═O)NR¹⁰R¹¹, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl; or
R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl; or Z² is 0 and R^{2B} is absent;
R^{2C} is hydrogen, halogen, or C1-C6 alkyl;
each R³ is independently halogen, cyano, —NR¹²R¹³, —OR¹², —C(═O)NR¹²R¹³, —C(═O)R¹², —C(═O)OR¹², —OC(═O)R¹², —NR¹²(C═O)NR¹³R¹⁴, —SR¹², —S(═O)R¹², —S(O₂)R¹², —S(O₂)NR¹²R¹³, —NR¹²S(O₂)NR¹³R¹⁴, —R²C(═O)R³, —NR¹²C(═O)R¹³, optionally substituted C1-C6 alkyl, optionally substituted C2-C6 alkenyl, optionally substituted C2-C6 alkynyl, optionally substituted C3-C6 cycloalkyl, optionally substituted phenyl, optionally substituted 4-6 membered heterocyclyl, or optionally substituted 5-6 membered heteroaryl;
L is an optionally substituted 4-6 membered heterocyclylene or an optionally substituted 5-6 membered heteroarylene;
m is 0, 1, or 2; and
each R⁴, R⁵, R⁶, R⁷, R⁸, R⁹, R¹⁰, R¹¹, R¹², R¹³, and R¹⁴ are independently hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, the compound of Formula (I), or a pharmaceutically acceptable salt thereof, is selected from the group consisting of:

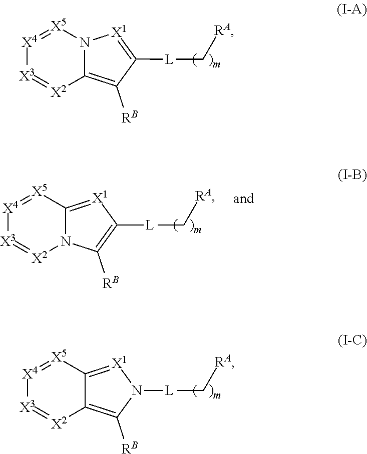

or a pharmaceutically acceptable salt of any of the foregoing.

In some embodiments, the compound of Formula (I), or a pharmaceutically acceptable salt thereof, is selected from the group consisting of:

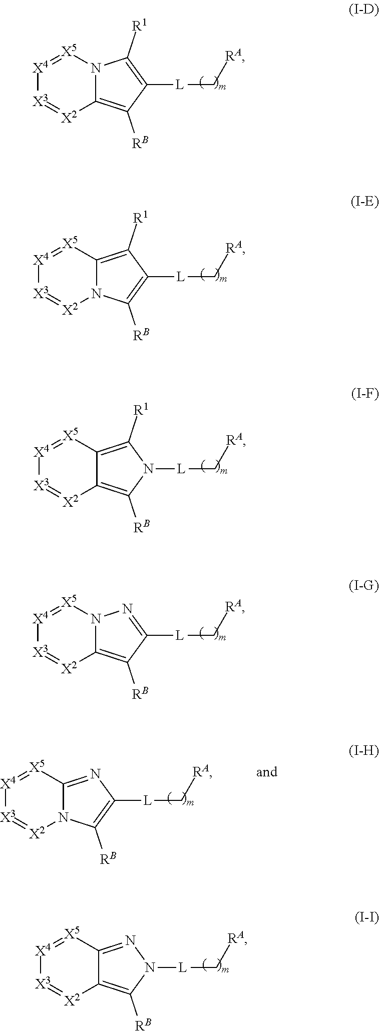

or a pharmaceutically acceptable salt of any of the foregoing.

In some embodiments, one of X², X³, X⁴, and X⁵ is N.

In some embodiments, two of X², X³, X⁴, and X⁵ are N.

In some embodiments, X¹ is CR¹.

In some embodiments, R¹ is hydrogen.

In some embodiments, R¹ is halogen.

In some embodiments, R¹ is cyano.

In some embodiments, R¹ is —OR⁴.

In some embodiments, R¹ is —NR⁴R⁵.

In some embodiments, R¹ is —C(═O)R⁴.

In some embodiments, R¹ is —OC(═O)R⁴.

In some embodiments, R¹ is —C(═O)OR⁴.

In some embodiments, R¹ is —C(═O)NR⁴R⁵.

In some embodiments, R¹ is —SR⁴.

In some embodiments, R¹ is S(═O)R⁴.

In some embodiments, R¹ is —S(O₂)R⁴.

In some embodiments, R¹ is —NR⁴C(═O)R⁵.

In some embodiments, R¹ is —R⁴C(═O)R⁵.

In some embodiments, R⁴ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁴ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁴ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁴ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁴ is hydrogen, C1-C6 alkyl, C3-C10 cycloalkyl, or 4-12 membered heterocyclyl. In some embodiments, R⁴ is hydrogen, C1-C3 alkyl, C3-C6 cycloalkyl, or 4-8 membered heterocyclyl.

In some embodiments, R⁵ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁵ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁵ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁵ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁵ is hydrogen, C1-C6 alkyl, C3-C10 cycloalkyl, or 4-12 membered heterocyclyl. In some embodiments, R⁵ is hydrogen, C1-C3 alkyl, C3-C6 cycloalkyl, or 4-8 membered heterocyclyl.

In some embodiments, when R⁴ and R⁵ are attached to the same nitrogen atom, R⁴ and R⁵ are the same. In some embodiments, when R⁴ and R⁵ are attached to the same nitrogen atom, R⁴ and R⁵ are different. In some embodiments, when R⁴ and R⁵ are attached to the same nitrogen atom, R⁴ and R⁵ are each hydrogen. In some embodiments, when R⁴ and R⁵ are attached to the same nitrogen atom, R⁴ and R⁵ are each an independently selected C1-C6 alkyl.

In some embodiments, when R⁴ and R⁵ are attached to the same nitrogen atom, one of R⁴ and R⁵ is hydrogen and the other of R⁴ and R⁵ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, when R⁴ and R⁵ are attached to the same nitrogen atom, one of R⁴ and R⁵ is hydrogen and the other of R⁴ and R⁵ is C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹ is optionally substituted C1-C6 alkyl. In some embodiments, R¹ is C1-C6 alkyl. In some embodiments, R¹ is methyl or ethyl.

In some embodiments, R¹ is optionally substituted C2-C6 alkenyl. In some embodiments, R¹ is C2-C6 alkenyl. In some embodiments, R¹ is optionally substituted C2-C3 alkenyl. In some embodiments, R¹ is C2-C3 alkenyl.

In some embodiments, R¹ is optionally substituted C2-C6 alkynyl. In some embodiments, R¹ is C2-C6 alkynyl. In some embodiments, R¹ is optionally substituted C2-C3 alkynyl. In some embodiments, R¹ is C2-C3 alkynyl.

In some embodiments, R¹ is optionally substituted C3-C6 cycloalkyl. In some embodiments, R¹ is C3-C6 cycloalkyl.

In some embodiments, R¹ is optionally substituted phenyl. In some embodiments, R¹ is phenyl.

In some embodiments, R¹ is optionally substituted 4-6 membered heterocyclyl. In some embodiments, R¹ is 4-6 membered heterocyclyl.

In some embodiments, R¹ is optionally substituted 5-6 membered heteroaryl. In some embodiments, R¹ is 5-6 membered heteroaryl.

In some embodiments, X¹ is N.

In some embodiments, the compound of Formula (I), or a pharmaceutically acceptable salt thereof, is selected from the group consisting of:

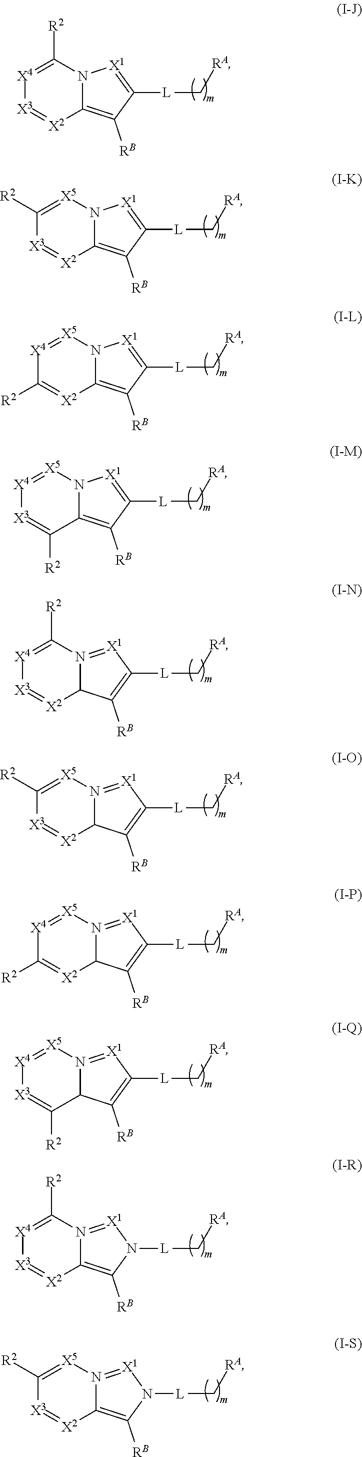

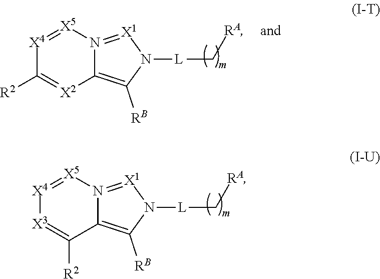

or a pharmaceutically acceptable salt of any of the foregoing.

In some embodiments, the compound of Formula (I), or a pharmaceutically acceptable salt thereof, is selected from the group consisting of:

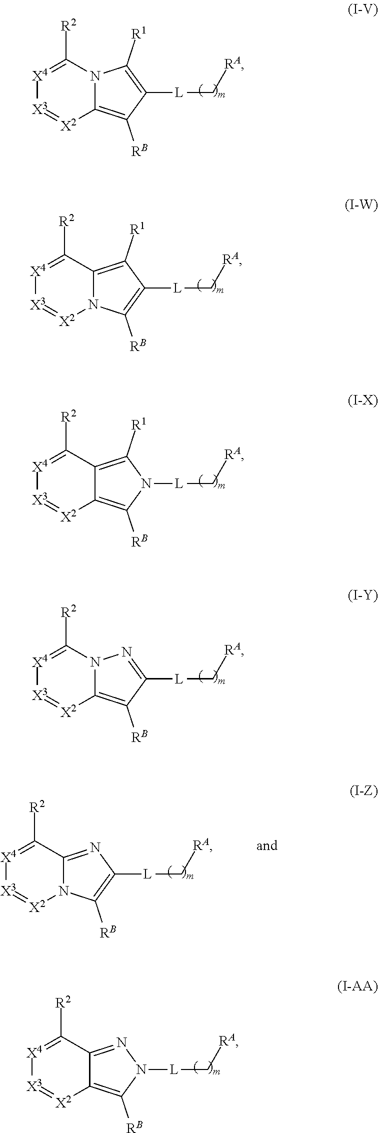

or a pharmaceutically acceptable salt of any of the foregoing.

In some embodiments, the compound of Formula (I), or a pharmaceutically acceptable salt thereof, is selected from the group consisting of:

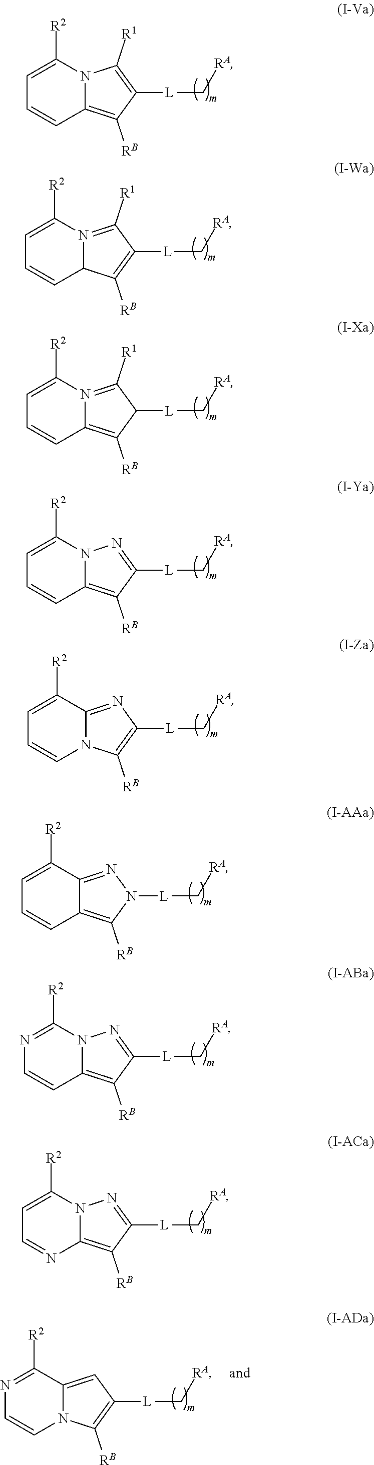

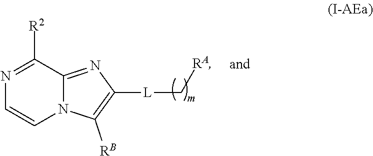

or a pharmaceutically acceptable salt of any of the foregoing.

In some embodiments, the compound of Formula (I), or a pharmaceutically acceptable salt thereof, is selected from the group consisting of:

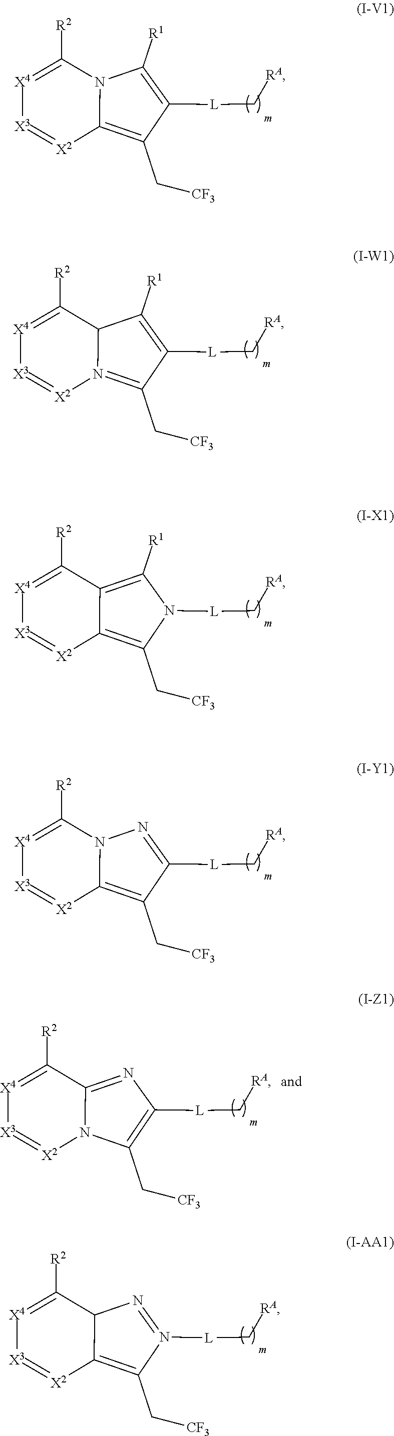

or a pharmaceutically acceptable salt of any of the foregoing.

In some embodiments, the compound of Formula (I), or a pharmaceutically acceptable salt thereof, is selected from the group consisting of:

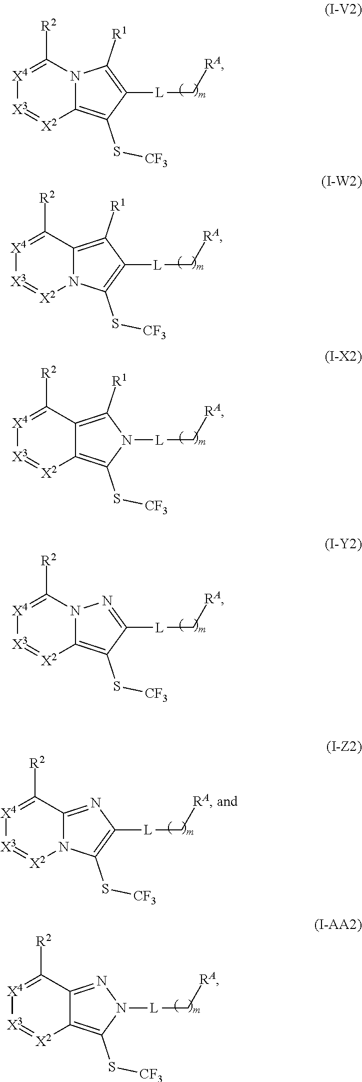

or a pharmaceutically acceptable salt of any of the foregoing.

In some embodiments, the compound of Formula (I), or a pharmaceutically acceptable salt thereof, is selected from the group consisting of:

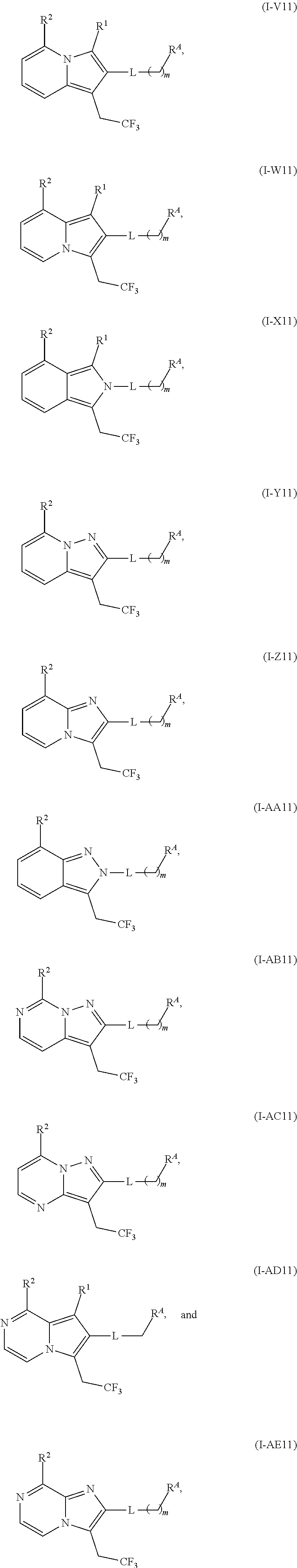

or a pharmaceutically acceptable salt of any of the foregoing.

In some embodiments, the compound of Formula (I), or a pharmaceutically acceptable salt thereof, is selected from the group consisting of:

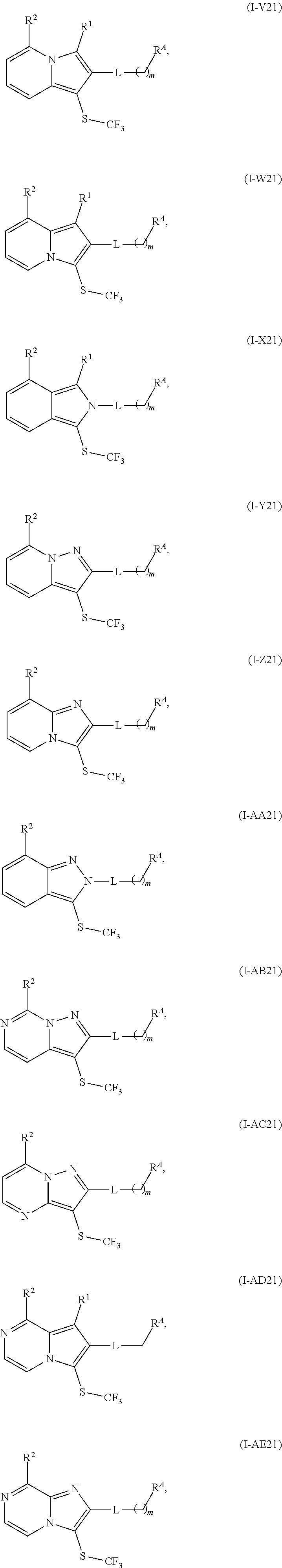

or a pharmaceutically acceptable salt of any of the foregoing. In some embodiments, the compound of Formula (I), or a pharmaceutically acceptable salt thereof, is

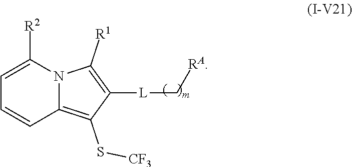

In some embodiments, the compound of Formula (I), or a pharmaceutically acceptable salt thereof, is

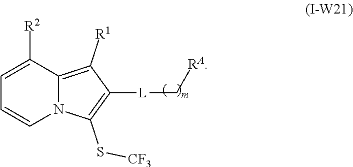

In some embodiments, the compound of Formula (I), or a pharmaceutically acceptable salt thereof, is

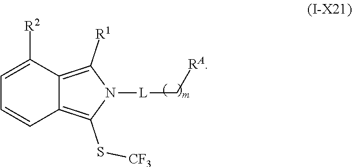

In some embodiments, the compound of Formula (I), or a pharmaceutically acceptable salt thereof, is

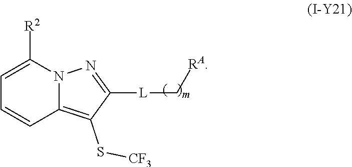

In some embodiments, the compound of Formula (I), or a pharmaceutically acceptable salt thereof, is

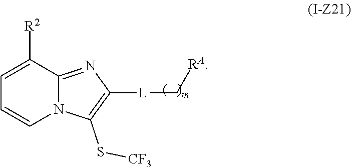

In some embodiments, the compound of Formula (I), or a pharmaceutically acceptable salt thereof, is

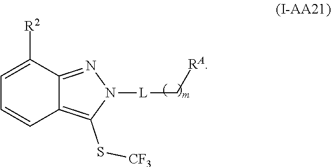

In some embodiments, the compound of Formula (I), or a pharmaceutically acceptable salt thereof, is

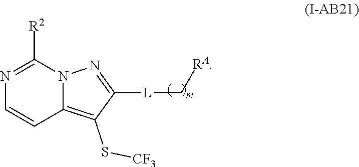

In some embodiments, the compound of Formula (I), or a pharmaceutically acceptable salt thereof, is

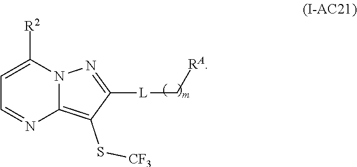

In some embodiments, the compound of Formula (I), or a pharmaceutically acceptable salt thereof, is

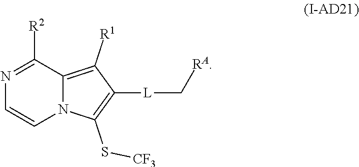

In some embodiments, the compound of Formula (I), or a pharmaceutically acceptable salt thereof, is

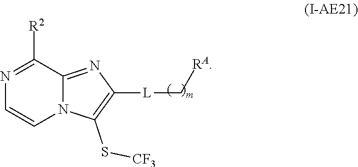

In some embodiments, the compound of Formula (I), or a pharmaceutically acceptable salt thereof, is selected from the group consisting of:

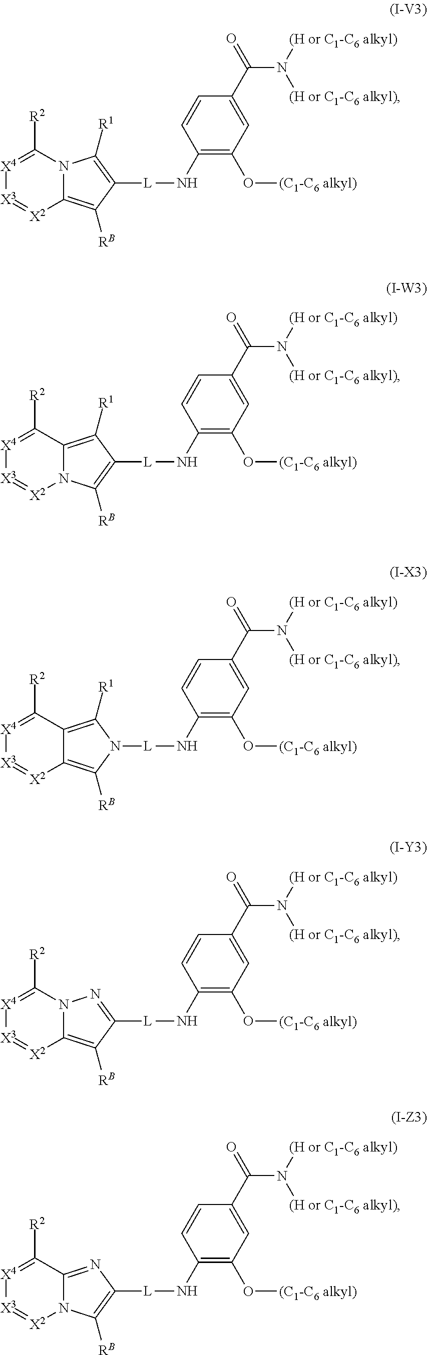

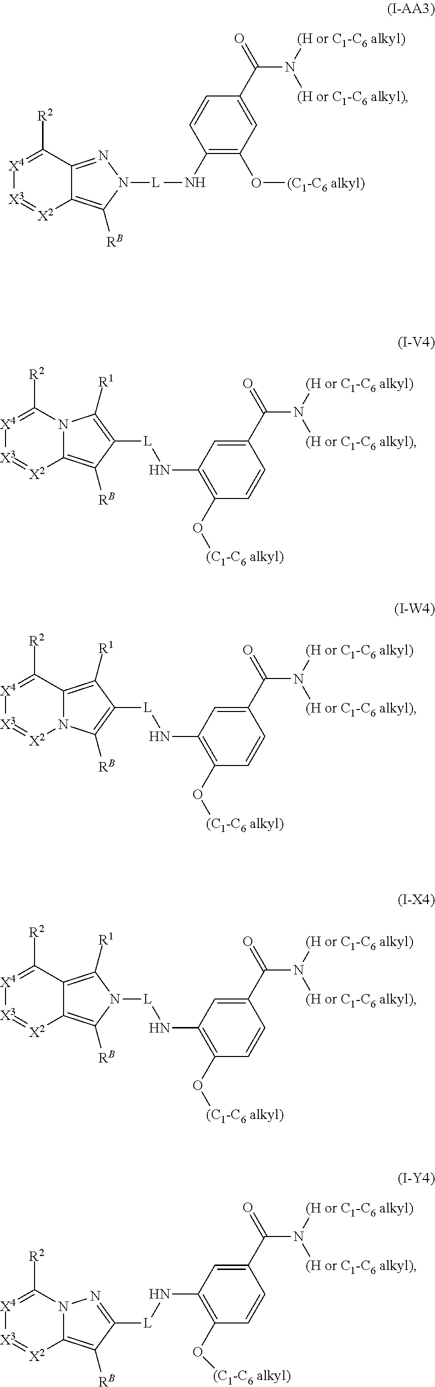

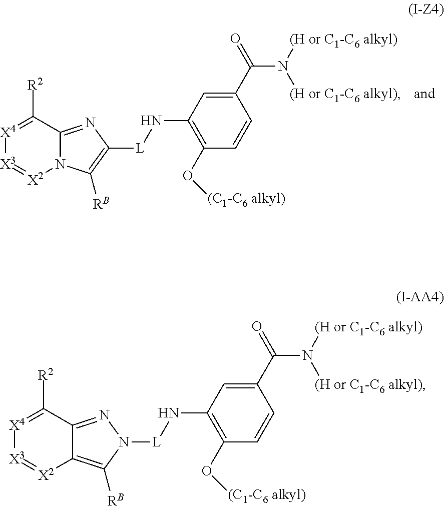

or a pharmaceutically acceptable salt of any of the foregoing.

In some embodiments, the compound of Formula (I), or a pharmaceutically acceptable salt thereof, is selected from the group consisting of

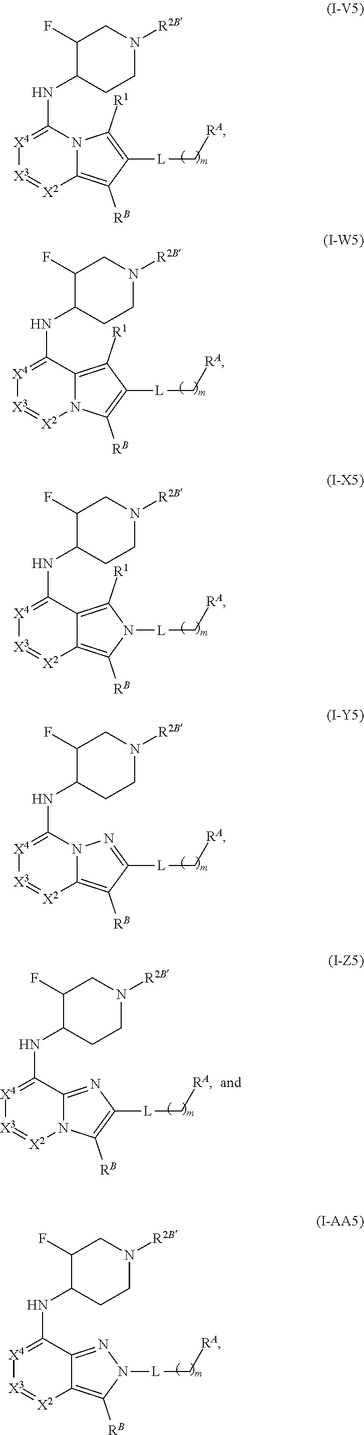

or a pharmaceutically acceptable salt of any of the foregoing, wherein R^{2B′} is hydrogen or an optionally substituted C1-C6 alkyl.

In some embodiments, the compound of Formula (I), or a pharmaceutically acceptable salt thereof, is selected from the group consisting of:

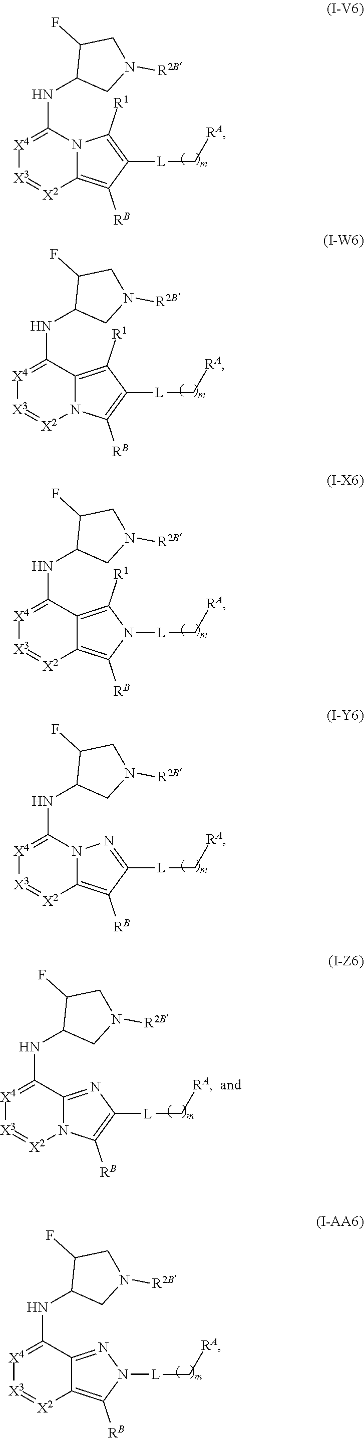

or a pharmaceutically acceptable salt of any of the foregoing, wherein R^{2B′} is hydrogen or an optionally substituted C1-C6 alkyl.

In some embodiments, the compound of Formula (I), or a pharmaceutically acceptable salt thereof, is selected from the group consisting of:

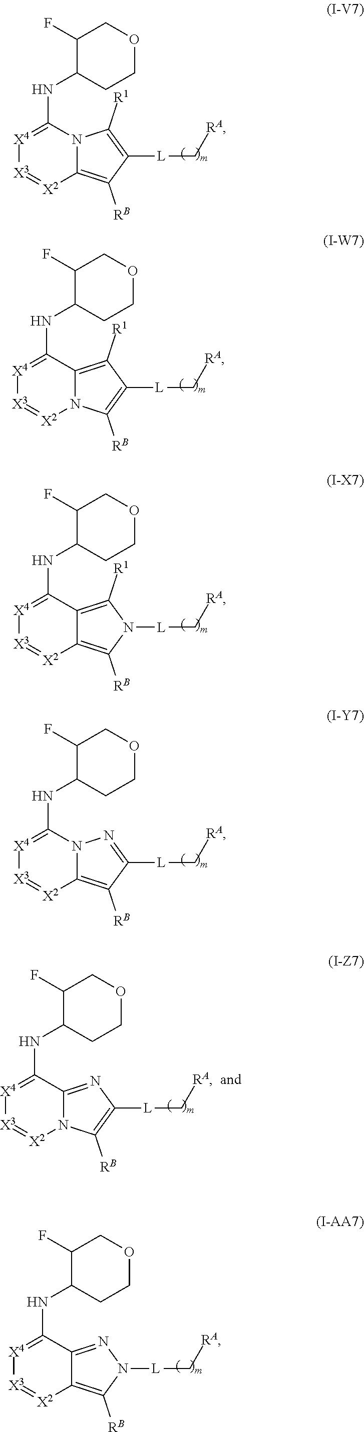

or a pharmaceutically acceptable salt of any of the foregoing.

In some embodiments, the compound of Formula (I), or a pharmaceutically acceptable salt thereof, is selected from the group consisting of:

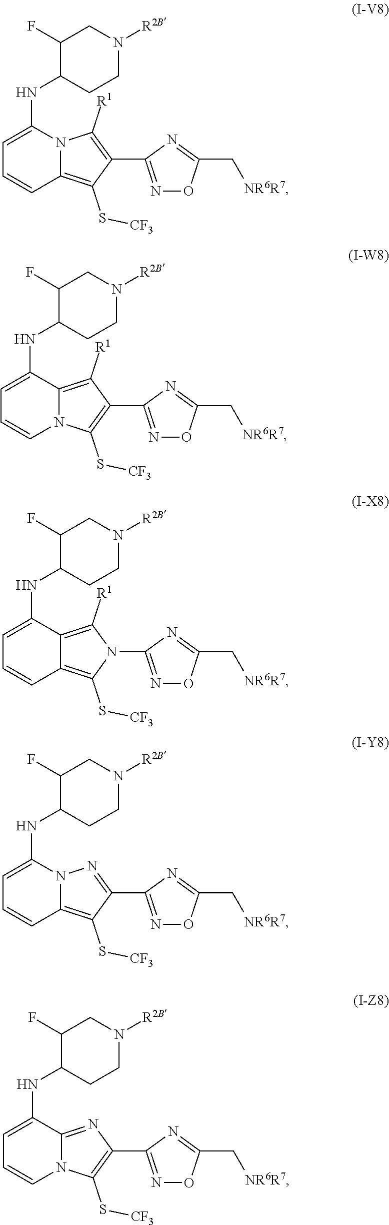

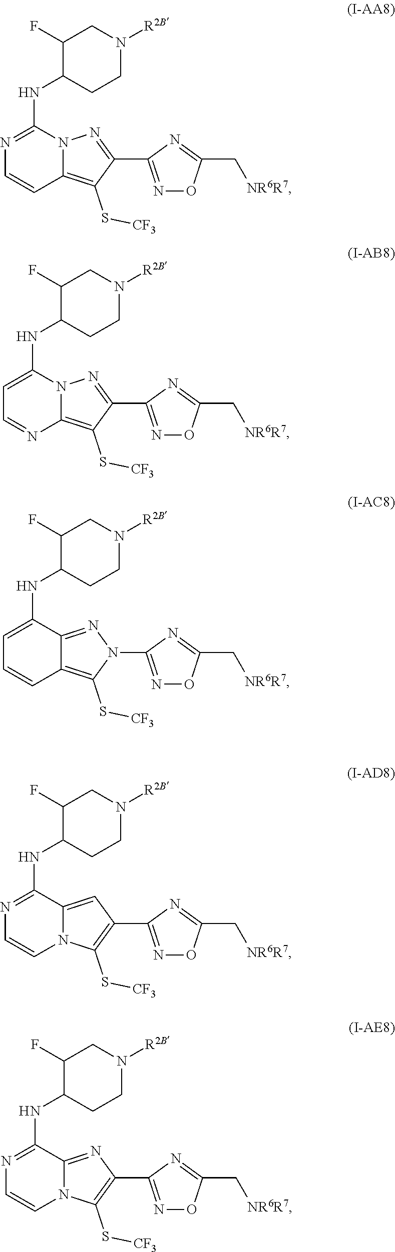

or a pharmaceutically acceptable salt of any of the foregoing, wherein R^{2B′} is hydrogen or an optionally substituted C1-C6 alkyl.

In some embodiments, the compound of Formula (I), or a pharmaceutically acceptable salt thereof, is selected from the group consisting of:

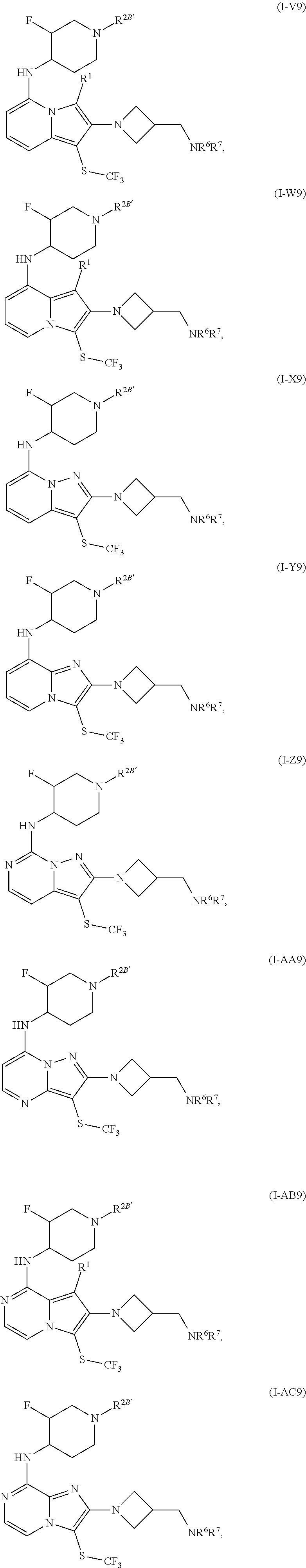

or a pharmaceutically acceptable salt of any of the foregoing, wherein R²³ is hydrogen or an optionally substituted C1-C6 alkyl.

In some embodiments, R^{A} is hydrogen.

In some embodiments, R^{A} is —OR⁶.

In some embodiments, R^{A} is NR⁶R⁷.

In some embodiments, R^{A} is —C(═O)R⁶.

In some embodiments, R^{A} is —R⁶C(═O)R⁷.

In some embodiments, R^{A} is —OC(═O)R⁶.

In some embodiments, R^{A} is —OC(═O)NR⁶.

In some embodiments, R^{A} is —C(═O)OR⁶.

In some embodiments, R^{A} is —NR⁶C(═O)OR⁷.

In some embodiments, R^{A} is —C(═O)NR⁶R⁷.

In some embodiments, R^{A} is —SR⁶.

In some embodiments, R^{A} is —S(═O)R⁶.

In some embodiments, R^{A} is —S(O₂)R⁶.

In some embodiments, R^{A} is —S(O₂)NR⁶.

In some embodiments, R^{A} is —NR⁶S(O₂)R⁷.

In some embodiments, R^{A} is —NR⁶C(═O)R⁷.

In some embodiments, R^{A} is —NR⁶C(═O)NR⁷.

In some embodiments, R^{A} is —SiR⁶R⁷R′.

In some embodiments, R⁶ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁶ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁶ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁶ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁶ is hydrogen, C1-C6 alkyl, C3-C10 cycloalkyl, or 4-12 membered heterocyclyl. In some embodiments, R⁶ is hydrogen, C1-C3 alkyl, C3-C6 cycloalkyl, or 4-8 membered heterocyclyl.

In some embodiments, R⁷ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁷ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁷ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁷ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁷ is hydrogen, C1-C6 alkyl, C3-C10 cycloalkyl, or 4-12 membered heterocyclyl. In some embodiments, R⁷ is hydrogen, C1-C3 alkyl, C3-C6 cycloalkyl, or 4-8 membered heterocyclyl.

In some embodiments, when R⁶ and R⁷ are attached to the same nitrogen atom, R⁶ and R⁷ are the same. In some embodiments, when R⁶ and R⁷ are attached to the same nitrogen atom, R⁶ and R⁷ are different. In some embodiments, when R⁶ and R⁷ are attached to the same nitrogen atom, R⁶ and R⁷ are each hydrogen. In some embodiments, when R⁶ and R⁷ are attached to the same nitrogen atom, R⁶ and R⁷ are each an independently selected C1-C6 alkyl.

In some embodiments, when R⁶ and R⁷ are attached to the same nitrogen atom, one of R⁶ and R⁷ is hydrogen and the other of R⁶ and R⁷ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, when R⁶ and R⁷ are attached to the same nitrogen atom, one of R⁶ and R⁷ is hydrogen and the other of R⁶ and R⁷ is optionally substituted phenyl or optionally substituted 5-10 membered heteroaryl. In some embodiments, when R⁶ and R⁷ are attached to the same nitrogen atom, one of R⁶ and R⁷ is hydrogen and the other of R⁶ and R⁷ is substituted phenyl or optionally 5-10 membered heteroaryl.

In some embodiments, when R⁶ and R⁷ are attached to the same nitrogen atom, one of R⁶ and R⁷ is hydrogen and the other of R⁶ and R⁷ is C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁸ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁸ is hydrogen, C1-C6 alkyl, C3-C10 cycloalkyl, or 8-12 membered heterocyclyl. In some embodiments, R⁸ is hydrogen, C1-C3 alkyl, C3-C6 cycloalkyl, or 4-8 membered heterocyclyl.

In some embodiments, R^{A} is optionally substituted C1-C6 alkyl. In some embodiments, R^{A} is C1-C6 alkyl. In some embodiments, R^{A} is methyl, ethyl, or n-propyl.

In some embodiments, R^{A} is C1-C6 haloalkyl. In some embodiments, R^{A} is C1-C3 haloalkyl. In some embodiments, R^{A} is trifluoromethyl.

In some embodiments, R^{A} is optionally substituted C2-C6 alkenyl. In some embodiments, R^{A} is C2-C6 alkenyl. In some embodiments, R^{A} is optionally substituted C2-C3 alkenyl. In some embodiments, R^{A} is C2-C3 alkenyl.

In some embodiments, R^{A} is optionally substituted C2-C6 alkynyl. In some embodiments, R^{A} is C2-C6 alkynyl. In some embodiments, R^{A} is optionally substituted C2-C3 alkynyl. In some embodiments, R^{A} is C2-C3 alkynyl.

In some embodiments, R^{A} is optionally substituted C3-C10 cycloalkyl. In some embodiments, R^{A} is optionally substituted C3-C6 cycloalkyl. In some embodiments, R^{A} is C3-C10 cycloalkyl. In some embodiments, R^{A} is C3-C6 cycloalkyl.

In some embodiments, R^{A} is optionally substituted phenyl. In some embodiments, R^{A} is phenyl.

In some embodiments, R^{A} is optionally substituted 3-12 membered heterocyclyl. In some embodiments, R^{A} is optionally substituted 4-8 membered heterocyclyl. In some embodiments, R^{A} is 3-12 membered heterocyclyl. In some embodiments, R^{A} is 4-8 membered heterocyclyl.

In some embodiments, R^{A} is optionally substituted 5-10 membered heteroaryl. In some embodiments, R^{A} is optionally substituted 5-6 membered heteroaryl. In some embodiments, R^{A} is 5-10 membered heteroaryl. In some embodiments, R^{A} is 5-6 membered heteroaryl. In some embodiments, R^{A} is an optionally substituted 9-10 membered heteroaryl. In some embodiments, R^{A} is a 9-10 membered heteroaryl.

In some embodiments, R^{A} is phenyl optionally substituted with 1-3 independently selected R^{A1}. In some embodiments, R^{A} is pyridinyl, pyrimidinyl, pyridizinyl, or pyrazinyl, each optionally substituted with 1-3 independently selected R^{A2}. In some embodiments, R^{A} is a 9 membered heteroaryl optionally substituted with 1-3 independently selected R^{A3}.

In some embodiments, each R^{A1} is independently selected from halogen, cyano, amino, hydroxyl, sulfhydryl, C1-C3 alkyl, C1-C3 alkoxy, (C1-C3 alkoxy)C1-C3 alkyl, (hydroxy)C1-C3 alkoxy, (C1-C3 alkoxy)C1-C3 alkoxy, (C1-C3 alkoxy)C1-C3 amino, 4-5 membered heterocyclyloxy, C-amido, S-sulfonamido, sulfenyl, sulfonyl, sulfinyl, sulfoximine, sulfonimidamindo, phosphoxide, and C-carboxy.

In some embodiments, each R^{A2} is independently selected from halogen, cyano, amino, hydroxyl, sulfhydryl, C1-C3 alkyl, C1-C3 alkoxy, (C1-C3 alkoxy)C1-C3 alkyl, (hydroxy)C1-C3 alkoxy, (C1-C3 alkoxy)C1-C3 alkoxy, 4-5 membered heterocyclyloxy, C-amido, S-sulfonamido, sulfenyl, sulfonyl, sulfinyl, sulfoximine, sulfonimidamindo, phosphine oxide, and C-carboxy.

In some embodiments, each R^{A3} is independently selected from halogen, cyano, amino, hydroxyl, sulfhydryl, C1-C3 alkyl, C1-C3 alkoxy, (C1-C3 alkoxy)C1-C3 alkyl, (hydroxy)C1-C3 alkoxy, (C1-C3 alkoxy)C1-C3 alkoxy, 4-5 membered heterocyclyloxy, C-amido, S-sulfonamido, sulfenyl, sulfonyl, sulfinyl, sulfoximine, sulfonimidamindo, phosphine oxide, and C-carboxy.

In some embodiments, each R^{A1}, R^{A2} and R^{A3} is independently selected from halogen, cyano, hydroxyl, C1-C3 alkyl, C1-C3 alkoxy, (C1-C3 alkoxy)C1-C3 alkyl, (hydroxy)C1-C3 alkoxy, (C1-C3 alkoxy)C1-C3 alkoxy, (C1-C3 alkoxy)C1-C3 amino, 4-5 membered heterocyclyloxy, —C(═O)NRR′, —CO₂H, —SO₂(C1-C3 alkyl), —S(═O)C1-C3 alkyl, —SO₂NRR′, —S(═O)(═NR)NRR′, or —P(═O)(C1-C3 alkyl)₂, wherein each R and R′ are independently hydrogen or C1-C3 alkyl.

In some embodiments, each R^{Aa} is independently selected from halogen, cyano, C1-C3 alkyl, C1-C3 alkoxy, 4-5 membered heterocyclyloxy, —SO₂(C1-C3 alkyl), —SO₂NRR′, or —P(═O)(C1-C3 alkyl)₂, wherein R and R′ are independently hydrogen or C1-C3 alkyl.

In some embodiments, R^{A} is

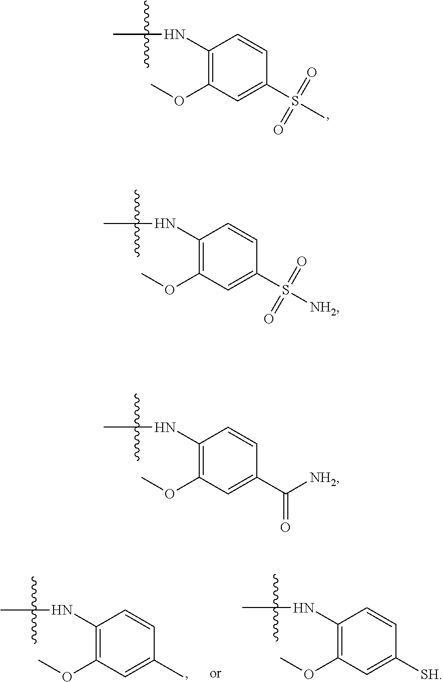 [SMILES: *Nc1ccc(S(C)(=O)=O)cc1OC ; *Nc1ccc(S(N)(=O)=O)cc1OC ; *Nc1ccc(C(N)=O)cc1OC ; *Nc1ccc(C)cc1OC ; *Nc1ccc(S)cc1OC]

In some embodiments, R^{A} is

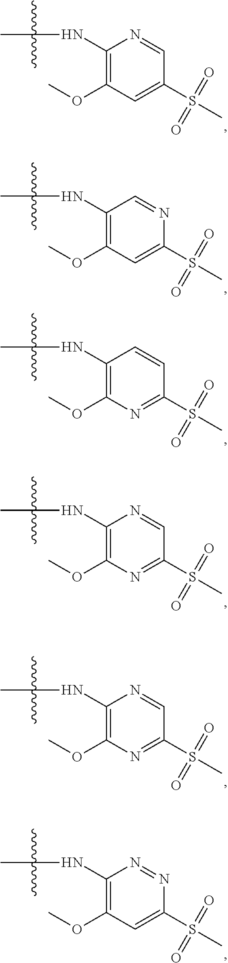 [SMILES: *Nc1ncc(S(C)(=O)=O)cc1OC ; *Nc1cnc(S(C)(=O)=O)cc1OC ; *Nc1ccc(S(C)(=O)=O)nc1OC ; *Nc1ncc(S(C)(=O)=O)nc1OC ; *Nc1ncc(S(C)(=O)=O)nc1OC ; *Nc1nnc(S(C)(=O)=O)cc1OC]

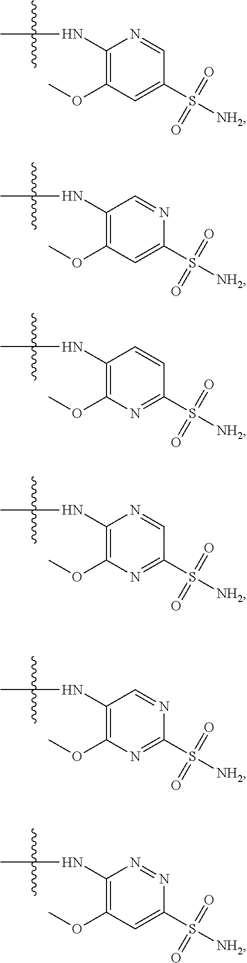 [SMILES: *Nc1ncc(S(N)(=O)=O)cc1OC ; *Nc1cnc(S(N)(=O)=O)cc1OC ; *Nc1ccc(S(N)(=O)=O)nc1OC ; *Nc1ncc(S(N)(=O)=O)nc1OC ; *Nc1cnc(S(N)(=O)=O)nc1OC ; *Nc1nnc(S(N)(=O)=O)cc1OC]

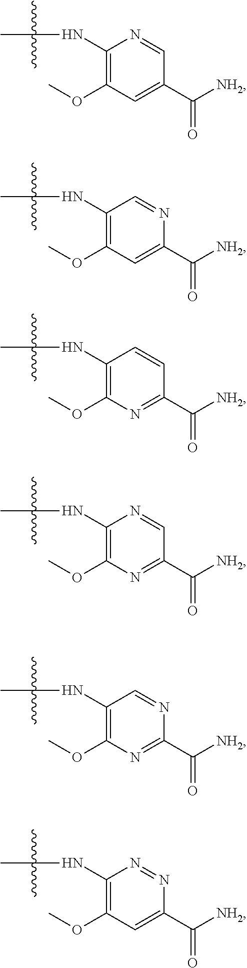 [SMILES: *Nc1ncc(C(N)=O)cc1OC ; *Nc1cnc(C(N)=O)cc1OC ; *Nc1ccc(C(N)=O)nc1OC ; *Nc1ncc(C(N)=O)nc1OC ; *Nc1cnc(C(N)=O)nc1OC ; *Nc1nnc(C(N)=O)cc1OC]

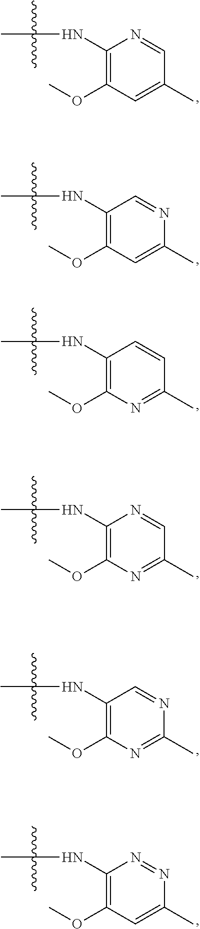 [SMILES: *Nc1ncc(C)cc1OC ; *Nc1cnc(C)cc1OC ; *Nc1ccc(C)nc1OC ; *Nc1ncc(C)nc1OC ; *Nc1cnc(C)nc1OC ; *Nc1nnc(C)cc1OC]

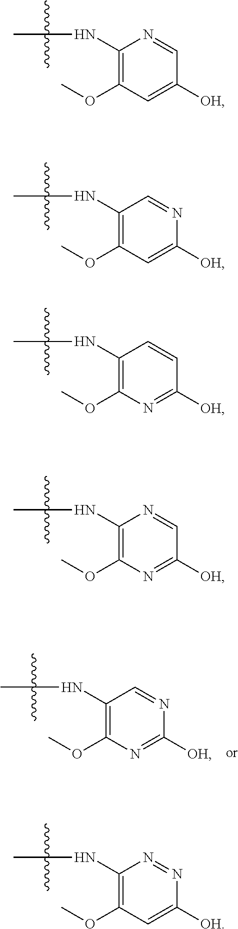 [SMILES: *Nc1ncc(O)cc1OC ; *Nc1cnc(O)cc1OC ; *Nc1ccc(O)nc1OC ; *Nc1ncc(O)nc1OC ; *Nc1cnc(O)nc1OC ; *Nc1nnc(O)cc1OC]

In some embodiments, R^{A} is

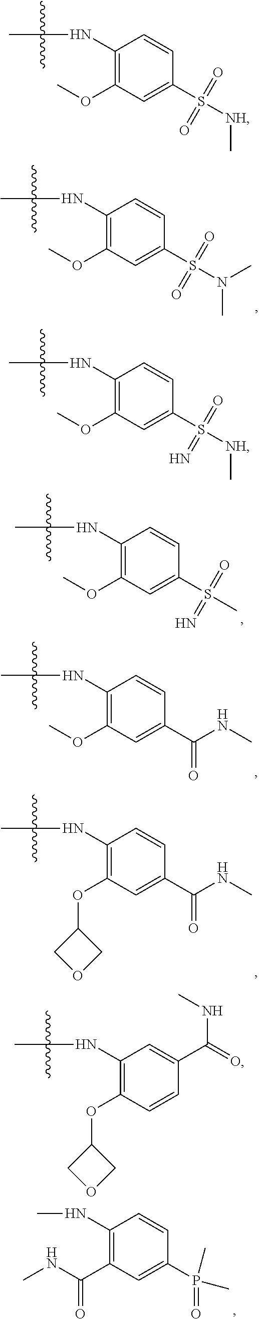

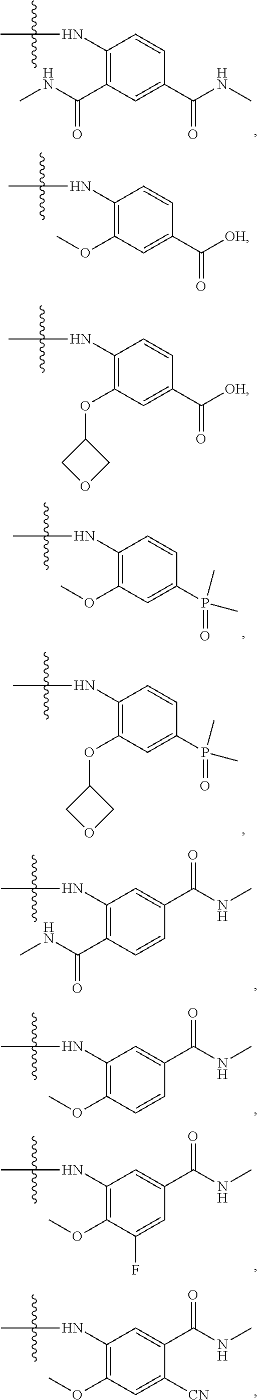

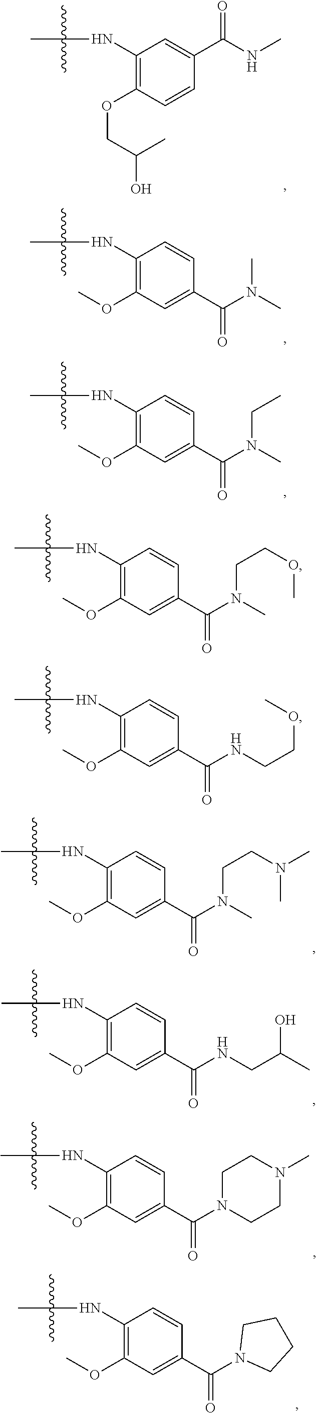

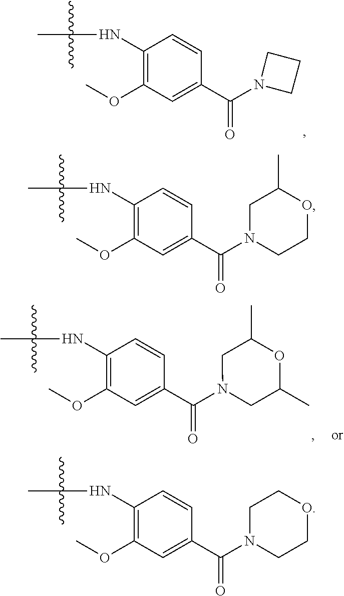

In some embodiments, R^{A} is

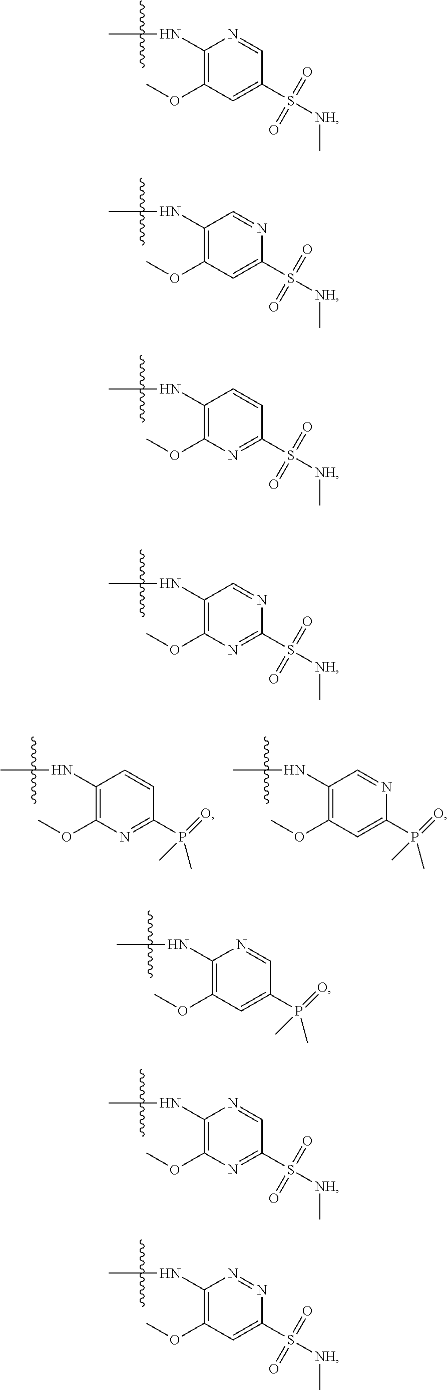

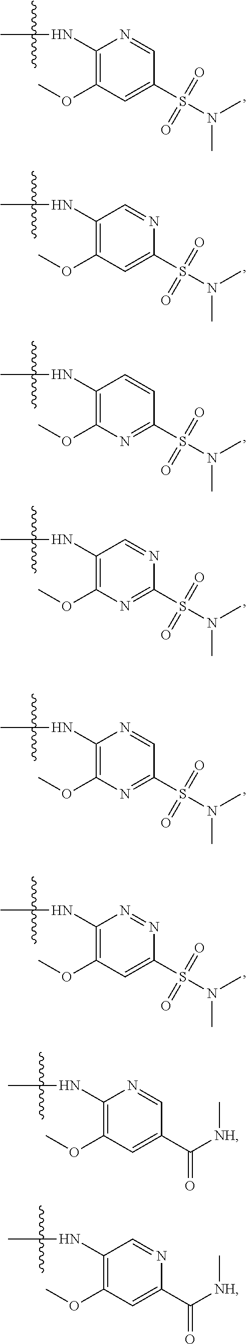

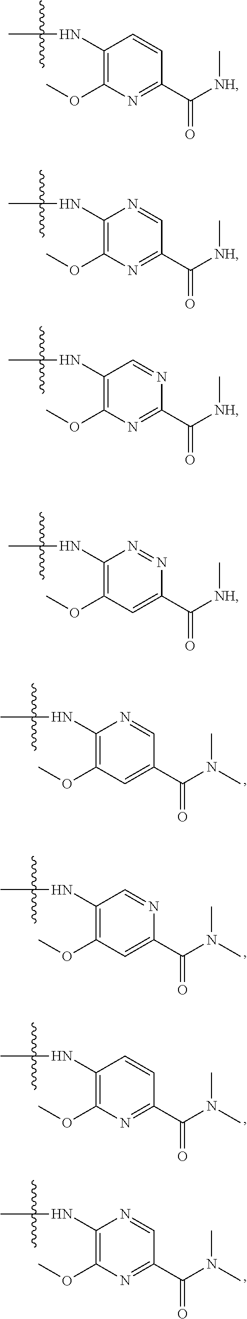

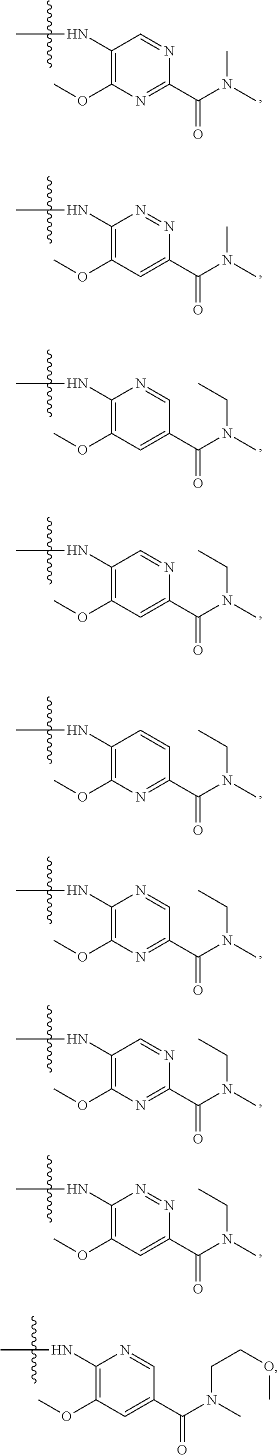

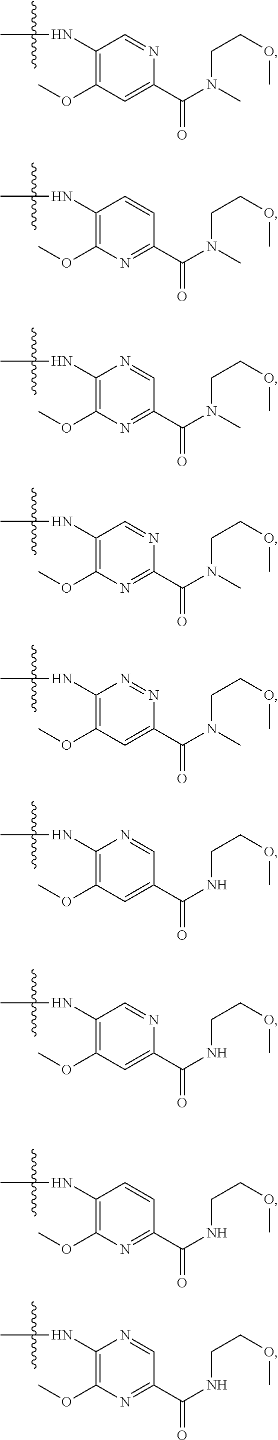

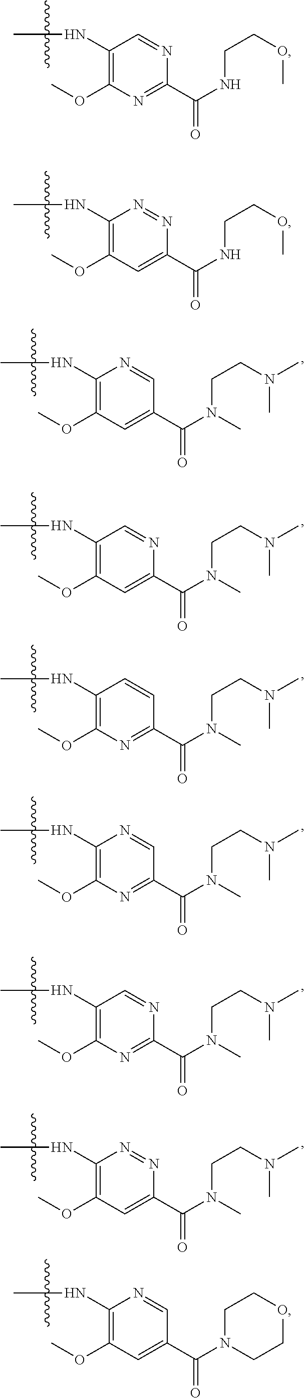

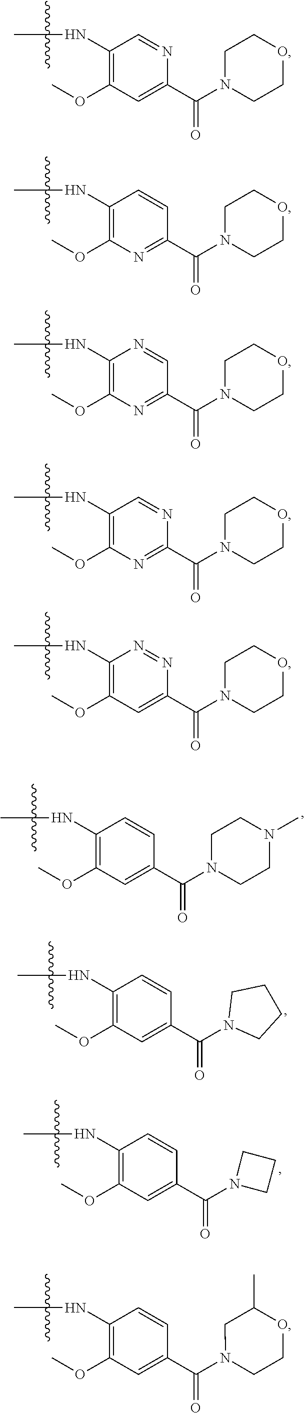

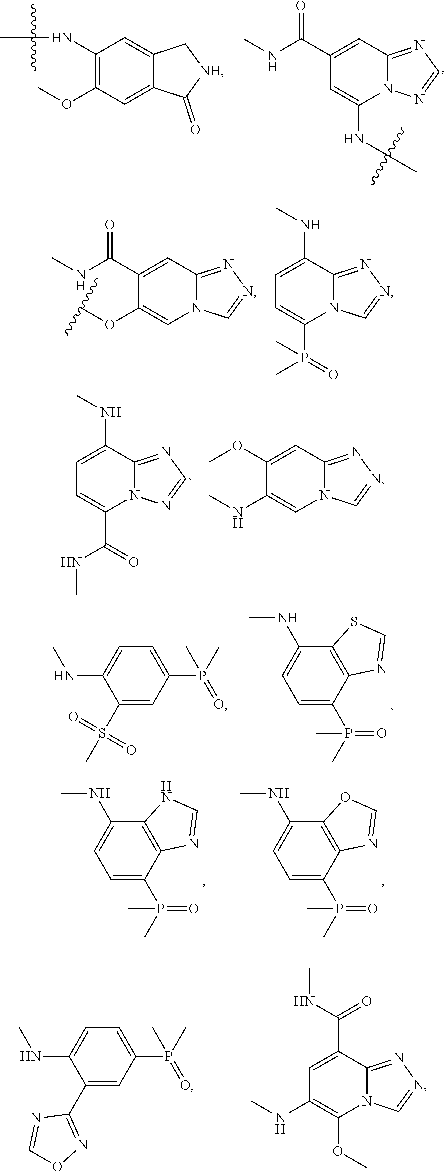

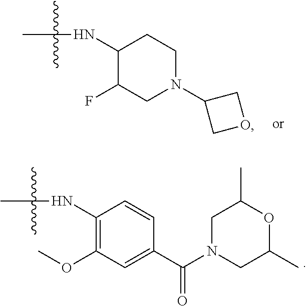

In some embodiments, R^{A} is

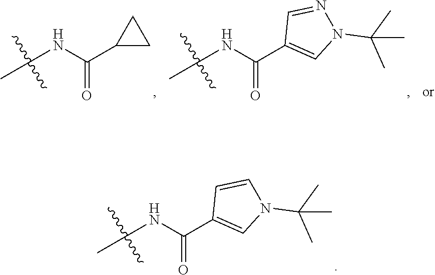 [SMILES: *NC(=O)C1CC1 ; *NC(=O)c1cnn(C(C)(C)C)c1 ; *NC(=O)c1ccn(C(C)(C)C)c1]

In some embodiments, R^{A} is

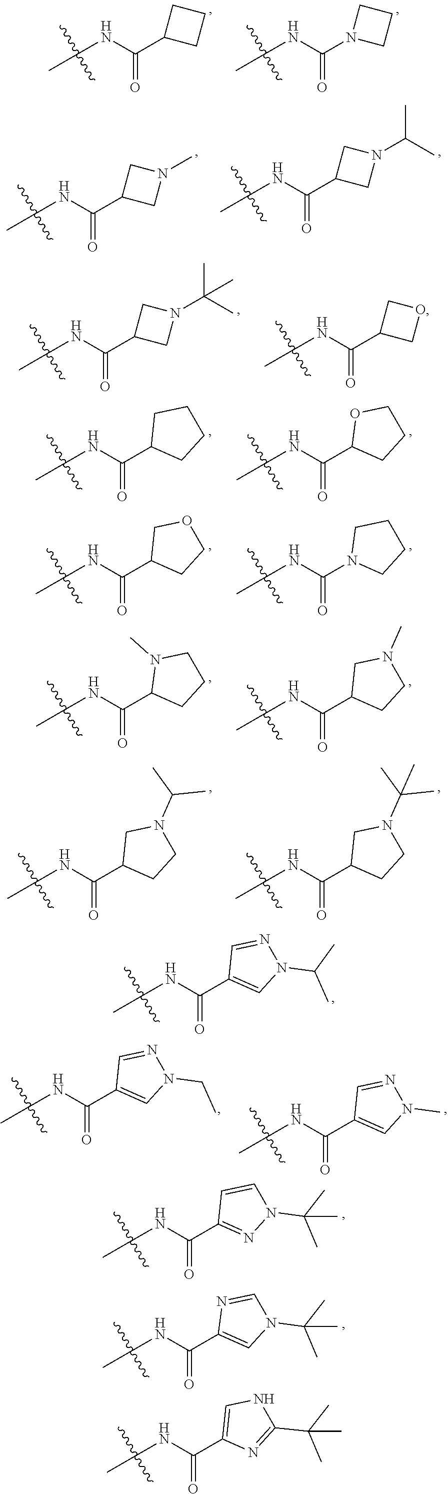

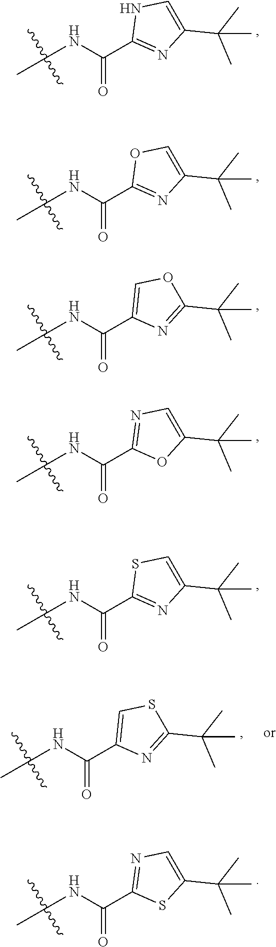

In some embodiments, R^{B} is halogen. In some embodiments, R^{B} is fluoro or chloro.

In some embodiments, R^{B} is cyano.

In some embodiments, R^{B} is hydroxyl.

In some embodiments, R^{B} is —NR⁸R⁹.

In some embodiments, R^{B} is —OR⁸.

In some embodiments, R^{B} is —C(═O)NR⁸R⁹.

In some embodiments, R^{B} is —C(═O)R⁸.

In some embodiments, R^{B} is —C(═O)OR⁸.

In some embodiments, R^{B} is —NR⁸C(═O)OR⁹

In some embodiments, R^{B} is OC(═O)R⁸.

In some embodiments, R^{B} is —OC(═O)NR⁸.

In some embodiments, R^{B} is —C(═O)NR⁸R⁹.

In some embodiments, R^{B} is —NR⁸C(═O)R⁹.

In some embodiments, R^{B} is —NR⁸C(═O)NR⁹.

In some embodiments, R^{B} is —SR⁸.

In some embodiments, R^{B} is —S(═O)R⁸.

In some embodiments, R^{B} is —S(O₂)R⁸.

In some embodiments, R^{B} is —S(O₂)NR^{B}.

In some embodiments, R^{B} is —NR⁸S(O₂)R⁹.

In some embodiments, R^{B} is —R⁸C(═O)R⁹.

In some embodiments, R^{B} is —NR⁸C(═O)R⁹.

In some embodiments, R^{B} is —NR⁸C(═O)NR⁹.

In some embodiments, R⁸ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁸ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁸ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁸ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁸ is hydrogen, C1-C6 alkyl, C3-C10 cycloalkyl, or 8-12 membered heterocyclyl. In some embodiments, R⁸ is hydrogen, C1-C3 alkyl, C3-C6 cycloalkyl, or 4-8 membered heterocyclyl.

In some embodiments, R⁹ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R⁹ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R⁹ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R⁹ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R⁹ is hydrogen, C1-C6 alkyl, C3-C10 cycloalkyl, or 4-12 membered heterocyclyl. In some embodiments, R⁹ is hydrogen, C1-C3 alkyl, C3-C6 cycloalkyl, or 4-8 membered heterocyclyl.

In some embodiments, when R⁸ and R⁹ are attached to the same nitrogen atom, R⁸ and R⁹ are the same. In some embodiments, when R⁸ and R⁹ are attached to the same nitrogen atom, R⁸ and R⁹ are different. In some embodiments, when R⁸ and R⁹ are attached to the same nitrogen atom, R⁸ and R⁹ are each hydrogen. In some embodiments, when R⁸ and R⁹ are attached to the same nitrogen atom, R⁸ and R⁹ are each an independently selected C1-C6 alkyl.

In some embodiments, when R⁸ and R⁹ are attached to the same nitrogen atom, one of R⁸ and R⁹ is hydrogen and the other of R⁸ and R⁹ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, when R⁸ and R⁹ are attached to the same nitrogen atom, one of R⁸ and R⁹ is hydrogen and the other of R⁸ and R⁹ is C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R^{B} is optionally substituted C1-C6 alkyl. In some embodiments, R^{B} is C1-C6 alkyl. In some embodiments, R^{B} is methyl, ethyl, or n-propyl.

In some embodiments, R^{B} is C1-C6 haloalkyl. In some embodiments, R^{B} is C1-C3 haloalkyl. In some embodiments, R^{B} is a C1-C3 fluoroalkyl. In some embodiments, R^{B} is trifluoromethyl or 2,2,2-trifluoroethyl. In some embodiments, R^{B} is 2,2,2-trifluoroethyl.

In some embodiments, R^{B} is optionally substituted C2-C6 alkenyl. In some embodiments, R^{B} is C2-C6 alkenyl. In some embodiments, R^{B} is optionally substituted C2-C3 alkenyl. In some embodiments, R^{B} is C2-C3 alkenyl.

In some embodiments, R^{B} is optionally substituted C2-C6 alkynyl. In some embodiments, R^{B} is C2-C6 alkynyl. In some embodiments, R^{B} is optionally substituted C2-C3 alkynyl. In some embodiments, R^{B} is C2-C3 alkynyl.

In some embodiments, R^{B} is optionally substituted C3-C10 cycloalkyl. In some embodiments, R^{B} is optionally substituted C3-C6 cycloalkyl. In some embodiments, R^{B} is C3-C10 cycloalkyl. In some embodiments, R^{B} is C3-C6 cycloalkyl.

In some embodiments, R^{B} is optionally substituted phenyl. In some embodiments, R^{B} is phenyl.

In some embodiments, R^{B} is optionally substituted 3-12 membered heterocyclyl. In some embodiments, R^{B} is optionally substituted 4-8 membered heterocyclyl. In some embodiments, R^{B} is 3-12 membered heterocyclyl. In some embodiments, R^{B} is 4-8 membered heterocyclyl.

In some embodiments, R^{B} is optionally substituted 5-10 membered heteroaryl. In some embodiments, R^{B} is optionally substituted 5-6 membered heteroaryl. In some embodiments, R^{B} is 5-10 membered heteroaryl. In some embodiments, R^{B} is 5-6 membered heteroaryl.

In some embodiments, R^{B} is methyl, ethyl, or n-propyl.

In some embodiments, R^{B} is hydroxyl,

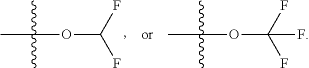 [SMILES: *OC(F)F ; *OC(F)(F)F]

In some embodiments, R^{B} is

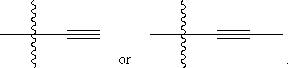

In some embodiments, R^{B} is

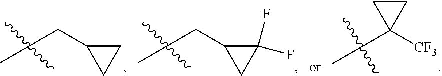 [SMILES: *CC1CC1 ; *CC1CC1(F)F ; *C1(C(F)(F)F)CC1]

In some embodiments, R^{B} is

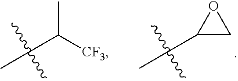

In some embodiments, R^{B} is

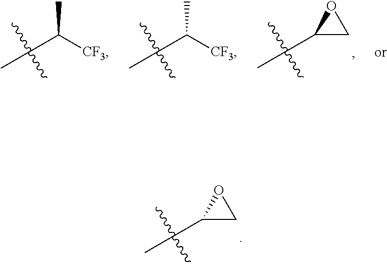

In some embodiments, R^{B} is

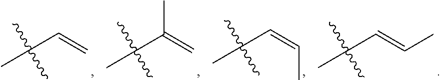

In some embodiments, R^{B} is

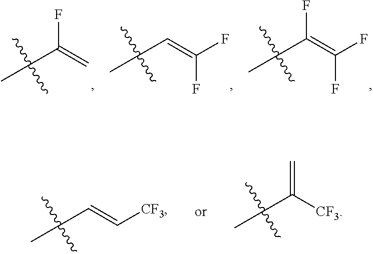

In some embodiments, R^{B} is

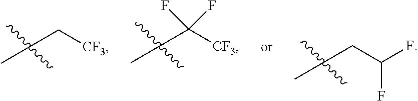 [SMILES: *CC(F)(F)F ; *C(F)(F)C(F)(F)F ; *CC(F)F]

In some embodiments, each R² is

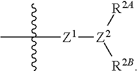

In some embodiments, one of X², X³, X⁴, and X⁵ is CR² and the remaining X², X³, X⁴, and X are CH, N, or CR³.

In some embodiments, Z¹ is a bond.

In some embodiments, Z¹ is —C═O—.

In some embodiments, Z¹ is —S(O₂)—.

In some embodiments, Z¹ is optionally substituted C1-C6 alkylene. In some embodiments, Z¹ is optionally substituted C1-C3 alkylene. In some embodiments, Z¹ is C1-C6 alkylene. In some embodiments, Z¹ is C1-C3 alkylene. In some embodiments, Z¹ is methylene or ethylene.

In some embodiments, Z¹ is optionally substituted C2-C6 alkenylene. In some embodiments, Z¹ is optionally substituted C2-C3 alkenylene. In some embodiments, Z¹ is C2-C6 alkenylene. In some embodiments, Z¹ is C2-C3 alkenylene.

In some embodiments, Z¹ is optionally substituted C2-C6 alkynylene. In some embodiments, Z¹ is optionally substituted C2-C3 alkynylene. In some embodiments, Z¹ is C2-C6 alkynylene. In some embodiments, Z¹ is C2-C3 alkynylene.

In some embodiments, Z¹ is an optionally substituted C3-C4 cycloalkylene. In some embodiments, Z¹ is C3-C4 cycloalkylene.

In some embodiments, Z² is N.

In some embodiments, Z² is O and R^{2B} is absent.

In some embodiments, Z² is a bond.

In some embodiments, Z² is CR^{2C}.

In some embodiments, R^{2C} is hydrogen.

In some embodiments, R^{2C} is halogen. In some embodiments, R^{2C} is fluoro or chloro.

In some embodiments, R^{2C} is C1-C6 alkyl. In some embodiments, R²C is C1-C3 alkyl. In some embodiments, R^{2C} is methyl.

In some embodiments, when Z¹ is a bond and Z² is a bond, R^{2B} is absent and R^{2A} is directly connected to Formula (I) via Z¹. In some embodiments, when Z² is a bond, R^{2B} is absent and R^{2A} is directly connected to Z¹. In some embodiments, Z² is 0 and R^{2B} is absent.

In some embodiments, R² is-NR^{2A}R^{2B}, i.e., Z¹ is a bond and Z² is N. In some embodiments, R² is R^{2B}, i.e., Z¹ and Z² are both a bond, R^{2A} is absent and R^{2B} is directly connected to Formula (I) via Z¹.

In some embodiments, when Z¹ is a not a bond and Z² is a bond, R^{2B} is absent and R^{2A} is directly connected to Z¹.

In some embodiments, R^{2A} is hydrogen.

In some embodiments, R^{2A} is —C(═O)R¹⁰.

In some embodiments, R^{2A} is —C(═O)OR¹⁰.

In some embodiments, R^{2A} is —C(═O)NR¹⁰R¹¹.

In some embodiments, R^{2A} is —S(═O)R¹⁰.

In some embodiments, R^{2A} is —S(O₂)R¹⁰.

In some embodiments, R¹⁰ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹⁰ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-10 membered heterocyclyl, or optionally substituted 5-6 membered heteroaryl.

In some embodiments, R¹⁰ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹⁰ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R¹⁰ is hydrogen, C1-C6 alkyl, C3-C10 cycloalkyl, or 4-8 membered heterocyclyl. In some embodiments, R¹⁰ is hydrogen, C1-C3 alkyl, C3-C6 cycloalkyl, or 4-10 membered heterocyclyl.

In some embodiments, R¹¹ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹¹ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R¹¹ is hydrogen, C1-C6 alkyl, C2-C6 alkenyl, C2-C6 alkynyl, C3-C10 cycloalkyl, phenyl, 4-8 membered heterocyclyl, or 5-10 membered heteroaryl.

In some embodiments, R¹¹ is hydrogen, CJ-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R¹¹ is hydrogen, C1-C6 alkyl, C3-C10 cycloalkyl, or 4-12 membered heterocyclyl. In some embodiments, R¹¹ is hydrogen, C1-C3 alkyl, C3-C6 cycloalkyl, or 4-8 membered heterocyclyl.

In some embodiments, when R¹⁰ and R¹¹ are attached to the same nitrogen atom, R¹⁰ and R¹¹ are the same. In some embodiments, when R¹⁰ and R¹¹ are attached to the same nitrogen atom, R¹⁰ and R¹¹ are different. In some embodiments, when R¹⁰ and R¹¹ are attached to the same nitrogen atom, R¹⁰ and R¹¹ are each hydrogen. In some embodiments, when R¹⁰ and R¹¹ are attached to the same nitrogen atom, R¹⁰ and R¹¹ are each an independently selected C1-C6 alkyl.

In some embodiments, when R¹⁰ and R¹¹ are attached to the same nitrogen atom, one of R¹⁰ and R¹¹ is hydrogen and the other of R¹⁰ and R¹¹ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-8 membered heterocyclyl, or optionally substituted 5-10 membered heteroaryl.

In some embodiments, when R¹⁰ and R¹¹ are attached to the same nitrogen atom, one of R¹⁰ and R¹¹ is hydrogen and the other of R¹⁰ and R¹¹ is C1-C6 alkyl, C2-C6 alkenyl, C2-C6 alkynyl, C3-C10 cycloalkyl, phenyl, 4-8 membered heterocyclyl, or 5-10 membered heteroaryl.

In some embodiments, R^{2A} is optionally substituted C1-C6 alkyl. In some embodiments, R^{2A} is C1-C6 alkyl. In some embodiments, R^{2A} is methyl or ethyl.

In some embodiments, R^{2A} is C1-C6 haloalkyl. In some embodiments, R^{2A} is C1-C3 haloalkyl. In some embodiments, R^{2A} is trifluoromethyl.

In some embodiments, R^{2A} is optionally substituted C2-C6 alkenyl. In some embodiments, R^{2A} is C2-C6 alkenyl. In some embodiments, R^{2A} is optionally substituted C2-C3 alkenyl. In some embodiments, R^{2A} is C2-C3 alkenyl.

In some embodiments, R^{2A} is optionally substituted C2-C6 alkynyl. In some embodiments, R^{2A} is C2-C6 alkynyl. In some embodiments, R^{2A} is optionally substituted C2-C3 alkynyl. In some embodiments, R^{2A} is C2-C3 alkynyl.

In some embodiments, R^{2A} is optionally substituted C3-C10 cycloalkyl. In some embodiments, R^{2A} is optionally substituted C3-C6 cycloalkyl. In some embodiments, R^{2A} is C3-C10 cycloalkyl. In some embodiments, R^{2A} is C3-C6 cycloalkyl.

In some embodiments, R^{2A} is optionally substituted phenyl. In some embodiments, R^{2A} is phenyl.

In some embodiments, R^{2A} is optionally substituted 3-12 membered heterocyclyl. In some embodiments, R^{2A} is optionally substituted 4-8 membered heterocyclyl. In some embodiments, R^{2A} is 3-12 membered heterocyclyl. In some embodiments, R^{2A} is 4-8 membered heterocyclyl.

In some embodiments, R^{2A} is optionally substituted 5-10 membered heteroaryl. In some embodiments, R^{2A} is optionally substituted 5-6 membered heteroaryl. In some embodiments, R^{2A} is 5-10 membered heteroaryl. In some embodiments, R^{2A} is 5-6 membered heteroaryl.

In some embodiments, R^{2B} is hydrogen.

In some embodiments, R^{2B} is —C(═O)R¹⁰.

In some embodiments, R^{2B} is —C(═O)OR¹⁰.

In some embodiments, R^{2B} is —C(═O)NR¹⁰R¹¹.

In some embodiments, R^{2B} is —S(═O)R¹⁰.

In some embodiments, R^{2B} is —S(O₂)R¹⁰.

In some embodiments, R^{2B} is optionally substituted C1-C6 alkyl. In some embodiments, R^{2B} is C1-C6 alkyl. In some embodiments, R^{2B} is methyl or ethyl.

In some embodiments, R^{2B} is C1-C6 haloalkyl. In some embodiments, R^{2B} is C1-C3 haloalkyl. In some embodiments, R^{2B} is trifluoromethyl.

In some embodiments, R^{2B} is optionally substituted C2-C6 alkenyl. In some embodiments, R^{2B} is C2-C6 alkenyl. In some embodiments, R^{2B} is optionally substituted C2-C3 alkenyl. In some embodiments, R²Bis C2-C3 alkenyl.

In some embodiments, R^{2B} is optionally substituted C2-C6 alkynyl. In some embodiments, R^{2B} is C2-C6 alkynyl. In some embodiments, R^{2B} is optionally substituted C2-C3 alkynyl. In some embodiments, R²Bis C2-C3 alkynyl.

In some embodiments, R^{2B} is optionally substituted C3-C10 cycloalkyl. In some embodiments, R^{2B} is optionally substituted C3-C6 cycloalkyl. In some embodiments, R^{2B} is C3-C10 cycloalkyl. In some embodiments, R^{2B} is C3-C6 cycloalkyl.

In some embodiments, R^{2B} is optionally substituted phenyl. In some embodiments, R^{2B} is phenyl.

In some embodiments, R^{2B} is optionally substituted 3-12 membered heterocyclyl. In some embodiments, R^{2B} is optionally substituted 4-8 membered heterocyclyl. In some embodiments, R^{2B} is 3-12 membered heterocyclyl. In some embodiments, R^{2B} is 4-8 membered heterocyclyl.

In some embodiments, R^{2B} is optionally substituted 5-10 membered heteroaryl. In some embodiments, R^{2B} is optionally substituted 5-6 membered heteroaryl. In some embodiments, R^{2B} is 5-10 membered heteroaryl. In some embodiments, R^{2B} is 5-6 membered heteroaryl.

In some embodiments, R^{2B} is —C(═O)R¹⁰, —C(═O)OR¹⁰, —C(═O)NR¹⁰R¹⁰, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl; and R^{2B} is hydrogen.

In some embodiments, one of R^{2A} and R^{2B} is hydrogen, C1-C6 alkyl, or C3-C10 cycloalkyl, and the other of R^{2A} and R^{2B} is hydrogen, —C(═O)R¹⁰, —C(═O)OR¹⁰, —C(═O)NR¹⁰R¹¹, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl.

In some embodiments, one of R^{2A} and R^{2B} is hydrogen and the other of R^{2A} and R^{2B} is an optionally substituted 4-12 membered heterocyclyl or an optionally substituted 5-6 membered heteroaryl. In some embodiments, one of R^{2A} and R^{2B} is hydrogen and the other of R^{2A} and R^{2B}is an optionally substituted 4-12 membered heterocyclyl. In some embodiments, one of R^{2A} and R^{2B} is hydrogen and the other of R^{2A} and R^{2B} is a substituted 4-12 membered heterocyclyl.

In some embodiments, R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl.

In some embodiments, R² is

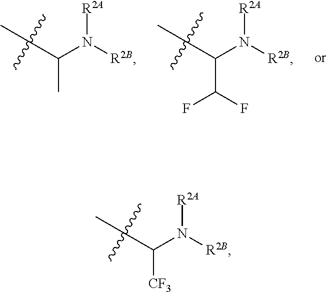

i.e., Z¹ is optionally substituted alkylene and Z² is N. In some embodiments, R² is

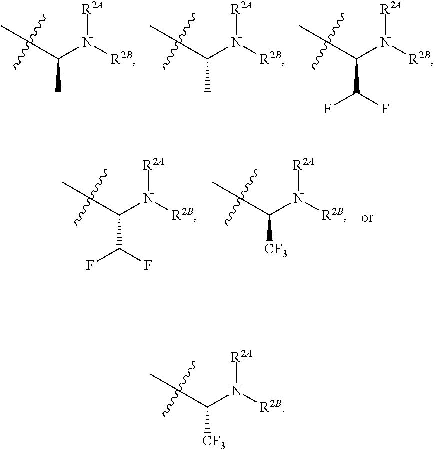

In some embodiments, R² is

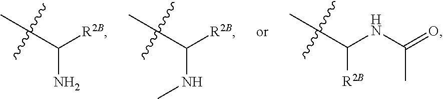

i.e., Z¹ is optionally substituted alkylene, Z² is a bond, and R^{2A} is absent. In some embodiments, R² is

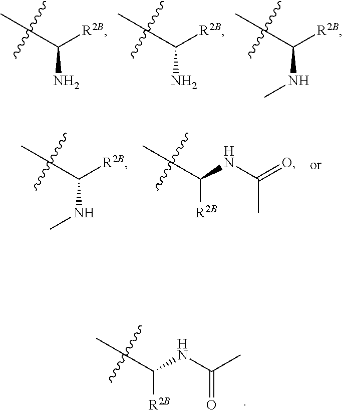

In some embodiments, R^{2A} is hydrogen.

In some embodiments, R^{2B} is

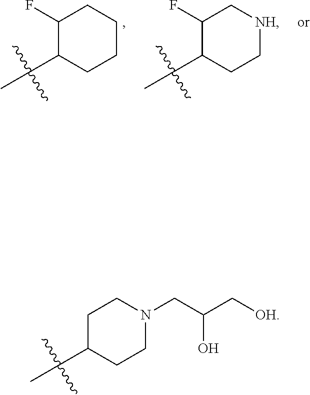 [SMILES: *C1CCCCC1F ; *C1CCNCC1F ; *C1CCN(CC(O)CO)CC1]

In some embodiments, R^{2B} is

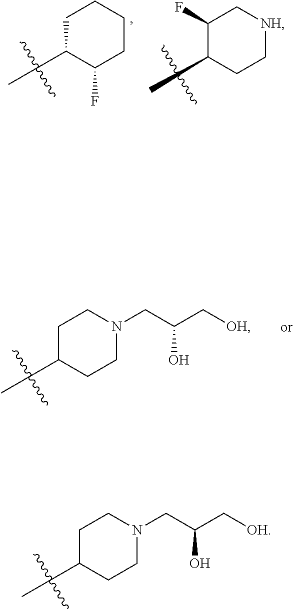

In some embodiments, R^{2B} is

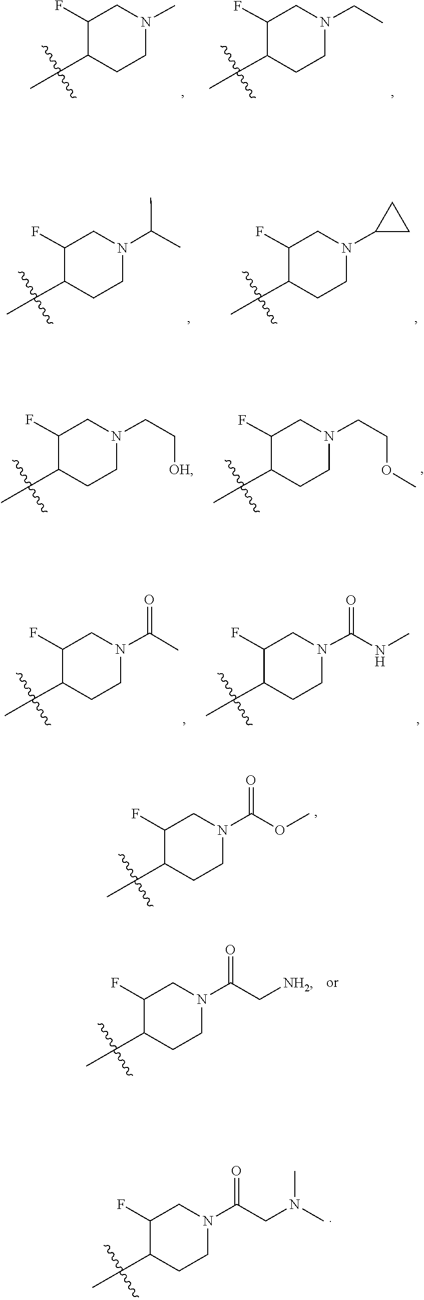

In some embodiments R^{2B} is

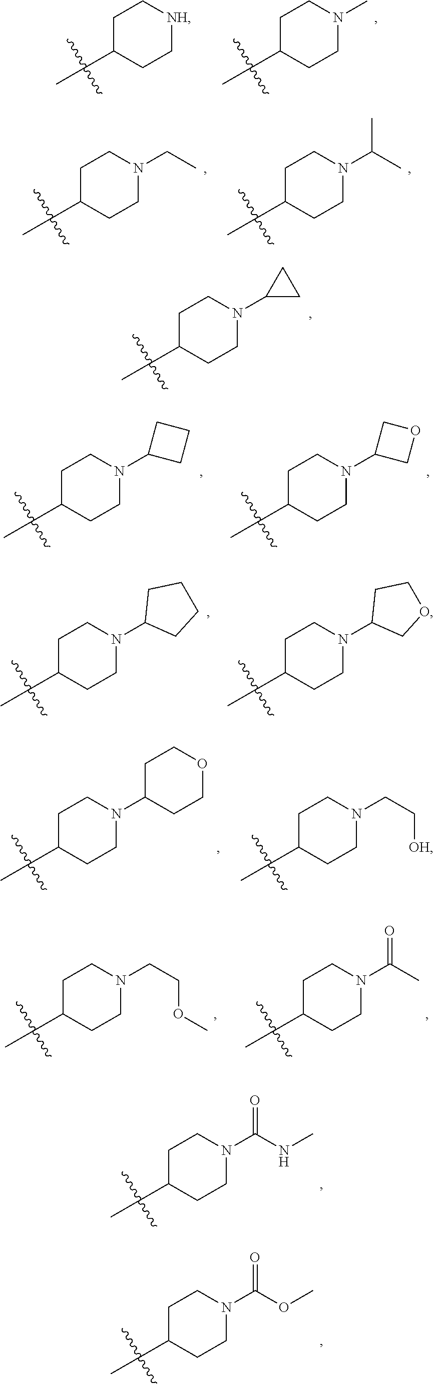

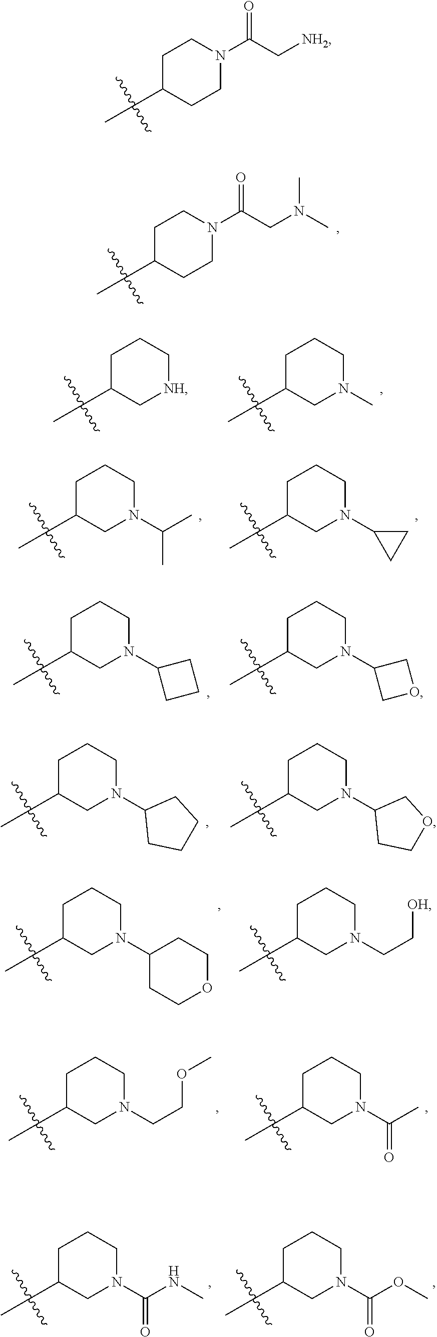

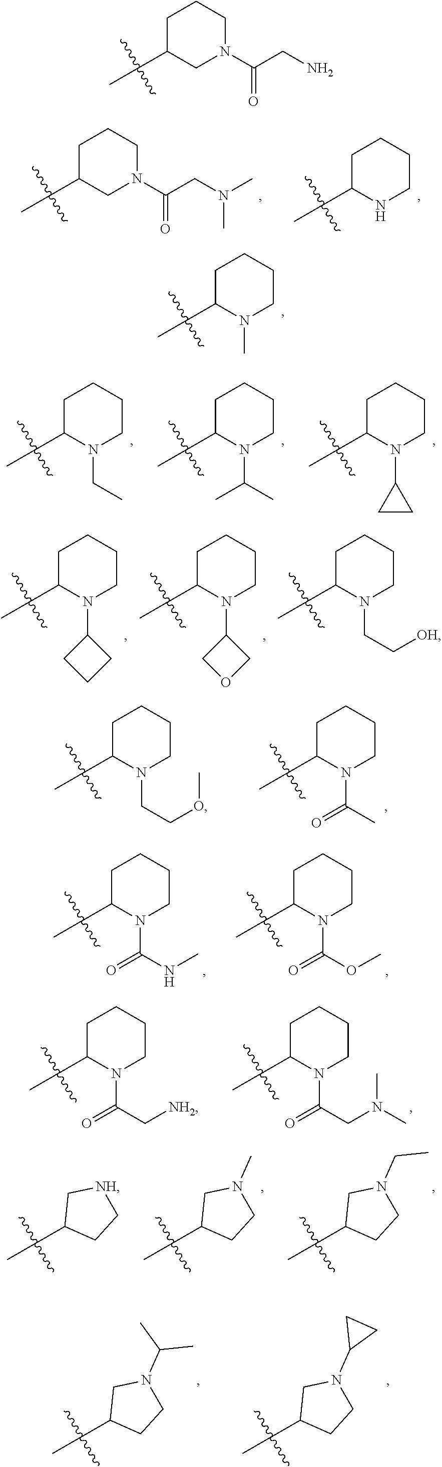

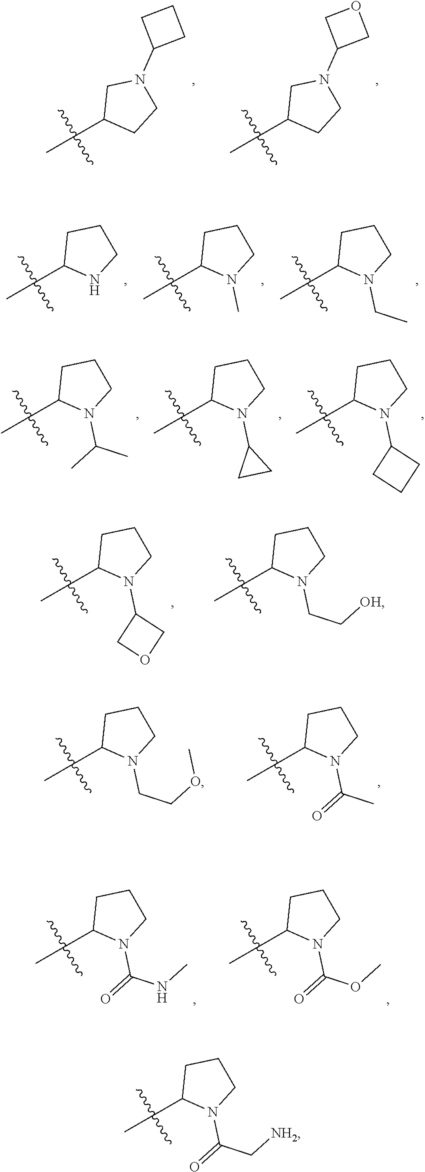

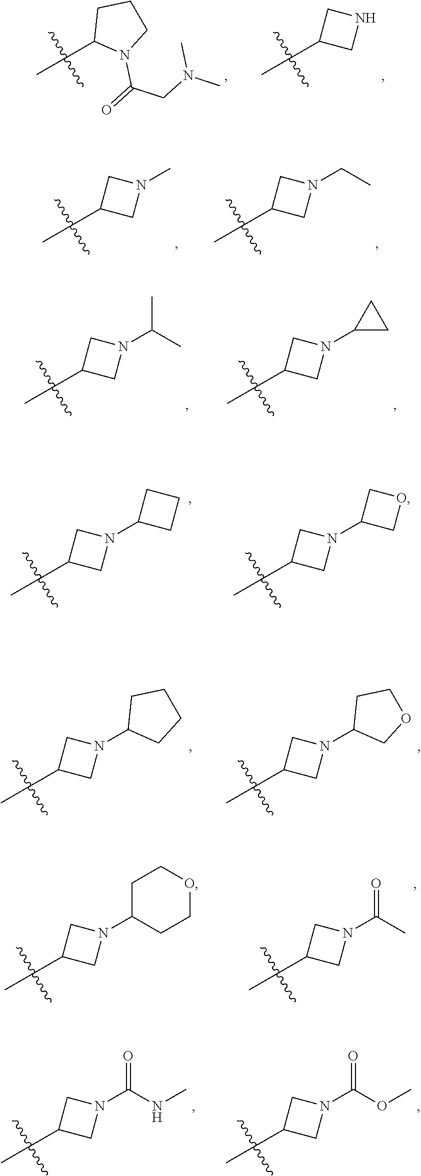

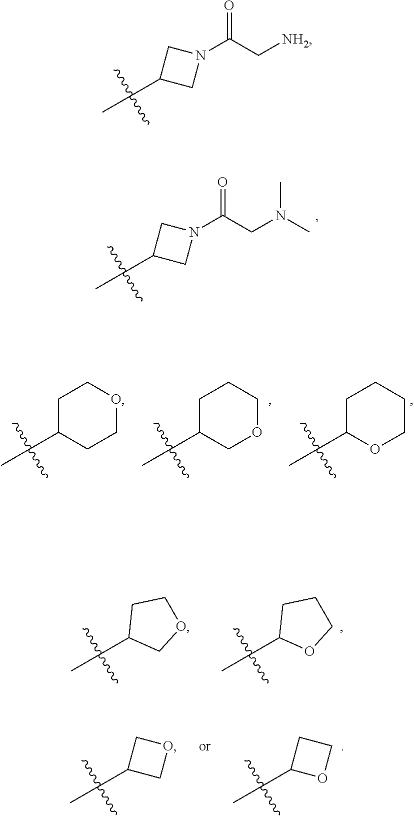

In some embodiments, R^{2B} is

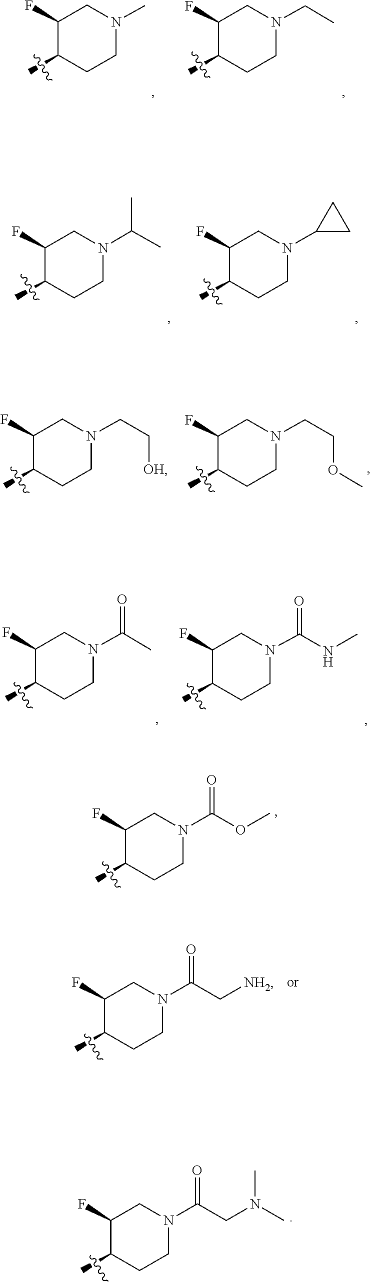

In some embodiments, R^{2B} is

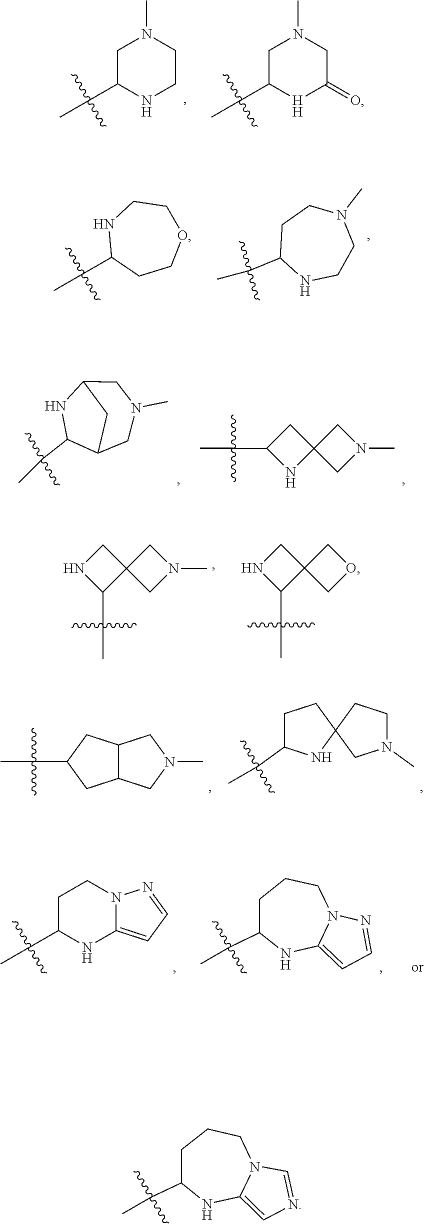

In some embodiments, R^{2B} is

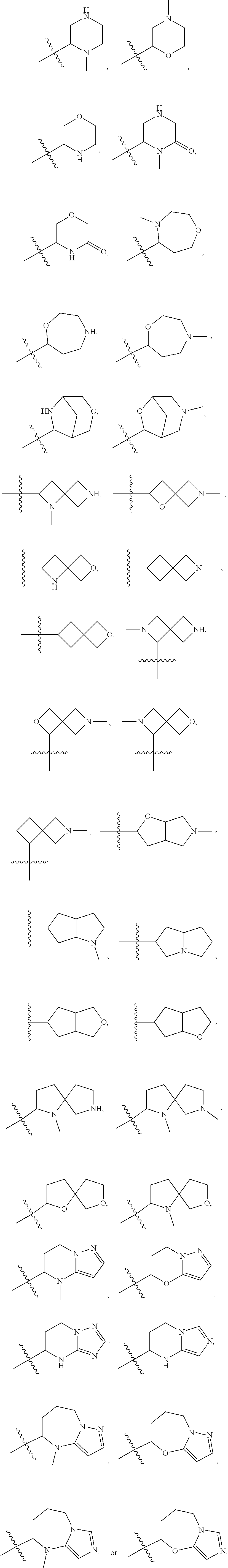

In some embodiments, R^{2B} is

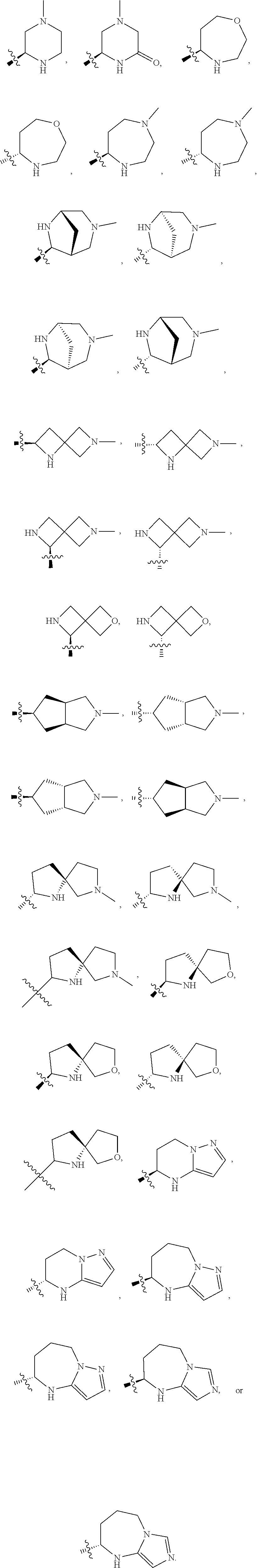

In some embodiments, R^{2B} is

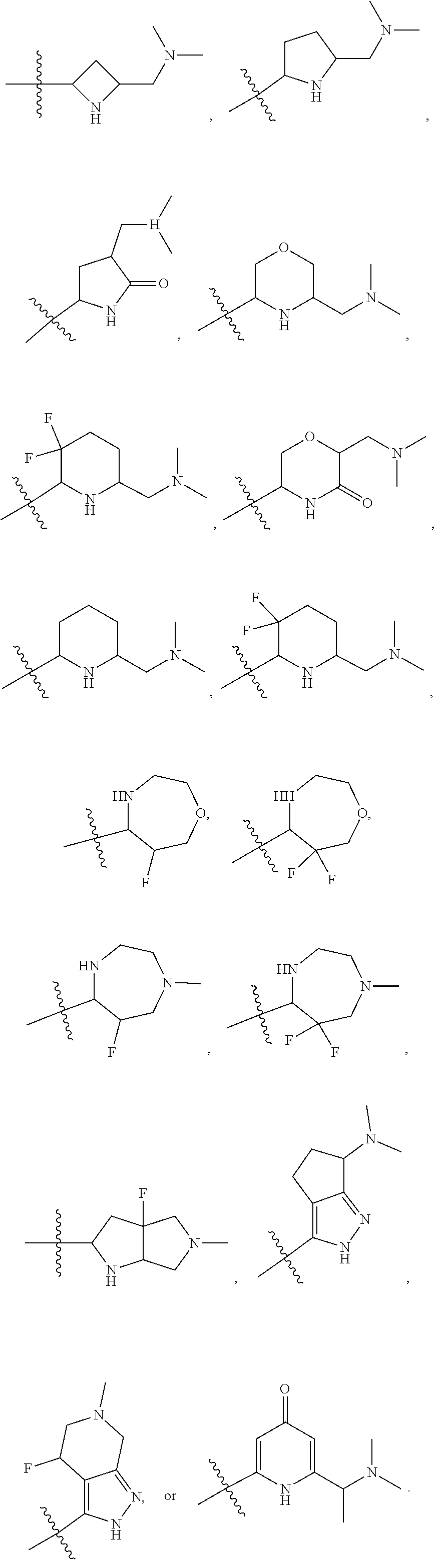

In some embodiments, R^{2B} is

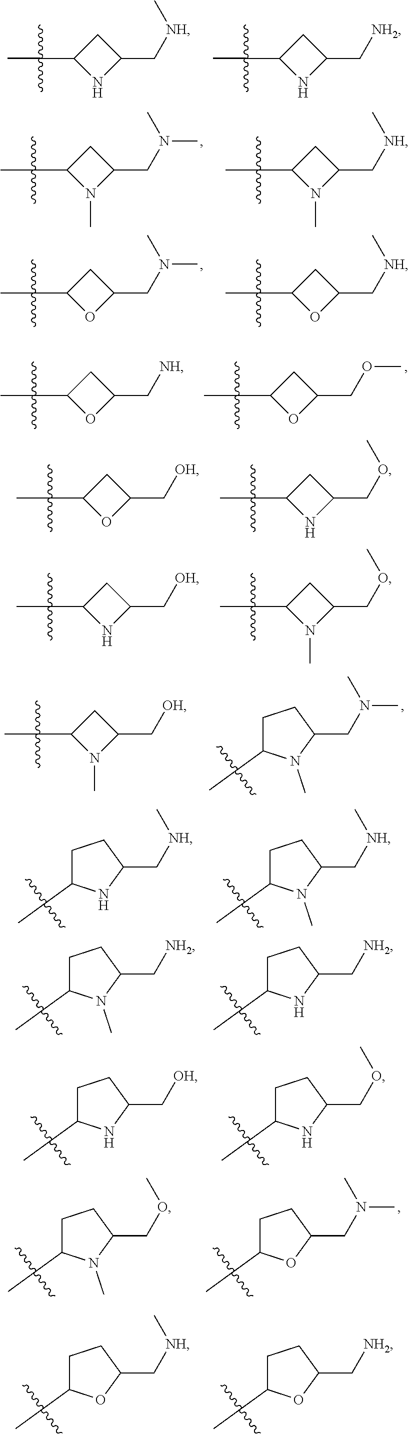

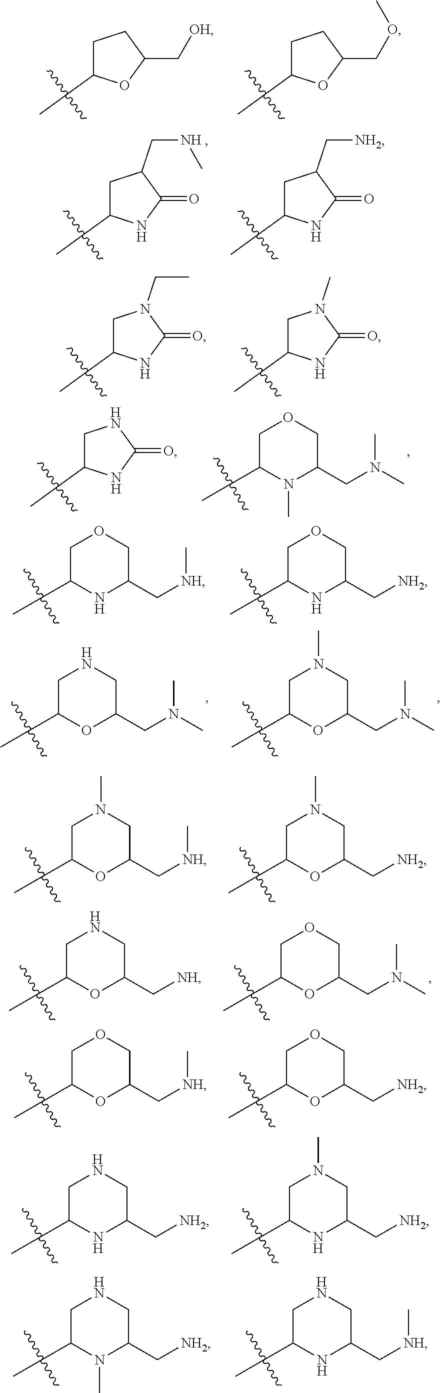

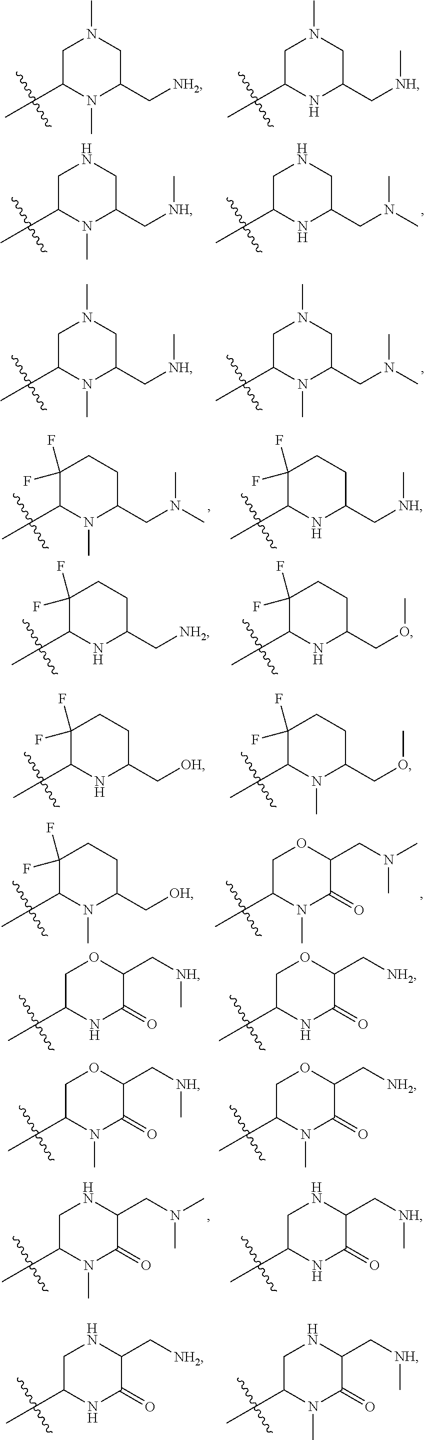

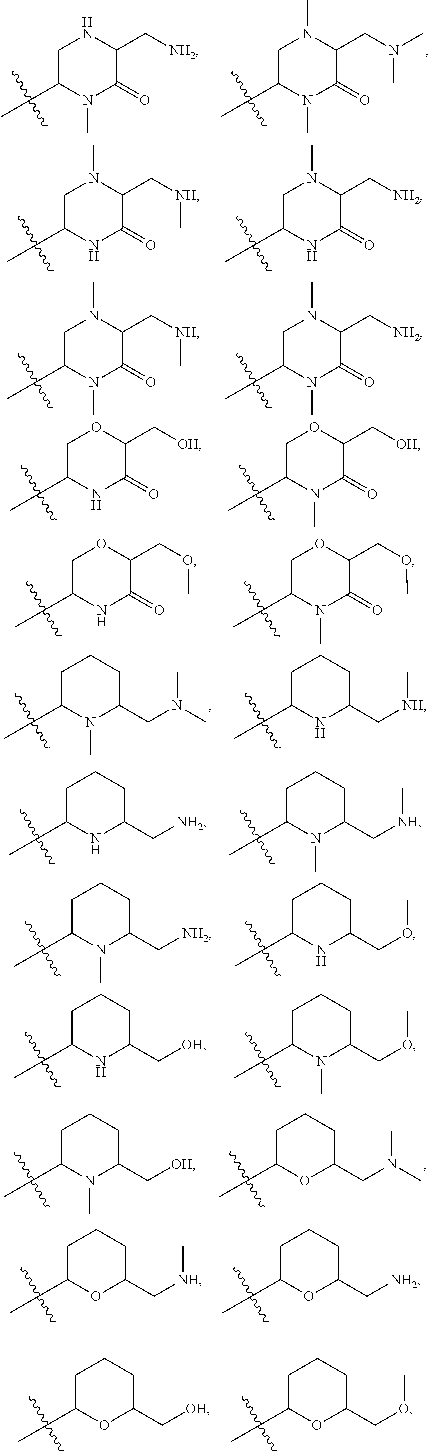

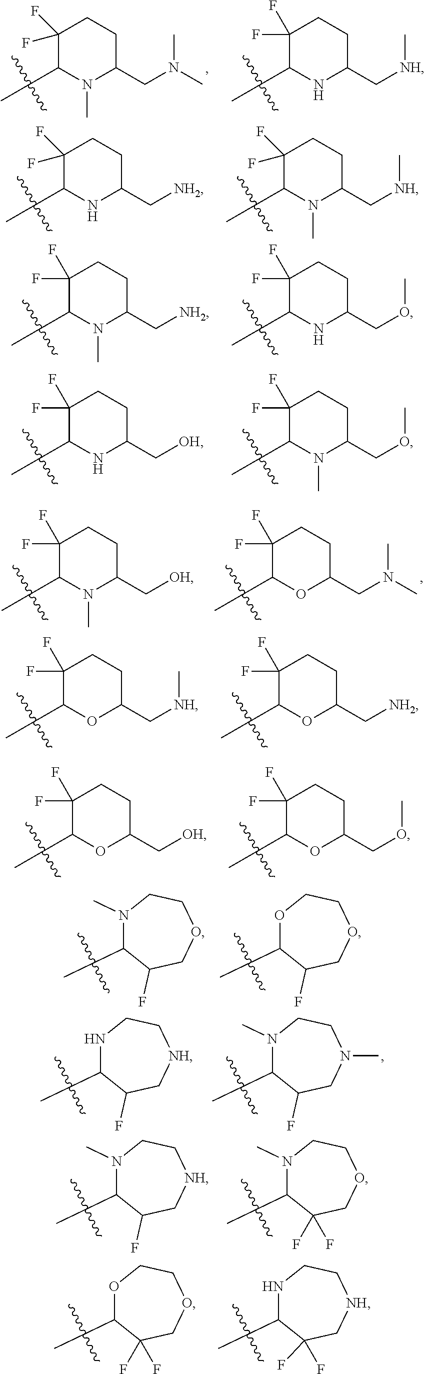

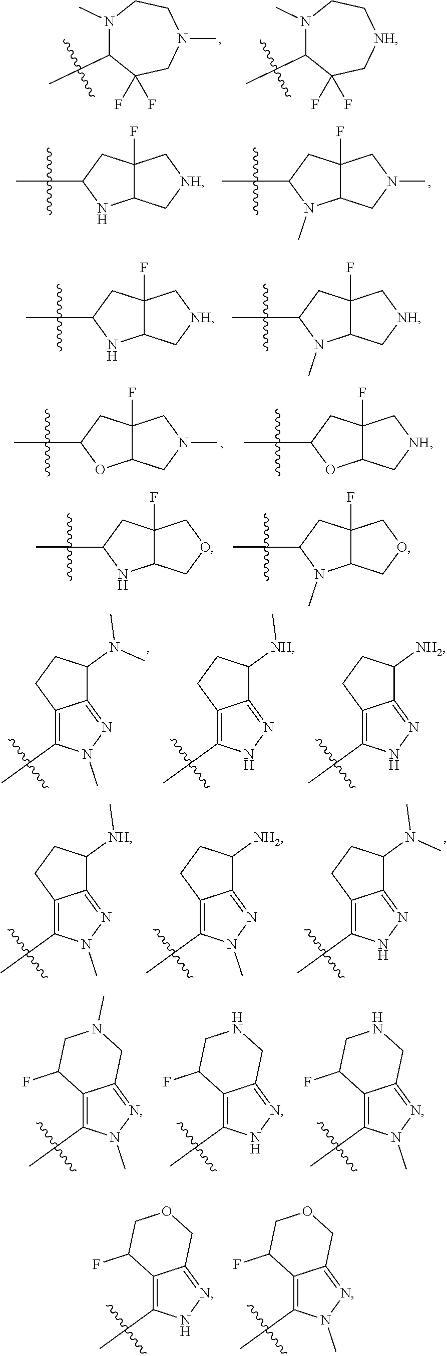

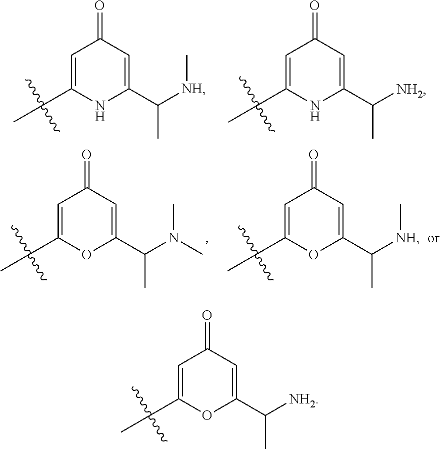

In some embodiments, R^{2B} is

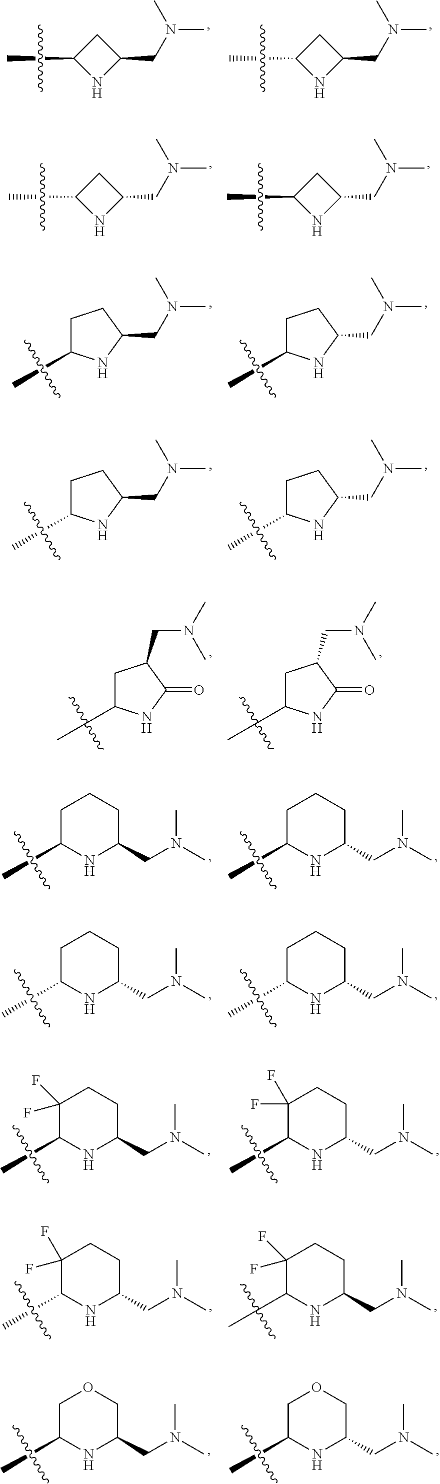

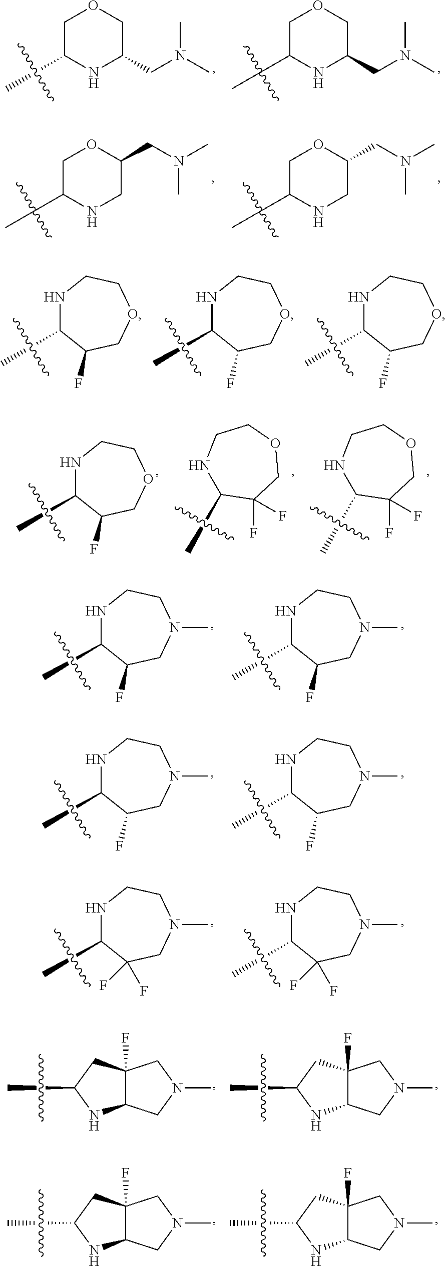

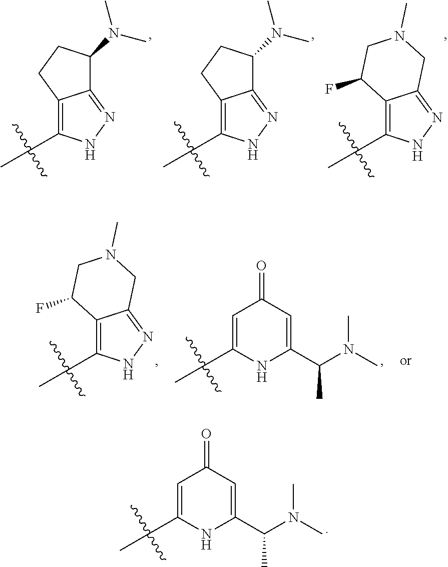 [SMILES: *c1[nH]nc2c1CC[C@H]2N(C)C ; *c1[nH]nc2c1CC[C@@H]2N(C)C ; *c1[nH]nc2c1[C@@H](F)CN(C)C2 ; *c1[nH]nc2c1[C@H](F)CN(C)C2 ; *c1cc(=O)cc([C@H](C)N(C)C)[nH]1 ; *c1cc(=O)cc([C@@H](C)N(C)C)[nH]1]

In some embodiments, R^{2B} is

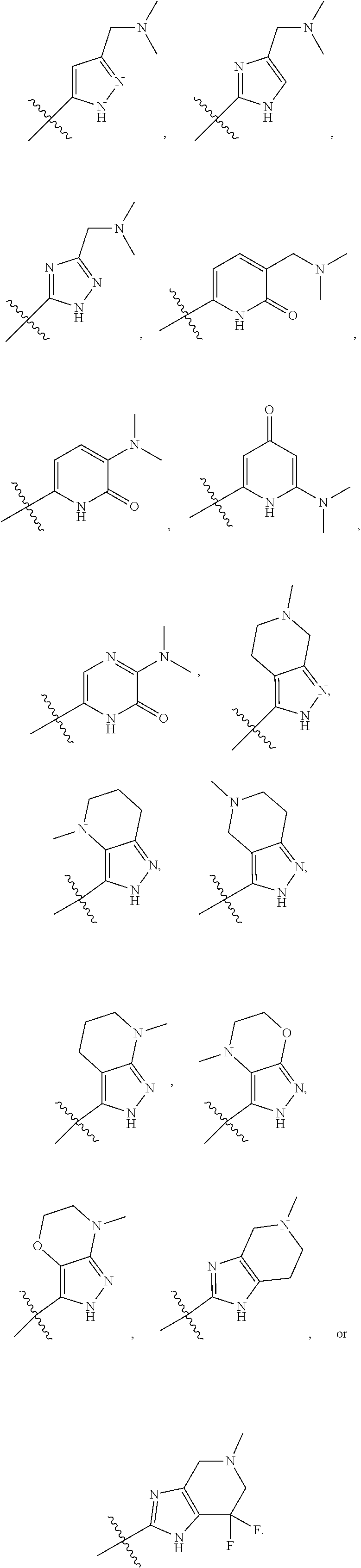

In some embodiments, R^{2B} is

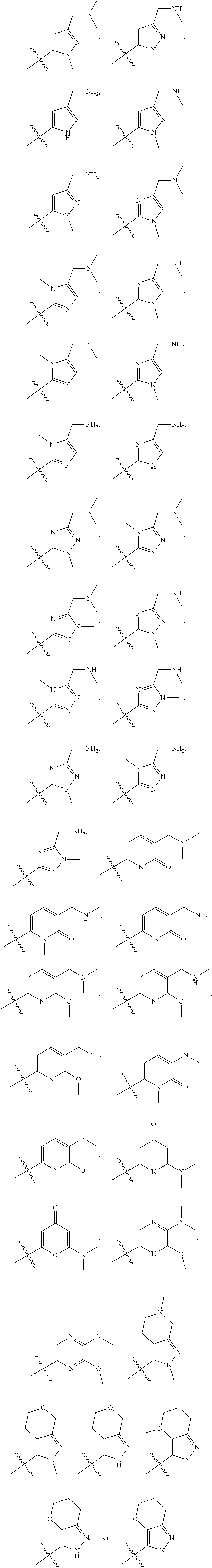

In some embodiments, R² as defined herein, comprises an α,β-unsaturated system or an electrophilic group.

In some embodiments, R² is selected from the group consisting of:

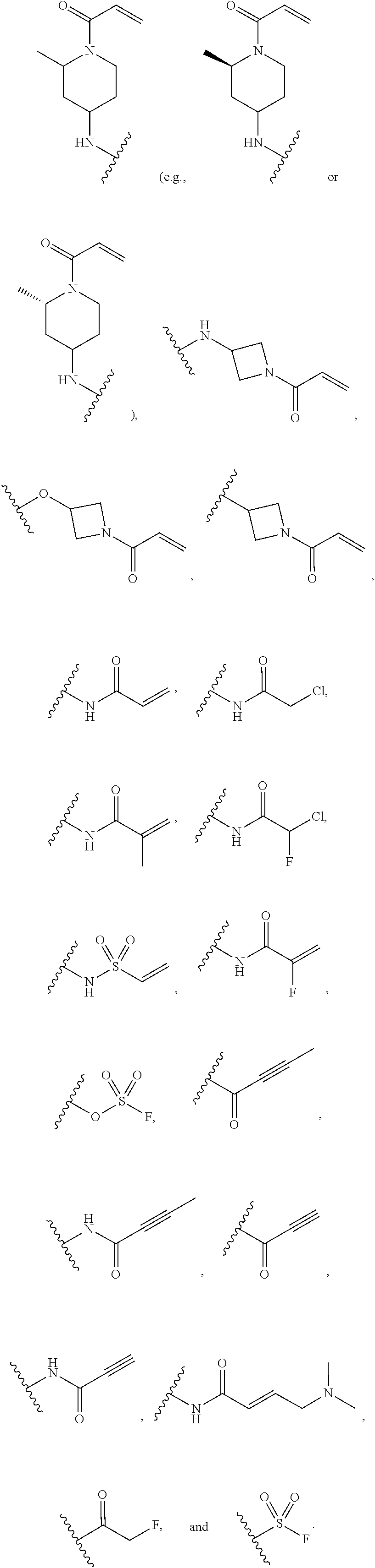

In some embodiments, X³ is CR² and R² as defined herein comprises an α,β-unsaturated system or an electrophilic group, as described herein.

In some embodiments, one of X², X³, X⁴, and X⁵ is CR², one of X², X³, X⁴, and X⁵ is CR³, and the remaining X², X³, X⁴, and X⁵ are CH or N.

In some embodiments, one of X², X³, X⁴, and X⁵ is CR², one of X², X³, X⁴, and X⁵ is CR³, and the remaining X², X³, X⁴, and X⁵ are CH.

In some embodiments, R³ is halogen. In some embodiments, R³ is fluoro. In some embodiments, R³ is chloro.

In some embodiments, R³ is cyano.

In some embodiments, R³ is —NR¹²R¹³.

In some embodiments, R³ is —OR¹².

In some embodiments, R³ is —C(═O)NR¹²R¹³.

In some embodiments, R³ is —C(═O)R¹².

In some embodiments, R³ is —C(═O)OR¹².

In some embodiments, R³ is —OC(═O)R¹².

In some embodiments, R³ is —NR¹²(C═O)NR¹²R¹³.

In some embodiments, R³ is —SR¹².

In some embodiments, R³ is —S(═O)R¹².

In some embodiments, R³ is —S(O₂)R¹².

In some embodiments, R³ is —S(O₂)NR¹²R¹³.

In some embodiments, R³ is —NR¹²S(O₂)NR¹³R¹⁴.

In some embodiments, R³ is —R¹²C(═O)R¹³.

In some embodiments, R³ is —NR¹²C(═O)R¹³.

In some embodiments, R¹² is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹² is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R¹² is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹² is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R¹² is hydrogen, C1-C6 alkyl, C3-C10 cycloalkyl, or 4-12 membered heterocyclyl. In some embodiments, R¹² is hydrogen, C1-C3 alkyl, C3-C6 cycloalkyl, or 4-8 membered heterocyclyl.

In some embodiments, R¹³ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹³ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R¹³ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹³ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R¹³ is hydrogen, C1-C6 alkyl, C3-C10 cycloalkyl, or 4-12 membered heterocyclyl. In some embodiments, R¹³ is hydrogen, C1-C3 alkyl, C3-C6 cycloalkyl, or 4-8 membered heterocyclyl.

In some embodiments, R¹⁴ is hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, R¹⁴ is hydrogen, optionally substituted C1-C3 alkyl, optionally substituted C2-C3 alkenyl, optionally substituted C2-C3 alkynyl, optionally substituted C3-C6 cycloalkyl, optionally substituted phenyl, optionally substituted 4-8 membered heterocyclyl, or optionally substituted 5-6 membered heteroaryl.

In some embodiments, R¹⁴ is hydrogen, C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R¹⁴ is hydrogen, C1-C3 alkyl, C2-C3 alkenyl, C2-C3 alkynyl, C3-C6 cycloalkyl, phenyl, 4-8 membered heterocyclyl, or 5-6 membered heteroaryl.

In some embodiments, R¹⁴ is hydrogen, C1-C6 alkyl, C3-C10 cycloalkyl, or 4-12 membered heterocyclyl. In some embodiments, R¹⁴ is hydrogen, C1-C3 alkyl, C3-C6 cycloalkyl, or 4-8 membered heterocyclyl.

In some embodiments, when R¹² and R¹³ are attached to the same nitrogen atom, R¹² and R¹³ are the same. In some embodiments, when R¹² and R¹³ are attached to the same nitrogen atom, R¹² and R¹³ are different. In some embodiments, when R¹² and R¹³ are attached to the same nitrogen atom, R¹² and R¹³ are each hydrogen. In some embodiments, when R¹² and R¹³ are attached to the same nitrogen atom, R¹² and R¹³ are each an independently selected C1-C6 alkyl.

In some embodiments, when R¹² and R¹³ are attached to the same nitrogen atom, one of R¹² and R¹³ is hydrogen and the other of R¹² and R¹³ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, when R¹² and R¹³ are attached to the same nitrogen atom, one of R¹² and R¹³ is hydrogen and the other of R¹² and R¹³ is C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, when R¹³ and R¹⁴ are attached to the same nitrogen atom, R¹³ and R⁴ are the same. In some embodiments, when R¹³ and R¹⁴ are attached to the same nitrogen atom, R¹³ and R¹⁴ are different. In some embodiments, when R¹³ and R¹⁴ are attached to the same nitrogen atom, R¹³ and R¹⁴ are each hydrogen. In some embodiments, when R¹³ and R¹⁴ are attached to the same nitrogen atom, R¹³ and R¹⁴ are each an independently selected C1-C6 alkyl.

In some embodiments, when R¹³ and R¹⁴ are attached to the same nitrogen atom, one of R¹³ and R¹⁴ is hydrogen and the other of R¹³ and R¹⁴ is optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

In some embodiments, when R¹³ and R″ are attached to the same nitrogen atom, one of R¹³ and R¹ is hydrogen and the other of R¹³ and R¹⁴ is C1-C6 alkyl, C2-C6 alkenyl, C2-C6 alkynyl, C3-C10 cycloalkyl, phenyl, 4-12 membered heterocyclyl, or 5-10 membered heteroaryl.

In some embodiments, R³ is optionally substituted C1-C6 alkyl. In some embodiments, R³ is C1-C6 alkyl. In some embodiments, R³ is methyl or ethyl.

In some embodiments, R³ is optionally substituted C2-C6 alkenyl. In some embodiments, R³ is C2-C6 alkenyl. In some embodiments, R³ is optionally substituted C2-C3 alkenyl. In some embodiments, R³ is C2-C3 alkenyl.

In some embodiments, R³ is optionally substituted C2-C6 alkynyl. In some embodiments, R³ is C2-C6 alkynyl. In some embodiments, R³ is optionally substituted C2-C3 alkynyl. In some embodiments, R³ is C2-C3 alkynyl.

In some embodiments, R³ is optionally substituted C3-C6 cycloalkyl. In some embodiments, R³ is C3-C6 cycloalkyl.

In some embodiments, R³ is optionally substituted phenyl. In some embodiments, R³ is phenyl.

In some embodiments, R³ is optionally substituted 4-6 membered heterocyclyl. In some embodiments, R³ is 4-6 membered heterocyclyl.

In some embodiments, R³ is optionally substituted 5-6 membered heteroaryl. In some embodiments, R³ is 5-6 membered heteroaryl.

In some embodiments, m is 0. In some embodiments, m is 1. In some embodiments, m is 2.

In some embodiments, L is an optionally substituted 5-6 membered heteroarylene. In some embodiments, L is a 5-6 membered heteroarylene. In some embodiments, L is a substituted 6 membered heteroarylene. In some embodiments, L is a 5 membered heteroarylene. In some embodiments, L is a 2,5-(1,3,4-oxadiazolyl)ene, a 2,5-(1,3,4-thiadiazolyl)ene, or a 3,5-(1,2,4-oxadiazolyl)ene. In some embodiments, L is

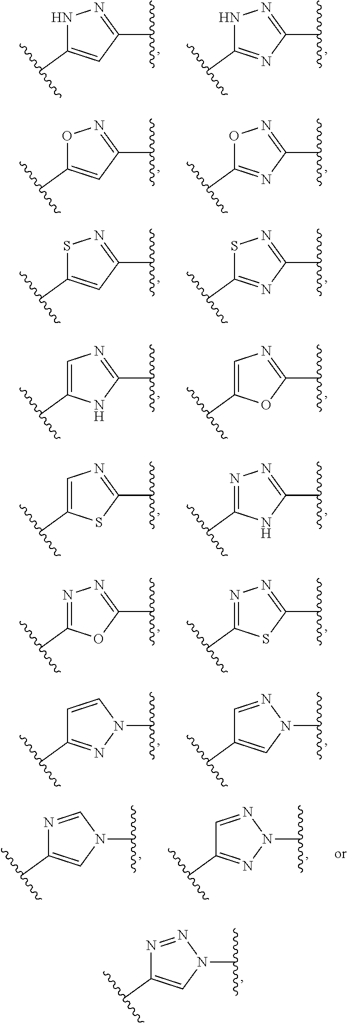

wherein each [symbol] represents a bond to either the carbon of the 5-membered ring of Formula (I) or the CH₂ or R^{A} of Formula (I).

In some embodiments, L is an optionally substituted 4-6 membered heterocyclylene. In some embodiments, L is a 4-6 membered heterocyclylene. In some embodiments, L is a 4 membered heterocyclylene.

### Non-Limiting Exemplary Compounds

In some embodiments, the compound is selected from the group consisting of the compounds delineated in Table 1, or a pharmaceutically acceptable salt thereof.

TABLE 1

| Example | |
|---|---|
| No. | Structure |
| 1 | 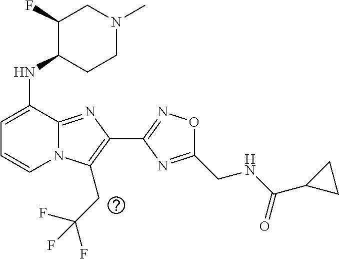 [SMILES: CN1CC[C@@H](Nc2cccn3c(CC(F)(F)F)c(-c4noc(CNC(=O)C5CC5)n4)nc23)[C@@H](F)C1] |
| 2 | 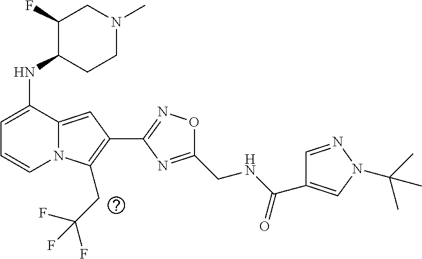 [SMILES: CN1CC[C@@H](Nc2cccn3c(CC(F)(F)F)c(-c4noc(CNC(=O)c5cnn(C(C)(C)C)c5)n4)cc23)[C@@H](F)C1] |
| 3 | 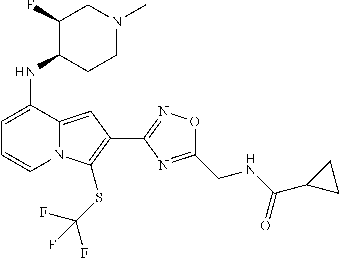 [SMILES: CN1CC[C@@H](Nc2cccn3c(SC(F)(F)F)c(-c4noc(CNC(=O)C5CC5)n4)cc23)[C@@H](F)C1] |
| 4 | 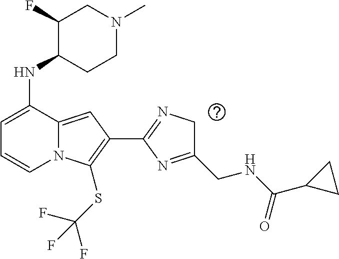 [SMILES: CN1CC[C@@H](Nc2cccn3c(SC(F)(F)F)c(C4=NCC(CNC(=O)C5CC5)=N4)cc23)[C@@H](F)C1] |
| 5 | 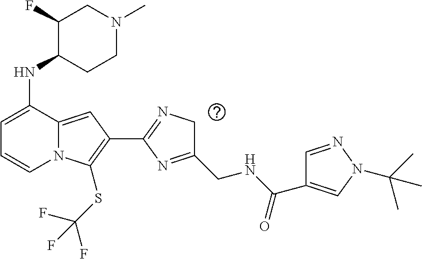 [SMILES: CN1CC[C@@H](Nc2cccn3c(SC(F)(F)F)c(C4=NCC(CNC(=O)c5cnn(C(C)(C)C)c5)=N4)cc23)[C@@H](F)C1] |
| 6 | 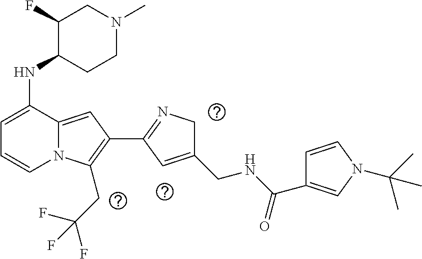 [SMILES: CN1CC[C@@H](Nc2cccn3c(CC(F)(F)F)c(C4=NCC(CNC(=O)c5ccn(C(C)(C)C)c5)=C4)cc23)[C@@H](F)C1] |
| 7 | 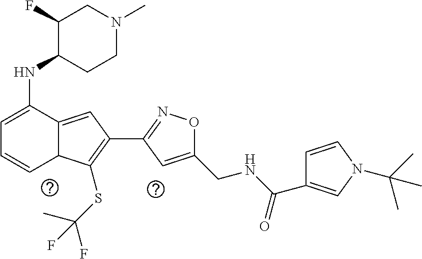 [SMILES: CN1CC[C@@H](NC2=CC=CC3C2=CC(c2cc(CNC(=O)c4ccn(C(C)(C)C)c4)on2)=C3SC(C)(F)F)[C@@H](F)C1] |
| 8 | 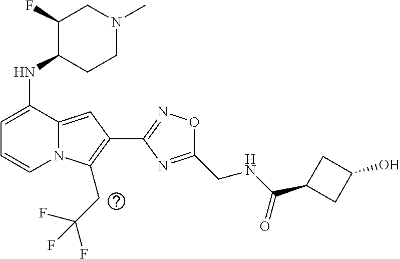 [SMILES: CN1CC[C@@H](Nc2cccn3c(CC(F)(F)F)c(-c4noc(CNC(=O)[C@H]5C[C@H](O)C5)n4)cc23)[C@@H](F)C1] |
| 9 | 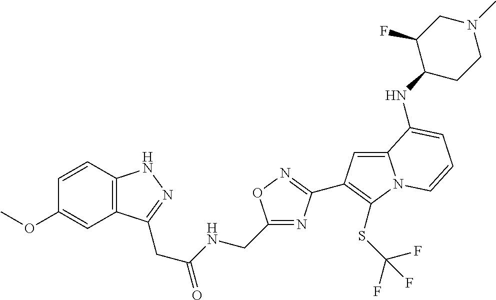 |
| 10 | 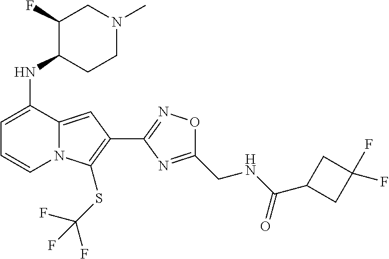 [SMILES: CN1CC[C@@H](Nc2cccn3c(SC(F)(F)F)c(-c4noc(CNC(=O)C5CC(F)(F)C5)n4)cc23)[C@@H](F)C1] |
| 11 | 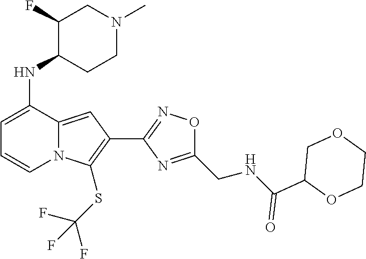 [SMILES: CN1CC[C@@H](Nc2cccn3c(SC(F)(F)F)c(-c4noc(CNC(=O)C5COCCO5)n4)cc23)[C@@H](F)C1] |
| 12 | 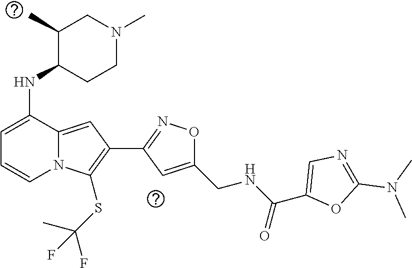 [SMILES: C[C@H]1CN(C)CC[C@H]1Nc1cccn2c(SC(C)(F)F)c(-c3cc(CNC(=O)c4cnc(N(C)C)o4)on3)cc12] |
| 13 | 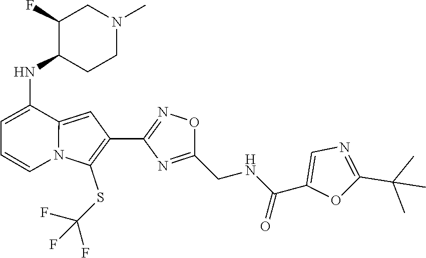 [SMILES: CN1CC[C@@H](Nc2cccn3c(SC(F)(F)F)c(-c4noc(CNC(=O)c5cnc(C(C)(C)C)o5)n4)cc23)[C@@H](F)C1] |
| 14 | 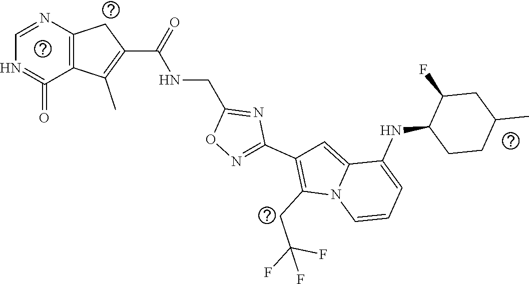 |
| 15 | 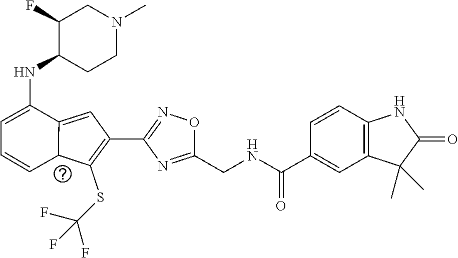 [SMILES: CN1CC[C@@H](NC2=CC=CC3C2=CC(c2noc(CNC(=O)c4ccc5c(c4)C(C)(C)C(=O)N5)n2)=C3SC(F)(F)F)[C@@H](F)C1] |
| 16 | 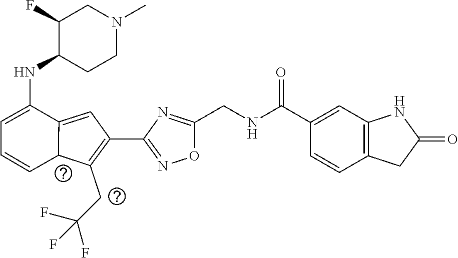 [SMILES: CN1CC[C@@H](NC2=CC=CC3C2=CC(c2noc(CNC(=O)c4ccc5c(c4)NC(=O)C5)n2)=C3CC(F)(F)F)[C@@H](F)C1] |
| 17 | 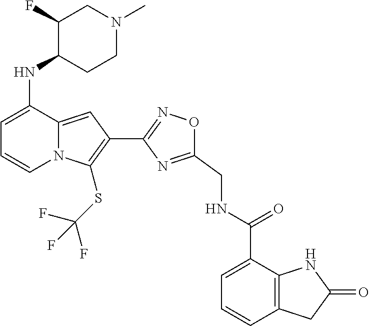 [SMILES: CN1CC[C@@H](Nc2cccn3c(SC(F)(F)F)c(-c4noc(CNC(=O)c5cccc6c5NC(=O)C6)n4)cc23)[C@@H](F)C1] |
| 18 | 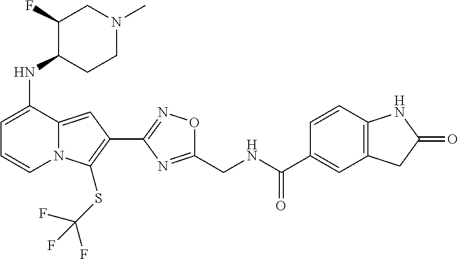 [SMILES: CN1CC[C@@H](Nc2cccn3c(SC(F)(F)F)c(-c4noc(CNC(=O)c5ccc6c(c5)CC(=O)N6)n4)cc23)[C@@H](F)C1] |
| 19 | 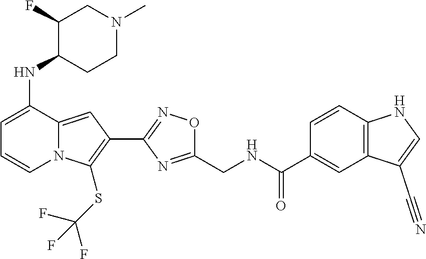 [SMILES: CN1CC[C@@H](Nc2cccn3c(SC(F)(F)F)c(-c4noc(CNC(=O)c5ccc6[nH]cc(C#N)c6c5)n4)cc23)[C@@H](F)C1] |
| 20 | 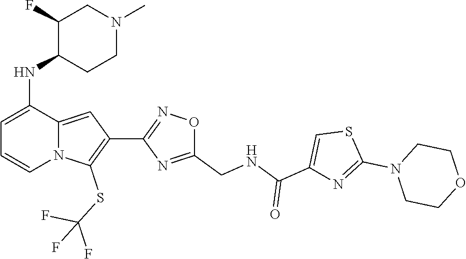 [SMILES: CN1CC[C@@H](Nc2cccn3c(SC(F)(F)F)c(-c4noc(CNC(=O)c5csc(N6CCOCC6)n5)n4)cc23)[C@@H](F)C1] |
| 21 | 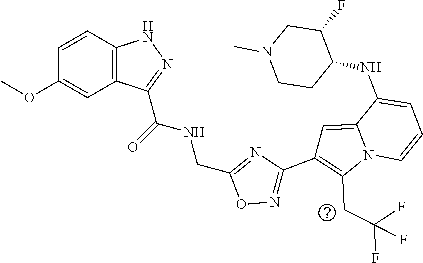 [SMILES: COc1ccc2[nH]nc(C(=O)NCc3nc(-c4cc5c(N[C@@H]6CCN(C)C[C@@H]6F)cccn5c4CC(F)(F)F)no3)c2c1] |
| 22 | 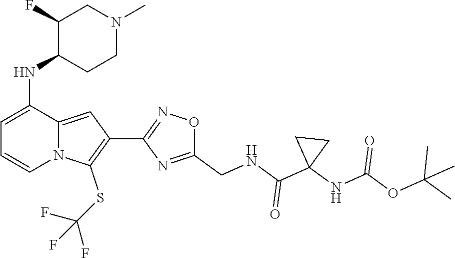 [SMILES: CN1CC[C@@H](Nc2cccn3c(SC(F)(F)F)c(-c4noc(CNC(=O)C5(NC(=O)OC(C)(C)C)CC5)n4)cc23)[C@@H](F)C1] |
| 23 | 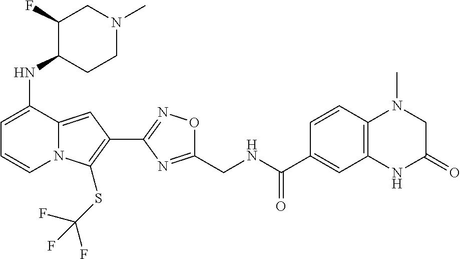 [SMILES: CN1CC[C@@H](Nc2cccn3c(SC(F)(F)F)c(-c4noc(CNC(=O)c5ccc6c(c5)NC(=O)CN6C)n4)cc23)[C@@H](F)C1] |
| 24 | 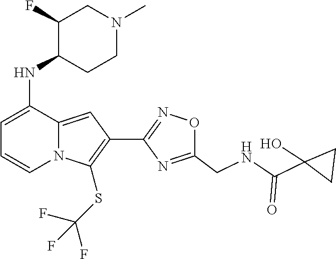 [SMILES: CN1CC[C@@H](Nc2cccn3c(SC(F)(F)F)c(-c4noc(CNC(=O)C5(O)CC5)n4)cc23)[C@@H](F)C1] |
| 25 | 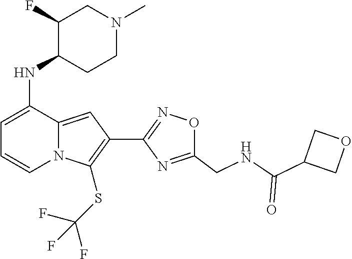 [SMILES: CN1CC[C@@H](Nc2cccn3c(SC(F)(F)F)c(-c4noc(CNC(=O)C5COC5)n4)cc23)[C@@H](F)C1] |
| 26 | 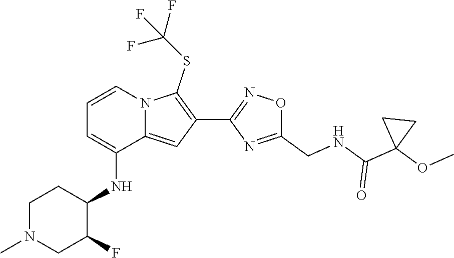 |
| 27 | 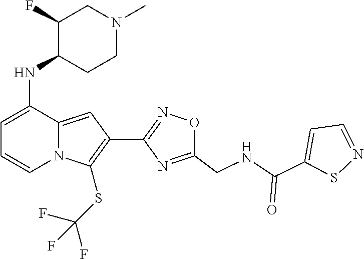 [SMILES: CN1CC[C@@H](Nc2cccn3c(SC(F)(F)F)c(-c4noc(CNC(=O)c5ccns5)n4)cc23)[C@@H](F)C1] |
| 28 | 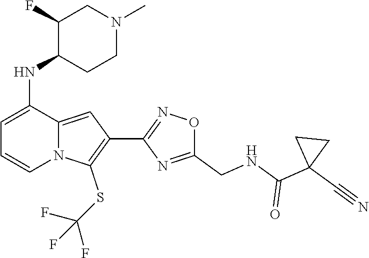 [SMILES: CN1CC[C@@H](Nc2cccn3c(SC(F)(F)F)c(-c4noc(CNC(=O)C5(C#N)CC5)n4)cc23)[C@@H](F)C1] |
| 29 | 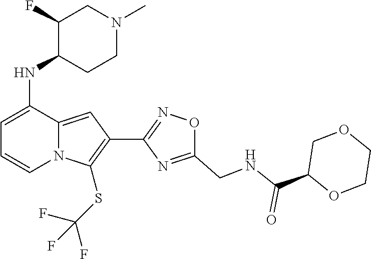 [SMILES: CN1CC[C@@H](Nc2cccn3c(SC(F)(F)F)c(-c4noc(CNC(=O)[C@H]5COCCO5)n4)cc23)[C@@H](F)C1] |
| 30 | 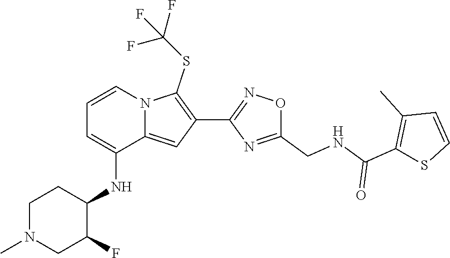 [SMILES: Cc1ccsc1C(=O)NCc1nc(-c2cc3c(N[C@@H]4CCN(C)C[C@@H]4F)cccn3c2SC(F)(F)F)no1] |
| 31 | 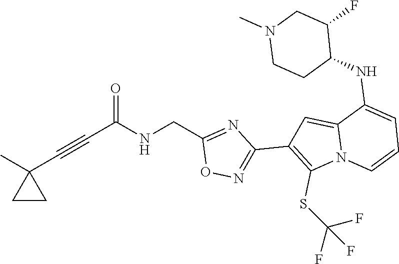 [SMILES: CN1CC[C@@H](Nc2cccn3c(SC(F)(F)F)c(-c4noc(CNC(=O)C#CC5(C)CC5)n4)cc23)[C@@H](F)C1] |
| 32 | 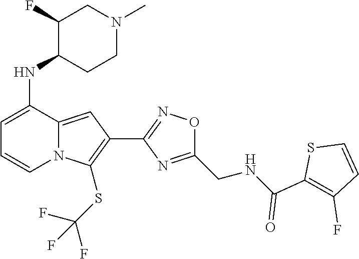 [SMILES: CN1CC[C@@H](Nc2cccn3c(SC(F)(F)F)c(-c4noc(CNC(=O)c5sccc5F)n4)cc23)[C@@H](F)C1] |
| 33 | 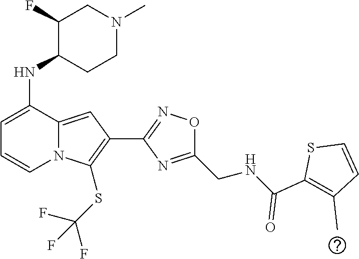 [SMILES: Cc1ccsc1C(=O)NCc1nc(-c2cc3c(N[C@@H]4CCN(C)C[C@@H]4F)cccn3c2SC(F)(F)F)no1] |
| 34 | 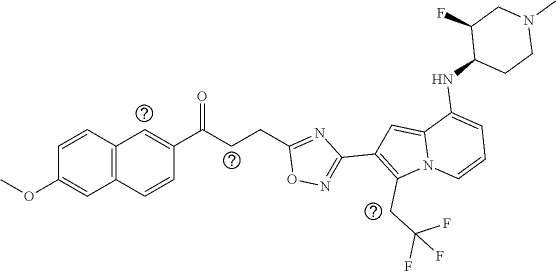 |
| 35 | 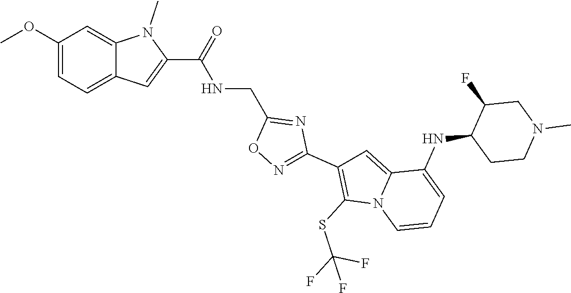 |
| 36 | 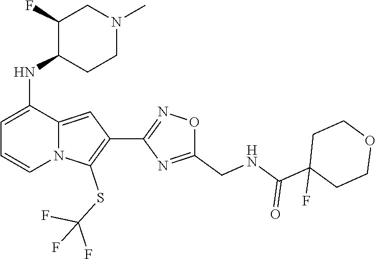 [SMILES: CN1CC[C@@H](Nc2cccn3c(SC(F)(F)F)c(-c4noc(CNC(=O)C5(F)CCOCC5)n4)cc23)[C@@H](F)C1] |
| 37 | 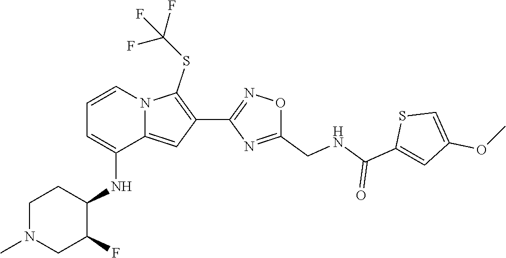 |
| 38 | 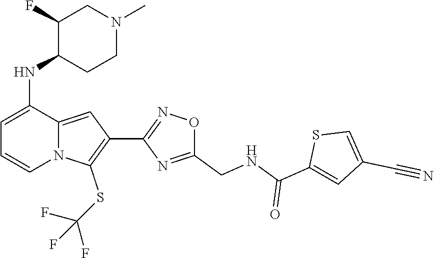 [SMILES: CN1CC[C@@H](Nc2cccn3c(SC(F)(F)F)c(-c4noc(CNC(=O)c5cc(C#N)cs5)n4)cc23)[C@@H](F)C1] |
| 39 | 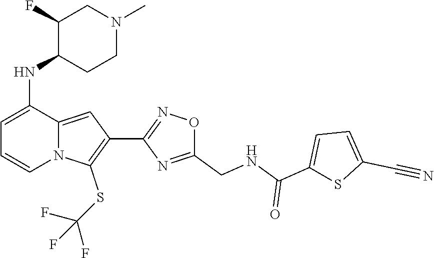 [SMILES: CN1CC[C@@H](Nc2cccn3c(SC(F)(F)F)c(-c4noc(CNC(=O)c5ccc(C#N)s5)n4)cc23)[C@@H](F)C1] |
| 40 | 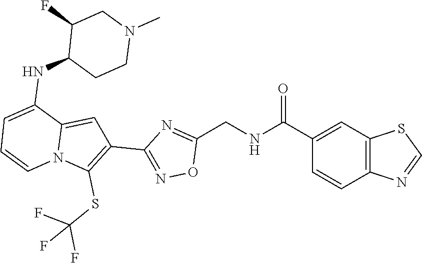 [SMILES: CN1CC[C@@H](Nc2cccn3c(SC(F)(F)F)c(-c4noc(CNC(=O)c5ccc6ncsc6c5)n4)cc23)[C@@H](F)C1] |
| 41 | 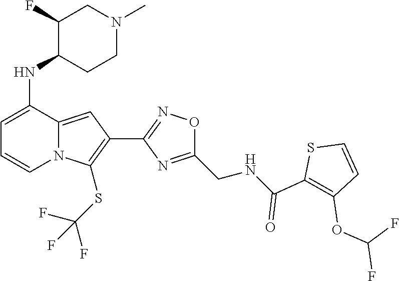 [SMILES: CN1CC[C@@H](Nc2cccn3c(SC(F)(F)F)c(-c4noc(CNC(=O)c5sccc5OC(F)F)n4)cc23)[C@@H](F)C1] |
| 42 | 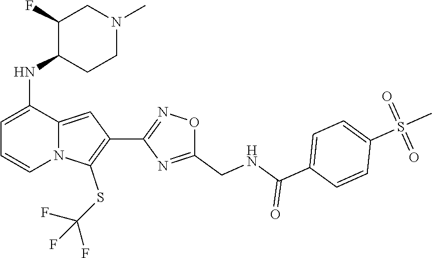 [SMILES: CN1CC[C@@H](Nc2cccn3c(SC(F)(F)F)c(-c4noc(CNC(=O)c5ccc(S(C)(=O)=O)cc5)n4)cc23)[C@@H](F)C1] |
| 43 | 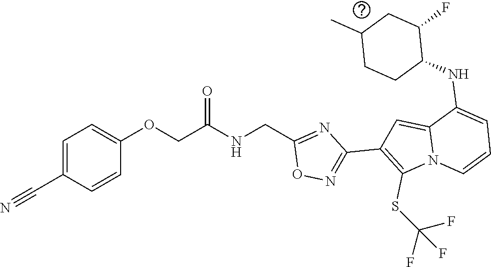 [SMILES: CC1CC[C@@H](Nc2cccn3c(SC(F)(F)F)c(-c4noc(CNC(=O)COc5ccc(C#N)cc5)n4)cc23)[C@@H](F)C1] |
| 44 | 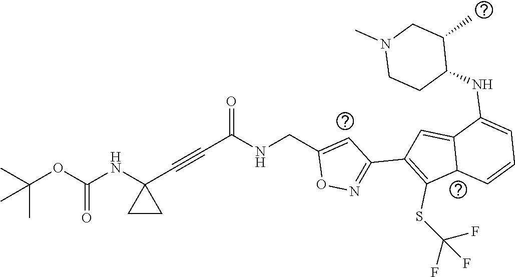 [SMILES: C[C@H]1CN(C)CC[C@H]1NC1=CC=CC2C1=CC(c1cc(CNC(=O)C#CC3(NC(=O)OC(C)(C)C)CC3)on1)=C2SC(F)(F)F] |
| 45 | 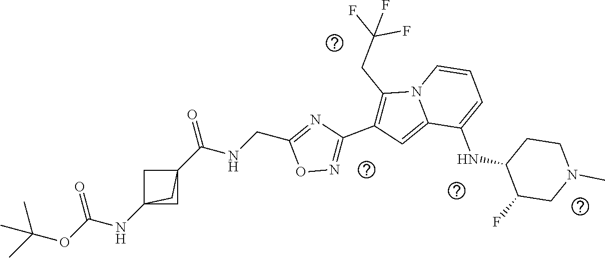 [SMILES: CN1CC[C@@H](Nc2cccn3c(CC(F)(F)F)c(-c4noc(CNC(=O)C56CC(NC(=O)OC(C)(C)C)(C5)C6)n4)cc23)[C@@H](F)C1] |
| 46 | 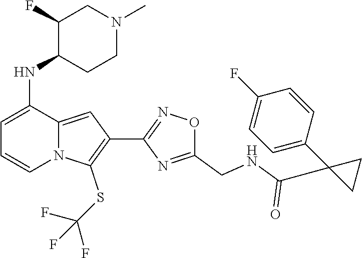 [SMILES: CN1CC[C@@H](Nc2cccn3c(SC(F)(F)F)c(-c4noc(CNC(=O)C5(c6ccc(F)cc6)CC5)n4)cc23)[C@@H](F)C1] |
| 47 | 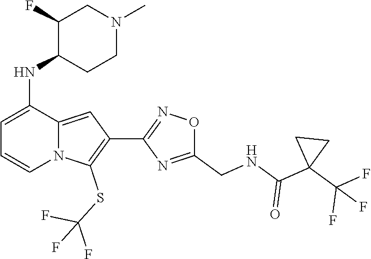 [SMILES: CN1CC[C@@H](Nc2cccn3c(SC(F)(F)F)c(-c4noc(CNC(=O)C5(C(F)(F)F)CC5)n4)cc23)[C@@H](F)C1] |
| 48 | 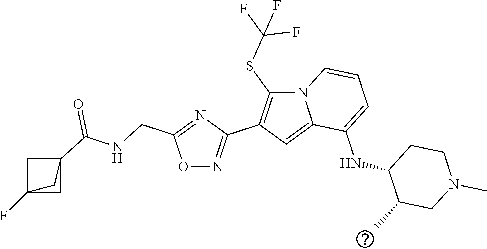 [SMILES: C[C@H]1CN(C)CC[C@H]1Nc1cccn2c(SC(F)(F)F)c(-c3noc(CNC(=O)C45CC(F)(C4)C5)n3)cc12] |
| 49 | 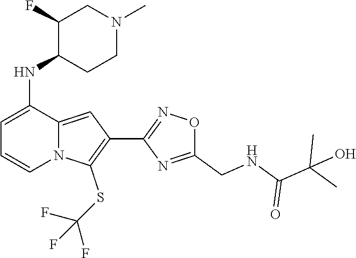 [SMILES: CN1CC[C@@H](Nc2cccn3c(SC(F)(F)F)c(-c4noc(CNC(=O)C(C)(C)O)n4)cc23)[C@@H](F)C1] |
| 50 | 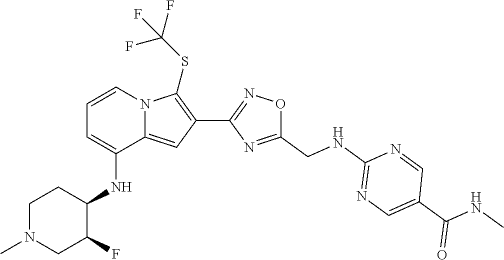 |
| 51 | 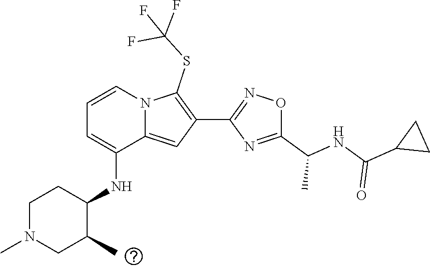 [SMILES: C[C@@H](NC(=O)C1CC1)c1nc(-c2cc3c(N[C@@H]4CCN(C)C[C@@H]4C)cccn3c2SC(F)(F)F)no1] |
| 52 | 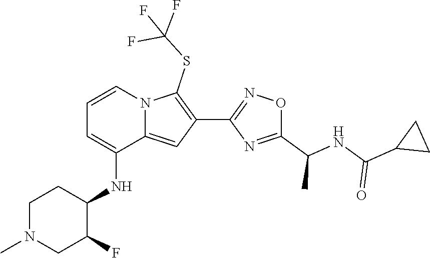 [SMILES: C[C@H](NC(=O)C1CC1)c1nc(-c2cc3c(N[C@@H]4CCN(C)C[C@@H]4F)cccn3c2SC(F)(F)F)no1] |
| 53 | 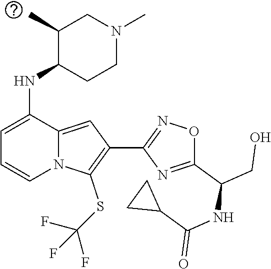 [SMILES: C[C@H]1CN(C)CC[C@H]1Nc1cccn2c(SC(F)(F)F)c(-c3noc([C@@H](CO)NC(=O)C4CC4)n3)cc12] |
| 54 | 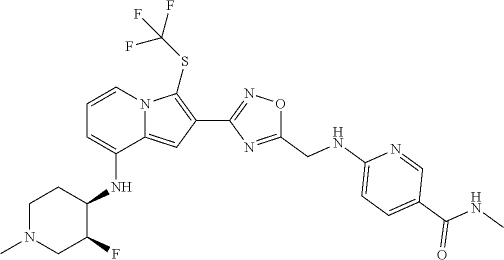 |
| 55 | 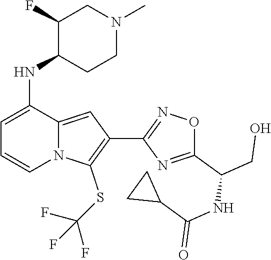 [SMILES: CN1CC[C@@H](Nc2cccn3c(SC(F)(F)F)c(-c4noc([C@H](CO)NC(=O)C5CC5)n4)cc23)[C@@H](F)C1] |
| 56 | 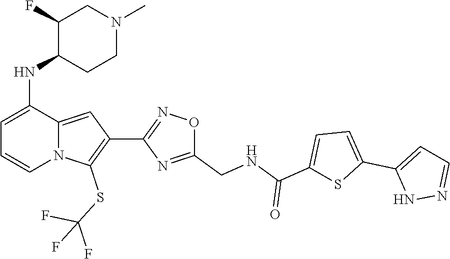 [SMILES: CN1CC[C@@H](Nc2cccn3c(SC(F)(F)F)c(-c4noc(CNC(=O)c5ccc(-c6ccn[nH]6)s5)n4)cc23)[C@@H](F)C1] |
| 57 | 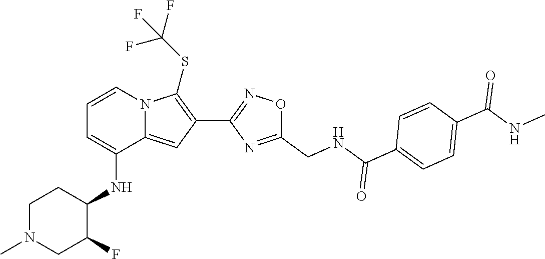 |
| 58 | 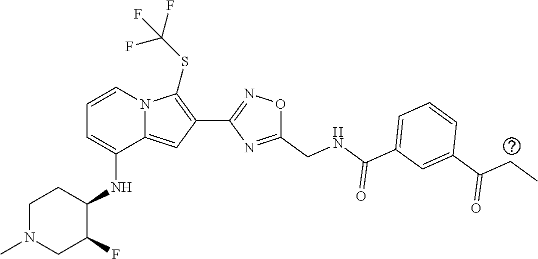 [SMILES: CCC(=O)c1cccc(C(=O)NCc2nc(-c3cc4c(N[C@@H]5CCN(C)C[C@@H]5F)cccn4c3SC(F)(F)F)no2)c1] |
| 59 | 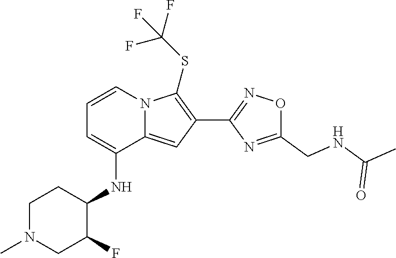 [SMILES: CC(=O)NCc1nc(-c2cc3c(N[C@@H]4CCN(C)C[C@@H]4F)cccn3c2SC(F)(F)F)no1] |
| 60 | 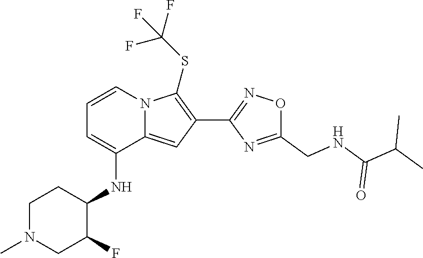 [SMILES: CC(C)C(=O)NCc1nc(-c2cc3c(N[C@@H]4CCN(C)C[C@@H]4F)cccn3c2SC(F)(F)F)no1] |
| 61 | 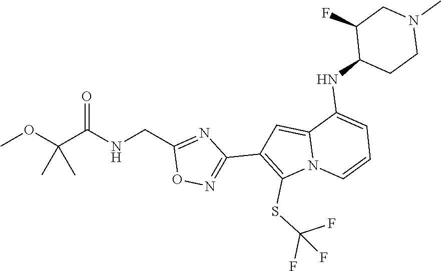 |
| 62 | 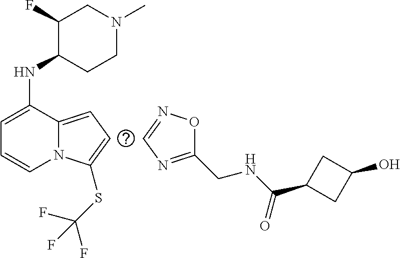 |
| 63 | 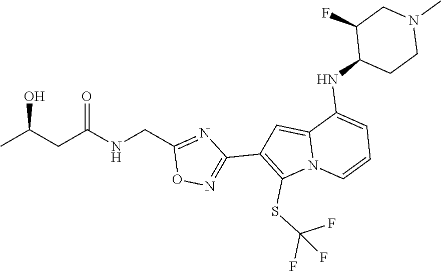 [SMILES: C[C@@H](O)CC(=O)NCc1nc(-c2cc3c(N[C@@H]4CCN(C)C[C@@H]4F)cccn3c2SC(F)(F)F)no1] |
| 64 | 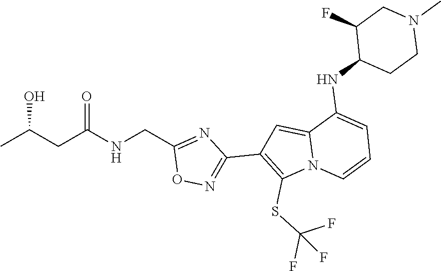 [SMILES: C[C@H](O)CC(=O)NCc1nc(-c2cc3c(N[C@@H]4CCN(C)C[C@@H]4F)cccn3c2SC(F)(F)F)no1] |
| 65 | 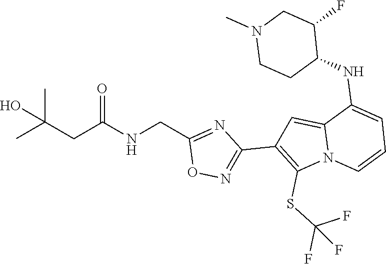 [SMILES: CN1CC[C@@H](Nc2cccn3c(SC(F)(F)F)c(-c4noc(CNC(=O)CC(C)(C)O)n4)cc23)[C@@H](F)C1] |
| 66 | 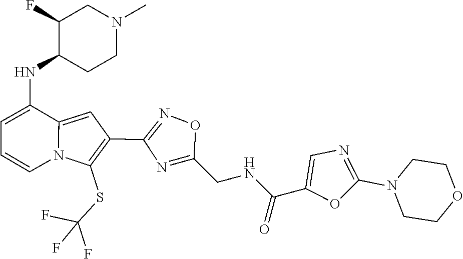 [SMILES: CN1CC[C@@H](Nc2cccn3c(SC(F)(F)F)c(-c4noc(CNC(=O)c5cnc(N6CCOCC6)o5)n4)cc23)[C@@H](F)C1] |
| 67 | 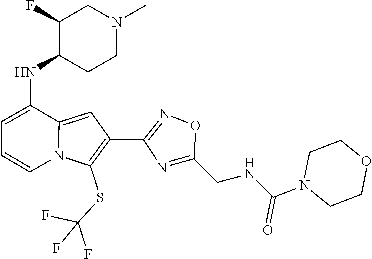 [SMILES: CN1CC[C@@H](Nc2cccn3c(SC(F)(F)F)c(-c4noc(CNC(=O)N5CCOCC5)n4)cc23)[C@@H](F)C1] |
| 68 | 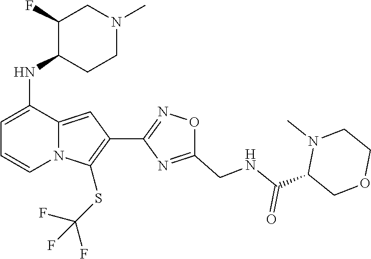 [SMILES: CN1CC[C@@H](Nc2cccn3c(SC(F)(F)F)c(-c4noc(CNC(=O)[C@H]5COCCN5C)n4)cc23)[C@@H](F)C1] |
| 69 | 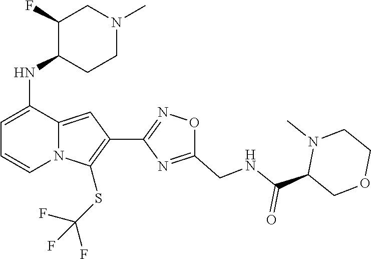 [SMILES: CN1CC[C@@H](Nc2cccn3c(SC(F)(F)F)c(-c4noc(CNC(=O)[C@@H]5COCCN5C)n4)cc23)[C@@H](F)C1] |
| 70 | 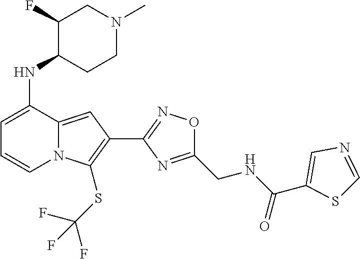 [SMILES: CN1CC[C@@H](Nc2cccn3c(SC(F)(F)F)c(-c4noc(CNC(=O)c5cncs5)n4)cc23)[C@@H](F)C1] |
| 71 | 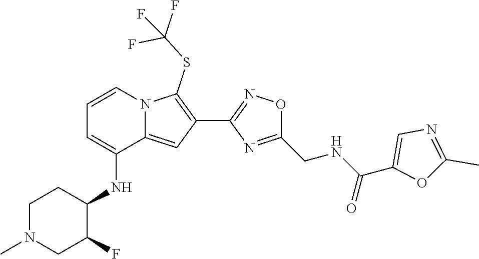 |
| 72 | 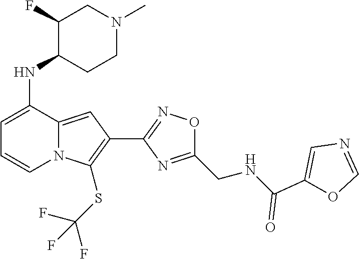 [SMILES: CN1CC[C@@H](Nc2cccn3c(SC(F)(F)F)c(-c4noc(CNC(=O)c5cnco5)n4)cc23)[C@@H](F)C1] |
| 73 | 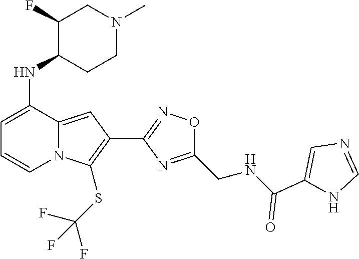 [SMILES: CN1CC[C@@H](Nc2cccn3c(SC(F)(F)F)c(-c4noc(CNC(=O)c5cnc[nH]5)n4)cc23)[C@@H](F)C1] |
| 74 | 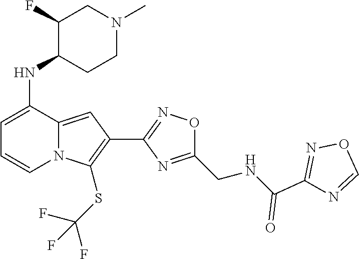 [SMILES: CN1CC[C@@H](Nc2cccn3c(SC(F)(F)F)c(-c4noc(CNC(=O)c5ncon5)n4)cc23)[C@@H](F)C1] |
| 75 | 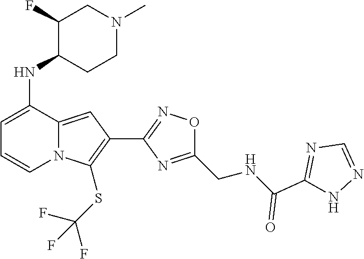 [SMILES: CN1CC[C@@H](Nc2cccn3c(SC(F)(F)F)c(-c4noc(CNC(=O)c5ncn[nH]5)n4)cc23)[C@@H](F)C1] |
| 76 | 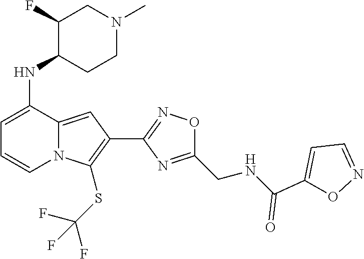 [SMILES: CN1CC[C@@H](Nc2cccn3c(SC(F)(F)F)c(-c4noc(CNC(=O)c5ccno5)n4)cc23)[C@@H](F)C1] |
| 77 | 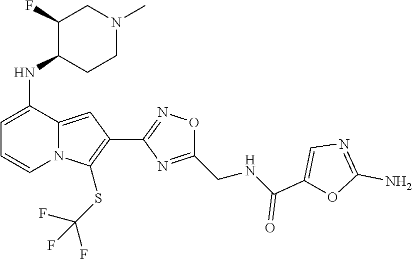 [SMILES: CN1CC[C@@H](Nc2cccn3c(SC(F)(F)F)c(-c4noc(CNC(=O)c5cnc(N)o5)n4)cc23)[C@@H](F)C1] |
| 78 | 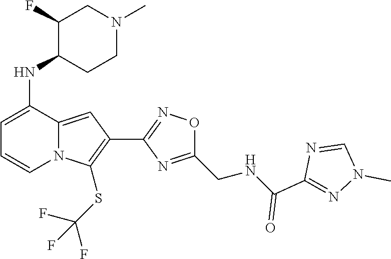 [SMILES: CN1CC[C@@H](Nc2cccn3c(SC(F)(F)F)c(-c4noc(CNC(=O)c5ncn(C)n5)n4)cc23)[C@@H](F)C1] |
| 79 | 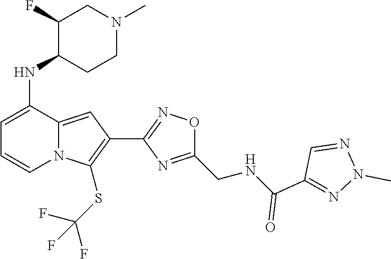 [SMILES: CN1CC[C@@H](Nc2cccn3c(SC(F)(F)F)c(-c4noc(CNC(=O)c5cnn(C)n5)n4)cc23)[C@@H](F)C1] |
| 80 | 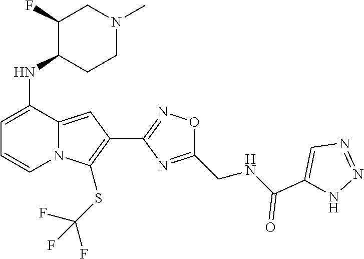 [SMILES: CN1CC[C@@H](Nc2cccn3c(SC(F)(F)F)c(-c4noc(CNC(=O)c5cnn[nH]5)n4)cc23)[C@@H](F)C1] |
| 81 | 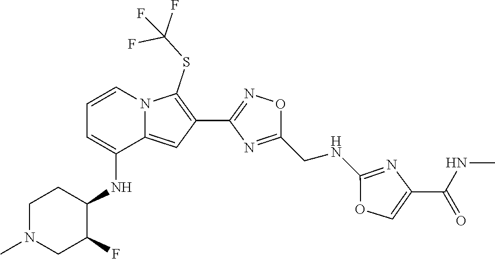 |
| 82 | 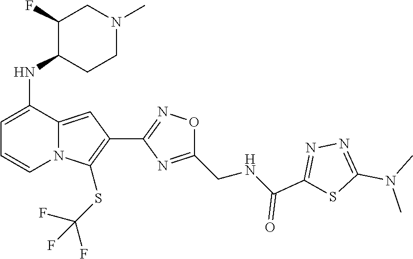 [SMILES: CN1CC[C@@H](Nc2cccn3c(SC(F)(F)F)c(-c4noc(CNC(=O)c5nnc(N(C)C)s5)n4)cc23)[C@@H](F)C1] |
| 83 | 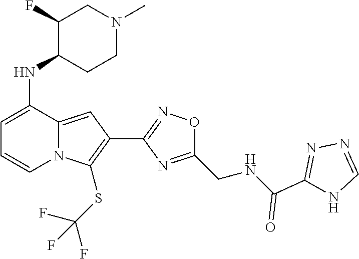 [SMILES: CN1CC[C@@H](Nc2cccn3c(SC(F)(F)F)c(-c4noc(CNC(=O)c5nnc[nH]5)n4)cc23)[C@@H](F)C1] |
| 84 | 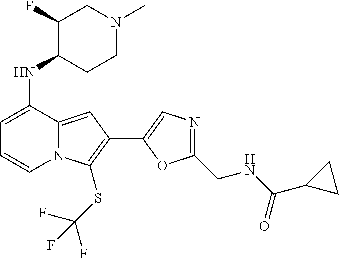 [SMILES: CN1CC[C@@H](Nc2cccn3c(SC(F)(F)F)c(-c4cnc(CNC(=O)C5CC5)o4)cc23)[C@@H](F)C1] |
| 85 | 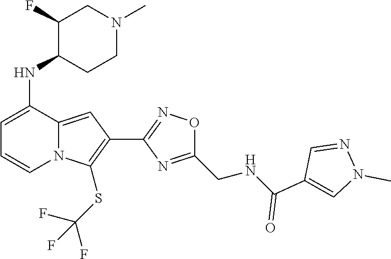 [SMILES: CN1CC[C@@H](Nc2cccn3c(SC(F)(F)F)c(-c4noc(CNC(=O)c5cnn(C)c5)n4)cc23)[C@@H](F)C1] |
| 86 | 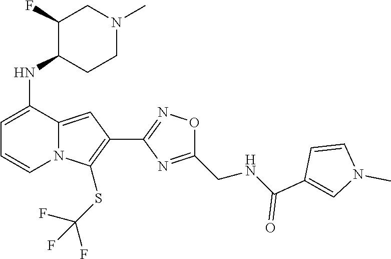 [SMILES: CN1CC[C@@H](Nc2cccn3c(SC(F)(F)F)c(-c4noc(CNC(=O)c5ccn(C)c5)n4)cc23)[C@@H](F)C1] |
| 87 | 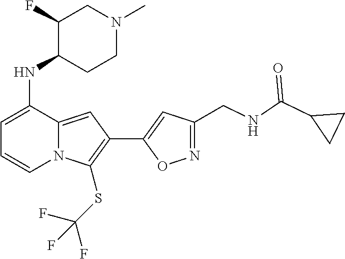 [SMILES: CN1CC[C@@H](Nc2cccn3c(SC(F)(F)F)c(-c4cc(CNC(=O)C5CC5)no4)cc23)[C@@H](F)C1] |
| 88 | 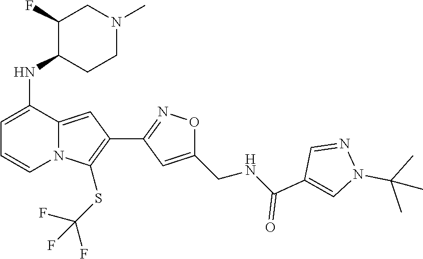 [SMILES: CN1CC[C@@H](Nc2cccn3c(SC(F)(F)F)c(-c4cc(CNC(=O)c5cnn(C(C)(C)C)c5)on4)cc23)[C@@H](F)C1] |
| 89 | 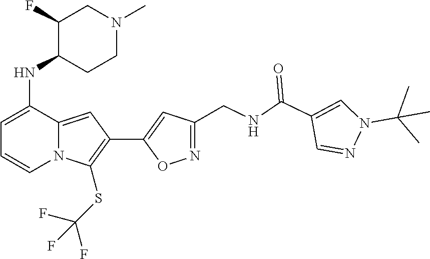 [SMILES: CN1CC[C@@H](Nc2cccn3c(SC(F)(F)F)c(-c4cc(CNC(=O)c5cnn(C(C)(C)C)c5)no4)cc23)[C@@H](F)C1] |
| 90 | 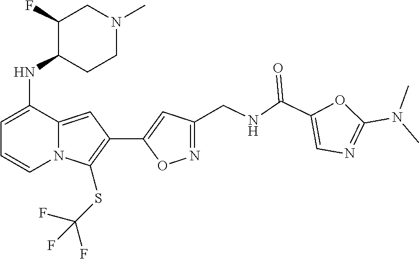 [SMILES: CN1CC[C@@H](Nc2cccn3c(SC(F)(F)F)c(-c4cc(CNC(=O)c5cnc(N(C)C)o5)no4)cc23)[C@@H](F)C1] |
| 91 | 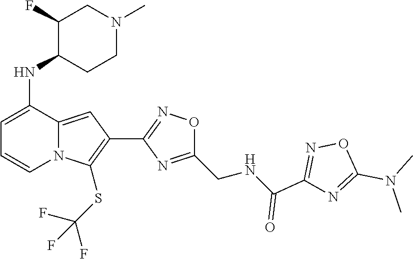 [SMILES: CN1CC[C@@H](Nc2cccn3c(SC(F)(F)F)c(-c4noc(CNC(=O)c5noc(N(C)C)n5)n4)cc23)[C@@H](F)C1] |
| 92 | 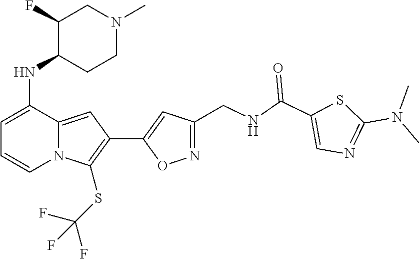 [SMILES: CN1CC[C@@H](Nc2cccn3c(SC(F)(F)F)c(-c4cc(CNC(=O)c5cnc(N(C)C)s5)no4)cc23)[C@@H](F)C1] |
| 93 | 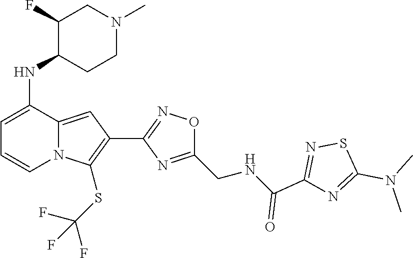 [SMILES: CN1CC[C@@H](Nc2cccn3c(SC(F)(F)F)c(-c4noc(CNC(=O)c5nsc(N(C)C)n5)n4)cc23)[C@@H](F)C1] |
| 94 | 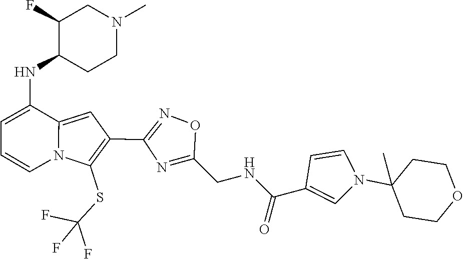 [SMILES: CN1CC[C@@H](Nc2cccn3c(SC(F)(F)F)c(-c4noc(CNC(=O)c5ccn(C6(C)CCOCC6)c5)n4)cc23)[C@@H](F)C1] |
| 95 | 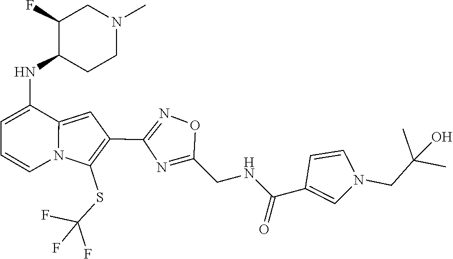 [SMILES: CN1CC[C@@H](Nc2cccn3c(SC(F)(F)F)c(-c4noc(CNC(=O)c5ccn(CC(C)(C)O)c5)n4)cc23)[C@@H](F)C1] |
| 96 | 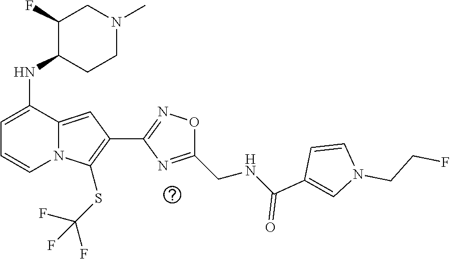 [SMILES: CN1CC[C@@H](Nc2cccn3c(SC(F)(F)F)c(-c4noc(CNC(=O)c5ccn(CCF)c5)n4)cc23)[C@@H](F)C1] |
| 97 | 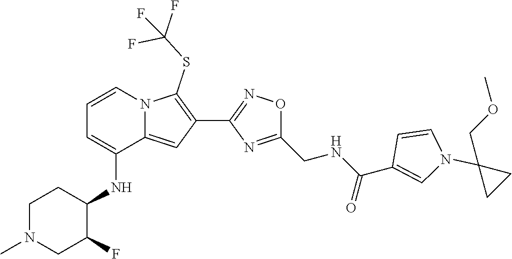 [SMILES: COCC1(n2ccc(C(=O)NCc3nc(-c4cc5c(N[C@@H]6CCN(C)C[C@@H]6F)cccn5c4SC(F)(F)F)no3)c2)CC1] |
| 98 | 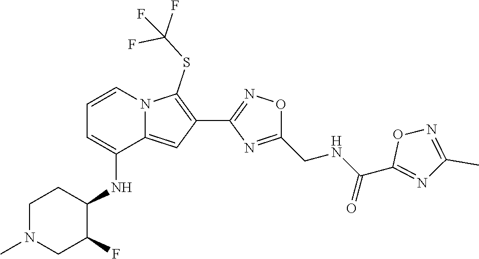 |
| 99 | 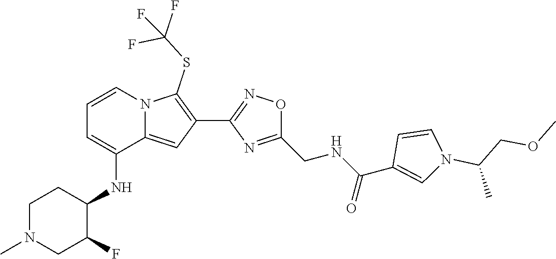 |
| 100 | 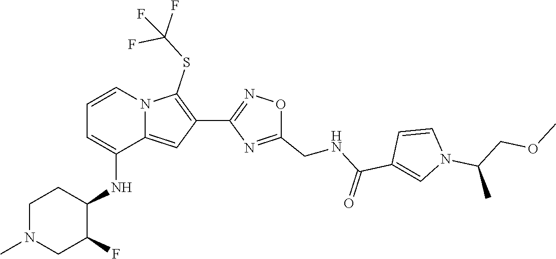 |
| 101 | 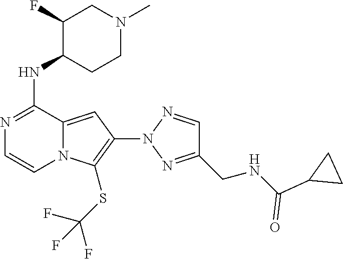 [SMILES: CN1CC[C@@H](Nc2nccn3c(SC(F)(F)F)c(-n4ncc(CNC(=O)C5CC5)n4)cc23)[C@@H](F)C1] |
| 102 | 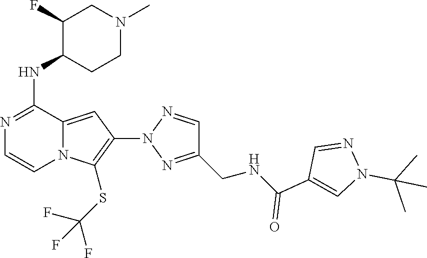 [SMILES: CN1CC[C@@H](Nc2nccn3c(SC(F)(F)F)c(-n4ncc(CNC(=O)c5cnn(C(C)(C)C)c5)n4)cc23)[C@@H](F)C1] |
| 103 | 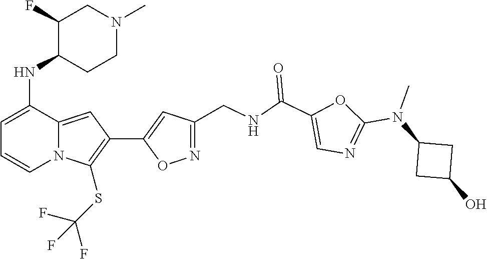 [SMILES: CN1CC[C@@H](Nc2cccn3c(SC(F)(F)F)c(-c4cc(CNC(=O)c5cnc(N(C)[C@H]6C[C@@H](O)C6)o5)no4)cc23)[C@@H](F)C1] |
| 104 | 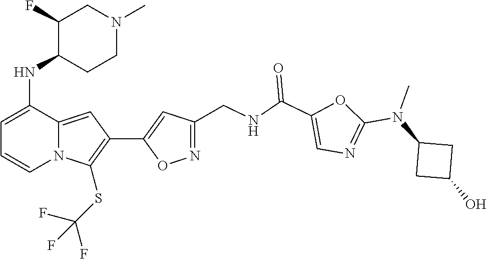 [SMILES: CN1CC[C@@H](Nc2cccn3c(SC(F)(F)F)c(-c4cc(CNC(=O)c5cnc(N(C)[C@H]6C[C@H](O)C6)o5)no4)cc23)[C@@H](F)C1] |
| 105 | 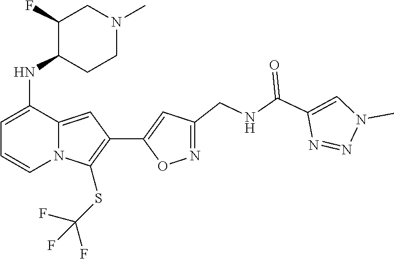 [SMILES: CN1CC[C@@H](Nc2cccn3c(SC(F)(F)F)c(-c4cc(CNC(=O)c5cn(C)nn5)no4)cc23)[C@@H](F)C1] |
| 106 | 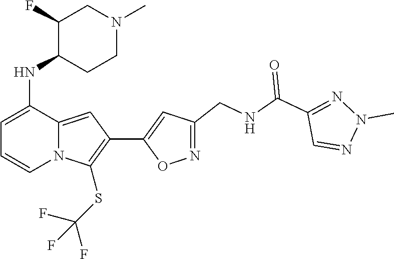 [SMILES: CN1CC[C@@H](Nc2cccn3c(SC(F)(F)F)c(-c4cc(CNC(=O)c5cnn(C)n5)no4)cc23)[C@@H](F)C1] |
| 107 | 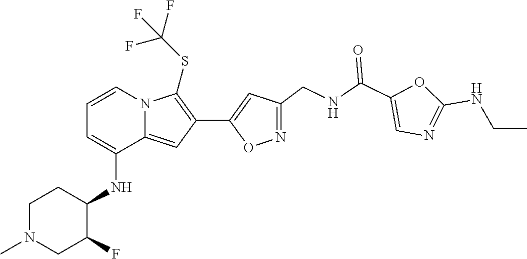 [SMILES: CCNc1ncc(C(=O)NCc2cc(-c3cc4c(N[C@@H]5CCN(C)C[C@@H]5F)cccn4c3SC(F)(F)F)on2)o1] |
| 108 | 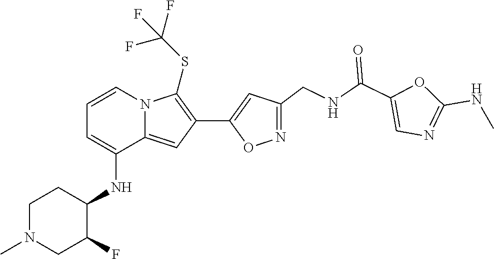 |
| 109 | 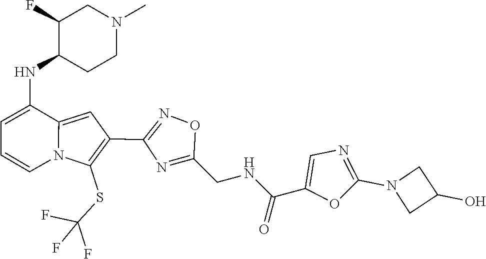 [SMILES: CN1CC[C@@H](Nc2cccn3c(SC(F)(F)F)c(-c4noc(CNC(=O)c5cnc(N6CC(O)C6)o5)n4)cc23)[C@@H](F)C1] |
| 110 | 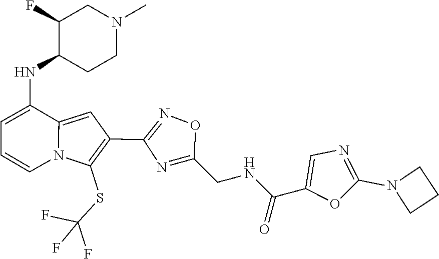 [SMILES: CN1CC[C@@H](Nc2cccn3c(SC(F)(F)F)c(-c4noc(CNC(=O)c5cnc(N6CCC6)o5)n4)cc23)[C@@H](F)C1] |

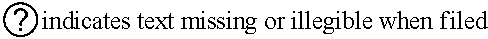

In some embodiments, the compound is selected from the group consisting of the compounds delineated in List 1, or a pharmaceutically acceptable salt thereof.

### List 1

N-((3-(8-(((3S,4R)-3-fluoro-1-methylpiperidin-4-yl)amino)-3-vinylimidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide; N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-vinyl-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-vinyl-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-vinylimidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-vinylimidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-vinylpyrazolo[1,5-a]pyridin-2-yl)-1,2,4-oxadiazol-5-yl)methyl)-1H-pyrrole-3-carboxamide; N-((5-(7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide; N-((5-(8-(((3S,4R)-3-fluoro-1-methylpiperidin-4-yl)amino)-3-vinylimidazo[1,2-a]pyridin-2-yl)-1,3,4-thiadiazol-2-yl)methyl)cyclopropanecarboxamide; N-((5-(7-(((3S,4R)-3-fluoro-1-methylpiperidin-4-yl)amino)-3-vinylpyrazolo[1,5-a]pyridin-2-yl)-1,3,4-thiadiazol-2-yl)methyl)cyclopropanecarboxamide; 1-(tert-butyl)-N-((5-(7-(((3S,4R)-3-fluoro-1-methylpiperidin-4-yl)amino)-3-vinyl-2H-indazol-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-vinyl-2H-indazol-2-yl)-1,3,4-thiadiazol-2-yl)methyl)-1H-pyrrole-3-carboxamide; 1-(tert-butyl)-N-((5-(8-(((3S,4R)-3-fluoro-1-methylpiperidin-4-yl)amino)-3-vinylimidazo[1,2-a]pyridin-2-yl)-1,3,4-thiadiazol-2-yl)methyl)-1H-pyrazole-4-carboxamide; 1-(tert-butyl)-N-((5-(8-(((3S,4R)-3-fluoro-1-methylpiperidin-4-yl)amino)-3-vinylimidazo[1,2-a]pyridin-2-yl)-1,3,4-thiadiazol-2-yl)methyl)-1H-pyrrole-3-carboxamide; 1-(tert-butyl)-N-((5-(7-(((3S,4R)-3-fluoro-1-methylpiperidin-4-yl)amino)-3-vinylpyrazolo[1,5-a]pyridin-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-vinylpyrazolo[1,5-a]pyridin-2-yl)-1,3,4-thiadiazol-2-yl)methyl)-1H-pyrrole-3-carboxamide; N-((3-(5-amino-7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-vinylimidazo[1,2-a]pyrazin-2-yl)-1,2,4-oxadiazol-5-yl)methyl)cyclopropanecarboxamide; N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)cyclopropanecarboxamide; N-((3-(5-amino-7-(((3S,4R)-3-fluoro-1-methylpiperidin-4-yl)amino)-3-vinyl-2H-indazol-2-yl)-1,2,4-oxadiazol-5-yl)methyl)-1-(tert-butyl)-1H-pyrazole-4-carboxamide; N-((3-(5-amino-7-(((3S,4R)-3-fluoro-1-methylpiperidin-4-yl)amino)-3-vinyl-2H-indazol-2-yl)-1,2,4-oxadiazol-5-yl)methyl)-1-(tert-butyl)-1H-pyrrole-3-carboxamide; 1-(tert-butyl)-N-((3-(8-(((3S,4R)-3-fluoro-1-methylpiperidin-4-yl)amino)-3-vinylimidazo[1,2-a]pyrazin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-vinylimidazo[1,2-a]pyrazin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-vinylpyrazolo[1,5-a]pyrazin-2-yl)-1,2,4-oxadiazol-5-yl)methyl)-1H-pyrrole-3-carboxamide; N-((5-(5-amino-7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide; N-((5-(8-(((3S,4R)-3-fluoro-1-methylpiperidin-4-yl)amino)-3-vinylimidazo[1,2-a]pyrazin-2-yl)-1,3,4-thiadiazol-2-yl)methyl)cyclopropanecarboxamide; N-((5-(7-(((3S,4R)-3-fluoro-1-methylpiperidin-4-yl)amino)-3-vinylpyrazolo[1,5-a]pyrazin-2-yl)-1,3,4-thiadiazol-2-yl)methyl)cyclopropanecarboxamide; N-((5-(5-amino-7-(((3S,4R)-3-fluoro-1-methylpiperidin-4-yl)amino)-3-vinyl-2H-indazol-2-yl)-1,3,4-thiadiazol-2-yl)methyl)-1-(tert-butyl)-1H-pyrazole-4-carboxamide; N-((5-(5-amino-7-(((3S,4R)-3-fluoro-1-methylpiperidin-4-yl)amino)-3-vinyl-2H-indazol-2-yl)-1,3,4-thiadiazol-2-yl)methyl)-1-(tert-butyl)-1H-pyrrole-3-carboxamide; 1-(tert-butyl)-N-((5-(8-(((3 S,4R)-3-fluoro-1-methylpiperidin-4-yl)amino)-3-vinylimidazo[1,2-a]pyrazin-2-yl)-1,3,4-thiadiazol-2-yl)methyl)-1H-pyrazole-4-carboxamide; 1-(tert-butyl)-N-((5-(8-(((3S,4R)-3-fluoro-1-methylpiperidin-4-yl)amino)-3-vinylimidazo[1,2-a]pyrazin-2-yl)-1,3,4-thiadiazol-2-yl)methyl)-1H-pyrrole-3-carboxamide; 1-(tert-butyl)-N-((5-(7-(((3S,4R)-3-fluoro-1-methylpiperidin-4-yl)amino)-3-vinylpyrazolo[1,5-a]pyrazin-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-vinylpyrazolo[1,5-a]pyrazin-2-yl)-1,3,4-thiadiazol-2-yl)methyl)-1H-pyrrole-3-carboxamide; N-((3-(5-fluoro-7-(((3S,4R)-3-fluoro-1-methylpiperidin-4-yl)amino)-3-vinyl-2H-indazol-2-yl)-1,2,4-oxadiazol-5-yl)methyl)cyclopropanecarboxamide; 1-(tert-butyl)-N-((3-(5-fluoro-7-(((3 S,4R)-3-fluoro-1-methylpiperidin-4-yl)amino)-3-vinyl-2H-indazol-2-yl)-1,2,4-oxadiazol-5-yl)methyl)-1H-pyrazole-4-carboxamide; 1-(tert-butyl)-N-((3-(5-fluoro-7-(((3S,4R)-3-fluoro-1-methylpiperidin-4-yl)amino)-3-vinyl-2H-indazol-2-yl)-1,2,4-oxadiazol-5-yl)methyl)-1H-pyrrole-3-carboxamide; N-((5-(5-fluoro-7-(((3S,4R)-3-fluoro-1-methylpiperidin-4-yl)amino)-3-vinyl-2H-indazol-2-yl)-1,3,4-thiadiazol-2-yl)methyl)cyclopropanecarboxamide; 1-(tert-butyl)-N-((5-(5-fluoro-7-(((3 S,4R)-3-fluoro-1-methylpiperidin-4-yl)amino)-3-vinyl-2H-indazol-2-yl)-1,3,4-thiadiazol-2-yl)methyl)-1H-pyrazole-4-carboxamide; 1-(tert-butyl)-N-((5-(5-fluoro-7-(((3 S,4R)-3-fluoro-1-methylpiperidin-4-yl)amino)-3-vinyl-2H-indazol-2-yl)-1,3,4-thiadiazol-2-yl)methyl)-1H-pyrrole-3-carboxamide; N-((3-(7-(((3S,4R)-3-fluoro-1-methylpiperidin-4-yl)amino)-3-(prop-1-en-2-yl)-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-(prop-1-en-2-yl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(prop-1-en-2-yl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-(prop-1-en-2-yl)imidazo[1,2-a]pyridin-2-yl)-1,2,4-oxadiazol-5-yl)methyl)-1H-pyrrole-3-carboxamide; 1-(tert-butyl)-N-((3-(7-(((3 S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)-1H-pyrazole-4-carboxamide; 1-(tert-butyl)-N-((3-(7-(((3 S,4R)-3-fluoro-1-methylpiperidin-4-yl)amino)-3-(prop-1-en-2-yl)pyrazolo[1,5-a]pyridin-2-yl)-1,2,4-oxadiazol-5-yl)methyl)-1H-pyrrole-3-carboxamide; N-((3-(7-(((3 S,4R)-3-fluoro-1-methylpiperidin-4-yl)amino)-3-(1-fluorovinyl)-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-(1-fluorovinyl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(1-fluorovinyl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-(1-fluorovinyl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(1-fluorovinyl)pyrazolo[1,5-a]pyridin-2-yl)-1,2,4-oxadiazol-5-yl)methyl)-1H-pyrrole-3-carboxamide; N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-((E)-prop-1-en-1-yl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-((E)-prop-1-en-1-yl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-((E)-prop-1-en-1-yl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-((E)-prop-1-en-1-yl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-((E)-prop-1-en-1-yl)pyrazolo[1,5-a]pyridin-2-yl)-1,2,4-oxadiazol-5-yl)methyl)-1H-pyrrole-3-carboxamide; N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)cyclopropanecarboxamide; N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)cyclopropanecarboxamide; N-((3-(3-(2,2-difluorovinyl)-7-(((3S,4R)-3-fluoro-1-methylpiperidin-4-yl)amino)pyrazolo[1,5-a]pyridin-2-yl)-1,2,4-oxadiazol-5-yl)methyl)cyclopropanecarboxamide; 1-(tert-butyl)-N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)-1H-pyrazole-4-carboxamide; 1-(tert-butyl)-N-((3-(3-(2,2-difluorovinyl)-7-(((3S,4R)-3-fluoro-1-methylpiperidin-4-yl)amino)-2H-indazol-2-yl)-1,2,4-oxadiazol-5-yl)methyl)-1H-pyrrole-3-carboxamide; 1-(tert-butyl)-N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)-1H-pyrazole-4-carboxamide; 1-(tert-butyl)-N-((3-(3-(2,2-difluorovinyl)-8-(((3S,4R)-3-fluoro-1-methylpiperidin-4-yl)amino)imidazo[1,2-a]pyridin-2-yl)-1,2,4-oxadiazol-5-yl)methyl)-1H-pyrrole-3-carboxamide; 1-(tert-butyl)-N-((3-(3-(2,2-difluorovinyl)-7-(((3S,4R)-3-fluoro-1-methylpiperidin-4-yl)amino)pyrazolo[1,5-a]pyridin-2-yl)-1,2,4-oxadiazol-5-yl)methyl)-1H-pyrazole-4-carboxamide; 1-(tert-butyl)-N-((3-(3-(2,2-difluorovinyl)-7-(((3S,4R)-3-fluoro-1-methylpiperidin-4-yl)amino)pyrazolo[1,5-a]pyridin-2-yl)-1,2,4-oxadiazol-5-yl)methyl)-1H-pyrrole-3-carboxamide; N-((3-(7-(((3S,4R)-3-fluoro-1-methylpiperidin-4-yl)amino)-3-(1,2,2-trifluorovinyl)-2H-indazol-2-yl)-1,2,4-oxadiazol-5-yl)methyl)cyclopropanecarboxamide; N-((3-(8-(((3 S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)cyclopropanecarboxamide, 2-(5-((((1-(tert-butyl)-1H-pyrazol-4-yl)(12-fluoraneylidene)methyl)amino)methyl)-1,2,4-oxadiazol-3-yl)-N-((3 S,4R)-3-fluoro-1-methylpiperidin-4-yl)-3-(1,2,2-trifluorovinyl)-2H-indazol-7-amine; 1-(tert-butyl)-N-((3-(7-(((3S,4R)-3-fluoro-1-methylpiperidin-4-yl)amino)-3-(1,2,2-trifluorovinyl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-(1,2,2-trifluorovinyl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(1,2,2-trifluorovinyl)pyrazolo[1,5-a]pyridin-2-yl)-1,2,4-oxadiazol-5-yl)methyl)-1H-pyrrole-3-carboxamide; N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-((R)-oxiran-2-yl)pyrazolo[1,5-a]pyridin-2-yl)-1,2,4-oxadiazol-5-yl)methyl)-1H-pyrrole-3-carboxamide; N-((3-(7-(((3S,4R)-3-fluoro-1-methylpiperidin-4-yl)amino)-3-((S)-oxiran-2-yl)-2H-indazol-2-yl)-1,2,4-oxadiazol-5-yl)methyl)cyclopropanecarboxamide, N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-((S)-oxiran-2-yl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-((R)-oxiran-2-yl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-((S)-oxiran-2-yl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-((R)-oxiran-2-yl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-((S)-oxiran-2-yl)pyrazolo[1,5-a]pyridin-2-yl)-1,2,4-oxadiazol-5-yl)methyl)-1H-pyrrole-3-carboxamide; N-((3-(6-acrylamido-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1-(tert-butyl)-1H-pyrazole-4-carboxamide; 1-(tert-butyl)-N-((3-(8-(((3S,4R)-3-fluoro-1-methylpiperidin-4-yl)amino)-6-methacrylamido-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(6-((E)-4-(dimethylamino)but-2-enamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-((E)-4-(4-methylpiperazin-1-yl)but-2-enamido)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-(4-(methylamino)but-2-ynamido)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-(2-fluoroacrylamido)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-propiolamido-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-((E)-4-(methylamino)but-2-enamido)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-6-((E)-4-morpholinobut-2-enamido)-3-(2,2,2-trifluoroethyl)imidazo[1,2-a]pyridin-2-yl)-1,2,4-oxadiazol-5-yl)methyl)-1H-pyrazole-4-carboxamide; N-((3-(6-(but-2-ynamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1-(tert-butyl)-1H-pyrazole-4-carboxamide; N-((3-(6-((E)-but-2-enamido)-8-(((3 S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1-(tert-butyl)-1H-pyrazole-4-carboxamide; 1-(tert-butyl)-N-((3-(6-((E)-4-(3,3-difluoroazetidin-1-yl)but-2-enamido)-8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(6-(4-(dimethylamino)but-2-ynamido)-8-(((3 S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide; N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide; N-((3-(8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)cyclopropanecarboxamide; N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrrole-3-carboxamide; 1-(tert-butyl)-N-((3-(8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-(2,2,2-trifluoroethyl)imidazo[1,2-a]pyridin-2-yl)-1,2,4-oxadiazol-5-yl)methyl)-1H-pyrrole-3-carboxamide; 1-(tert-butyl)-N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(2,2,2-trifluoroethyl)pyrazolo[1,5-a]pyridin-2-yl)-1,2,4-oxadiazol-5-yl)methyl)-1H-pyrrole-3-carboxamide; N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide; 1-(tert-butyl)-N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrrole-3-carboxamide; N-((5-(8-(((3S,4R)-3-fluoro-1-methylpiperidin-4-yl)amino)-3-(2,2,2-trifluoroethyl)imidazo[1,2-a]pyridin-2-yl)-1,3,4-thiadiazol-2-yl)methyl)cyclopropanecarboxamide; 1-(tert-butyl)-N-((5-(8-(((3S,4R)-3-fluoro-1-methylpiperidin-4-yl)amino)-3-(2,2,2-trifluoroethyl)imidazo[1,2-a]pyridin-2-yl)-1,3,4-thiadiazol-2-yl)methyl)-1H-pyrazole-4-carboxamide; 1-(tert-butyl)-N-((5-(8-(((3S,4R)-3-fluoro-1-methylpiperidin-4-yl)amino)-3-(2,2,2-trifluoroethyl)imidazo[1,2-a]pyridin-2-yl)-1,3,4-thiadiazol-2-yl)methyl)-1H-pyrrole-3-carboxamide; N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)cyclopropanecarboxamide; 1-(tert-butyl)-N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-(2,2,2-trifluoroethyl)pyrazolo[1,5-a]pyridin-2-yl)-1,3,4-thiadiazol-2-yl)methyl)-1H-pyrrole-3-carboxamide; N-((3-(5-amino-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide; N-((3-(5-amino-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1-(tert-butyl)-1H-pyrazole-4-carboxamide; N-((3-(5-amino-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1-(tert-butyl)-1H-pyrrole-3-carboxamide; N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)cyclopropanecarboxamide; 1-(tert-butyl)-N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)-1H-pyrazole-4-carboxamide; 1-(tert-butyl)-N-((3-(8-(((3S,4R)-3-fluoro-1-methylpiperidin-4-yl)amino)-3-(2,2,2-trifluoroethyl)imidazo[1,2-a]pyrazin-2-yl)-1,2,4-oxadiazol-5-yl)methyl)-1H-pyrrole-3-carboxamide; N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(2,2,2-trifluoroethyl)pyrazolo[1,5-a]pyrazin-2-yl)-1,2,4-oxadiazol-5-yl)methyl)-1H-pyrrole-3-carboxamide; N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide; N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1-(tert-butyl)-1H-pyrrole-3-carboxamide; N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)cyclopropanecarboxamide, 1-(tert-butyl)-N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)-1H-pyrazole-4-carboxamide; 1-(tert-butyl)-N-((5-(8-(((3S,4R)-3-fluoro-1-methylpiperidin-4-yl)amino)-3-(2,2,2-trifluoroethyl)imidazo[1,2-a]pyrazin-2-yl)-1,3,4-thiadiazol-2-yl)methyl)-1H-pyrrole-3-carboxamide; N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)cyclopropanecarboxamide; N-((5-(5-amino-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1-(tert-butyl)-1H-pyrazole-4-carboxamide; 1-(tert-butyl)-N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)-1H-pyrazole-4-carboxamide; 1-(tert-butyl)-N-((5-(7-(((3S,4R)-3-fluoro-1-methylpiperidin-4-yl)amino)-3-(2,2,2-trifluoroethyl)pyrazolo[1,5-a]pyrazin-2-yl)-1,3,4-thiadiazol-2-yl)methyl)-1H-pyrrole-3-carboxamide; N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)cyclopropanecarboxamide; 1-(tert-butyl)-N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrazole-4-carboxamide; 1-(tert-butyl)-N-((3-(5-fluoro-7-(((3S,4R)-3-fluoro-1-methylpiperidin-4-yl)amino)-3-(2,2,2-trifluoroethyl)-2H-indazol-2-yl)-1,2,4-oxadiazol-5-yl)methyl)-1H-pyrrole-3-carboxamide; N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)cyclopropanecarboxamide; 1-(tert-butyl)-N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrazole-4-carboxamide; 1-(tert-butyl)-N-((5-(5-fluoro-7-(((3S,4R)-3-fluoro-1-methylpiperidin-4-yl)amino)-3-(2,2,2-trifluoroethyl)-2H-indazol-2-yl)-1,3,4-thiadiazol-2-yl)methyl)-1H-pyrrole-3-carboxamide; N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)cyclopropanecarboxamide; 1-(tert-butyl)-N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)-1H-pyrazole-4-carboxamide; 1-(tert-butyl)-N-((3-(7-(((3S,4R)-3-fluoro-1-methylpiperidin-4-yl)amino)-3-(perfluoroethyl)-2H-indazol-2-yl)-1,2,4-oxadiazol-5-yl)methyl)-1H-pyrrole-3-carboxamide; N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)cyclopropanecarboxamide; 1-(tert-butyl)-N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)-1H-pyrazole-4-carboxamide; and 1-(tert-butyl)-N-((5-(7-(((3S,4R)-3-fluoro-1-methylpiperidin-4-yl)amino)-3-(perfluoroethyl)pyrazolo[1,5-a]pyridin-2-yl)-1,3,4-thiadiazol-2-yl)methyl)-1H-pyrrole-3-carboxamide.

### Pharmaceutical Compositions

Some embodiments provide a pharmaceutical composition comprising a compound of Formula (I), or a pharmaceutically acceptable salt thereof, and one or more pharmaceutically acceptable excipients.

### Methods of Treatment

Provided herein are methods for restoring p53 function, encoded by TP53 gene. For example, provided herein are compounds that restore p53 function that are useful for treating or preventing diseases associated with dysregulation of a TP53 gene, a p53 protein, or the activity of any of the same (i.e., a p53-associated disease), such as cancer (e.g., p53-associated cancer).

The terms “restore” or “restoration of” means to increase the activity and/or function of the specified target by a measurable amount. For example, restoration of a mutant p53 with a compound of Formula (I) refers to increasing the function of the mutant p53 in the presence of the compound to a higher level than the function of the mutant p53 in the absence of the compound.

The ability of test compounds to act as a p53 restorer may be demonstrated by assays known in the art. The activity of the compounds and compositions provided herein as p53 restorers can be assayed in vitro, in vivo, or in a cell line. In vitro assays include assays that determine activation of the protein and/or a change in its conformation. Potency of a p53 restorer as provided herein can be determined by EC₅₀ value. A compound with a lower EC₅₀ value, as determined under substantially similar conditions, is a more potent p53 restorer relative to a compound with a higher EC₅₀ value.

### Indications

Compounds of Formula (I), or pharmaceutically acceptable salts thereof, are useful for treating diseases which can be treated with a p53 restorer, such as p53-associated diseases, e.g., proliferative disorders such as cancers, including hematological cancers and solid tumors (e.g., advanced or metastatic solid tumors). In some embodiments, the p53-associated disease or disorder is Li-Fraumeni syndrome.

Some embodiments provide a method of treating cancer in a subject in need thereof, comprising administering to the subject a therapeutically effective amount of a compound of Formula (I), or a pharmaceutically acceptable salt thereof. In some embodiments, the cancer is a p53-associated cancer.

Some embodiments provide a method of treating cancer in a subject that has been identified or diagnosed as having a p53-associated cancer, comprising administering to the subject a therapeutically effective amount of a compound of Formula (I), or a pharmaceutically acceptable salt thereof. In some embodiments, the p53-associated cancer harbors a Y220C mutation.

Some embodiments provide a method of treating a p53-associated cancer in a subject in need thereof, comprising administering to the subject a therapeutically effective amount of any one of the compound of Examples 1-110, or a pharmaceutically acceptable salt thereof. In some embodiments, the p53-associated cancer harbors a Y220C mutation.

Some embodiments provide a method of treating cancer in a subject in need thereof, comprising (a) determining that the subject has a p53-associated cancer, and (b) administering to the subject a therapeutically effective amount of a compound of Formula (I), or a pharmaceutically acceptable salt thereof.

Some embodiments provide a method of treating Li-Fraumeni syndrome in a subject in need thereof, comprising administering to the subject a therapeutically effective amount of a compound of Formula (I), or a pharmaceutically acceptable salt thereof.

Some embodiments provide a method of treating Li-Fraumeni syndrome in a subject that has been identified or diagnosed as having Li-Fraumeni syndrome, comprising administering to the subject a therapeutically effective amount of a compound of Formula (I), or a pharmaceutically acceptable salt thereof.

Some embodiments provide a method of treating Li-Fraumeni syndrome in a subject in need thereof, comprising (a) determining that the subject has Li-Fraumeni syndrome, and (b) administering to the subject a therapeutically effective amount of a compound of Formula (I), or a pharmaceutically acceptable salt thereof.

In some embodiments, a compound of Formula (I), or a pharmaceutically acceptable salt thereof, is administered prophylactically to a subject with Li-Fraumeni syndrome. In some embodiments, a therapeutically effective amount of Formula (I), or a pharmaceutically acceptable salt thereof, is administered prophylactically to a subject with Li-Fraumeni syndrome.

The term “p53-associated disease” as used herein refers to diseases associated with or having a dysregulation of a TP53 gene, a p53 protein, or the activity of any (e.g., one or more) of the same (e.g., any of the types of dysregulation of a TP53 gene, or a p53 protein, or the activity of any of the same described herein). Non-limiting examples of a p53-associated disease include, for example, cancer (e.g., p53-associated cancer).

The term “p53-associated cancer” as used herein refers to cancers associated with or having a dysregulation of a TP53 gene, a p53 protein, or activity of any of the same. Non-limiting examples of p53-associated cancers are described herein.

The term “wild type” or “wild-type” describes a nucleic acid (e.g., a TP53 gene or a p53 mRNA) or protein (e.g., a p53) sequence that is typically found in a subject that does not have a cancer related to the reference nucleic acid or protein.

Provided herein is a method of treating cancer (e.g., a p53-associated cancer) in a subject in need of such treatment, the method comprising administering to the subject a therapeutically effective amount of a compound of Formula (I), or a pharmaceutically acceptable salt thereof, or a pharmaceutical composition thereof. For example, provided herein are methods for treating p53-associated cancer in a subject in need of such treatment, the method comprising a) detecting a dysregulation of TP53 gene, a p53 protein, or the activity of any of the same in a sample from the subject; and b) administering a therapeutically effective amount of a compound of Formula (I), or a pharmaceutically acceptable salt thereof. In some embodiments, the dysregulation of a TP53 gene, a p53 protein, or the activity of any of the same includes one or more a p53 protein substitutions/point mutations/insertions. Non-limiting examples of p53 protein substitutions/insertions/deletions are described in Table 1.

In some embodiments, the p53 protein substitution/insertion/deletion is Y220X, where X is any amino acid other than Y. In some embodiments, the p53 protein substitution/insertion/deletion is selected from the group consisting of Y220C, Y220S, Y220N, Y220D, and combinations thereof. In some embodiments, the p53 protein substitution/insertion/deletion is selected from the group consisting of Y220C or Y220S, or a combination thereof. In some embodiments, the p53 protein substitution/insertion/deletion is Y220C. In some embodiments, the p53 protein substitution/insertion/deletion is Y220S.

In some embodiments, the dysregulation of a TP53 gene, a p53 protein, or activity of any of the same, includes at least one point mutation in a TP53 gene that results in the production of a p53 protein that has one or more amino acid substitutions or insertions or deletions in a TP53 gene that results in the production of a p53 protein that has one or more amino acids inserted or removed, as compared to the wild type p53 protein. In some cases, the resulting mutant p53 protein has reduced function, as compared to a wild type p53 protein or a p53 protein not including the same mutation. In some embodiments, the compounds described herein restore the resulting mutant p53 protein function relative to the mutant p53 protein function in the absence of the compounds described herein, for example, by stabilizing the mutant protein into an active conformation.

Exemplary Sequence of Human p53 (UniProtKB entry
P04637-1)
(SEQ ID NO: 1)
MEEPQSDPSVEPPLSQETFSDLWKLLPENNVLSPLPSQAMDDLMLSPDDI
EQWFTEDPGPDEAPRMPEAAPPVAPAPAAPTPAAPAPAPSWPLSSSVPSQ
KTYQGSYGFRLGFLHSGTAKSVTCTYSPALNKMFCQLAKTCPVQLWVDST
PPPGTRVRAMAIYKQSQHMTEVVRRCPHHERCSDSDGLAPPQHLIRVEGN
LRVEYLDDRNTFRHSVVVPYEPPEVGSDCTTIHYNYMCNSSCMGGMNRRP
ILTIITLEDSSGNLLGRNSFEVRVCACPGRDRRTEEENLRKKGEPHHELP
PGSTKRALPNNTSSSPQPKKKPLDGEYFTLQIRGRERFEMFRELNEALEL
KDAQAGKEPGGSRAHSSHLKSKKGQSTSRHKKLMFKTEGPDSD

In some embodiments, compounds of Formula (I), or pharmaceutically acceptable thereof, are useful for treating a cancer that has been identified as having one or more p53 mutations. Accordingly, provided herein are methods for treating a subject diagnosed with (or identified as having) a cancer that include administering to the subject a therapeutically effective amount of a compound of Formula (I), or a pharmaceutically acceptable salt thereof.

Also provided herein are methods for treating a subject identified or diagnosed as having a p53-associated cancer that include administering to the subject a therapeutically effective amount of a compound of Formula (I), or a pharmaceutically acceptable salt thereof, or a pharmaceutical composition thereof. In some embodiments, the subject that has been identified or diagnosed as having a p53-associated cancer through the use of a regulatory agency-approved, e.g., FDA-approved test or assay for identifying dysregulation of a TP53 gene, a p53 protein, or activity of any of the same, in a subject or a biopsy sample from the subject or by performing any of the non-limiting examples of assays described herein. In some embodiments, the test or assay is provided as a kit. In some embodiments, the cancer is an p53-associated cancer.

Also provided are methods for treating cancer in a subject in need thereof, the method comprising: (a) detecting a p53-associated cancer in the subject; and (b) administering to the subject a therapeutically effective amount of a compound of Formula (I), or a pharmaceutically acceptable salt thereof, or a pharmaceutical composition thereof. Some embodiments of these methods further include administering to the subject another anticancer agent (e.g., an immunotherapy). In some embodiments, the subject was previously treated with another anticancer treatment, e.g., at least partial resection of the tumor or radiation therapy. In some embodiments, the subject is determined to have a p53-associated cancer through the use of a regulatory agency-approved, e.g., FDA-approved test or assay for identifying dysregulation of a TP53 gene, a p53 protein, or activity of any of the same, in a subject or a biopsy sample from the subject or by performing any of the non-limiting examples of assays described herein. In some embodiments, the test or assay is provided as a kit. In some embodiments, the cancer is an p53-associated cancer.

Also provided is a compound of Formula (I), or a pharmaceutically acceptable salt thereof, or a pharmaceutical composition thereof, for use in treating a p53-associated cancer in a subject identified or diagnosed as having a p53-associated cancer through a step of performing an assay (e.g., an in vitro assay) on a sample obtained from the subject to determine whether the subject has a dysregulation of a TP53 gene, a p53 protein, or activity of any of the same, where the presence of a dysregulation of a TP53 gene, a p53 protein, or activity of any of the same, identifies that the subject has a p53-associated cancer.

Also provided is a compound of Formula (I), or a pharmaceutically acceptable salt thereof, for use in the treatment of a cancer in a subject in need thereof, or a subject identified or diagnosed as having a p53-associated cancer. Also provided is the use of a compound of Formula (I), or a pharmaceutically acceptable salt thereof, for the manufacture of a medicament for treating a cancer in a subject identified or diagnosed as having a p53-associated cancer. In some embodiments, a subject is identified or diagnosed as having a p53-associated cancer through the use of a regulatory agency-approved, e.g., FDA-approved, kit for identifying dysregulation of a TP53 gene, a p53 protein, or activity of any of the same, in a subject or a biopsy sample from the subject. As provided herein, a p53-associated cancer includes those described herein and known in the art.

In some embodiments of any of the methods or uses described herein, the subject has been identified or diagnosed as having a cancer with a dysregulation of a TP53 gene, a p53 protein, or activity of any of the same. In some embodiments of any of the methods or uses described herein, the subject has a tumor that is positive for a dysregulation of a TP53 gene, a p53 protein, or activity of any of the same. In some embodiments of any of the methods or uses described herein, the subject can be a subject with a tumor(s) that is positive for a dysregulation of a TP53 gene, a p53 protein, or activity of any of the same. In some embodiments of any of the methods or uses described herein, the subject can be a subject whose tumors have a dysregulation of a TP53 gene, a p53 protein, or activity of any of the same. In some embodiments of any of the methods or uses described herein, the subject is suspected of having a p53-associated cancer. In some embodiments, provided herein are methods for treating a p53-associated cancer in a subject in need of such treatment, the method comprising a) detecting a dysregulation of a TP53 gene, a p53 protein, or the activity of any of the same in a sample from the subject; and b) administering a therapeutically effective amount of a compound of Formula (I), or a pharmaceutically acceptable salt thereof. In some embodiments, the dysregulation of a TP53 gene, a p53 protein, or the activity of any of the same includes one or more p53 protein point mutations/insertions/deletions, as described herein. In some embodiments, the cancer with a dysregulation of a TP53 gene, a p53 protein, or activity of any of the same is determined using a regulatory agency-approved, e.g., FDA-approved, assay or kit. In some embodiments, the tumor with a dysregulation of a TP53 gene, a p53 protein, or activity of any of the same is determined using a regulatory agency-approved, e.g., FDA-approved, assay or kit.

In some embodiments of any of the methods or uses described herein, the subject has a clinical record indicating that the subject has a tumor that has a dysregulation of a TP53 gene, a p53 protein, or activity of any of the same. Also provided are methods of treating a subject that include administering a therapeutically effective amount of a compound of Formula (I), or a pharmaceutically acceptable salt thereof, to a subject having a clinical record that indicates that the subject has a dysregulation of a TP53 gene, a p53 protein, or activity of any of the same.

Also provided is a method for restoring p53 function in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant p53 function. In some embodiments, the cell is a cancer cell. In some embodiments, the cancer cell is any cancer as described herein. In some embodiments, the cancer cell is a p53-associated cancer cell. As used herein, the term “contacting” refers to the bringing together of indicated moieties in an in vitro system or an in vivo system. For example, “contacting” a p53 protein with a compound provided herein includes the administration of a compound provided herein to an individual or subject, such as a human, having a p53 protein, as well as, for example, introducing a compound provided herein into a sample containing a cellular or purified preparation containing the p53 protein.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Further provided herein is a method of increase cell death, in vitro or in vivo, the method comprising contacting a cell with an effective amount of a compound of Formula (I), or a pharmaceutically acceptable salt thereof, or a pharmaceutical composition thereof as defined herein. Also provided herein is a method of increasing tumor cell death in a subject. The method comprises administering to the subject an effective compound of Formula (I), or a pharmaceutically acceptable salt thereof, in an amount effective to increase tumor cell death.

In some embodiments of any of the methods or uses described herein, the cancer (e.g., p53-associated cancer) is selected from a hematological cancer and a solid tumor.

In some embodiments of any of the methods or uses described herein, the cancer (e.g., p53-associated cancer) is a hematological cancer. In some embodiments, the hematological cancer is a leukemia. In some embodiments, the hematological cancer is a lymphoma. In some embodiments, the hematological cancer is acute myeloid leukemia (AML), chronic lymphocytic leukemia (CLL), or hairy cell leukemia (HCL). In some embodiments, the hematological cancer is acute myeloid leukemia (AML).

In some embodiments of any of the methods or uses described herein, the cancer (e.g., p53-associated cancer) is selected from brain cancer, bladder cancer, breast cancer, colorectal cancer, skin cancer, esophageal cancer, lung cancer, gastric cancer, kidney cancer, uterine cancer, ovarian cancer, liver cancer, pancreatic cancer, prostate cancer, leiomyosarcoma, and head and neck squamous cell carcinoma.

In some embodiments of any of the methods or uses described herein, the cancer (e.g., p53-associated cancer) is selected from colorectal cancer, ovarian cancer, pancreatic cancer, breast cancer, non-small cell lung cancer, small cell lung cancer, endometrial cancer, and bladder cancer.

In some embodiments, the brain cancer is astrocytoma, oligoastrocytoma, oligodendroglioma, or glioblastoma multiforme.

In some embodiments, the bladder cancer is bladder urothelial carcinoma.

In some embodiments, the esophageal cancer is esophageal adenocarcinoma or esophageal squamous cell carcinoma.

In some embodiments, the skin cancer is cutaneous melanoma.

In some embodiments, the lung cancer is small cell lung cancer (SCLC) or non-small cell lung cancer (NSCLC). In some embodiments, the lung cancer is small cell lung cancer (SCLC). In some embodiments, the lung cancer is non-small cell lung cancer (NSCLC). In some embodiments, the lung cancer is lung adenocarcinoma or lung squamous cell carcinoma.

In some embodiments, the gastric cancer is mucinous stomach adenocarcinoma or intestinal type stomach adenocarcinoma.

In some embodiments, the breast cancer is breast invasive ductal carcinoma.

In some embodiments, the uterine cancer is uterine mixed endometrial carcinoma, uterine endometrioid carcinoma, uterine serous carcinoma, or uterine papillary serous carcinoma.

In some embodiments, the ovarian cancer is serous ovarian cancer.

In some embodiments, the kidney cancer is chromophobe renal cell carcinoma.

In some embodiments, the colorectal cancer is colon adenocarcinoma.

In some embodiments, the liver cancer is hepatocellular carcinoma.

In some embodiments, the pancreatic cancer is pancreatic adenocarcinoma.

In some embodiments, the cancer is prostate cancer.

In some embodiments of any of the methods or uses described herein, the p53-associated cancer is breast cancer. In some embodiments of any of the methods or uses described herein, the p53-associated cancer is colorectal cancer. In some embodiments of any of the methods or uses described herein, the p53-associated cancer is endometrial cancer. In some embodiments of any of the methods or uses described herein, the p53-associated cancer is lung cancer.

In some embodiments of any of the methods or uses described herein, the p53-associated cancer is selected from the cancers described in Table 1.

TABLE 1
p53 Protein Amino Acid Substitutions/Insertions/Deletions^{A}

| Amino Acid | Non-Limiting | Non-Limiting Exemplary p53 |
|---|---|---|
| Position | Exemplary Mutations | Associated Cancer(s) |
| 1 | M1 | Head and Neck Squamous Cell Carcinoma |
| | (translation start site) | |
| 4 | P4L | Mucinous Stomach Adenocarcinoma |
| 10 | X10_splice (splice site) | Acute Myeloid Leukemia |
| 11 | E11* (nonsense mutation) | Breast Invasive Ductal Carcinoma |
| | E11K | Renal Clear Cell Carcinoma |
| 20 | S20* | Bladder Urothelial Carcinoma |
| | S20Qfs*24 | Intestinal Type Stomach Adenocarcinoma |
| | (frame shift, additional | |
| | amino acids) | |
| 22 | L22Yfs*22 | Bladder Urothelial Carcinoma |
| 23 | W23* | Cervical Squamous Cell Carcinoma |
| 25 | X25_splice | Adrenocortical Carcinoma |
| | | Head and Neck Squamous Cell Carcinoma |
| | | Tubular Stomach Adenocarcinoma |
| 27 | P27L | Adrenocortical Carcinoma |
| | P27Lfs*17 | Astrocytoma |
| | P27S | Breast Invasive Ductal Carcinoma |
| | | Chromophobe Renal Cell Carcinoma |
| | | Colon Adenocarcinoma |
| | | Cutaneous Melanoma |
| | | Head and Neck Squamous Cell Carcinoma |
| | | Mucinous Carcinoma |
| | | Pancreatic Adenocarcinoma |
| | | Serous Ovarian Cancer |
| | | Uterine Carcinosarcoma/Uterine Malignant Mixed Mullerian |
| | | Tumor |
| 30 | N30del | Colon Adenocarcinoma |
| | (in frame deletion) | |
| 32 | L32Cfs*12 | Astrocytoma |
| | X32_splice | Chromophobe Renal Cell Carcinoma |
| | | Glioblastoma Multiforme |
| | | Head and Neck Squamous Cell Carcinoma |
| | | Head and Neck Squamous Cell Carcinoma |
| | | Lung Squamous Cell Carcinoma |
| | | Oligoastrocytoma |
| | | Renal Clear Cell Carcinoma |
| 33 | S33Ffs*10 | Astrocytoma |
| | X33_splice | Bladder Urothelial Carcinoma |
| | | Breast Invasive Ductal Carcinoma |
| | | Glioblastoma Multiforme |
| | | Head and Neck Squamous Cell Carcinoma |
| | | Hepatocellular Carcinoma |
| | | Lung Adenocarcinoma |
| | | Lung Squamous Cell Carcinoma |
| | | Oligoastrocytoma |
| | | Prostate Adenocarcinoma |
| | | Rectal Adenocarcinoma |
| | | Serous Ovarian Cancer |
| | | Uterine Carcinosarcoma/Uterine Malignant Mixed Mullerian |
| | | Tumor |
| 35 | L35Cfs*9 | Bladder Urothelial Carcinoma |
| | L35Ffs*7 | Colon Adenocarcinoma |
| | L35Ffs*8 | Lung Adenocarcinoma |
| | L35Pfs*10 | Lung Squamous Cell Carcinoma |
| | | Pancreatic Adenocarcinoma |
| | | Papillary Stomach Adenocarcinoma |
| | | Rectal Adenocarcinoma |
| | | Tubular Stomach Adenocarcinoma |
| | | Uterine Endometrioid Carcinoma |
| 36 | P36Wfs*4 | Serous Ovarian Cancer |
| 37 | S37Vfs*6 | Lung Squamous Cell Carcinoma |
| 38 | Q38* | Colon Adenocarcinoma |
| | Q38Kfs*6 | Head and Neck Squamous Cell Carcinoma |
| | | Lung Squamous Cell Carcinoma |
| | | Pancreatic Adenocarcinoma |
| | | Uterine Carcinosarcoma/Uterine Malignant Mixed Mullerian |
| | | Tumor |
| 40 | M40Lfs*7 | Acute Myeloid Leukemia |
| 42 | D42Ifs*2 | Esophageal Squamous Cell Carcinoma |
| 43 | L43* | Hepatocellular Carcinoma |
| | L43Afs*7 | Serous Ovarian Cancer |
| | | Uterine Endometrioid Carcinoma |
| 44 | M44Ifs*79 | Bladder Urothelial Carcinoma |
| | M44Tfs*75 | Lung Squamous Cell Carcinoma |
| 46 | S46* | Lung Squamous Cell Carcinoma |
| | S46Tfs*5 | |
| 47 | P47Rfs*76 | Serous Ovarian Cancer |
| 48 | D48Gfs*4 | Cutaneous Melanoma |
| | D48N | Head and Neck Squamous Cell Carcinoma |
| | D48Nfs*72 | Lung Adenocarcinoma |
| | D48Tfs*75 | Lung Squamous Cell Carcinoma |
| 49 | D49Sfs*69 | Lung Squamous Cell Carcinoma |
| 51 | E51* | Breast Invasive Ductal Carcinoma |
| | E51Gfs*6 | Esophageal Adenocarcinoma |
| | | Serous Ovarian Cancer |
| 52 | Q52* | Breast Invasive Ductal Carcinoma |
| | | Head and Neck Squamous Cell Carcinoma |
| | | Lung Adenocarcinoma |
| 53 | W53* | Bladder Urothelial Carcinoma |
| | W53Cfs*4 | Diffuse Type Stomach Adenocarcinoma |
| | W53Mfs*4 | Glioblastoma Multiforme |
| | | Lung Adenocarcinoma |
| | | Lung Squamous Cell Carcinoma |
| | | Rectal Adenocarcinoma |
| | | Serous Ovarian Cancer |
| | | Uterine Serous Carcinoma/Uterine Papillary Serous Carcinoma |
| 54 | F54Sfs*69 | Lung Adenocarcinoma |
| 56 | E56* | Adrenocortical Carcinoma |
| | E56Kfs*67 | Breast Invasive Ductal Carcinoma |
| | | Head and Neck Squamous Cell Carcinoma |
| | | Hepatocellular Carcinoma |
| | | Lung Squamous Cell Carcinoma |
| 57 | D57Kfs*67 | Lung Adenocarcinoma |
| | D57N | Pancreatic Adenocarcinoma |
| 58 | P58Qfs*65 | Head and Neck Squamous Cell Carcinoma |
| 61 | D61* | Uterine Endometrioid Carcinoma |
| | D61G | Uterine Mixed Endometrial Carcinoma |
| 62 | E62* | Cervical Squamous Cell Carcinoma |
| | E62K | Lung Squamous Cell Carcinoma |
| | E62Kfs*61 | Pancreatic Adenocarcinoma |
| | | Serous Ovarian Cancer |
| 64 | P64Qfs*84 | Esophageal Squamous Cell Carcinoma |
| | P64Sfs*59 | Lung Squamous Cell Carcinoma |
| | P64T | Uterine Endometrioid Carcinoma |
| 65 | R65* | Head and Neck Squamous Cell Carcinoma |
| | R65Efs*58 | Lung Adenocarcinoma |
| | R65Qfs*84 | Lung Squamous Cell Carcinoma |
| 66 | M66Tfs*60 | Serous Ovarian Cancer |
| 67 | P67S | Uterine Endometrioid Carcinoma |
| 68 | E68* | Bladder Urothelial Carcinoma |
| | | Head and Neck Squamous Cell Carcinoma |
| | | Lung Adenocarcinoma |
| | | Lung Squamous Cell Carcinoma |
| 69 | A69Gfs*80 | Lung Adenocarcinoma |
| 72 | P72Rfs*51 | Cutaneous Melanoma |
| | P72S | Diffuse Type Stomach Adenocarcinoma |
| | | Lung Adenocarcinoma |
| 73 | V73Rfs*76 | Adrenocortical Carcinoma |
| | V73Wfs*50 | Colon Adenocarcinoma |
| | | Head and Neck Squamous Cell Carcinoma |
| | | Leiomyosarcoma |
| | | Lung Squamous Cell Carcinoma |
| | | Pancreatic Adenocarcinoma |
| | | Rectal Adenocarcinoma |
| | | Tubular Stomach Adenocarcinoma |
| 74 | A74T | Prostate Adenocarcinoma |
| 75 | P75Lfs*48 | Lung Squamous Cell Carcinoma |
| 76 | A76Hfs*47 | Serous Ovarian Cancer |
| 77 | P77Cfs*73 | Breast Invasive Ductal Carcinoma |
| | P77Gfs*65 | Lung Adenocarcinoma |
| | P77L | |
| 79 | A79Pfs*70 | Colon Adenocarcinoma |
| | A79V | Head and Neck Squamous Cell Carcinoma |
| 82 | P82L | Prostate Adenocarcinoma |
| | P82Rfs*41 | Stomach Adenocarcinoma |
| 83 | A83Gfs*66 | Uterine Serous Carcinoma/Uterine Papillary Serous Carcinoma |
| 84 | A84Pfs*39 | Head and Neck Squamous Cell Carcinoma |
| | | Lung Squamous Cell Carcinoma |
| 85 | P85Lfs*38 | Head and Neck Squamous Cell Carcinoma |
| 86 | A86Cfs*63 | Breast Invasive Ductal Carcinoma |
| | A86Pfs*34 | Prostate Adenocarcinoma |
| | A86Vfs*55 | Serous Ovarian Cancer |
| 90 | S90Ffs*53 | Astrocytoma |
| | S90Pfs*33 | Breast Invasive Ductal Carcinoma |
| | S90Pfs*34 | Head and Neck Squamous Cell Carcinoma |
| | S90Vfs*55 | Hepatocellular Carcinoma |
| | | Lung Adenocarcinoma |
| | | Prostate Adenocarcinoma |
| | | Serous Ovarian Cancer |
| | | Uterine Endometrioid Carcinoma |
| 91 | W91* | Astrocytoma |
| | | Bladder Urothelial Carcinoma |
| | | Breast Invasive Ductal Carcinoma |
| | | Colon Adenocarcinoma |
| | | Esophageal Adenocarcinoma |
| | | Glioblastoma Multiforme |
| | | Head and Neck Squamous Cell Carcinoma |
| | | Leiomyosarcoma |
| | | Lung Adenocarcinoma |
| | | Lung Squamous Cell Carcinoma |
| | | Papillary Renal Cell Carcinoma |
| | | Stomach Adenocarcinoma |
| 92 | P92Afs*57 | Bladder Urothelial Carcinoma |
| | P92S | Uterine Endometrioid Carcinoma |
| 93 | L93Vfs*55 | Breast Invasive Ductal Carcinoma |
| 94 | S94* | Bladder Urothelial Carcinoma |
| | S94Cfs*30 | Colon Adenocarcinoma |
| | | Esophageal Squamous Cell Carcinoma |
| | | Lung Squamous Cell Carcinoma |
| | | Oligoastrocytoma |
| | | Rectal Adenocarcinoma |
| | | Stomach Adenocarcinoma |
| 96 | S96Ffs*25 | Diffuse Type Stomach Adenocarcinoma |
| 97 | V97D | Cutaneous Melanoma |
| | V97Efs*48 | Hepatocellular Carcinoma |
| | V97Sfs*26 | Tubular Stomach Adenocarcinoma |
| | | Uterine Endometrioid Carcinoma |
| 98 | P98Afs*51 | Stomach Adenocarcinoma |
| 99 | S99Rfs*23 | Head and Neck Squamous Cell Carcinoma |
| | | Serous Ovarian Cancer |
| 100 | Q100* | Colon Adenocarcinoma |
| | Q100Gfs*37 | Lung Squamous Cell Carcinoma |
| | | Serous Ovarian Cancer |
| | | Uterine Carcinosarcoma/Uterine Malignant Mixed Mullerian |
| | | Tumor |
| 101 | K101* | Head and Neck Squamous Cell Carcinoma |
| 102 | T102Pfs*21 | Glioblastoma Multiforme |
| | | Serous Ovarian Cancer |
| 103 | Y103* | Head and Neck Squamous Cell Carcinoma |
| | Y103Lfs*46 | Hepatocellular Carcinoma |
| | Y103Tfs*20 | Lung Squamous Cell Carcinoma |
| | | Stomach Adenocarcinoma |
| 104 | Q104* | Esophageal Adenocarcinoma |
| | Q104H | Head and Neck Squamous Cell Carcinoma |
| | Q104Tfs*20 | Lung Adenocarcinoma |
| | | Lung Squamous Cell Carcinoma |
| | | Pancreatic Adenocarcinoma |
| | | Scrous Ovarian Cancer |
| 105 | G105C | Bladder Urothelial Carcinoma |
| | G105D | Glioblastoma Multiforme |
| | G105R | Head and Neck Squamous Cell Carcinoma |
| | G105S | Lung Adenocarcinoma |
| | G105V | Lung Squamous Cell Carcinoma |
| | | Serous Ovarian Cancer |
| 106 | S106R | Esophageal Squamous Cell Carcinoma |
| | | Head and Neck Squamous Cell Carcinoma |
| | | Hepatocellular Carcinoma |
| | | Lung Squamous Cell Carcinoma |
| 107 | Y107Cfs*16 | Breast Invasive Ductal Carcinoma |
| | Y107D | Colon Adenocarcinoma |
| | | Stomach Adenocarcinoma |
| 108 | G108Ffs*40 | Breast Invasive Ductal Carcinoma |
| | G108S | Glioblastoma Multiforme |
| | G108Vfs*15 | Uterine Endometrioid Carcinoma |
| 109 | F109C | Colon Adenocarcinoma |
| | F109Lfs*36 | Esophageal Squamous Cell Carcinoma |
| | F109S | Lung Adenocarcinoma |
| | F109V | Lung Squamous Cell Carcinoma |
| | | Oligoastrocytoma |
| | | Oligodendroglioma |
| | | Rectal Adenocarcinoma |
| 110 | R110_G112del | Bladder Urothelial Carcinoma |
| | R110C | Breast Invasive Ductal Carcinoma |
| | R110del | Cutaneous Melanoma |
| | R110L | Esophageal Squamous Cell Carcinoma |
| | R110P | Head and Neck Squamous Cell Carcinoma |
| | R110Sfs*14 | Lung Adenocarcinoma |
| | R110Vfs*13 | Lung Squamous Cell Carcinoma |
| | R110Wfs*12 | Oligoastrocytoma |
| | | Pancreatic Adenocarcinoma |
| | | Serous Ovarian Cancer |
| 111 | L111P | Bladder Urothelial Carcinoma |
| | L111Q | Breast Invasive Ductal Carcinoma |
| | L111R | Diffuse Type Stomach Adenocarcinoma |
| | | Lung Squamous Cell Carcinoma |
| | | Papillary Renal Cell Carcinoma |
| 113 | F113C | Astrocytoma |
| | F113del | Breast Invasive Carcinoma (NOS) |
| | F113L | Breast Invasive Ductal Carcinoma |
| | F113V | Colon Adenocarcinoma |
| | | Esophageal Adenocarcinoma |
| | | Hepatocellular Carcinoma |
| | | Intestinal Type Stomach Adenocarcinoma |
| | | Leiomyosarcoma |
| | | Oligodendroglioma |
| 114 | L114* | Breast Invasive Carcinoma (NOS) |
| 115 | H115Ifs*8 | Breast Invasive Ductal Carcinoma |
| 118 | T118Dfs*31 | Breast Invasive Ductal Carcinoma |
| | T118Qfs*5 | Head and Neck Squamous Cell Carcinoma |
| | | Serous Ovarian Cancer |
| 120 | K120E | Cutaneous Melanoma |
| | K120Sfs*3 | Lung Adenocarcinoma |
| | K120Tfs*2 | Lung Squamous Cell Carcinoma |
| | | Oligoastrocytoma |
| | | Undifferentiated Pleomorphic Sarcoma/Malignant Fibrous |
| | | Histiocytoma/High-Grade Spindle Cell Sarcoma |
| 121 | S121Cfs*27 | Bladder Urothelial Carcinoma |
| | S121Y | Uterine Carcinosarcoma/Uterine Malignant Mixed Mullerian |
| | | Tumor |
| 122 | V122Cfs*27 V122Dfs*26 | Astrocytoma |
| | V122Lfs*25 | Breast Invasive Ductal Carcinoma |
| | | Colon Adenocarcinoma |
| | | Esophageal Squamous Cell Carcinoma |
| | | Leiomyosarcoma |
| | | Oligoastrocytoma |
| | | Oligodendroglioma |
| | | Pancreatic Adenocarcinoma |
| | | Rectal Adenocarcinoma |
| | | Serous Ovarian Cancer |
| 123 | T123Dfs*26 | Glioblastoma Multiforme |
| | T123Lfs*47 | |
| | T123Rfs*48 | Pancreatic Adenocarcinoma |
| | | Serous Ovarian Cancer |
| 124 | C124* | Breast Invasive Ductal Carcinoma |
| | C124Afs*46 | Colon Adenocarcinoma |
| | C124G | Esophageal Squamous Cell Carcinoma |
| | C124Wfs*25 | Prostate Adenocarcinoma |
| | | Serous Ovarian Cancer |
| 125 | T125= | Acute Myeloid Leukemia |
| | (splice region) | Adrenocortical Carcinoma |
| | T125M | Astrocytoma |
| | T125P | Bladder Urothelial Carcinoma |
| | X125_splice | Breast Invasive Ductal Carcinoma |
| | | Colon Adenocarcinoma |
| | | Cutaneous Melanoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Lung Adenocarcinoma |
| | | Lung Squamous Cell Carcinoma |
| | | Mucinous Adenocarcinoma of the Colon and Rectum |
| | | Mucinous Stomach Adenocarcinoma |
| | | Oligoastrocytoma |
| | | Oligodendroglioma |
| | | Pancreatic Adenocarcinoma |
| | | Rectal Adenocarcinoma |
| | | Serous Ovarian Cancer |
| | | Stomach Adenocarcinoma |
| | | Tubular Stomach Adenocarcinoma |
| | | Undifferentiated Pleomorphic Sarcoma/Malignant Fibrous |
| | | Histiocytoma/High-Grade Spindle Cell Sarcoma |
| | | Uterine Endometrioid Carcinoma |
| | | Uterine Serous Carcinoma/Uterine Papillary Serous Carcinoma |
| 126 | Y126_M133del | Acute Myeloid Leukemia |
| | Y126C | Breast Invasive Ductal Carcinoma |
| | Y126D | Cutaneous Melanoma |
| | Y126H | Esophageal Squamous Cell Carcinoma |
| | Y126N | Glioblastoma Multiforme |
| | Y126S | Head and Neck Squamous Cell Carcinoma |
| | X126_splice | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Stomach Adenocarcinoma |
| | | Oligodendroglioma |
| | | Pancreatic Adenocarcinoma |
| | | Prostate Adenocarcinoma |
| | | Rectal Adenocarcinoma |
| | | Serous Ovarian Cancer |
| | | Signet Ring Cell Carcinoma of the Stomach |
| | | Stomach Adenocarcinoma |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| 127 | S127C | Astrocytoma |
| | S127F | Breast Invasive Ductal Carcinoma |
| | S127P | Colon Adenocarcinoma |
| | S127T | Cutaneous Melanoma |
| | S127Y | Glioblastoma Multiforme |
| | S127Yfs*43 | Head and Neck Squamous Cell Carcinoma |
| | | Lung Adenocarcinoma |
| | | Pancreatic Adenocarcinoma |
| | | Serous Ovarian Cancer |
| | | Uterine Endometrioid Carcinoma |
| | | Uterine Serous Carcinoma/Uterine Papillary Serous Carcinoma |
| 128 | P128del | Astrocytoma |
| | P128Lfs*42 | Breast Invasive Ductal Carcinoma |
| | | Esophageal Squamous Cell Carcinoma |
| | | Head and Neck Squamous Cell Carcinoma |
| | | Lung Squamous Cell Carcinoma |
| 129 | A129Vfs*20 | Lung Squamous Cell Carcinoma |
| 130 | L130Cfs*20 | Astrocytoma |
| | L130F | Breast Invasive Lobular Carcinoma |
| | L130P | Colon Adenocarcinoma |
| | L130R | Diffuse Type Stomach Adenocarcinoma |
| | L130V | Esophageal Squamous Cell Carcinoma |
| | | Glioblastoma Multiforme |
| | | Lung Adenocarcinoma |
| | | Serous Ovarian Cancer |
| | | Tubular Stomach Adenocarcinoma |
| | | Uterine Mixed Endometrial Carcinoma |
| 131 | N131del | Astrocytoma |
| | N131I | Breast Invasive Ductal Carcinoma |
| | N131Kfs*39 | Lung Adenocarcinoma |
| | N131Qfs*17 | Lung Squamous Cell Carcinoma |
| | N131S | Pancreatic Adenocarcinoma |
| | N131Tfs*39 | Serous Ovarian Cancer |
| | | Stomach Adenocarcinoma |
| | | Uterine Mixed Endometrial Carcinoma |
| 132 | K132E | Adrenocortical Carcinoma |
| | K132M | Astrocytoma |
| | K132N | Bladder Urothelial Carcinoma |
| | K132Q | Breast Invasive Ductal Carcinoma |
| | K132R | Cervical Squamous Cell Carcinoma |
| | K132T | Colon Adenocarcinoma |
| | K132Vfs*15 | Glioblastoma Multiforme |
| | | Head and Neck Squamous Cell Carcinoma |
| | | Leiomyosarcoma |
| | | Lung Adenocarcinoma |
| | | Lung Squamous Cell Carcinoma |
| | | Oligoastrocytoma |
| | | Pleural Mesothelioma, Epithelioid Type |
| | | Serous Ovarian Cancer |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| 133 | M133K | Breast Invasive Carcinoma (NOS) |
| | M133R | Head and Neck Squamous Cell Carcinoma |
| | | Mucinous Adenocarcinoma of the Colon and Rectum |
| 134 | F134L | Cutaneous Melanoma |
| | F134V | Esophageal Squamous Cell Carcinoma |
| | | Head and Neck Squamous Cell Carcinoma |
| | | Serous Ovarian Cancer |
| | | Tubular Stomach Adenocarcinoma |
| | | Uterine Endometrioid Carcinoma |
| 135 | C135* | Adrenocortical Carcinoma |
| | C135Afs*35 | Bladder Urothelial Carcinoma |
| | C135F | Breast Invasive Ductal Carcinoma |
| | C135Lfs*14 | Colon Adenocarcinoma |
| | C135R | Diffuse Type Stomach Adenocarcinoma |
| | C135W | Esophageal Adenocarcinoma |
| | C135Y | Esophageal Squamous Cell Carcinoma |
| | | Head and Neck Squamous Cell Carcinoma |
| | | Hepatocellular Carcinoma |
| | | Lung Adenocarcinoma |
| | | Lung Squamous Cell Carcinoma |
| | | Pancreatic Adenocarcinoma |
| | | Papillary Stomach Adenocarcinoma |
| | | Prostate Adenocarcinoma |
| | | Serous Ovarian Cancer |
| | | Tubular Stomach Adenocarcinoma |
| | | Undifferentiated Pleomorphic Sarcoma/Malignant Fibrous |
| | | Histiocytoma/High-Grade Spindle Cell Sarcoma |
| | | Uterine Endometrioid Carcinoma |
| 136 | Q136* | Astrocytoma |
| | Q136_C141del | Breast Invasive Ductal Carcinoma |
| | Q136del | Glioblastoma Multiforme |
| | Q136E | Head and Neck Squamous Cell Carcinoma |
| | Q136H | Hepatocellular Carcinoma |
| | Q136P | Intestinal Type Stomach Adenocarcinoma |
| | | Leiomyosarcoma |
| | | Lung Adenocarcinoma |
| | | Lung Squamous Cell Carcinoma |
| | | Metaplastic Breast Cancer |
| | | Mucinous Stomach Adenocarcinoma |
| | | Oligodendroglioma |
| | | Rectal Adenocarcinoma |
| | | Serous Ovarian Cancer |
| | | Stomach Adenocarcinoma |
| 137 | L137Pfs*32 | Head and Neck Squamous Cell Carcinoma |
| | L137Q | |
| 138 | A138_L145dcl | Breast Invasive Ductal Carcinoma |
| | A138_P142del | Esophageal Adenocarcinoma |
| | A138Cfs*27 | Glioblastoma Multiforme |
| | A138T | Lung Adenocarcinoma |
| | A138V | Rectal Adenocarcinoma |
| | | Serous Ovarian Cancer |
| | | Uterine Endometrioid Carcinoma |
| 139 | K139_P142del | Bladder Urothelial Carcinoma |
| | K139_P142delinsT | Breast Invasive Carcinoma (NOS) |
| | K139Afs*5 | Esophageal Squamous Cell Carcinoma |
| | K139Cfs*6 | Glioblastoma Multiforme |
| | K139N | Head and Neck Squamous Cell Carcinoma |
| | K139Nfs*9 | Hepatocellular Carcinoma |
| | K139Rfs*31 | Lung Adenocarcinoma |
| | | Uterine Endometrioid Carcinoma |
| 140 | T140Mfs*28 | Serous Ovarian Cancer |
| 141 | C141* | Acute Myeloid Leukemia |
| | C141Afs*29 | Astrocytoma |
| | C141F | Bladder Urothelial Carcinoma |
| | C141G | Breast Invasive Ductal Carcinoma |
| | C141R | Chromophobe Renal Cell Carcinoma |
| | C141S | Colon Adenocarcinoma |
| | C141W | Cutaneous Melanoma |
| | C141Y | Head and Neck Squamous Cell Carcinoma |
| | | Intrahepatic Cholangiocarcinoma |
| | | Lung Adenocarcinoma |
| | | Lung Squamous Cell Carcinoma |
| | | Oligoastrocytoma |
| | | Prostate Adenocarcinoma |
| | | Serous Ovarian Cancer |
| | | Stomach Adenocarcinoma |
| | | Uterine Endometrioid Carcinoma |
| | | Uterine Serous Carcinoma/Uterine Papillary Serous Carcinoma |
| 142 | P142del | Lung Squamous Cell Carcinoma |
| | P142L | Oligoastrocytoma |
| | P142Lfs*28 | Uterine Mixed Endometrial Carcinoma |
| 143 | V143A | Bladder Urothelial Carcinoma |
| | V143Afs*29 | Colon Adenocarcinoma |
| | V143E | Cutaneous Melanoma |
| | V143G | Esophageal Adenocarcinoma |
| | V143M | Head and Neck Squamous Cell Carcinoma |
| | V143Rfs*18 | Hepatocellular Carcinoma |
| | | Oligoastrocytoma |
| | | Pancreatic Adenocarcinoma |
| | | Uterine Carcinosarcoma/Uterine Malignant Mixed Mullerian |
| | | Tumor |
| 144 | Q144* | Bladder Urothelial Carcinoma |
| | Q144_L145dup | Breast Invasive Lobular Carcinoma |
| | Q144Afs*16 | Colon Adenocarcinoma |
| | Q144Gfs*24 | Head and Neck Squamous Cell Carcinoma |
| | Q144H | Hepatocellular Carcinoma |
| | Q144L | Lung Adenocarcinoma |
| | Q144Lfs*5 | Lung Squamous Cell Carcinoma |
| | Q144P | Prostate Adenocarcinoma |
| | Q144Tfs*5 | Serous Ovarian Cancer |
| | | Stomach Adenocarcinoma |
| | | Uterine Serous Carcinoma/Uterine Papillary Serous Carcinoma |
| 145 | L145P | Colon Adenocarcinoma |
| | L145Q | Oligoastrocytoma |
| | L145R | Serous Ovarian Cancer |
| | | Signet Ring Cell Carcinoma of the Stomach |
| 146 | W146* | Astrocytoma |
| | W146S | Bladder Urothelial Carcinoma |
| | W146Vfs*3 | Esophageal Squamous Cell Carcinoma |
| | | Head and Neck Squamous Cell Carcinoma |
| | | Intestinal Type Stomach Adenocarcinoma |
| | | Lung Adenocarcinoma |
| | | Lung Squamous Cell Carcinoma |
| | | Oligodendroglioma |
| | | Rectal Adenocarcinoma |
| | | Serous Ovarian Cancer |
| | | Uterine Endometrioid Carcinoma |
| | | Uterine Serous Carcinoma/Uterine Papillary Serous Carcinoma |
| 147 | V147_D148dup | Breast Invasive Ductal Carcinoma |
| | V147G | Esophageal Adenocarcinoma |
| | V147Lfs*23 | Serous Ovarian Cancer |
| 148 | D148Ifs*22 | Bladder Urothelial Carcinoma |
| | D148N | Lung Adenocarcinoma |
| 149 | S149Ffs*32 | Pancreatic Adenocarcinoma |
| | S149Pfs*21 | Prostate Adenocarcinoma |
| | | Serous Ovarian Cancer |
| 150 | T150Afs*16 | Stomach Adenocarcinoma |
| 151 | P151A | Astrocytoma |
| | P151H | Bladder Urothelial Carcinoma |
| | P151L | Breast Invasive Ductal Carcinoma |
| | P151R | Colon Adenocarcinoma |
| | P151Rfs*27 | Cutaneous Melanoma |
| | P151S | Esophageal Squamous Cell Carcinoma |
| | P151T | Head and Neck Squamous Cell Carcinoma |
| | | Hepatocellular Carcinoma |
| | | Leiomyosarcoma |
| | | Lung Adenocarcinoma |
| | | Lung Squamous Cell Carcinoma |
| | | Oligoastrocytoma |
| | | Pancreatic Adenocarcinoma |
| | | Rectal Adenocarcinoma |
| | | Serous Ovarian Cancer |
| | | Stomach Adenocarcinoma |
| | | Tubular Stomach Adenocarcinoma |
| | | Uterine Carcinosarcoma/Uterine Malignant Mixed Mullerian |
| | | Tumor |
| | | Uterine Mixed Endometrial Carcinoma |
| | | Uterine Serous Carcinoma/Uterine Papillary Serous Carcinoma |
| 152 | P152Afs*14 | Astrocytoma |
| | P152L | Bladder Urothelial Carcinoma |
| | P152Lfs*18 | Breast Invasive Ductal Carcinoma |
| | P152Q | Cutaneous Melanoma |
| | P152Rfs*18 | Diffuse Type Stomach Adenocarcinoma |
| | P152S | Glioblastoma Multiforme |
| | P152Wfs*10 | Head and Neck Squamous Cell Carcinoma |
| | | Intestinal Type Stomach Adenocarcinoma |
| | | Lung Adenocarcinoma |
| | | Lung Squamous Cell Carcinoma |
| | | Oligoastrocytoma |
| | | Pancreatic Adenocarcinoma |
| | | Serous Ovarian Cancer |
| | | Signet Ring Cell Carcinoma of the Stomach |
| | | Tubular Stomach Adenocarcinoma |
| 153 | P153Afs*28 | Bladder Urothelial Carcinoma |
| | P153Rfs*18 | Lung Squamous Cell Carcinoma |
| | | Oligoastrocytoma |
| | | Stomach Adenocarcinoma |
| 154 | G154Afs*16 | Bladder Urothelial Carcinoma |
| | G154Rfs*27 | Esophageal Squamous Cell Carcinoma |
| | G154V | Head and Neck Squamous Cell Carcinoma |
| | G154Wfs*10 | Lung Adenocarcinoma |
| | | Lung Squamous Cell Carcinoma |
| | | Oligodendroglioma |
| | | Pancreatic Adenocarcinoma |
| | | Serous Ovarian Cancer |
| 155 | T155_A161del | Astrocytoma |
| | T155_R156del | Bladder Urothelial Carcinoma |
| | T155_R156delinsN | Breast Invasive Ductal Carcinoma |
| | T155_R158del | Colon Adenocarcinoma |
| | T155_R158delinsC | Glioblastoma Multiforme |
| | T155I | Head and Neck Squamous Cell Carcinoma |
| | T155N | Lung Adenocarcinoma |
| | T155P | Lung Squamous Cell Carcinoma |
| | | Oligoastrocytoma |
| | | Serous Ovarian Cancer |
| 156 | R156Afs*12 | Astrocytoma |
| | R156C | Bladder Urothelial Carcinoma |
| | R156del | Breast Invasive Ductal Carcinoma |
| | R156G | Glioblastoma Multiforme |
| | R156H | Head and Neck Squamous Cell Carcinoma |
| | R156Hfs*26 | Hepatocellular Carcinoma |
| | R156P | Leiomyosarcoma |
| | R156Pfs*13 | Lung Adenocarcinoma |
| | | Serous Ovarian Cancer |
| | | Uterine Serous Carcinoma/Uterine Papillary Serous Carcinoma |
| 157 | V157_P177del | Breast Invasive Ductal Carcinoma |
| | V157_R158dup | Colon Adenocarcinoma |
| | V157Afs*24 | Esophageal Squamous Cell Carcinoma |
| | V157D | Glioblastoma Multiforme |
| | V157F | Head and Neck Squamous Cell Carcinoma |
| | V157G | Hepatocellular Carcinoma |
| | V157Hfs*21 | Lung Adenocarcinoma |
| | V157L | Lung Squamous Cell Carcinoma |
| | V157Pfs*23 | Oligodendroglioma |
| | V157Sfs*13 | Prostate Adenocarcinoma |
| | | Serous Ovarian Cancer |
| | | Uterine Endometrioid Carcinoma |
| | | Uterine Mixed Endometrial Carcinoma |
| | | Uterine Serous Carcinoma/Uterine Papillary Serous Carcinoma |
| 158 | R158Afs*12 | Astrocytoma |
| | R158C | Bladder Urothelial Carcinoma |
| | R158G | Breast Invasive Ductal Carcinoma |
| | R158H | Colon Adenocarcinoma |
| | R158L | Diffuse Type Stomach Adenocarcinoma |
| | R158P | Esophageal Squamous Cell Carcinoma |
| | R158S | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Oligoastrocytoma |
| | | Oligodendroglioma |
| | | Prostate Adenocarcinoma |
| | | Stomach Adenocarcinoma |
| | | Uterine Endometrioid Carcinoma |
| 159 | A159P | Astrocytoma |
| | A159V | Bladder Urothelial Carcinoma |
| | | Cervical Squamous Cell Carcinoma |
| | | Chromophobe Renal Cell Carcinoma |
| | | Colon Adenocarcinoma |
| | | Cutaneous Melanoma |
| | | Head and Neck Squamous Cell Carcinoma |
| | | Lung Adenocarcinoma |
| | | Lung Squamous Cell Carcinoma |
| | | Rectal Adenocarcinoma |
| | | Serous Ovarian Cancer |
| | | Uterine Endometrioid Carcinoma |
| | | Uterine Serous Carcinoma/Uterine Papillary Serous Carcinoma |
| 160 | M160I | Lung Squamous Cell Carcinoma |
| 161 | A161_I162del | Astrocytoma |
| | A161D | Bladder Urothelial Carcinoma |
| | A161Gfs*3 | Breast Invasive Ductal Carcinoma |
| | A161P | Colon Adenocarcinoma |
| | A161S | Cutaneous Melanoma |
| | A161T | Head and Neck Squamous Cell Carcinoma |
| | A161V | Hepatocellular Carcinoma |
| | | Lung Adenocarcinoma |
| | | Lung Squamous Cell Carcinoma |
| | | Oligoastrocytoma |
| | | Serous Ovarian Cancer |
| 162 | I162_Y163delinsN | Breast Invasive Ductal Carcinoma |
| | I162dup | Colon Adenocarcinoma |
| | I162F | Lung Squamous Cell Carcinoma |
| | I162Hfs*12 | Tubular Stomach Adenocarcinoma |
| | I162N | Uterine Serous Carcinoma/Uterine Papillary Serous Carcinoma |
| | I162Tfs*8 | |
| | I162Wfs*10 | |
| 163 | Y163* | Astrocytoma |
| | Y163D | Breast Invasive Ductal Carcinoma |
| | Y163H | Colon Adenocarcinoma |
| | Y163Lfs*18 | Esophageal Squamous Cell Carcinoma |
| | Y163N | Head and Neck Squamous Cell Carcinoma |
| | | Lung Adenocarcinoma |
| | | Lung Squamous Cell Carcinoma |
| | | Malignant Peripheral Nerve Sheath Tumor |
| | | Oligoastrocytoma |
| | | Prostate Adenocarcinoma |
| | | Serous Ovarian Cancer |
| | | Uterine Carcinosarcoma/Uterine Malignant Mixed Mullerian |
| | | Tumor |
| 164 | K164E | Esophageal Squamous Cell Carcinoma |
| | K164Sfs*5 | Glioblastoma Multiforme |
| | K164Sfs*6 | Lung Squamous Cell Carcinoma |
| | | Mucinous Carcinoma |
| | | Rectal Adenocarcinoma |
| | | Serous Ovarian Cancer |
| 165 | Q165* | Breast Invasive Ductal Carcinoma |
| | Q165Afs*6 | Lung Adenocarcinoma |
| | Q165Hfs*17 | Prostate Adenocarcinoma |
| 166 | S166* | Head and Neck Squamous Cell Carcinoma |
| | | Lung Adenocarcinoma |
| | | Pancreatic Adenocarcinoma |
| | | Uterine Serous Carcinoma/Uterine Papillary Serous Carcinoma |
| 167 | Q167* | Bladder Urothelial Carcinoma |
| | Q167Hfs*3 | Breast Invasive Ductal Carcinoma |
| | Q167Tfs*14 | Esophageal Adenocarcinoma |
| | | Head and Neck Squamous Cell Carcinoma |
| | | Lung Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Endometrioid Carcinoma |
| 168 | H168Cfs*8 | Adrenocortical Carcinoma |
| | H168L | Breast Invasive Ductal Carcinoma |
| | H168P | Esophageal Squamous Cell Carcinoma |
| | H168R | Head and Neck Squamous Cell Carcinoma |
| | | Lung Adenocarcinoma |
| | | Stomach Adenocarcinoma |
| 169 | M169I | Bladder Urothelial Carcinoma |
| | M169T | Breast Invasive Lobular Carcinoma |
| | | Stomach Adenocarcinoma |
| 171 | E171* | Bladder Urothelial Carcinoma |
| | E171Gfs*3 | Breast Invasive Ductal Carcinoma |
| | E171Gfs*4 | Cutaneous Melanoma |
| | E171K | Diffuse Large B-Cell Lymphoma, NOS |
| | E171Lfs*2 | Glioblastoma Multiforme |
| | E171Rfs*3 | Head and Neck Squamous Cell Carcinoma |
| | | Hepatocellular Carcinoma |
| | | Intestinal Type Stomach Adenocarcinoma |
| | | Lung Adenocarcinoma |
| | | Lung Squamous Cell Carcinoma |
| | | Oligoastrocytoma |
| | | Pleural Mesothelioma, Epithelioid Type |
| | | Rectal Adenocarcinoma |
| 172 | V172D | Esophageal Adenocarcinoma |
| | V172F | Glioblastoma Multiforme |
| | V172G | Head and Neck Squamous Cell Carcinoma |
| | V172Lfs*2 | Lung Squamous Cell Carcinoma |
| | | Pleural Mesothelioma, Epithelioid Type |
| 173 | V173* | Acute Myeloid Leukemia |
| | V173_R175del | Adrenocortical Carcinoma |
| | V173A | Astrocytoma |
| | V173Afs*69 | Breast Invasive Ductal Carcinoma |
| | V173dup | Colon Adenocarcinoma |
| | V173Efs*7 | Esophageal Adenocarcinoma |
| | V173G | Head and Neck Squamous Cell Carcinoma |
| | V173L | Lung Adenocarcinoma |
| | V173M | Lung Squamous Cell Carcinoma |
| | | Oligoastrocytoma |
| | | Oligodendroglioma |
| | | Pancreatic Adenocarcinoma |
| | | Serous Ovarian Cancer |
| | | Signet Ring Cell Carcinoma of the Stomach |
| | | Stomach Adenocarcinoma |
| | | Tubular Stomach Adenocarcinoma |
| | | Uterine Mixed Endometrial Carcinoma |
| | | Uterine Serous Carcinoma/Uterine Papillary Serous Carcinoma |
| 174 | R174Sfs*73 | Breast Invasive Ductal Carcinoma |
| | R174W | Esophageal Squamous Cell Carcinoma |
| | | Hepatocellular Carcinoma |
| | | Uterine Endometrioid Carcinoma |
| 175 | R175C | Astrocytoma |
| | R175G | Bladder Urothelial Carcinoma |
| | R175H | Breast Invasive Ductal Carcinoma |
| | R175L | Colon Adenocarcinoma |
| | | Cutaneous Melanoma |
| | | Diffuse Type Stomach Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Adenocarcinoma of the Colon and Rectum |
| | | Mucinous Stomach Adenocarcinoma |
| | | Oligoastrocytoma |
| | | Pancreatic Adenocarcinoma |
| | | Prostate Adenocarcinoma |
| | | Rectal Adenocarcinoma |
| | | Serous Ovarian Cancer |
| | | Stomach Adenocarcinoma |
| | | Tubular Stomach Adenocarcinoma |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Endometrioid Carcinoma |
| | | Uterine Serous Carcinoma/Uterine Papillary Serous Carcinoma |
| 176 | C176* | Acute Myeloid Leukemia |
| | C176dup | Bladder Urothelial Carcinoma |
| | C176F | Breast Invasive Carcinoma (NOS) |
| | C176G | Breast Invasive Ductal Carcinoma |
| | C176R | Chromophobe Renal Cell Carcinoma |
| | C176S | Colon Adenocarcinoma |
| | C176Sfs*71 | Diffuse Type Stomach Adenocarcinoma |
| | C176W | Esophageal Adenocarcinoma |
| | C176Y | Esophageal Squamous Cell Carcinoma |
| | | Head and Neck Squamous Cell Carcinoma |
| | | Hepatocellular Carcinoma |
| | | Lung Adenocarcinoma |
| | | Lung Squamous Cell Carcinoma |
| | | Mucinous Stomach Adenocarcinoma |
| | | Oligoastrocytoma |
| | | Oligodendroglioma |
| | | Prostate Adenocarcinoma |
| | | Serous Ovarian Cancer |
| | | Stomach Adenocarcinoma |
| | | Tubular Stomach Adenocarcinoma |
| | | Uterine Carcinosarcoma/Uterine Malignant Mixed Mullerian |
| | | Tumor |
| | | Uterine Serous Carcinoma/Uterine Papillary Serous Carcinoma |
| 177 | P177_C182del | Astrocytoma |
| | P177_H178del | Colon Adenocarcinoma |
| | P177H | Cutaneous Melanoma |
| | P177L | Esophageal Adenocarcinoma |
| | P177R | Head and Neck Squamous Cell Carcinoma |
| | P177S | Lung Adenocarcinoma |
| | P177T | Prostate Adenocarcinoma |
| | | Serous Ovarian Cancer |
| 178 | H178_S183del | Bladder Urothelial Carcinoma |
| | H178D | Chromophobe Renal Cell Carcinoma |
| | H178Pfs*3 | Colon Adenocarcinoma |
| | H178Pfs*70 | Glioblastoma Multiforme |
| | H178Q | Head and Neck Squamous Cell Carcinoma |
| | H178Qfs*3 | Lung Adenocarcinoma |
| | H178Sfs*69 | Lung Squamous Cell Carcinoma |
| | H178Tfs*69 | Pancreatic Adenocarcinoma |
| | | Renal Clear Cell Carcinoma |
| | | Stomach Adenocarcinoma |
| | | Uterine Endometrioid Carcinoma |
| 179 | H179D | Acute Myeloid Leukemia |
| | H179L | Astrocytoma |
| | H179N | Bladder Urothelial Carcinoma |
| | H179P | Breast Invasive Ductal Carcinoma |
| | H179Q | Colon Adenocarcinoma |
| | H179R | Cutaneous Melanoma |
| | H179Y | Diffuse Type Stomach Adenocarcinoma |
| | | Esophageal |
| | | Adenocarcinoma |
| | | Esophageal Squamous Cell Carcinoma |
| | | Glioblastoma Multiforme |
| | | Head and Neck Squamous Cell Carcinoma |
| | | Leiomyosarcoma |
| | | Lung Adenocarcinoma |
| | | Lung Squamous Cell Carcinoma |
| | | Oligoastrocytoma |
| | | Oligodendroglioma |
| | | Pancreatic Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Endometrioid Carcinoma |
| | | Uterine Serous Carcinoma/Uterine Papillary Serous Carcinoma |
| 180 | E180* | Adrenocortical Carcinoma |
| | E180_D184del | Bladder Urothelial Carcinoma |
| | E180_S185del | Head and Neck Squamous Cell Carcinoma |
| | E180K | Lung Adenocarcinoma |
| | | Lung Squamous Cell Carcinoma |
| | | Rectal Adenocarcinoma |
| 181 | R181C | Astrocytoma |
| | R181H | Bladder Urothelial Carcinoma |
| | R181P | Colon Adenocarcinoma |
| | | Lung Squamous Cell Carcinoma |
| | | Prostate Adenocarcinoma |
| | | Uterine Carcinosarcoma/Uterine Malignant Mixed Mullerian |
| | | Tumor |
| | | Uterine Endometrioid Carcinoma |
| 183 | S183* | Bladder Urothelial Carcinoma |
| | | Breast Invasive Ductal Carcinoma |
| | | Cervical Squamous Cell Carcinoma |
| | | Lung Adenocarcinoma |
| | | Lung Squamous Cell Carcinoma |
| | | Uterine Carcinosarcoma/Uterine Malignant Mixed Mullerian |
| | | Tumor |
| 184 | D184Afs*62 | Head and Neck Squamous Cell Carcinoma |
| 186 | D186Vfs*61 | Pancreatic Adenocarcinoma |
| 187 | G187S | Bladder Urothelial Carcinoma |
| | X187_splice | Breast Invasive Carcinoma (NOS) |
| | | Breast Invasive Ductal Carcinoma |
| | | Chromophobe Renal Cell Carcinoma |
| | | Colon Adenocarcinoma |
| | | Cutaneous Melanoma |
| | | Esophageal Squamous Cell Carcinoma |
| | | Glioblastoma Multiforme |
| | | Head and Neck Squamous Cell Carcinoma |
| | | Leiomyosarcoma |
| | | Lung Adenocarcinoma |
| | | Lung Squamous Cell Carcinoma |
| | | Oligoastrocytoma |
| | | Pancreatic Adenocarcinoma |
| | | Rectal Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Endometrioid Carcinoma |
| 188 | L188Gfs*20 | Lung Squamous Cell Carcinoma |
| | L188Tfs*21 | Serous Ovarian Cancer |
| 189 | A189Dfs*14 | Bladder Urothelial Carcinoma |
| | A189Pfs*58 | Leiomyosarcoma |
| | | Lung Squamous Cell Carcinoma |
| 190 | P190L | Astrocytoma |
| | P190R | Chromophobe Renal Cell Carcinoma |
| | | Colon Adenocarcinoma |
| | | Glioblastoma Multiforme |
| | | Lung Squamous Cell Carcinoma |
| 191 | P191del | Astrocytoma |
| | P191L | Colon Adenocarcinoma |
| | P191Qfs*51 | Cutaneous Melanoma |
| | | Diffuse Type Stomach Adenocarcinoma |
| | | Head and Neck Squamous Cell Carcinoma |
| | | Hepatocellular Carcinoma |
| | | Lung Adenocarcinoma |
| | | Oligodendroglioma |
| | | Serous Ovarian Cancer |
| | | Stomach Adenocarcinoma |
| 192 | Q192* | Bladder Urothelial Carcinoma |
| | Q192_H193del | Breast Invasive Ductal Carcinoma |
| | Q192del | Cervical Squamous Cell Carcinoma |
| | Q192R | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Pancreatic Adenocarcinoma |
| | | Papillary Thyroid Cancer |
| | | Serous Ovarian Cancer |
| | | Tubular Stomach Adenocarcinoma |
| 193 | H193D | Acute Myeloid Leukemia |
| | H193L | Astrocytoma |
| | H193N | Bladder Urothelial Carcinoma |
| | H193P | Breast Invasive Carcinoma (NOS) |
| | H193R | Breast Invasive Ductal Carcinoma |
| | H193Y | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Lung Adenocarcinoma |
| | | Lung Squamous Cell Carcinoma |
| | | Metaplastic Breast Cancer |
| | | Oligoastrocytoma |
| | | Papillary Renal Cell Carcinoma |
| | | Prostate Adenocarcinoma |
| | | Renal Clear Cell Carcinoma |
| | | Serous Ovarian Cancer |
| | | Stomach Adenocarcinoma |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| | | Uterine Serous Carcinoma/Uterine Papillary Serous Carcinoma |
| 194 | L194Efs*51 | Bladder Urothelial Carcinoma |
| | L194F | Breast Invasive Ductal Carcinoma |
| | L194H | Endometrioid Carcinoma |
| | L194P | Glioblastoma Multiforme |
| | L194Pfs*13 | Head and Neck Squamous Cell Carcinoma |
| | L194R | Hepatocellular Carcinoma |
| | | Leiomyosarcoma |
| | | Lung Adenocarcinoma |
| | | Lung Squamous Cell Carcinoma |
| | | Oligoastrocytoma |
| | | Pancreatic Adenocarcinoma |
| | | Rectal Adenocarcinoma |
| | | Serous Ovarian Cancer |
| | | Thymoma |
| | | Uterine Serous Carcinoma/Uterine Papillary Serous Carcinoma |
| 195 | I195F | Acute Myeloid Leukemia |
| | I195Ffs*52 | Astrocytoma |
| | I195Lfs*53 | Breast Invasive Ductal Carcinoma |
| | I195M | Colon Adenocarcinoma |
| | I195N | Cutaneous Melanoma |
| | I195Nfs*14 | Esophageal Squamous Cell Carcinoma |
| | I195S | Glioblastoma Multiforme |
| | I195Sfs*52 | Head and Neck Squamous Cell Carcinoma |
| | I195T | Hepatocellular Carcinoma |
| | I195Yfs*14 | Leiomyosarcoma |
| | | Lung Adenocarcinoma |
| | | Lung Squamous Cell Carcinoma |
| | | Oligoastrocytoma |
| | | Oligodendroglioma |
| | | Rectal Adenocarcinoma |
| | | Serous Ovarian Cancer |
| | | Stomach Adenocarcinoma |
| | | Tubular Stomach Adenocarcinoma |
| | | Uterine Carcinosarcoma/Uterine Malignant Mixed Mullerian |
| | | Tumor |
| | | Uterine Serous Carcinoma/Uterine Papillary Serous Carcinoma |
| 196 | R196* | Astrocytoma |
| | R196P | Breast Invasive Carcinoma (NOS) |
| | | Breast Invasive Ductal Carcinoma |
| | | Cervical Squamous Cell Carcinoma |
| | | Chromophobe Renal Cell Carcinoma |
| | | Colon Adenocarcinoma |
| | | Cutaneous Melanoma |
| | | Esophageal Adenocarcinoma |
| | | Glioblastoma Multiforme |
| | | Head and Neck Squamous Cell Carcinoma |
| | | Intestinal Type Stomach Adenocarcinoma |
| | | Lung Adenocarcinoma |
| | | Lung Squamous Cell Carcinoma |
| | | Mucinous Adenocarcinoma of the Colon and Rectum |
| | | Oligodendroglioma |
| | | Pancreatic Adenocarcinoma |
| | | Pleural Mesothelioma, Epithelioid Type |
| | | Rectal Adenocarcinoma |
| | | Serous Ovarian Cancer |
| | | Stomach Adenocarcinoma |
| | | Tubular Stomach Adenocarcinoma |
| | | Uterine Serous Carcinoma/Uterine Papillary Serous Carcinoma |
| 197 | V197G | Breast Invasive Ductal Carcinoma |
| | V197Ifs*42 | Esophageal Squamous Cell Carcinoma |
| | V197L | Head and Neck Squamous Cell Carcinoma |
| | V197M | Hepatocellular Carcinoma |
| | | Lung Squamous Cell Carcinoma |
| | | Pancreatic Adenocarcinoma |
| | | Uterine Serous Carcinoma/Uterine Papillary Serous Carcinoma |
| 198 | E198* | Breast Invasive Ductal Carcinoma |
| | E198Gfs*11 | Colon Adenocarcinoma |
| | | Cutaneous Melanoma |
| | | Esophageal Adenocarcinoma |
| | | Esophageal Squamous Cell Carcinoma |
| | | Lung Adenocarcinoma |
| | | Lung Squamous Cell Carcinoma |
| | | Serous Ovarian Cancer |
| | | Tubular Stomach Adenocarcinoma |
| 199 | G199* | Bladder Urothelial Carcinoma |
| | G199Efs*48 | Breast Invasive Ductal Carcinoma |
| | G199Ifs*47 | Colon Adenocarcinoma |
| | G199V | Head and Neck Squamous Cell Carcinoma |
| | | Pancreatic Adenocarcinoma |
| | | Prostate Adenocarcinoma |
| | | Uterine Endometrioid Carcinoma |
| 200 | N200Ifs*47 | Head and Neck Squamous Cell Carcinoma |
| | | Oligoastrocytoma |
| 201 | L201* | Breast Invasive Ductal Carcinoma |
| | L201Ffs*8 | Head and Neck Squamous Cell Carcinoma |
| 202 | R202_L206del | Head and Neck Squamous Cell Carcinoma |
| 203 | V203Gfs*44 | Head and Neck Squamous Cell Carcinoma |
| | V203Gfs*5 | Leiomyosarcoma |
| | V203L | Mucinous Adenocarcinoma of the Colon and Rectum |
| | V203Wfs*44 | Prostate Adenocarcinoma |
| 204 | E204* | Bladder Urothelial Carcinoma |
| | E204D | Breast Invasive Ductal Carcinoma |
| | E204Q | Esophageal Squamous Cell Carcinoma |
| | E204Sfs*43 | Head and Neck Squamous Cell Carcinoma |
| | E204Vfs*4 | Hepatocellular Carcinoma |
| | | Lung Squamous Cell Carcinoma |
| | | Serous Ovarian Cancer |
| | | Uterine Endometrioid Carcinoma |
| | | Uterine Mixed Endometrial Carcinoma |
| 205 | Y205* | Astrocytoma |
| | Y205C | Breast Invasive Ductal Carcinoma |
| | Y205D | Chromophobe Renal Cell Carcinoma |
| | Y205F | Colon Adenocarcinoma |
| | Y205H | Esophageal Squamous Cell Carcinoma |
| | Y205N | Glioblastoma Multiforme |
| | Y205S | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Pancreatic Adenocarcinoma |
| | | Rectal Adenocarcinoma |
| | | Serous Ovarian Cancer |
| | | Stomach Adenocarcinoma |
| | | Uterine Carcinosarcoma/Uterine Malignant Mixed Mullerian |
| | | Tumor |
| | | Uterine Endometrioid Carcinoma |
| | | Uterine Mixed Endometrial Carcinoma |
| | | Uterine Serous Carcinoma/Uterine Papillary Serous Carcinoma |
| 206 | L206Wfs*41 | Head and Neck Squamous Cell Carcinoma |
| 207 | D207Ffs*35 | Lung Squamous Cell Carcinoma |
| | D207Mfs*40 | |
| 208 | D208G | Bladder Urothelial Carcinoma |
| | D208N | Breast Invasive Ductal Carcinoma |
| | D208V | Oligoastrocytoma |
| | | Serous Ovarian Cancer |
| 209 | R209* | Bladder Urothelial Carcinoma |
| | R209Hfs*5 | Breast Invasive Carcinoma (NOS) |
| | R209I | Breast Invasive Ductal Carcinoma |
| | R209K | Esophageal Adenocarcinoma |
| | R209Kfs*6 | Esophageal Squamous Cell Carcinoma |
| | | Head and Neck Squamous Cell Carcinoma |
| | | Leiomyosarcoma |
| | | Lung Adenocarcinoma |
| | | Prostate Adenocarcinoma |
| | | Serous Ovarian Cancer |
| | | Signet Ring Cell Carcinoma of the Stomach |
| | | Uterine Endometrioid Carcinoma |
| 210 | N210Tfs*37 | Serous Ovarian Cancer |
| 211 | T211_H214del | Breast Invasive Carcinoma (NOS) |
| | T211A | Breast Invasive Ductal Carcinoma |
| | T211Ffs*4 | Colon Adenocarcinoma |
| | T211I | Glioblastoma Multiforme |
| | T211Lfs*36 | Head and Neck Squamous Cell Carcinoma |
| | | Lung Squamous Cell Carcinoma |
| | | Rectal Adenocarcinoma |
| | | Uterine Endometrioid Carcinoma |
| 212 | F212Sfs*3 | Astrocytoma |
| | F212Yfs*34 | Breast Invasive Ductal Carcinoma |
| | | Glioblastoma Multiforme |
| | | Lung Squamous Cell Carcinoma |
| 213 | R213* | Adrenocortical Carcinoma |
| | R213Dfs*34 | Astrocytoma |
| | R213G | Bladder Urothelial Carcinoma |
| | R213L | Breast Invasive Carcinoma (NOS) |
| | R213P | Breast Invasive Ductal Carcinoma |
| | R213Q | Chromophobe Renal Cell Carcinoma |
| | | Colon Adenocarcinoma |
| | | Cutaneous Melanoma |
| | | Dedifferentiated Liposarcoma |
| | | Diffuse Type Stomach Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Lung Adenocarcinoma |
| | | Lung Squamous Cell Carcinoma |
| | | Mucinous Adenocarcinoma of the Colon and Rectum |
| | | Myxofibrosarcoma |
| | | Oligoastrocytoma |
| | | Oligodendroglioma |
| | | Pancreatic Adenocarcinoma |
| | | Rectal Adenocarcinoma |
| | | Renal Clear Cell Carcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Endometrioid Carcinoma |
| 214 | H214L | Astrocytoma |
| | H214Qfs*33 | Bladder Urothelial Carcinoma |
| | H214R | Chromophobe Renal Cell Carcinoma |
| | | Colon Adenocarcinoma |
| | | Glioblastoma Multiforme |
| | | Head and Neck Squamous Cell Carcinoma |
| | | Lung Adenocarcinoma |
| | | Lung Squamous Cell Carcinoma |
| | | Oligoastrocytoma |
| | | Pancreatic Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Serous Carcinoma/Uterine Papillary Serous Carcinoma |
| 215 | S215G | Acute Myeloid Leukemia |
| | S215I | Astrocytoma |
| | S215Kfs*7 | Breast Invasive Ductal Carcinoma |
| | S215N | Colon Adenocarcinoma |
| | S215R | Cutaneous Melanoma |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Pancreatic Adenocarcinoma |
| | | Rectal Adenocarcinoma |
| | | Serous Ovarian Cancer |
| | | Uterine Endometrioid Carcinoma |
| 216 | V216E | Astrocytoma |
| | V216G | Breast Invasive Ductal Carcinoma |
| | V216L | Colon Adenocarcinoma |
| | V216M | Esophageal Adenocarcinoma |
| | | Glioblastoma Multiforme |
| | | Head and Neck Squamous Cell Carcinoma |
| | | Lung Adenocarcinoma |
| | | Lung Squamous Cell Carcinoma |
| | | Pleural Mesothelioma, Biphasic Type |
| | | Serous Ovarian Cancer |
| | | Uterine Carcinosarcoma/Uterine Malignant Mixed Mullerian |
| | | Tumor |
| | | Uterine Serous Carcinoma/Uterine Papillary Serous Carcinoma |
| 217 | V217Gfs*31 | Pancreatic Adenocarcinoma |
| 218 | V218Cfs*29 | Bladder Urothelial Carcinoma |
| | V218del | Cutaneous Melanoma |
| | V218E | Head and Neck Squamous Cell Carcinoma |
| | V218G | Lung Squamous Cell Carcinoma |
| | | Serous Ovarian Cancer |
| 219 | P219Afs*3 | Bladder Urothelial Carcinoma |
| | P219Lfs*2 | Head and Neck Squamous Cell Carcinoma |
| | P219T | Rectal Adenocarcinoma |
| 220 | Y220* | Astrocytoma |
| | Y220C | Bladder Urothelial Carcinoma |
| | Y220D | Breast Invasive Carcinoma (NOS) |
| | Y220H | Breast Invasive Ductal Carcinoma |
| | Y220Mfs*27 | Cervical Squamous Cell Carcinoma |
| | Y220S | Chromophobe Renal Cell Carcinoma |
| | | Colon Adenocarcinoma |
| | | Cutaneous Melanoma |
| | | Dedifferentiated Liposarcoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Stomach Adenocarcinoma |
| | | Oligoastrocytoma |
| | | Oligodendroglioma |
| | | Pancreatic Adenocarcinoma |
| | | Papillary Renal Cell Carcinoma |
| | | Serous Ovarian Cancer |
| | | Uterine Carcinosarcoma/Uterine Malignant Mixed Mullerian |
| | | Tumor |
| | | Uterine Endometrioid Carcinoma |
| | | Uterine Mixed Endometrial Carcinoma |
| | | Uterine Serous Carcinoma/Uterine Papillary Serous Carcinoma |
| 221 | E221* | Bladder Urothelial Carcinoma |
| | E221Afs*2 | Breast Invasive Ductal Carcinoma |
| | | Hepatocellular Carcinoma |
| | | Intestinal Type Stomach Adenocarcinoma |
| | | Lung Squamous Cell Carcinoma |
| | | Serous Ovarian Cancer |
| 223 | P223* | Acute Myeloid Leukemia |
| | P223Rfs*4 | Serous Ovarian Cancer |
| | X223_splice | Tubular Stomach Adenocarcinoma |
| | | Uterine Serous Carcinoma/Uterine Papillary Serous Carcinoma |
| 224 | E224* | Astrocytoma |
| | E224D | Bladder Urothelial Carcinoma |
| | E224Gfs*4 | Breast Invasive Ductal Carcinoma |
| | X224_splice | Colon Adenocarcinoma |
| | | Cutaneous Melanoma |
| | | Diffuse Type Stomach Adenocarcinoma |
| | | Esophageal Squamous Cell Carcinoma |
| | | Head and Neck Squamous Cell Carcinoma |
| | | Hepatocellular Carcinoma |
| | | Leiomyosarcoma |
| | | Lung Adenocarcinoma |
| | | Lung Squamous Cell Carcinoma |
| | | Myxofibrosarcoma |
| | | Oligoastrocytoma |
| | | Serous Ovarian Cancer |
| | | Tubular Stomach Adenocarcinoma |
| 225 | V225Rfs*23 | Acute Myeloid Leukemia |
| | X225_splice | Astrocytoma |
| | | Breast Invasive Ductal Carcinoma |
| | | Chromophobe Renal Cell Carcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Lung Adenocarcinoma |
| | | Lung Squamous Cell Carcinoma |
| | | Pleural Mesothelioma, Epithelioid Type |
| | | Serous Ovarian Cancer |
| | | Uterine Endometrioid Carcinoma |
| | | Uterine Mixed Endometrial Carcinoma |
| | | Uterine Serous Carcinoma/Uterine Papillary Serous Carcinoma |
| 226 | G226Afs*21 | Serous Ovarian Cancer |
| | | Tubular Stomach Adenocarcinoma |
| 227 | S227* | Oligoastrocytoma |
| 228 | D228* | Bladder Urothelial Carcinoma |
| | D228E | Hepatocellular Carcinoma |
| | D228N | Serous Ovarian Cancer |
| | D228Vfs*18 | Tubular Stomach Adenocarcinoma |
| 229 | C229* | Bladder Urothelial Carcinoma |
| | C229_I232del | Chromophobe Renal Cell Carcinoma |
| | C229Lfs*18 | Colon Adenocarcinoma |
| | C229Yfs*10 | Esophageal Squamous Cell Carcinoma |
| | | Head and Neck Squamous Cell Carcinoma |
| | | Lung Adenocarcinoma |
| | | Serous Ovarian Cancer |
| 230 | T230_T231del | Hepatocellular Carcinoma |
| | T230Hfs*9 | Lung Squamous Cell Carcinoma |
| | T230P | Oligoastrocytoma |
| 232 | I232F | Astrocytoma |
| | I232N | Breast Invasive Ductal Carcinoma |
| | I232S | Colon Adenocarcinoma |
| | I232T | Diffuse Large B-Cell Lymphoma, NOS |
| | | Lung Adenocarcinoma |
| | | Lung Squamous Cell Carcinoma |
| | | Rectal Adenocarcinoma |
| | | Serous Ovarian Cancer |
| | | Uterine Endometrioid Carcinoma |
| 234 | Y234* | Adrenocortical Carcinoma |
| | Y234C | Astrocytoma |
| | Y234H | Breast Invasive Ductal Carcinoma |
| | Y234N | Colon Adenocarcinoma |
| | Y234Pfs*7 | Glioblastoma Multiforme |
| | Y234S | Head and Neck Squamous Cell Carcinoma |
| | Y234Tfs*11 | Intrahepatic Cholangiocarcinoma |
| | | Leiomyosarcoma |
| | | Lung Squamous Cell Carcinoma |
| | | Oligoastrocytoma |
| | | Pleural Mesothelioma, Biphasic Type |
| | | Rectal Adenocarcinoma |
| | | Renal Clear Cell Carcinoma |
| | | Serous Ovarian Cancer |
| 236 | Y236* | Astrocytoma |
| | Y236C | Bladder Urothelial Carcinoma |
| | Y236D | Colon Adenocarcinoma |
| | Y236del | Head and Neck Squamous Cell Carcinoma |
| | Y236H | Lung Adenocarcinoma |
| | | Lung Squamous Cell Carcinoma |
| | | Oligoastrocytoma |
| | | Serous Ovarian Cancer |
| | | Uterine Mixed Endometrial Carcinoma |
| | | Uterine Serous Carcinoma/Uterine Papillary Serous Carcinoma |
| 237 | M237_N239del | Astrocytoma |
| | M237Cfs*10 | Bladder Urothelial Carcinoma |
| | M237Gfs*20 | Breast Invasive Ductal Carcinoma |
| | M237I | Colon Adenocarcinoma |
| | M237K | Dedifferentiated Liposarcoma |
| | M237L | Esophageal Adenocarcinoma |
| | M237V | Head and Neck Squamous Cell Carcinoma |
| | | Hepatocellular Carcinoma |
| | | Intestinal Type Stomach Adenocarcinoma |
| | | Intrahepatic Cholangiocarcinoma |
| | | Lung Adenocarcinoma |
| | | Lung Squamous Cell Carcinoma |
| | | Myxofibrosarcoma |
| | | Oligoastrocytoma |
| | | Prostate Adenocarcinoma |
| | | Serous Ovarian Cancer |
| | | Tubular Stomach Adenocarcinoma |
| | | Uterine Endometrioid Carcinoma |
| | | Uterine Serous Carcinoma/Uterine Papillary Serous Carcinoma |
| 238 | C238* | Astrocytoma |
| | C238_M243del | Bladder Urothelial Carcinoma |
| | C238_N239delinsY | Breast Invasive Ductal Carcinoma |
| | C238F | Endocervical Adenocarcinoma |
| | C238G | Esophageal Squamous Cell Carcinoma |
| | C238Lfs*9 | Glioblastoma Multiforme |
| | C238R | Head and Neck Squamous Cell Carcinoma |
| | C238S | Hepatocellular Carcinoma |
| | C238W | Intestinal Type Stomach Adenocarcinoma |
| | C238Y | Leiomyosarcoma |
| | | Lung Adenocarcinoma |
| | | Lung Squamous Cell Carcinoma |
| | | Oligodendroglioma |
| | | Pancreatic Adenocarcinoma |
| | | Pleural Mesothelioma, Epithelioid Type |
| | | Rectal Adenocarcinoma |
| | | Serous Ovarian Cancer |
| | | Uterine Carcinosarcoma/Uterine Malignant Mixed Mullerian |
| | | Tumor |
| | | Uterine Endometrioid Carcinoma |
| 239 | N239* | Astrocytoma |
| | N239_S240del | Breast Invasive Ductal Carcinoma |
| | N239_S241del | Chromophobe Renal Cell Carcinoma |
| | N239D | Colon Adenocarcinoma |
| | N239Qfs*24 | Esophageal Adenocarcinoma |
| | N239S | Glioblastoma Multiforme |
| | | Head and Neck Squamous Cell Carcinoma |
| | | Lung Adenocarcinoma |
| | | Pancreatic Adenocarcinoma |
| | | Prostate Adenocarcinoma |
| | | Serous Ovarian Cancer |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| 240 | S240_C242del | Bladder Urothelial Carcinoma |
| | S240G | Colon Adenocarcinoma |
| | S240Gfs*20 | Head and Neck Squamous Cell Carcinoma |
| | S240Kfs*24 | Stomach Adenocarcinoma |
| | S240R | Uterine Endometrioid Carcinoma |
| 241 | S241A | Bladder Urothelial Carcinoma |
| | S241C | Breast Invasive Ductal Carcinoma |
| | S241dup | Cervical Squamous Cell Carcinoma |
| | S241F | Cutaneous Melanoma |
| | S241P | Esophageal Adenocarcinoma |
| | S241Pfs*6 | Glioblastoma Multiforme |
| | S241Y | Head and Neck Squamous Cell Carcinoma |
| | | Invasive Breast Carcinoma |
| | | Lung Adenocarcinoma |
| | | Lung Squamous Cell Carcinoma |
| | | Oligoastrocytoma |
| | | Pancreatic Adenocarcinoma |
| | | Pancreatic Adenocarcinoma |
| | | Papillary Renal Cell Carcinoma |
| | | Papillary Stomach Adenocarcinoma |
| | | Perihilar Cholangiocarcinoma |
| | | Serous Ovarian Cancer |
| | | Serous Ovarian Cancer |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Endometrioid Carcinoma |
| | | Uterine Serous Carcinoma/Uterine Papillary Serous Carcinoma |
| 242 | C242* | Bladder Urothelial Carcinoma |
| | C242Afs*5 | Breast Invasive Ductal Carcinoma |
| | C242F | Colon Adenocarcinoma |
| | C242G | Diffuse Type Stomach Adenocarcinoma |
| | C242R | Esophageal Adenocarcinoma |
| | C242S | Esophageal Squamous Cell Carcinoma |
| | C242Y | Glioblastoma Multiforme |
| | | Head and Neck Squamous Cell Carcinoma |
| | | Intestinal Type Stomach Adenocarcinoma |
| | | Lung Adenocarcinoma |
| | | Lung Squamous Cell Carcinoma |
| | | Pancreatic Adenocarcinoma |
| | | Pancreatic Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Carcinosarcoma/Uterine Malignant Mixed Mullerian |
| | | Tumor |
| | | Uterine Serous Carcinoma/Uterine Papillary Serous Carcinoma |
| 243 | M243I | Bladder Urothelial Carcinoma |
| | | Lung Squamous Cell Carcinoma |
| | | Oligoastrocytoma |
| | | Serous Ovarian Cancer |
| | | Uterine Carcinosarcoma/Uterine Malignant Mixed Mullerian |
| | | Tumor |
| 244 | G244* | Colon Adenocarcinoma |
| | G244C | Glioblastoma Multiforme |
| | G244D | Head and Neck Squamous Cell Carcinoma |
| | G244S | Leiomyosarcoma |
| | G244V | Lung Adenocarcinoma |
| | | Lung Squamous Cell Carcinoma |
| | | Pleural Mesothelioma, Epithelioid Type |
| | | Serous Ovarian Cancer |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| | | Uterine Serous Carcinoma/Uterine Papillary Serous Carcinoma |
| 245 | G245C | Astrocytoma |
| | G245D | Bladder Urothelial Carcinoma |
| | G245R | Breast Invasive Ductal Carcinoma |
| | G245S | Colon Adenocarcinoma |
| | G245V | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Lung Adenocarcinoma |
| | | Lung Squamous Cell Carcinoma |
| | | Oligoastrocytoma |
| | | Oligodendroglioma |
| | | Pancreatic Adenocarcinoma |
| | | Pleural Mesothelioma, Epithelioid Type |
| | | Prostate Adenocarcinoma |
| | | Rectal Adenocarcinoma |
| | | Serous Ovarian Cancer |
| | | Signet Ring Cell Carcinoma of the Stomach |
| | | Stomach Adenocarcinoma |
| | | Uterine Carcinosarcoma/Uterine Malignant Mixed Mullerian |
| | | Tumor |
| | | Uterine Endometrioid Carcinoma |
| 246 | M246I | Bladder Urothelial Carcinoma |
| | M246R | Breast Invasive Lobular Carcinoma |
| | M246T | Chromophobe Renal Cell Carcinoma |
| | M246V | Esophageal Adenocarcinoma |
| | | Glioblastoma Multiforme |
| | | Head and Neck Squamous Cell Carcinoma |
| | | Hepatocellular Carcinoma |
| | | Oligoastrocytoma |
| | | Pancreatic Adenocarcinoma |
| | | Tubular Stomach Adenocarcinoma |
| | | Uterine Mixed Endometrial Carcinoma |
| 247 | N247I | Breast Invasive Ductal Carcinoma |
| | | Lung Adenocarcinoma |
| 248 | R248G | Acute Myeloid Leukemia |
| | R248Hfs*13 | Astrocytoma |
| | R248L | Bladder Urothelial Carcinoma |
| | R248P | Breast Invasive Carcinoma (NOS) |
| | R248Q | Breast Invasive Ductal Carcinoma |
| | R248W | Cholangiocarcinoma |
| | | Colon Adenocarcinoma |
| | | Cutaneous Melanoma |
| | | Diffuse Large B-Cell Lymphoma, NOS |
| | | Diffuse Type Stomach Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Adenocarcinoma of the Colon and Rectum |
| | | Oligoastrocytoma |
| | | Oligodendroglioma |
| | | Pancreatic Adenocarcinoma |
| | | Prostate Adenocarcinoma |
| | | Rectal Adenocarcinoma |
| | | Renal Clear Cell Carcinoma |
| | | Serous Ovarian Cancer |
| | | Stomach Adenocarcinoma |
| | | Thymoma |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| | | Uterine Mixed Endometrial Carcinoma |
| | | Uterine Serous Carcinoma/Uterine Papillary Serous Carcinoma |
| 249 | R249Ffs*96 | Astrocytoma |
| | R249G | Bladder Urothelial Carcinoma |
| | R249Gfs*96 | Esophageal Squamous Cell Carcinoma |
| | R249M | Glioblastoma Multiforme |
| | R249S | Head and Neck Squamous Cell Carcinoma |
| | R249T | Hepatocellular Carcinoma |
| | R249W | Lung Adenocarcinoma |
| | | Lung Squamous Cell Carcinoma |
| | | Mucinous Adenocarcinoma of the Colon and Rectum |
| | | Prostate Adenocarcinoma |
| | | Serous Ovarian Cancer |
| | | Stomach Adenocarcinoma |
| | | Uterine Carcinosarcoma/Uterine Malignant Mixed Mullerian |
| | | Tumor |
| | | Uterine Serous Carcinoma/Uterine Papillary Serous Carcinoma |
| 250 | P250_I251delinsL | Astrocytoma |
| | P250Hfs*13 | Breast Invasive Ductal Carcinoma |
| | P250L | Chromophobe Renal Cell Carcinoma |
| | P250R | Esophageal Squamous Cell Carcinoma |
| | | Glioblastoma Multiforme |
| | | Head and Neck Squamous Cell Carcinoma |
| | | Lung Adenocarcinoma |
| | | Lung Squamous Cell Carcinoma |
| | | Mucinous Stomach Adenocarcinoma |
| | | Oligoastrocytoma |
| | | Pancreatic Adenocarcinoma |
| | | Rectal Adenocarcinoma |
| | | Serous Ovarian Cancer |
| 251 | I251F | Breast Invasive Ductal Carcinoma |
| | I251N | Head and Neck Squamous Cell Carcinoma |
| | I251S | Hepatocellular Carcinoma |
| | I251Sfs*94 | Lung Squamous Cell Carcinoma |
| | | Serous Ovarian Cancer |
| 252 | L252_I254del | Breast Invasive Ductal Carcinoma |
| | L252_T253delinsP | Esophageal Squamous Cell Carcinoma |
| | L252del | Lung Squamous Cell Carcinoma |
| | L252P | Myxofibrosarcoma |
| | L252Sfs*93 | Uterine Mixed Endometrial Carcinoma |
| 253 | T253A | Breast Invasive Ductal Carcinoma |
| | T253dup | Esophageal Adenocarcinoma |
| | T253Hfs*11 | Hepatocellular Carcinoma |
| | T253I | Papillary Stomach Adenocarcinoma |
| | T253N | Serous Ovarian Cancer |
| 254 | I254S | Glioblastoma Multiforme |
| | | Intestinal Type Stomach Adenocarcinoma |
| | | Pancreatic Adenocarcinoma |
| 255 | I255del | Breast Invasive Lobular Carcinoma |
| | I255F | Esophageal Adenocarcinoma |
| | I255N | Esophageal Squamous Cell Carcinoma |
| | I255S | Glioblastoma Multiforme |
| | I255T | Glioblastoma Multiforme |
| | | Lung Adenocarcinoma |
| | | Oligodendroglioma |
| | | Pancreatic Adenocarcinoma |
| | | Papillary Renal Cell Carcinoma |
| 256 | T256del | Breast Invasive Ductal Carcinoma |
| | T256Hfs*8 | Head and Neck Squamous Cell Carcinoma |
| | T256I | Prostate Adenocarcinoma |
| | T256Ifs*90 | Serous Ovarian Cancer |
| | T256Nfs*8 | Undifferentiated Pleomorphic Sarcoma/Malignant Fibrous |
| | | Histiocytoma/High-Grade Spindle Cell Sarcoma |
| | | Uterine Endometrioid Carcinoma |
| 257 | L257P | Bladder Urothelial Carcinoma |
| | L257Pfs*7 | Breast Invasive Ductal Carcinoma |
| | L257Q | Esophageal Adenocarcinoma |
| | L257R | Hepatocellular Carcinoma |
| | | Serous Ovarian Cancer |
| 258 | E258* | Bladder Urothelial Carcinoma |
| | E258A | Breast Invasive Ductal Carcinoma |
| | E258D | Esophageal Adenocarcinoma |
| | E258G | Head and Neck Squamous Cell Carcinoma |
| | E258K | Hepatocellular Carcinoma |
| | E258Q | Lung Adenocarcinoma |
| | E258Qfs*3 | Lung Squamous Cell Carcinoma |
| | X258_splice | Prostate Adenocarcinoma |
| | | Rectal Adenocarcinoma |
| | | Serous Ovarian Cancer |
| 259 | D259Efs*86 | Bladder Urothelial Carcinoma |
| | D259N | Colon Adenocarcinoma |
| | D259V | Head and Neck Squamous Cell Carcinoma |
| | D259Y | Leiomyosarcoma |
| | | Lung Adenocarcinoma |
| | | Lung Squamous Cell Carcinoma |
| | | Pancreatic Adenocarcinoma |
| | | Tubular Stomach Adenocarcinoma |
| 260 | S260_L264dup | Serous Ovarian Cancer |
| | S260Lfs*4 | |
| 261 | S261Vfs*84 | Breast Invasive Ductal Carcinoma |
| | X261_splicc | Esophageal Adenocarcinoma |
| | | Esophageal Squamous Cell Carcinoma |
| | | Head and Neck Squamous Cell Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Oligoastrocytoma |
| | | Renal Clear Cell Carcinoma |
| | | Serous Ovarian Cancer |
| | | Signet Ring Cell Carcinoma of the Stomach |
| | | Stomach Adenocarcinoma |
| | | Uterine Carcinosarcoma/Uterine Malignant Mixed Mullerian |
| | | Tumor |
| 262 | G262del | Head and Neck Squamous Cell Carcinoma |
| | G262V | Hepatocellular Carcinoma |
| | G262Vfs*83 | Leiomyosarcoma |
| | | Lung Adenocarcinoma |
| | | Lung Squamous Cell Carcinoma |
| | | Rectal Adenocarcinoma |
| | | Serous Ovarian Cancer |
| | | Uterine Endometrioid Carcinoma |
| 263 | N263Ifs*82 | Hepatocellular Carcinoma |
| | N263Kfs*9 | Oligoastrocytoma |
| | N263Sfs*8 | |
| 264 | L264Yfs*81 | Glioblastoma Multiforme |
| 265 | L265P | Astrocytoma |
| | L265R | Bladder Urothelial Carcinoma |
| | L265Tfs*7 | Breast Invasive Ductal Carcinoma |
| | | Head and Neck Squamous Cell Carcinoma |
| | | Lung Squamous Cell Carcinoma |
| | | Oligoastrocytoma |
| | | Serous Ovarian Cancer |
| | | Tubular Stomach Adenocarcinoma |
| 266 | G266* | Breast Invasive Ductal Carcinoma |
| | G266Dfs*79 | Colon Adenocarcinoma |
| | G266E | Cutaneous Melanoma |
| | G266R | Glioblastoma Multiforme |
| | G266V | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Oligodendroglioma |
| | | Pancreatic Adenocarcinoma |
| | | Prostate Adenocarcinoma |
| | | Rectal Adenocarcinoma |
| | | Serous Ovarian Cancer |
| | | Uterine Carcinosarcoma/Uterine Malignant Mixed Mullerian |
| | | Tumor |
| | | Uterine Mixed Endometrial Carcinoma |
| | | Uterine Serous Carcinoma/Uterine Papillary Serous Carcinoma |
| 267 | R267G | Astrocytoma |
| | R267L | Chromophobe Renal Cell Carcinoma |
| | R267P | Colon Adenocarcinoma |
| | R267Q | Glioblastoma Multiforme |
| | R267W | Head and Neck Squamous Cell Carcinoma |
| | | Lung Adenocarcinoma |
| | | Lung Squamous Cell Carcinoma |
| | | Mucinous Adenocarcinoma of the Colon and Rectum |
| | | Oligoastrocytoma |
| | | Tubular Stomach Adenocarcinoma |
| | | Uterine Endometrioid Carcinoma |
| 268 | N268* | Head and Neck Squamous Cell Carcinoma |
| | N268_R273dcl | Lung Squamous Cell Carcinoma |
| | N268Kfs*77 | Undifferentiated Pleomorphic Sarcoma/Malignant Fibrous |
| | N268Tfs*77 | Histiocytoma/High-Grade Spindle Cell Sarcoma |
| | | Uterine Carcinosarcoma/Uterine Malignant Mixed Mullerian |
| | | Tumor |
| 269 | S269_E271delinsK | Oligodendroglioma |
| | S269Ifs*2 | Pancreatic Adenocarcinoma |
| 270 | F270C | Astrocytoma |
| | F270I | Breast Invasive Ductal Carcinoma |
| | F270L | Head and Neck Squamous Cell Carcinoma |
| | F270Lfs*2 | Intestinal Type Stomach Adenocarcinoma |
| | F270S | Leiomyosarcoma |
| | F270V | Lung Adenocarcinoma |
| | | Oligodendroglioma |
| | | Serous Ovarian Cancer |
| | | Stomach Adenocarcinoma |
| | | Tubular Stomach Adenocarcinoma |
| 271 | E271* | Bladder Urothelial Carcinoma |
| | E271K | Breast Invasive Ductal Carcinoma |
| | E271Q | Colon Adenocarcinoma |
| | E271V | Endometrioid Carcinoma |
| | | Glioblastoma Multiforme |
| | | Head and Neck Squamous Cell Carcinoma |
| | | Hepatocellular Carcinoma |
| | | Lung Adenocarcinoma |
| | | Lung Squamous Cell Carcinoma |
| | | Prostate Adenocarcinoma |
| | | Serous Ovarian Cancer |
| | | Tubular Stomach Adenocarcinoma |
| 272 | V272A | Astrocytoma |
| | V272Cfs*73 | Breast Invasive Carcinoma (NOS) |
| | V272G | Breast Invasive Ductal Carcinoma |
| | V272L | Cervical Squamous Cell Carcinoma |
| | V272M | Colon Adenocarcinoma |
| | | Esophageal Squamous Cell Carcinoma |
| | | Head and Neck Squamous Cell Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Pancreatic Adenocarcinoma |
| | | Rectal Adenocarcinoma |
| | | Renal Clear Cell Carcinoma |
| | | Serous Ovarian Cancer |
| | | Stomach Adenocarcinoma |
| | | Uterine Serous Carcinoma/Uterine Papillary Serous Carcinoma |
| 273 | R273C | Acute Myeloid Leukemia |
| | R273G | Adrenocortical Carcinoma |
| | R273H | Astrocytoma |
| | R273L | Bladder Urothelial Carcinoma |
| | R273Lfs*72 | Breast Invasive Carcinoma (NOS) |
| | R273P | Breast Invasive Ductal Carcinoma |
| | R273S | Colon Adenocarcinoma |
| | | Diffuse Large B-Cell Lymphoma, NOS |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Adenocarcinoma of the Colon and Rectum |
| | | Oligoastrocytoma |
| | | Oligodendroglioma |
| | | Pancreatic Adenocarcinoma |
| | | Pleural Mesothelioma, Biphasic Type |
| | | Prostate Adenocarcinoma |
| | | Rectal Adenocarcinoma |
| | | Serous Ovarian Cancer |
| | | Stomach Adenocarcinoma |
| | | Thymoma |
| | | Tubular Stomach Adenocarcinoma |
| | | Uterine Carcinosarcoma/Uterine Malignant Mixed Mullerian |
| | | Tumor |
| | | Uterine Endometrioid Carcinoma |
| | | Uterine Mixed Endometrial Carcinoma |
| | | Uterine Serous Carcinoma/Uterine Papillary Serous Carcinoma |
| 274 | V274_C275dup | Astrocytoma |
| | V274_G279del | Breast Invasive Ductal Carcinoma |
| | V274D | Colon Adenocarcinoma |
| | V274dup | Esophageal Adenocarcinoma |
| | V274F | Esophageal Squamous Cell Carcinoma |
| | V274G | Head and Neck Squamous Cell Carcinoma |
| | V274L | Intestinal Type Stomach Adenocarcinoma |
| | | Lung Adenocarcinoma |
| | | Lung Squamous Cell Carcinoma |
| | | Oligoastrocytoma |
| | | Pancreatic Adenocarcinoma |
| | | Serous Ovarian Cancer |
| 275 | C275* | Adrenocortical Carcinoma |
| | C275_R282delinsW | Astrocytoma |
| | C275F | Bladder Urothelial Carcinoma |
| | C275G | Breast Invasive Ductal Carcinoma |
| | C275Lfs*67 | Chromophobe Renal Cell Carcinoma |
| | C275Lfs*70 | Colon Adenocarcinoma |
| | C275R | Cutaneous Melanoma |
| | C275S | Esophageal Squamous Cell Carcinoma |
| | C275Vfs*70 | Glioblastoma Multiforme |
| | C275W | Head and Neck Squamous Cell Carcinoma |
| | C275Y | Hepatocellular Carcinoma |
| | | Leiomyosarcoma |
| | | Low-Grade Glioma (NOS) |
| | | Lung Adenocarcinoma |
| | | Lung Squamous Cell Carcinoma |
| | | Pancreatic Adenocarcinoma |
| | | Rectal Adenocarcinoma |
| | | Serous Ovarian Cancer |
| | | Uterine Serous Carcinoma/Uterine Papillary Serous Carcinoma |
| 276 | A276D | Breast Invasive Ductal Carcinoma |
| | A276G | Cutaneous Melanoma |
| | A276Lfs*29 | Head and Neck Squamous Cell Carcinoma |
| | A276Lfs*31 | Hepatocellular Carcinoma |
| | A276P | Lung Squamous Cell Carcinoma |
| | | Pleural Mesothelioma, Biphasic Type |
| | | Serous Ovarian Cancer |
| | | Uterine Serous Carcinoma/Uterine Papillary Serous Carcinoma |
| 277 | C277* | Astrocytoma |
| | C277dup | Bladder Urothelial Carcinoma |
| | C277F | Head and Neck Squamous Cell Carcinoma |
| | C277G | Lung Adenocarcinoma |
| | C277Vfs*68 | Oligoastrocytoma |
| | C277W | Oligodendroglioma |
| | C277Y | Serous Ovarian Cancer |
| | | Serous Ovarian Cancer |
| 278 | P278A | Astrocytoma |
| | P278H | Bladder Urothelial Carcinoma |
| | P278L | Breast Invasive Ductal Carcinoma |
| | P278Lfs*67 | Colon Adenocarcinoma |
| | P278Lfs*68 | Cutaneous Melanoma |
| | P278R | Esophageal Squamous Cell Carcinoma |
| | P278S | Head and Neck Squamous Cell Carcinoma |
| | P278T | Lung Adenocarcinoma |
| | | Lung Squamous Cell Carcinoma |
| | | Metaplastic Breast Cancer |
| | | Mucinous Adenocarcinoma of the Colon and Rectum |
| | | Mucinous Carcinoma |
| | | Myxofibrosarcoma |
| | | Pancreatic Adenocarcinoma |
| | | Rectal Adenocarcinoma |
| | | Serous Ovarian Cancer |
| | | Uterine Serous Carcinoma/Uterine Papillary Serous Carcinoma |
| 279 | G279E | Bladder Urothelial Carcinoma |
| | G279Pfs*69 | Breast Invasive Ductal Carcinoma |
| | G279R | Lung Squamous Cell Carcinoma |
| | | Oligoastrocytoma |
| | | Prostate Adenocarcinoma |
| | | Serous Ovarian Cancer |
| | | Uterine Endometrioid Carcinoma |
| 280 | R280* | Acute Myeloid Leukemia |
| | R280Efs*65 | Astrocytoma |
| | R280G | Bladder Urothelial Carcinoma |
| | R280I | Breast Invasive Carcinoma (NOS) |
| | R280K | Breast Invasive Ductal Carcinoma |
| | R280Kfs*59 | Cervical Squamous Cell Carcinoma |
| | R280S | Cutaneous Melanoma |
| | R280T | Esophageal Adenocarcinoma |
| | | Head and Neck Squamous Cell Carcinoma |
| | | Hepatocellular Carcinoma |
| | | Lung Adenocarcinoma |
| | | Lung Squamous Cell Carcinoma |
| | | Metaplastic Breast Cancer |
| | | Oligoastrocytoma |
| | | Oligodendroglioma |
| | | Serous Ovarian Cancer |
| | | Uterine Carcinosarcoma/Uterine Malignant Mixed Mullerian |
| | | Tumor |
| 281 | D281A | Astrocytoma |
| | D281Afs*62 | Bladder Urothelial Carcinoma |
| | D281Afs*64 | Breast Invasive Ductal Carcinoma |
| | D281Afs*66 | Diffuse Type Stomach Adenocarcinoma |
| | D281E | Glioblastoma Multiforme |
| | D281Efs*26 | Head and Neck Squamous Cell Carcinoma |
| | D281Gfs*63 | Hepatocellular Carcinoma |
| | D281H | Leiomyosarcoma |
| | D281N | Lung Adenocarcinoma |
| | D281V | Lung Squamous Cell Carcinoma |
| | D281Y | Metaplastic Breast Cancer |
| | | Renal Clear Cell Carcinoma |
| | | Serous Ovarian Cancer |
| | | Stomach Adenocarcinoma |
| | | Thymoma |
| | | Uterine Carcinosarcoma/Uterine Malignant Mixed Mullerian |
| | | Tumor |
| | | Uterine Serous Carcinoma/Uterine Papillary Serous Carcinoma |
| 282 | R282_R283del | Astrocytoma |
| | R282G | Bladder Urothelial Carcinoma |
| | R282Gfs*63 | Breast Invasive Carcinoma (NOS) |
| | R282Q | Breast Invasive Ductal Carcinoma |
| | R282W | Cervical Squamous Cell Carcinoma |
| | | Chromophobe Renal Cell Carcinoma |
| | | Colon Adenocarcinoma |
| | | Diffuse Type Stomach Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Intestinal Type Stomach Adenocarcinoma |
| | | Lung Adenocarcinoma |
| | | Lung Squamous Cell Carcinoma |
| | | Mucinous Adenocarcinoma of the Colon and Rectum |
| | | Mucinous Stomach Adenocarcinoma |
| | | Oligoastrocytoma |
| | | Oligodendroglioma |
| | | Pancreatic Adenocarcinoma |
| | | Prostate Adenocarcinoma |
| | | Rectal Adenocarcinoma |
| | | Serous Ovarian Cancer |
| | | Signet Ring Cell Carcinoma of the Stomach |
| | | Stomach Adenocarcinoma |
| | | Uterine Endometrioid Carcinoma |
| | | Uterine Mixed Endometrial Carcinoma |
| | | Uterine Serous Carcinoma/Uterine Papillary Serous Carcinoma |
| 283 | R283Afs*62 | Bladder Urothelial Carcinoma |
| | R283H | Head and Neck Squamous Cell Carcinoma |
| | R283P | Lung Adenocarcinoma |
| | | Lung Squamous Cell Carcinoma |
| | | Pancreatic Adenocarcinoma |
| 284 | T284Hfs*22 | Serous Ovarian Cancer |
| | T284Kfs*61 | Tubular Stomach Adenocarcinoma |
| | T284Qfs*61 | |
| 285 | E285* | Bladder Urothelial Carcinoma |
| | E285K | Breast Invasive Ductal Carcinoma |
| | E285Q | Breast Invasive Lobular Carcinoma |
| | E285Rfs*54 | Cervical Squamous Cell Carcinoma |
| | E285V | Colon Adenocarcinoma |
| | | Diffuse Type Stomach Adenocarcinoma |
| | | Esophageal Squamous Cell Carcinoma |
| | | Head and Neck Squamous Cell Carcinoma |
| | | Lung Adenocarcinoma |
| | | Lung Squamous Cell Carcinoma |
| | | Mucinous Carcinoma |
| | | Pheochromocytoma |
| | | Prostate Adenocarcinoma |
| | | Rectal Adenocarcinoma |
| | | Stomach Adenocarcinoma |
| 286 | E286* | Acute Myeloid Leukemia |
| | E286_E287del | Bladder Urothelial Carcinoma |
| | E286A | Breast Invasive Carcinoma (NOS) |
| | E286G | Breast Invasive Ductal Carcinoma |
| | E286K | Colon Adenocarcinoma |
| | E286Q | Cutaneous Melanoma |
| | E286V | Diffuse Type Stomach Adenocarcinoma |
| | | Head and Neck Squamous Cell Carcinoma |
| | | Hepatocellular Carcinoma |
| | | Lung Adenocarcinoma |
| | | Lung Squamous Cell Carcinoma |
| | | Oligoastrocytoma |
| | | Pancreatic Adenocarcinoma |
| | | Rectal Adenocarcinoma |
| | | Serous Ovarian Cancer |
| | | Stomach Adenocarcinoma |
| | | Undifferentiated Pleomorphic Sarcoma/Malignant Fibrous |
| | | Histiocytoma/High-Grade Spindle Cell Sarcoma |
| | | Uterine Endometrioid Carcinoma |
| 287 | E287* | Bladder Urothelial Carcinoma |
| | E287D | Breast Invasive Ductal Carcinoma |
| | E287Pfs*9 | Cervical Squamous Cell Carcinoma |
| | E287Q | Lung Squamous Cell Carcinoma |
| | | Rectal Adenocarcinoma |
| 288 | N288Efs*18 | Astrocytoma |
| | N288Kfs*59 | Myxofibrosarcoma |
| 289 | L289F | Breast Invasive Ductal Carcinoma |
| 290 | R290C | Colon Adenocarcinoma |
| | R290Kfs*53 | Cutaneous Melanoma |
| | R290Sfs*56 | Head and Neck Squamous Cell Carcinoma |
| 291 | K291Sfs*51 | Lung Squamous Cell Carcinoma |
| 292 | K292* | Breast Invasive Ductal Carcinoma |
| | K292Gfs*52 | Chromophobe Renal Cell Carcinoma |
| | | Hepatocellular Carcinoma |
| 294 | E294* | Astrocytoma |
| | | Bladder Urothelial Carcinoma |
| | | Breast Invasive Ductal Carcinoma |
| | E294Sfs*51 | Esophageal Squamous Cell Carcinoma |
| | | Head and Neck Squamous Cell Carcinoma |
| | | Lung Adenocarcinoma |
| | | Lung Squamous Cell Carcinoma |
| | | Pancreatic Adenocarcinoma |
| | | Serous Ovarian Cancer |
| | | Tubular Stomach Adenocarcinoma |
| | | Uterine Endometrioid Carcinoma |
| 296 | H296Tfs*49 | Lung Adenocarcinoma |
| 297 | H297Pfs*48 | Astrocytoma |
| | | Head and Neck Squamous Cell Carcinoma |
| 298 | E298* | Bladder Urothelial Carcinoma |
| | | Head and Neck Squamous Cell Carcinoma |
| | | Lung Adenocarcinoma |
| | | Lung Squamous Cell Carcinoma |
| | | Pancreatic Adenocarcinoma |
| | | Prostate Adenocarcinoma |
| | | Undifferentiated Pleomorphic Sarcoma/Malignant Fibrous |
| | | Histiocytoma/High-Grade Spindle Cell Sarcoma |
| 299 | L299Afs*7 | Breast Invasive Ductal Carcinoma |
| 301 | P301Qfs*44 | Bladder Urothelial Carcinoma |
| | P301T | Colon Adenocarcinoma |
| | | Cutaneous Melanoma |
| | | Lung Squamous Cell Carcinoma |
| | | Stomach Adenocarcinoma |
| 302 | G302Afs*31 | Colon Adenocarcinoma |
| | G302Efs*3 | Glioblastoma Multiforme |
| | G302Rfs*4 | Head and Neck Squamous Cell Carcinoma |
| | | Hepatocellular Carcinoma |
| | | Pancreatic Adenocarcinoma |
| 303 | S303Afs*42 | Breast Invasive Ductal Carcinoma |
| 304 | T304lfs*41 | Breast Invasive Ductal Carcinoma |
| 305 | K305* | Head and Neck Squamous Cell Carcinoma |
| | K305Efs*39 | Lung Adenocarcinoma |
| | X305_splice | Lung Squamous Cell Carcinoma |
| | | Serous Ovarian Cancer |
| 306 | R306* | Astrocytoma |
| | R306Afs*31 | Bladder Urothelial Carcinoma |
| | | Breast Invasive Lobular Carcinoma |
| | | Colon Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Lung Squamous Cell Carcinoma |
| | | Mucinous Adenocarcinoma of the Colon and Rectum |
| | | Mucinous Stomach Adenocarcinoma |
| | | Oligoastrocytoma |
| | | Pancreatic Adenocarcinoma |
| | | Rectal Adenocarcinoma |
| | | Serous Ovarian Cancer |
| | | Stomach Adenocarcinoma |
| | | Uterine Endometrioid Carcinoma |
| 307 | X307_splice | Adrenocortical Carcinoma |
| | | Astrocytoma |
| | | Breast Invasive Carcinoma (NOS) |
| | | Chromophobe Renal Cell Carcinoma |
| | | Colon Adenocarcinoma |
| | | Diffuse Type Stomach Adenocarcinoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Hepatocellular Carcinoma |
| | | Lung Adenocarcinoma |
| | | Lung Squamous Cell Carcinoma |
| | | Prostate Adenocarcinoma |
| | | Serous Ovarian Cancer |
| 308 | L308Afs*28 | Breast Invasive Ductal Carcinoma |
| | L308Qfs*27 | Esophageal Adenocarcinoma |
| 310 | N310Tfs*35 | Diffuse Type Stomach Adenocarcinoma |
| 313 | S313Afs*32 | Glioblastoma Multiforme |
| | | Lung Adenocarcinoma |
| | | Uterine Endometrioid Carcinoma |
| 315 | S315C | Lung Squamous Cell Carcinoma |
| | S315Lfs*30 | Uterine Endometrioid Carcinoma |
| 316 | P316Sfs*21 | Serous Ovarian Cancer |
| 317 | Q317* | Acute Myeloid Leukemia |
| | Q317Afs*19 | Astrocytoma |
| | Q317Pfs*20 | Bladder Urothelial Carcinoma |
| | | Cutaneous Melanoma |
| | | Esophageal Squamous Cell Carcinoma |
| | | Head and Neck Squamous Cell Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Serous Ovarian Cancer |
| | | Uterine Serous Carcinoma/Uterine Papillary Serous Carcinoma |
| 318 | P318Tfs*15 | Breast Invasive Ductal Carcinoma |
| 319 | K319* | Bladder Urothelial Carcinoma |
| | K319Afs*19 | Cervical Squamous Cell Carcinoma |
| | K319N | Prostate Adenocarcinoma |
| | K319Rfs*26 | Rectal Adenocarcinoma |
| 320 | K320E | Head and Neck Squamous Cell Carcinoma |
| | K320Gfs*22 | Lung Squamous Cell Carcinoma |
| | K320Nfs*19 | Serous Ovarian Cancer |
| | K320Rfs*25 | |
| 321 | K321* | Astrocytoma |
| | K321Efs*16 | Serous Ovarian Cancer |
| 322 | P322Hfs*23 | Colon Adenocarcinoma |
| 325 | G325* | Esophageal Adenocarcinoma |
| 327 | Y327* | Cutaneous Melanoma |
| | | Glioblastoma Multiforme |
| | | Serous Ovarian Cancer |
| 328 | F328Sfs*17 | Breast Invasive Ductal Carcinoma |
| | | Hepatocellular Carcinoma |
| 329 | T329Hfs*8 | Head and Neck Squamous Cell Carcinoma |
| | T329Rfs*14 | Hepatocellular Carcinoma |
| 330 | L330Ffs*15 | Cutaneous Melanoma |
| | L330I | |
| | L330R | Head and Neck Squamous Cell Carcinoma |
| | | Serous Ovarian Cancer |
| 331 | Q331* | Astrocytoma |
| | Q331H | Bladder Urothelial Carcinoma |
| | Q331Sfs*6 | Breast Invasive Ductal Carcinoma |
| | X331_splice | Colon Adenocarcinoma |
| | | Cutaneous Melanoma |
| | | Head and Neck Squamous Cell Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Mucinous Adenocarcinoma of the Colon and Rectum |
| | | Mucinous Carcinoma |
| | | Pancreatic Adenocarcinoma |
| | | Pleural Mesothelioma, Epithelioid Type |
| | | Prostate Adenocarcinoma |
| | | Rectal Adenocarcinoma |
| | | Serous Ovarian Cancer |
| | | Uterine Serous Carcinoma/Uterine Papillary Serous Carcinoma |
| 332 | I332F | Astrocytoma |
| | X332_splice | Diffuse Large B-Cell Lymphoma, NOS |
| | | Esophageal Squamous Cell Carcinoma |
| | | Head and Neck Squamous Cell Carcinoma |
| | | Hepatocellular Carcinoma |
| | | Lung Adenocarcinoma |
| | | Lung Squamous Cell Carcinoma |
| | | Mucinous Adenocarcinoma of the Colon and Rectum |
| | | Oligoastrocytoma |
| | | Serous Ovarian Cancer |
| | | Undifferentiated Pleomorphic Sarcoma/Malignant Fibrous |
| | | Histiocytoma/High-Grade Spindle Cell Sarcoma |
| | | Uterine Serous Carcinoma/Uterine Papillary Serous Carcinoma |
| 333 | R333C | Breast Invasive Ductal Carcinoma |
| | R333Vfs*12 | Cervical Squamous Cell Carcinoma |
| | | Mucinous Adenocarcinoma of the Colon and Rectum |
| | | Serous Ovarian Cancer |
| 334 | G334V | Lung Adenocarcinoma |
| | G334W | Lung Squamous Cell Carcinoma |
| | | Seminoma |
| | | Uterine Serous Carcinoma/Uterine Papillary Serous Carcinoma |
| 335 | R335Lfs*10 | Adrenocortical Carcinoma |
| | R335Qfs*2 | Colon Adenocarcinoma |
| | R335Vfs*10 | Oligoastrocytoma |
| | | Rectal Adenocarcinoma |
| | | Undifferentiated Pleomorphic Sarcoma/Malignant Fibrous |
| | | Histiocytoma/High-Grade Spindle Cell Sarcoma |
| 336 | E336* | Bladder Urothelial Carcinoma |
| | E336_R337del | Colon Adenocarcinoma |
| | E336Afs*10 | Head and Neck Squamous Cell Carcinoma |
| | E336Sfs*9 | Leiomyosarcoma |
| | | Lung Adenocarcinoma |
| 337 | R337C | Acute Myeloid Leukemia |
| | R337H | Adrenocortical Carcinoma |
| | R337L | Bladder Urothelial Carcinoma |
| | R337P | Cervical Squamous Cell Carcinoma |
| | R337S | Chromophobe Renal Cell Carcinoma |
| | | Colon Adenocarcinoma |
| | | Glioblastoma Multiforme |
| | | Head and Neck Squamous Cell Carcinoma |
| | | Intestinal Type Stomach Adenocarcinoma |
| | | Leiomyosarcoma |
| | | Lung Adenocarcinoma |
| | | Lung Squamous Cell Carcinoma |
| | | Oligodendroglioma |
| | | Pancreatic Adenocarcinoma |
| | | Prostate Adenocarcinoma |
| | | Rectal Adenocarcinoma |
| | | Stomach Adenocarcinoma |
| 338 | F338I | Chromophobe Renal Cell Carcinoma |
| | F338Lfs*7 | Serous Ovarian Cancer |
| 339 | E339* | Adrenocortical Carcinoma |
| | E339Afs*8 | |
| | E339Rfs*6 | Breast Invasive Ductal Carcinoma |
| | | Head and Neck Squamous Cell Carcinoma |
| | | Lung Adenocarcinoma |
| | | Oligodendroglioma |
| | | Stomach Adenocarcinoma |
| 340 | M340Sfs*8 | Serous Ovarian Cancer |
| 341 | F341Efs*7 | Head and Neck Squamous Cell Carcinoma |
| | F341L | Hepatocellular Carcinoma |
| | F341V | Lung Adenocarcinoma |
| 342 | R342* | Acute Myeloid Leukemia |
| | R342Efs*2 | Astrocytoma |
| | R342Efs*3 | Breast Invasive Ductal Carcinoma |
| | R342P | Colon Adenocarcinoma |
| | | Cutancous Melanoma |
| | | Esophageal Adenocarcinoma |
| | | Esophageal Squamous Cell Carcinoma |
| | | Glioblastoma Multiforme |
| | | Head and Neck Squamous Cell Carcinoma |
| | | Intestinal Type Stomach Adenocarcinoma |
| | | Lung Adenocarcinoma |
| | | Lung Squamous Cell Carcinoma |
| | | Myxofibrosarcoma |
| | | Oligoastrocytoma |
| | | Pancreatic Adenocarcinoma |
| | | Prostate Adenocarcinoma |
| | | Rectal Adenocarcinoma |
| | | Serous Ovarian Cancer |
| | | Stomach Adenocarcinoma |
| | | Uterine Endometrioid Carcinoma |
| | | Uterine Serous Carcinoma/Uterine Papillary Serous Carcinoma |
| 343 | E343* | Esophageal Squamous Cell Carcinoma |
| | E343Gfs*2 | Head and Neck Squamous Cell Carcinoma |
| | | Lung Adenocarcinoma |
| | | Serous Ovarian Cancer |
| 345 | N345D | Mucinous Stomach Adenocarcinoma |
| | N345Mfs*25 | Serous Ovarian Cancer |
| | N345Sfs*2 | Tubular Stomach Adenocarcinoma |
| 346 | E346* | Lung Adenocarcinoma |
| | | Oligoastrocytoma |
| 347 | A347V | Uterine Mixed Endometrial Carcinoma |
| 348 | L348* | Head and Neck Squamous Cell Carcinoma |
| | L348F | Lung Squamous Cell Carcinoma |
| | L348S | Serous Ovarian Cancer |
| | L348Wfs*22 | |
| 351 | K351* | Leiomyosarcoma |
| | K351E | Lung Adenocarcinoma |
| | | Lung Squamous Cell Carcinoma |
| 354 | Q354* | Lung Adenocarcinoma |
| 355 | A355T | Lung Adenocarcinoma |
| 367 | X367_splice | Bladder Urothelial Carcinoma |
| | | Head and Neck Squamous Cell Carcinoma |
| | | Uterine Mixed Endometrial Carcinoma |
| 375 | Q375* | Bladder Urothelial Carcinoma |
| | Q375K | Glioblastoma Multiforme |
| | | Papillary Thyroid Cancer |
| 376 | S376C | Cervical Squamous Cell Carcinoma |
| 379 | R379C | Uterine Serous Carcinoma/Uterine Papillary Serous Carcinoma |
| 382 | K382Nfs*40 | Bladder Urothelial Carcinoma |
| | | Oligoastrocytoma |
| | | Uterine Endometrioid Carcinoma |
| | | Uterine Mixed Endometrial Carcinoma |
| 383 | L383Cfs*38 | Head and Neck Squamous Cell Carcinoma |
| 385 | F385L | Bladder Urothelial Carcinoma |
| 390 | P390Qfs*32 | Esophageal Squamous Cell Carcinoma |
| 392 | S392Tfs*76 | Intestinal Type Stomach Adenocarcinoma |

A Unless noted otherwise, the mutations of Table 1 are found in cBioPortal database derived from
Cerami et al. The cBio Cancer Genomics Portal: An Open Platform for Exploring
Multidimensional Cancer Genomics Data. Cancer Discovery. May 2012 2; 401; and Gao et al.
Integrative analysis of complex cancer genomics and clinical profiles using the cBioPortal. Sci.
Signal. 6, pl1 (2013).

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as kinase inhibitors, signal transduction inhibitors and/or monoclonal antibodies, or combinations of any of the foregoing. For example, a surgery may be open surgery or minimally invasive surgery. Compounds of Formula (I), or pharmaceutically acceptable salts thereof, therefore may also be useful as adjuvants to cancer treatment, that is, they can be used in combination with one or more additional therapies or therapeutic agents, for example, a chemotherapeutic agent that works by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered a compound of Formula (I), or a pharmaceutically acceptable salt thereof, for a period of time and under one or more rounds of radiation therapy. In some embodiments, the treatment with a compound of Formula (I), or a pharmaceutically acceptable salt thereof, reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject has undergone prior therapy. In some embodiments, a subject is naïve to p53 restoration therapy. In some embodiments, a subject is not naïve to p53 restoration therapy. In some embodiments, a subject is kinase inhibitor naïve. In some embodiments, a subject is not kinase inhibitor naïve.

In some embodiments of any the methods described herein, the compound of Formula (I) (or a pharmaceutically acceptable salt thereof) is administered in combination with a therapeutically effective amount of at least one additional therapeutic agent selected from one or more additional therapies or therapeutic (e.g., chemotherapeutic) agents described herein. For example, in some embodiments, the compound of Formula (I) (or a pharmaceutically acceptable salt thereof) is administered in combination with one, two, or three independently selected additional therapeutic agents as described herein.

Non-limiting examples of additional therapeutic agents include small molecules, antibodies, and antibody-drug conjugates such as EGFR inhibitors, HER2 inhibitors, RAS pathway targeted therapeutic agents (as described herein), PARP inhibitors, CDK4/6 inhibitors, FGFR inhibitors, ALK inhibitors, NTRK/ROS inhibitors, MET inhibitors, RET inhibitors, other kinase inhibitors (e.g., receptor tyrosine kinase-targeted therapeutic agents (e.g., multi-kinase inhibitors)), selective estrogen receptor modulators or degraders (SERMs/SERDs), anti-androgens, checkpoint inhibitors; cytotoxic chemotherapeutics, angiogenesis-targeted therapies, immune-targeted agents, including immunotherapy, and radiotherapy.

In some embodiments, the CDK4/6 inhibitor is palbociclib (IBRANCE®, PD-0332991), ribociclib (KISQALI®, LEE-011), abemaciclib (VERZENIO®, LY-2835219), trilaciclib (COSELA™, G1T28), lerociclib (G1T38), dalpiciclib (SHR-6390), or BPI-16350.

In some embodiments, the FGFR inhibitor is pemigatinib (PEMAZYRE®, INCB-054828), infigratinib (TRUSELTIQ®, BGJ-398, NVP-BGJ398), futibatinib (LYTGOBI®, TAS-120), erdafitinib (BALVERSA®, JNJ-42756493), AZD4547, derazantinib (ARQ-087), AZD4547, ferulic acid-13C3, FGFR-IN-7, PP58, FGFR3-IN-1, ENMD-2076 tartrate, R1530, FGFR3-IN-3, ryrosine kinase-IN-1, SU4984, roblitinib (FGF-401), PD173074, FGFR4-IN-8, lucitanib (E-3810), masitinib (AB1010), zoligratinib (debio 1347, CH5183284), FGFR4-IN-4, BLU9931, SM1-71, TG 100801, FGFR1 inhibitor-6, or TG 100572.

In some embodiments, the ALK inhibitor is crizotinib (XALKORI®, PF-02341066), ceritinib (ZYKADIA®, LDK-378), alectinib (ALECENSA®, CH5424802, R05424802, AF802), brigatinib (ALUNBRIG®, AP-26113), lorlatinib (LORBRENA®, PF-06463922), entrectinib (NMS-E628, RXDX-101, ROZLYTREK®), ASP3026, TSR-011, PF-06463922, ensartinib (X-396), or CEP-37440.

In some embodiments, the NTRK/ROS inhibitor is entrectinib (NMS-E628, RXDX-101, ROZIL.YTREK®), taletrectinib (DS-6051b, AB-106), or repotrectinib (TPX-0005), In some embodiments, the MET inhibitor is capmatinib (TABRECTA®, INC280; INCB28060), tepotinib (TEPMETKO®), tivantinib (ARQ197), savolitinib (ORPATHYS®, Volitinib, HMPL-504, AZD-6094), foretinib (XL880, GSK1363089, GSK089, EXEL-2880), pamufetinib (TAS-115), c-Met-IN-2, PHA-665752, SU11274, SYNI143, or amuvatinib hydrochloride (MP470 hydrochloride, HPK 56 hydrochloride).

In some embodiments, the RET inhibitor is selpercatinib (RETEVMO®, LOXO-292), zeteletinib (BOS-172738, DS-5010), GSK3179106, amuvatinib hydrochloride (MP470 hydrochloride, HPK 56 hydrochloride), TPX-0046, or pralsetinib (GAVRETO®, BLU-667).

In some embodiments, the EGFR inhibitor is osimertinib (AZD9291, merelectinib, TAGRISSO™), erlotinib (TARCEVA®), gefitinib (IRESSA®), cetuximab (ERBITUX®), necitumumab (PORTRAZZA™, IMC-111F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), mavelertinib (PF-06747775), icotinib (BPI-2009H), afatinib (BIBW 2992, GILOTRIF®), dacomitinib (PF-00299804, PF-804, PF-299, PF-299804), avitinib (ACO0010), ACOOOMA EA1045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (C1-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Exemplary HER2 inhibitors include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF (ARAF, BRAF, CRAF), MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K, AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor.

Non-limiting examples of a KRAS-targeted therapeutic agents (e.g., KRAS inhibitors) include sotorasib (AMG510, LUMAKRAS®), BI 1701963, BI 1823911, ARS-853, ARS-3248, ARS-1620, AZD4785, SML-8-73-1, SML-10-70-1, VSA9, GDC-6036, D-1553, AA12, JDQ443, and adagrasib (MRTX-849).

Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the BRAF inhibitor is vemurafenib (ZELBORAF®), dabrafenib (TAFINLAR®), and encorafenib (BRAFTOVI®), BMS-908662 (XL281), sorafenib, PLX3603, RAF265, R05185426, GSK2118436, ARQ 736, GDC-0879, PLX-4720, AZ304, PLX-8394, H1M95573, RO5126766, LXH254, or a combination thereof.

In some embodiments, the MEK inhibitor is trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, R05126766, CS3006, WX-554, PD98059, C11040 (PD184352), hypothemycin, or a combination thereof.

In some embodiments, the ERK inhibitor is FRI-20 (ON-01060), VTX-Ile, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, ONC201, or a combination thereof.

In some embodiments, the PI3K inhibitor is selected from buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH₅₁₃₂₇₉₉, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, ONC201, or a combination thereof.

In some embodiments, the mTOR inhibitor is selected from MLN0128, vistusertib (AZD-2014), onatasertib (CC-223), CC-115, everolimus (RAD001), temsirolimus (CCI-779), ridaforolimus (AP-23573), sirolimus (rapamycin), ridaforolimus (MK-8669), or a combination thereof.

In some embodiments, a chemotherapeutic agent includes an anthracycline, a topoisomerase inhibitors, an antimetabolite, an alkylating agent, a taxane, a platinum-based agent, mitomycin, eribulin (HALAVEN™), or combinations thereof.

In some embodiments, the topoisomerase inhibitor is irinotecan (CAMPTOSAR®), camptothecin, topotecan, etoposide, or teniposide.

In some embodiments, the alkylating agent is cyclophosphamide, Melphalan, chlorambucil, ifosfamide, bendamustine, carmustine, lomustine, or busulfan. In some embodiments, the alkylating agent is cyclophosphamide.

In some embodiments, the antimetabolite is methotrexate, pemetrexed (ALIMTA®), 5-fluorouracil (5-FU), 6-Mercaptopurine (6-MP), capecitabine (XELODA®), cytarabine (Ara-C®), floxuridine, fludarabine, gemcitabine (GEMZAR®), hydroxycarbamide, phototrexate, or a combination of any of the foregoing. In some embodiments, the antimetabolite is methotrexate, pemetrexed, or 5-FU.

Non-limiting examples of a taxane include paclitaxel, docetaxel, abraxane, and taxotere.

In some embodiments, the anthracycline is selected from daunorubicin, doxorubicin, epirubicin, idarubicin, aclarubicin, and combinations thereof.

In some embodiments, the platinum-based agent is selected from carboplatin, cisplatin, oxaliplatin, nedplatin, triplatin tetranitrate, phenanthriplatin, picoplatin, satraplatin and combinations thereof.

Non-limiting examples of PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

Non-limiting examples of selective estrogen receptor modulators or degraders (SERMs/SERDs) include tamoxifen, fulvestrant, brilanestrant, elacestrant, giredestrant, amcenestrant (SAR439859), AZD9833, rintodestrant, LSZ102, LY3484356, ZN-c5, D-0502, and SHR9549.

Non-limiting examples of anti-androgens include enzalutamide (XTANDI®), leuprolide (LUPRON®, ELIGARD®), goserelin (ZOLDEX®), triptorelin (TRELSTAR®), leuprolide mesylate (CAMCEVI®), flutamide (EULEXIN®), bicalutamide (CASXODEX®), nilutamide (NILANDRON®), degarelix (FIRMAGON®), relugolix (ORGOVYX®), and abiraterone (ZYTIGA®).

Non-limiting examples of immunotherapy include immune checkpoint therapies, such as inhibitors that target CTLA-4, PD-1, PD-L1, BTLA, LAG-3, A2AR, TIM-3, B7-H3, VISTA, IDO, and combinations thereof. In some embodimetnts the CTLA-4 inhibitor is ipilimumab (YERVOY®). In some embodiments, the PD-1 inhibitor is selected from pembrolizumab (KEYTRUDA®), nivolumab (OPDIVO®), cemiplimab (LIBTAYO®), dostarlimab (JEMPERLI®), vopratelimab (JTX-4014), spartalizumab (PDR001), camrelizumab (SHR1210), sintilimab (I13I308), tislelizumab (BGB-A317), toripalimab (JS 001), INCMGA00012, AMP-224, AMP-514 (MEDI0680), or combinations thereof. In some embodiments, the PD-LI inhibitor is selected from atezolizumab (TECENTRIQ®), avelumab (BAVENCIO®), durvalumab (IMFINZI®), KN035, cosibelimab (CK-301), AUNP12, CA-170, BMS-986189, or combinations thereof. In some embodiments, the LAG-3 inhibitor is IMP701 (LAG525). In some embodiments, the A2AR inhibitor is CPI-444. In some embodiments, the TIM-3 inhibitor is MBG453. In some embodiments, the B7-H3 inhibitor is enoblituzumab. In some embodiments, the VISTA inhibitor is JNJ-61610588. In some embodiments, the IDO inhibitor is indoximod. See, for example, Marin-Acevedo, et al., J Hematol Oncol. 11: 39 (2018).

In some embodiments, the additional therapy or therapeutic agent is selected from 5-FU, irinotecan, cisplatin, carboplatin, oxaliplatin, doxorubicin, epirubicin, gemcitabine, methotrexate, pemetrexed, cyclophosphamide, olaparib, rucaparib, niraparib, pembrolizumab (KEYTRUDA®), nivolumab (OPDIVO®), cemiplimab (LIBTAYO®), dostarlimab (JEMPERLI®), atezolizumab (TECENTRIQ®), avelumab (BAVENCIO®), durvalumab (IMFINZI®), radiation therapy, and combinations of any of the foregoing.

In some embodiments, additional therapeutic agents may also be administered to treat potential side-effects for particular anticancer therapies and/or as palliative therapy, for example, opioids and corticosteroids.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994), Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

The compounds described herein can be synthesized, for example, using the procedure shown in Scheme 1, Scheme 2, or Scheme 3 below, using different coupling partners from diversifiable intermediate 8, intermediate 16, or intermediate 29.

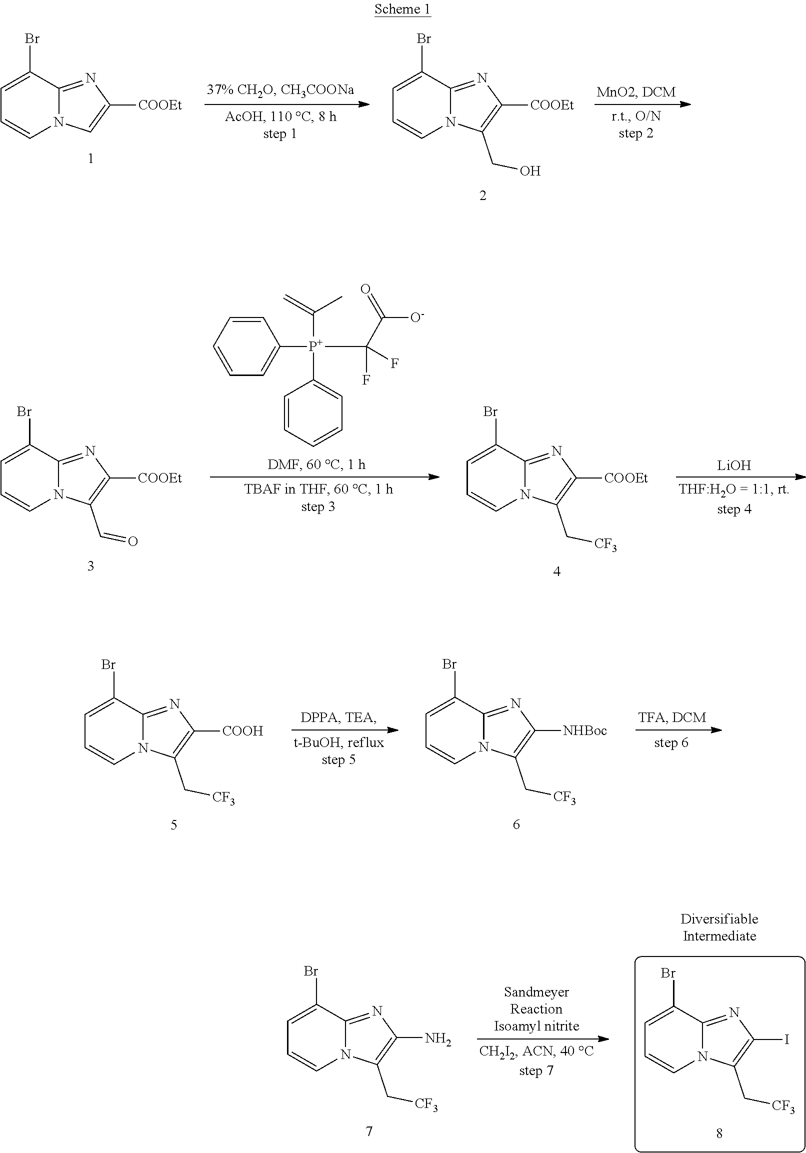

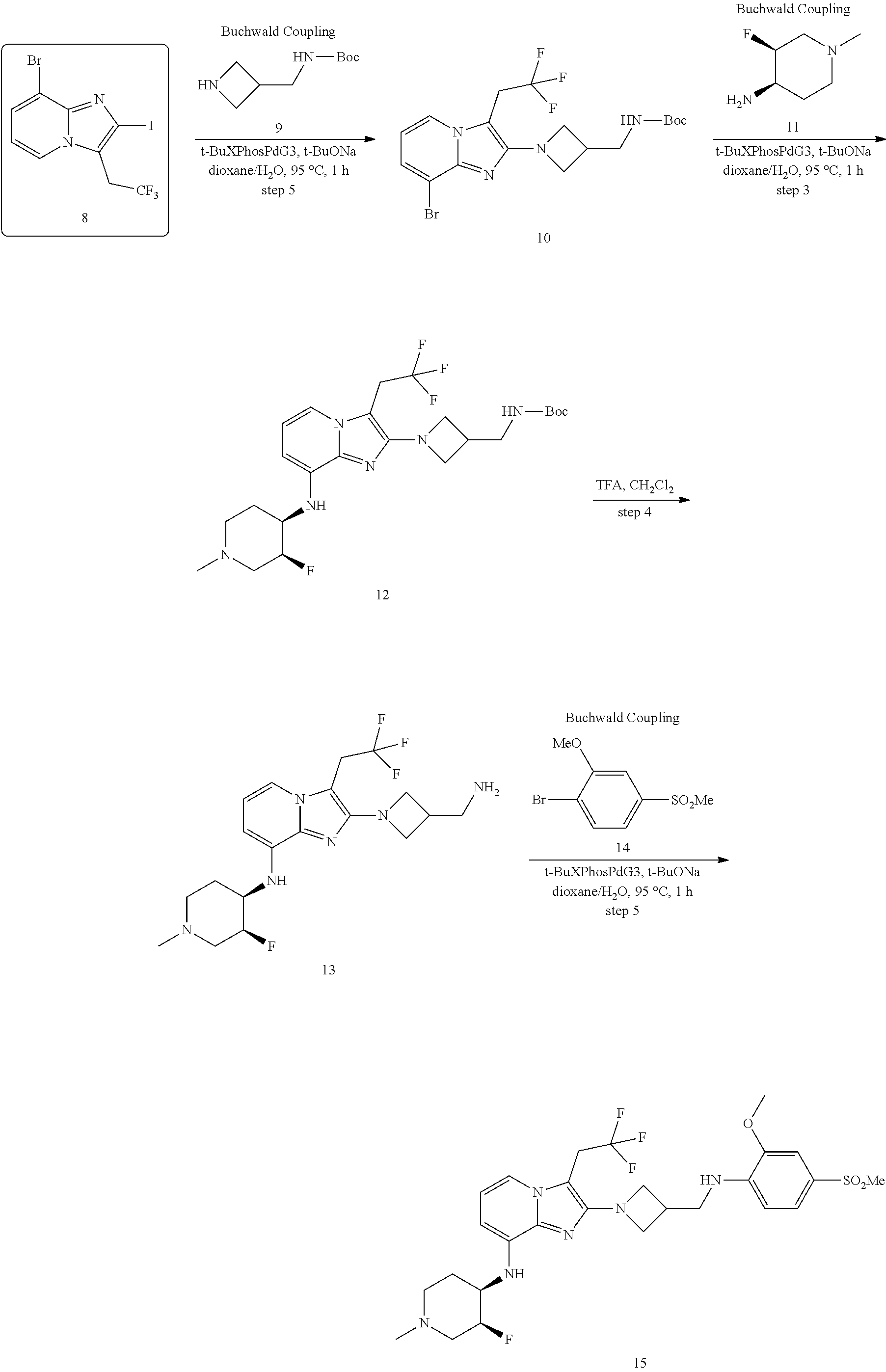

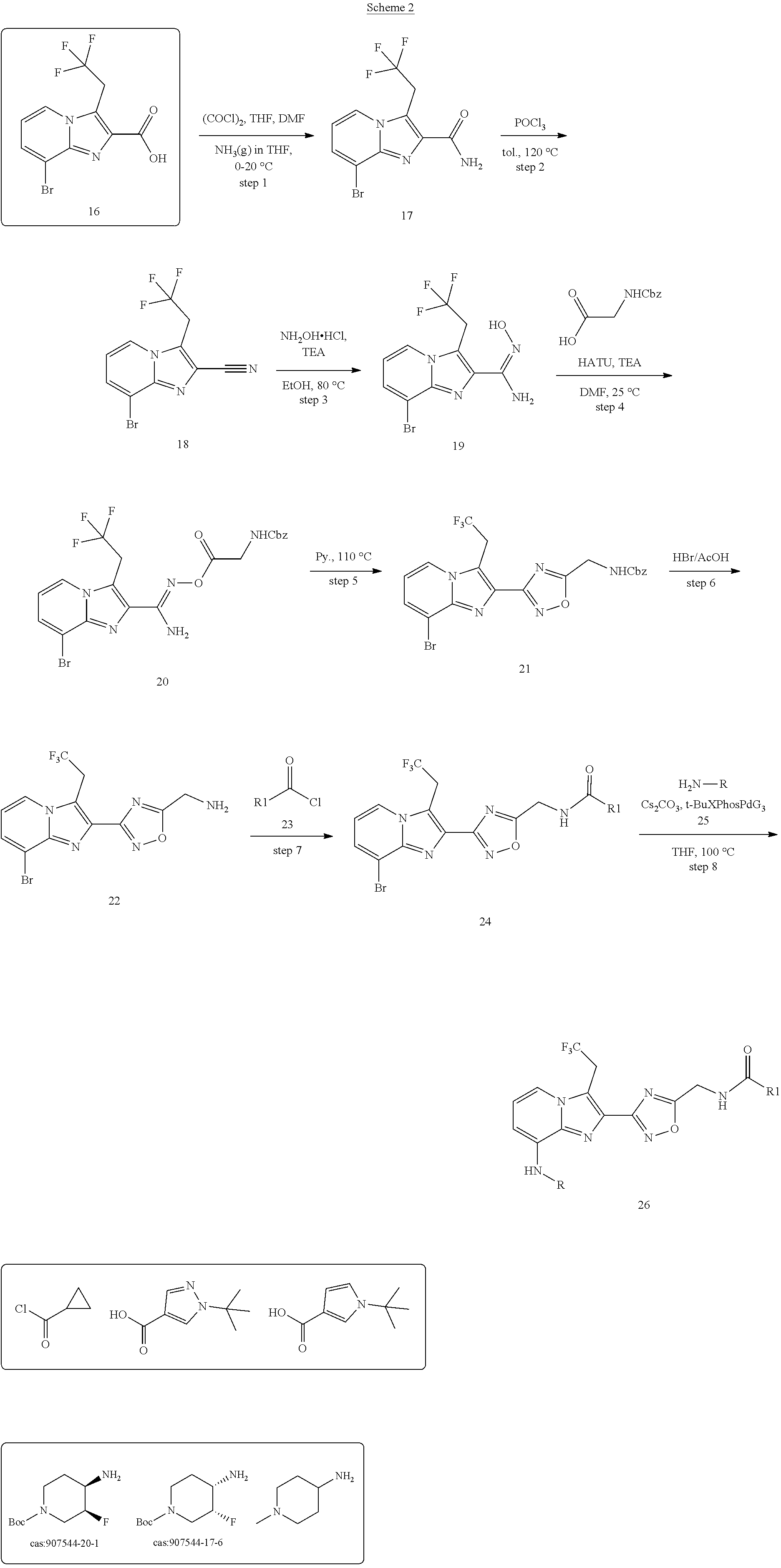

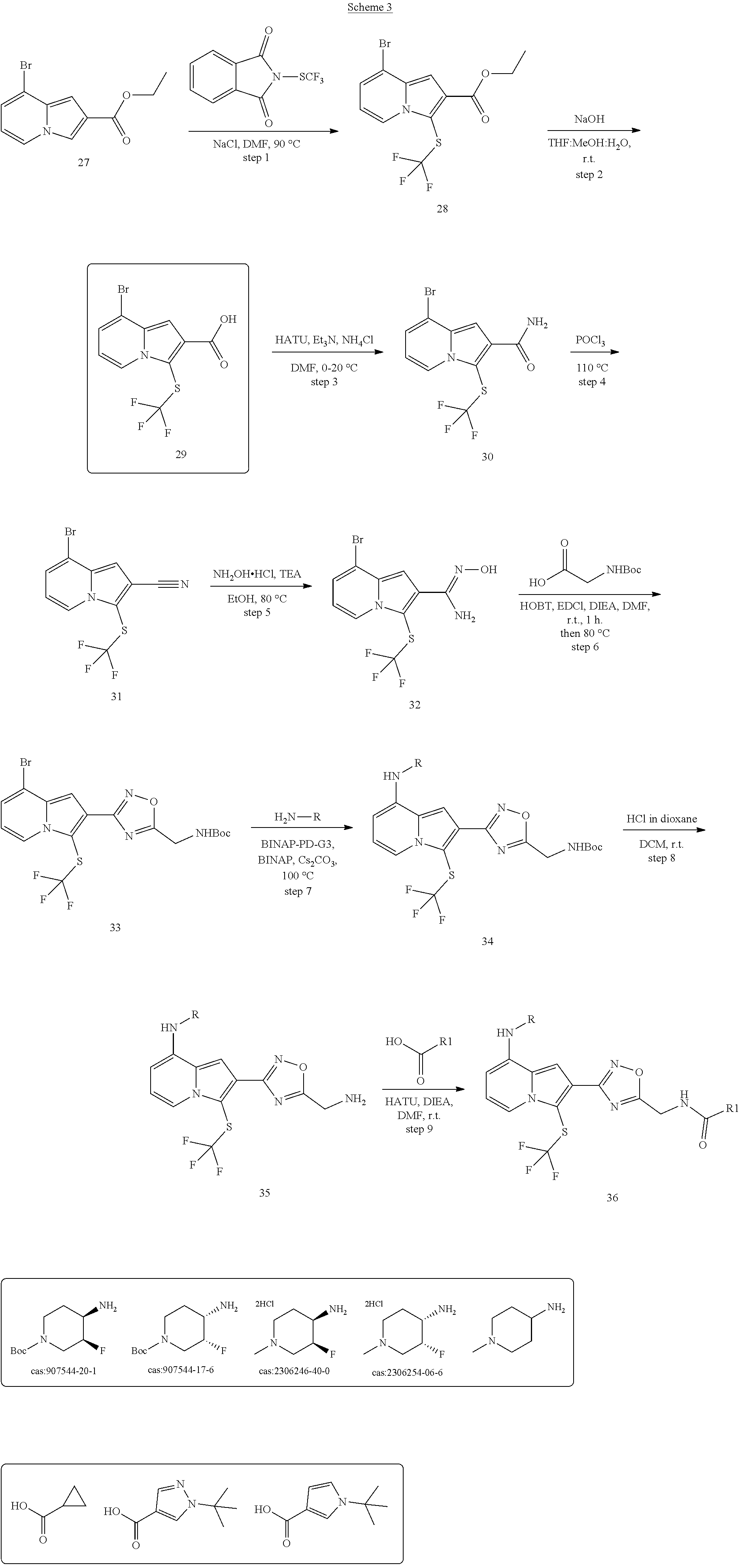

### Example 1—Preparation of N-{[3-(8-{[(3S,4R)-3-fluoro-1-methylpiperidin-4-yl]amino}-3-[(trifluoromethyl)sulfanyl]imidazo[1,2-a]pyridin-2-yl)-1,2,4-oxadiazol-5-yl]methyl}cyclopropanecarboxamide

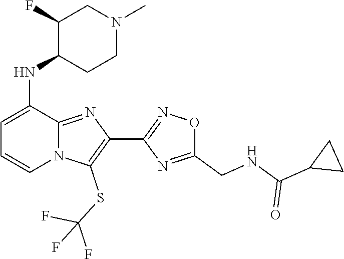 [SMILES: CN1CC[C@@H](Nc2cccn3c(SC(F)(F)F)c(-c4noc(CNC(=O)C5CC5)n4)nc23)[C@@H](F)C1]

Step 1: To a stirred solution of 8-bromo-3-[(trifluoromethyl)sulfanyl]imidazo[1,2-a]pyridine-2-carboxylic acid (1.00 g, 2.93 mmol, 1.00 equiv) in DMF (3.00 mL) was added HATU (1.67 g, 4.39 mmol, 1.50 equiv), NaHCO₃ (1.23 g, 14.66 mmol, 5.00 equiv) and NH₄Cl (470.5 mg, 8.79 mmol, 3.00 equiv). The resulting mixture was stirred for 1 h at room temperature. The resulted solution was purified reverse flash chromatography (Mobile Phase A: Water, Mobile Phase B: ACN; Flow rate: 60 mL/min; Gradient: 0% B to 100% B in 30 min; 254/220 nm) to afford 8-bromo-3-[(trifluoromethyl)sulfanyl]imidazo[1,2-a]pyridine-2-carboxamide (800 mg, 80.23%) as a white solid. LC-MS: [M+H]⁺ found found 339.9.

Step 2: A solution of 8-bromo-3-[(trifluoromethyl)sulfanyl]imidazo[1,2-a]pyridine-2-carboxamide (780.0 mg, 2.29 mmol, 1.00 equiv) in POCl₃ (5 mL) was stirred for 30 min at 110 °C.

The solvent was removed under vacuum. The residue was quenched by ice and basified to pH 7 with saturated NaHCO₃ at 0 °C. The resulted mixture was poured into water and extracted with EA. The organic layer was washed with brine, dried over Na₂SO₄ and evaporated to give 8-bromo-3-[(trifluoromethyl)sulfanyl]imidazo[1,2-a]pyridine-2-carbonitrile (700 mg, 94.76%) as a yellow solid. LC-MS: [M+H]⁺ found found 321.8.

Step 3: To a solution of 8-bromo-3-[(trifluoromethyl)sulfanyl]imidazo[1,2-a]pyridine-2-carbonitrile (500.0 mg, 1.55 mmol, 1.00 equiv) in EtOH (5.00 mL) was added hydroxylamine (50%, 205.1 mg, 3.10 mmol, 2.00 equiv). The reaction mixture was stirred at room temperature for 1 h. The resulting mixture was concentrated under vacuum. This resulted in 8-bromo-N′-hydroxy-3-[(trifluoromethyl)sulfanyl]imidazo[1,2-a]pyridine-2-carboximidamide (500 mg, 90.7%) as a white solid. LC-MS: [M+H]⁺ found found 354.9.

Step 4: To a stirred solution of 8-bromo-N′-hydroxy-3-[(trifluoromethyl)sulfanyl]imidazo[1,2-a]pyridine-2-carboximidamide (500.0 mg, 1.40 mmol, 1.00 equiv) in DMF (6.0 mL) were added (tert-butoxycarbonyl)glycine (369.3 mg, 2.11 mmol, 1.50 equiv), HATU (801.8 mg, 2.11 mmol, 1.50 equiv) and TEA (569.9 mg, 5.63 mmol, 4.00 equiv). After stirring at room temperature for 1 h, the reaction mixture was heated at 100 °C for another 4 h. The resulted solution was purified by reverse flash chromatography (Mobile Phase A: Water, Mobile Phase B: ACN; Flow rate: 60 mL/min; Gradient: 0% B to 100% B in 30 min; 254/220 nm). This resulted in tert-butyl N-[(3-{8-bromo-3-[(trifluoromethyl)sulfanyl]imidazo[1,2-a]pyridin-2-yl}-1,2,4-oxadiazol-5-yl)methyl]carbamate (300 mg, 43.11%) as a yellow solid. LC-MS: [M+H]⁻ found 494.1.

Step 5: To a stirred solution of tert-butyl N-[(3-{8-bromo-3-[(trifluoromethyl)sulfanyl]imidazo[1,2-a]pyridin-2-yl}-1,2,4-oxadiazol-5-yl)methyl]carbamate (280.0 mg, 0.56 mmol, 1.00 equiv) in THE (3.0 mL) were added (3S,4R)-3-fluoro-1-methylpiperidin-4-amine dihydrochloride (232.4 mg, 1.13 mmol, 2.00 equiv), tBuXPhos Pd G3 (90.0 mg, 0.11 mmol, 0.20 equiv) and t-BuONa (272.2 mg, 2.83 mmol, 5.00 equiv). The resulting mixture was stirred for 2 h at 65 °C under nitrogen atmosphere, then purified by reverse flash chromatography (Mobile Phase A: Water, Mobile Phase B: ACN; Flow rate: 60 mL/min; Gradient: 0% B to 100% B in 30 min; 254/220 nm). This resulted in tert-butyl N-{[3-(8-{[(3S,4R)-3-fluoro-1-methylpiperidin-4-yl]amino}-3-[(trifluoromethyl)sulfanyl]imidazo[1,2-a]pyridin-2-yl)-1,2,4-oxadiazol-5-yl]methyl}carbamate (40 mg, 12.94%) as a yellow oil. LC-MS: [M+H]⁺ found 546.3.

Step 6: To a stirred solution of tert-butyl N-{[3-(8-{[(3S,4R)-3-fluoro-1-methylpiperidin-4-yl]amino}-3-[(trifluoromethyl)sulfanyl]imidazo[1,2-a]pyridin-2-yl)-1,2,4-oxadiazol-5-yl]methyl}carbamate (40.0 mg, 0.06 mmol, 1.00 equiv) in MeOH (1.00 mL) was added HCl (4M in dioxane, 3.00 mL). The resulting mixture was stirred for 1 h at room temperature. The solvent was removed under vacuum. This resulted in (3S,4R)-N-{2-[5-(aminomethyl)-1,2,4-oxadiazol-3-yl]-3-[(trifluoromethyl)sulfanyl]imidazo[1,2-a]pyridin-8-yl}-3-fluoro-1-methylpiperidin-4-amine hydrochloride (40 mg, crude) as a yellow solid. LC-MS: [M+H]⁺ found 446.1.

Step 7: To a stirred solution of (3S,4R)-N-{2-[5-(aminomethyl)-1,2,4-oxadiazol-3-yl]-3-[(trifluoromethyl)sulfanyl]imidazo[1,2-a]pyridin-8-yl}-3-fluoro-1-methylpiperidin-4-amine hydrochloride (40.0 mg, 0.09 mmol, 1.00 equiv) in DMF (3.0 mL) were added cyclopropanecarboxylic acid (10.5 mg, 0.12 mmol, 1.30 equiv), HATU (45.6 mg, 0.12 mmol, 1.30 equiv) and NaHCO₃ (47.1 mg, 0.55 mmol, 6 equiv). The resulting mixture was stirred for 1 h at room temperature. The resulted solution was purified by reverse flash chromatography (Mobile Phase A: Water, Mobile Phase B: ACN; Flow rate: 60 mL/min; Gradient: 0% B to 100% B in 30 min; 254/220 nm) and Prep-HPLC (Column: XBridge Prep OBD C18 Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 23% B to 53% B in 7 min; Wave Length: 254 nm/220 nm nm; RT(min): 6.75). This resulted in N-{[3-(8-{[(3S,4R)-3-fluoro-1-methylpiperidin-4-yl]amino}-3-[(trifluoromethyl)sulfanyl]imidazo[1,2-a]pyridin-2-yl)-1,2,4-oxadiazol-5-yl]methyl}cyclopropanecarboxamide (12.8 mg, 26.24%) as a white solid. LC-MS: [M+H]⁺ found 514.25. ¹H NMR (400 MHz, DMSO-d₆) δ 9.02 (t, J=5.8 Hz, 1H), 8.05 (d, J=6.7 Hz, 1H), 7.13 (t, J=7.2 Hz, 1H), 6.72 (d, J=7.7 Hz, 1H), 5.62 (d, J=9.1 Hz, 1H), 4.89 (d, J=49.3 Hz, 1H), 4.70 (d, J=5.7 Hz, 2H), 3.81 (dt, J=27.0, 8.4 Hz, 1H), 3.08 (t, J=11.5 Hz, 1H), 2.80 (d, J=10.8 Hz, 1H), 2.36-2.23 (m, 1H), 2.21 (s, 3H), 2.13 (td, J=10.9, 4.0 Hz, 1H), 1.88 (td, J=10.5, 4.0 Hz, 2H), 1.68 (m, 1H), 0.79-0.67 (m, 4H).

### Example 3—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclopropanecarboxamide

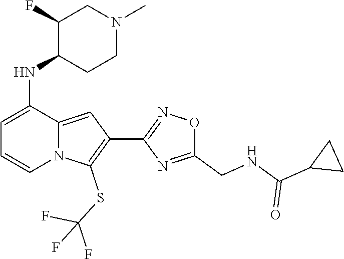 [SMILES: CN1CC[C@@H](Nc2cccn3c(SC(F)(F)F)c(-c4noc(CNC(=O)C5CC5)n4)cc23)[C@@H](F)C1]

Step 1: A mixture of ethyl 8-bromoindolizine-2-carboxylate (2 g, 7.46 mmol, 1 equiv), 2-[(trifluoromethyl) sulfanyl]isoindole-1,3-dione (2.21 g, 8.95 mmol, 1.20 equiv), NaCl (87.19 mg, 1.49 mmol, 0.2 equiv) in DMF (10 mL) was stirred for 4 h at 90 °C under nitrogen atmosphere.

The resulting mixture was diluted with EtOAc (100 mL), washed with brine, and dried over anhydrous Na₂SO₄. After filtration, the filtrate was concentrated under reduced pressure. The residue was purified by silica gel column chromatography, eluted with PE/EA (100:1) to afford ethyl 8-bromo-3-[(trifluoromethyl)sulfanyl]indolizine-2-carboxylate (1.8 g, 65.54%) as a light yellow solid. LC-MS: (M+H)⁺ found 368.1.

Step 2: A solution of ethyl 8-bromo-3-[(trifluoromethyl)sulfanyl]indolizine-2-carboxylate (1.8 g, 4.89 mmol, 1 equiv) and caustic soda (1 g, 24.45 mmol, 5 equiv) in MeOH (7 mL)/THF (7 mL)/H₂O (6 mL) was stirred for 1 h at room temperature. The resulting mixture was concentrated under reduced pressure. The mixture was acidified to pH 2 with HCl (3N). The precipitated solids were collected by filtration and dried under vacuum to afford 8-bromo-3-[(trifluoromethyl)sulfanyl]indolizine-2-carboxylic acid (1.8 g) as a light yellow solid. LC-MS: (M+H)⁺ found 340.1.

Step 3: To a stirred solution of 8-bromo-3-[(trifluoromethyl)sulfanyl]indolizine-2-carboxylic acid (1 g, 2.94 mmol, 1 equiv), Et₃N (0.89 g, 8.82 mmol, 3 equiv), and NH₄Cl (1.57 g, 29.40 mmol, 10 equiv) in DMF (10 mL) was added HATU (1.68 g, 4.41 mmol, 1.5 equiv) at 0 °C.

The reaction mixture was stirred for 1 h at room temperature. The resulting mixture was diluted with ethyl acetate (100 mL), then washed with 3*100 mL of brine. The organic phase was dried over anhydrous sodium sulfate and concentrated under vacuum. The residue was washed with MeOH (3 mL) and filtered to give 8-bromo-3-[(trifluoromethyl)sulfanyl]indolizine-2-carboxamide (850 mg, 85.25%) as a white solid. LC-MS: (M+H)⁺ found 339.2.

Step 4: A mixture of 8-bromo-3-[(trifluoromethyl)sulfanyl]indolizine-2-carboxamide (1 g, 2.95 mmol, 1 equiv) and POCl₃ (0.99 g, 6.49 mmol, 2.2 equiv) in toluene (10 mL) was stirred at 110 °C for 1 h. The resulting mixture was cooled to room temperature and concentrated under vacuum. The residue was purified by silica gel column chromatography, eluted with PE/EA (10:1) to afford 8-bromo-3-[(trifluoromethyl)sulfanyl]indolizine-2-carbonitrile (0.8 g, 84.49%) as a white solid. LC-MS: (M+H)⁺ found 321.1.

Step 5: A mixture of 8-bromo-3-[(trifluoromethyl)sulfanyl]indolizine-2-carbonitrile (1 g, 3.11 mmol, 1 equiv), NH₂OH—HCl (770 mg, 4.67 mmol, 1.5 equiv) and Et₃N (0.95 g, 9.34 mmol, 3 equiv) in EtOH (10 mL) was stirred for 1 h at 80 °C. The resulting mixture was concentrated under vacuum. The residue was purified by silica gel column chromatography, eluted with PE/EA (5:1) to afford (Z)-8-bromo-N′-hydroxy-3-[(trifluoromethyl)sulfanyl]indolizine-2-carboximidamide (700 mg, 63.47%) as a white solid. LC-MS: (M+H)⁺ found 354.2.

Step 6: To a stirred solution of (Z)-8-bromo-N′-hydroxy-3-[(trifluoromethyl)sulfanyl]indolizine-2-carboximidamide (2 g, 5.65 mmol, 1 equiv), DIEA (7.30 g, 56.47 mmol, 10 equiv), and [(tert-butoxycarbonyl)amino]acetic acid (1.19 g, 6.77 mmol, 1.2 equiv) in DMF (20 mL) were added HOBT (1.53 g, 11.29 mmol, 2 equiv) and EDCI (2.17 g, 11.29 mmol, 2 equiv) at 0 °C. The reaction mixture was stirred at room temperature for 1 h and then 80 °C for 16 h. The mixture was purified by reversed-phase flash chromatography directly (column, C18 silica gel; mobile phase, MeCN in water (0.1% FA), 0% to 100% gradient in 20 min; detector, UV 254 nm) to give tert-butyl N-[(3-{8-bromo-3-[(trifluoromethyl)sulfanyl]indolizin-2-yl}-1,2,4-oxadiazol-5-yl)methyl]carbamate (1.8 g, 64.61%) as a white solid. LC-MS: (M+H)⁺ found 493.3.

Step 7: A mixture of tert-butyl N-[(3-{8-bromo-3-[(trifluoromethyl)sulfanyl]indolizin-2-yl}-1,2,4-oxadiazol-5-yl)methyl]carbamate vanadium (500 mg, 0.92 mmol, 1 equiv), (3S,4R)-3-fluoro-1-methylpiperidin-4-amine dihydrochloride (283 mg, 1.38 mmol, 1.5 equiv), RAC-BINAP-PD-G3 (183 mg, 0.18 mmol, 0.2 equiv), BINAP (228 mg, 0.37 mmol, 0.4 equiv), and Cs₂CO₃ (1.5 g, 4.59 mmol, 5 equiv) in dioxane (5 mL) were added was stirred for 6 h at 100 °C under nitrogen atmosphere. The resulting mixture was concentrated under reduced pressure. The residue was purified by silica gel column chromatography, eluted with CH₂Cl₂/MeOH (10:1) to afford tert-butyl N-{[3-(8-{[(3S)-3-fluoro-1-methylpiperidin-4-yl]amino}-3-[(trifluoromethyl) sulfanyl]indolizin-2-yl)-1,2,4-oxadiazol-5-yl]methyl}carbamate (300 mg, 59.96%) as a light brown solid. LC-MS: (M+H)⁺ found 545.2.

Step 8: A solution of tert-butyl N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}carbamate (280 mg, 0.51 mmol, 1 equiv) and 4M HCl (gas) in 1,4-dioxane (0.6 mL) in DCM (3 mL) was stirred for 0.5 h at room temperature. The resulting mixture was concentrated under reduced pressure to afford crude product 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (280 mg, crude) as a brown solid. LC-MS: (M+H)⁺ found 454.2.

Step 9: To a stirred solution of cyclopropanecarboxylic acid (14.5 mg, 0.17 mmol, 1.5 equiv), 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), and DIEA (0.10 mL, 0.56 mmol, 5 equiv) in DMF (1 mL) was added HATU (64.2 mg, 0.17 mmol, 1.5 equiv) at 0 °C.

The resulting solution was stirred for additional 1 h at room temperature, then diluted with EtOAc (20 mL) and washed with brine (2*30 mL). The organic layer was dried over anhydrous Na₂SO₄.

After filtration, the filtrate was concentrated under reduced pressure. The residue was purified by silica gel column chromatography (CH₂Cl₂/MeOH=10:1) and Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 32% B to 57% B in 8 min; Wave Length: 220 nm nm; RT1 (min): 7.73) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclopropanecarboxamide (20.4 mg, 35.10%) as a light yellow solid. LC-MS: (M+H)⁺ found 513.15. ¹H NMR (400 MHz, DMSO-d₆) δ 8.99 (t, J=5.8 Hz, 1H), 8.02 (d, J=6.9 Hz, 1H), 7.79 (s, 1H), 6.85 (t, J=7.2 Hz, 1H), 6.29 (d, J=8.0 Hz, 1H), 6.21 (d, J=7.6 Hz, 1H), 4.87 (d, J=49.5 Hz, 1H), 4.67 (d, J=5.7 Hz, 2H), 3.63 (d, J=29.1 Hz, 1H), 3.05 (t, J=11.2 Hz, 1H), 2.83 (s, 1H), 2.20 (s, 4H), 2.08 (t, J=8.9 Hz, 2H), 1.76-1.60 (m, 2H), 0.73 (m, 4H).

### Example 4—Preparation of N-{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}cyclopropanecarboxamide

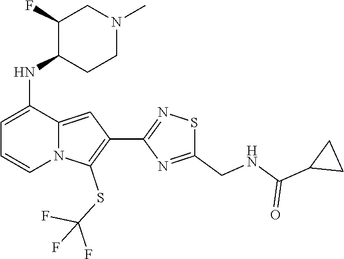 [SMILES: CN1CC[C@@H](Nc2cccn3c(SC(F)(F)F)c(-c4nsc(CNC(=O)C5CC5)n4)cc23)[C@@H](F)C1]

Step 1: A solution of 8-bromo-3-[(trifluoromethyl)sulfanyl]indolizine-2-carboxamide (2 g, 5.89 mmol, 1 equiv) and chloro(chlorosulfanyl)methanone (803.3 mg, 6.13 mmol, 1.04 equiv) in THE (20 mL) was stirred for 1 h at 65 °C. The resulting solution was diluted with water (100 mL) and extracted with EtOAc (3*100 mL). The combined organic layers were dried over anhydrous Na₂SO₄. After filtration, the filtrate was concentrated under reduced pressure. The residue was purified by silica gel column chromatography, eluted with PE/EA (100:1) to afford 5-{8-bromo-3-[(trifluoromethyl)sulfanyl]indolizin-2-yl}-1,3,4-oxathiazol-2-one (800 mg, 34.15%) as a light yellow solid. LC-MS: (M+H)⁺ found 397.3.

Step 2: A solution of 5-{8-bromo-3-[(trifluoromethyl)sulfanyl]indolizin-2-yl}-1,3,4-oxathiazol-2-one (800 mg, 2.01 mmol, 1 equiv) and 2-ethoxy-2-oxoacetonitrile (399.2 mg, 4.03 mmol, 2.00 equiv) in M-xylene (4 mL) was irradiated with microwave radiation for 15 min at 180 °C. The solution was allowed to cool down to room temperature and concentrated under vacuum. The residue was purified by silica gel column chromatography, eluted with PE/EA (10:1) to afford ethyl 3-{8-bromo-3-[(trifluoromethyl)sulfanyl]indolizin-2-yl}-1,2,4-thiadiazole-5-carboxylate (600 mg, 65.87%) as a light yellow solid. LC-MS: (M+H)⁺ found 451.1.

Step 3: A mixture of NaBH₄ (501.9 mg, 13.27 mmol, 10 equiv) in THE (5 mL) was stirred for 5 min at 0 °C. To the above mixture was added ethyl 3-{8-bromo-3-[(trifluoromethyl)sulfanyl]indolizin-2-yl}-1,2,4-thiadiazole-5-carboxylate (600 mg, 1.33 mmol, 1 equiv) in portions. The resulting mixture was stirred overnight at room temperature, then quenched with MeOH at 0 °C and concentrated under reduced pressure. The residue was purified by reversed-phase flash chromatography (column, C18 silica gel; mobile phase, MeOH in water (0.1% FA), 0% to 70% gradient in 20 min; detector, UV 254 nm) to afford (3-{8-bromo-3-[(trifluoromethyl)sulfanyl]indolizin-2-yl}-1,2,4-thiadiazol-5-yl)methanol (400 mg, 73.50%) as a light yellow solid. LC-MS: (M+H)⁺ found 410.2.

Step 4: A solution of (3-{8-bromo-3-[(trifluoromethyl)sulfanyl]indolizin-2-yl}-1,2,4-thiadiazol-5-yl)methanol (400 mg, 0.98 mmol, 1 equiv), TEA (296.0 mg, 2.93 mmol, 3 equiv) and TsCl (185.9 mg, 0.98 mmol, 1 equiv) in DCM (3 mL) was stirred for 1 h at room temperature. The resulting mixture was diluted with water (100 mL) and extracted with EtOAc (3*100 mL). The combined organic layers were dried over anhydrous Na₂SO₄. After filtration, the filtrate was concentrated under reduced pressure. The residue was dissolved in DCM (3 mL) and added to a stirred NH₃(g) in MeOH (6 mL) at room temperature. The resulting solution was stirred for 3 h at 40 °C under, then concentrated under reduced pressure to afford 1-(3-{8-bromo-3-[(trifluoromethyl) sulfanyl]indolizin-2-yl}-1,2,4-thiadiazol-5-yl)methanamine (300 mg, 75.18%) as a light yellow solid. LC-MS: (M+H)⁺ found 409.1.

Step 5: A solution of 1-(3-{8-bromo-3-[(trifluoromethyl)sulfanyl]indolizin-2-yl}-1,2,4-thiadiazol-5-yl)methanamine (100 mg, 0.24 mmol, 1 equiv), cyclopropanecarboxylic acid (25.2, 0.29 mmol, 1.2 equiv), DIEA (94.8 mg, 0.73 mmol, 3.00 equiv) and HATU (92.9 mg, 0.244 mmol, 1.00 equiv) in DMF (1 mL) was stirred for 1 h at room temperature. The resulting solution was diluted with water (100 mL) and extracted with EtOAc (3*100 mL). The combined organic layers were dried over anhydrous Na₂SO₄. After filtration, the filtrate was concentrated under reduced pressure. The residue was purified by Prep-TLC (CH₂Cl₂/MeOH 15:1) to afford N-(3-{8-bromo-3-[(trifluoromethyl)sulfanyl]indolizin-2-yl}-1,2,4-thiadiazol-5-yl)methyl]cyclopropanecarboxamide (80 mg, 68.59%) as a light brown solid. LC-MS: (M+H)⁺ found 477.1.

Step 6: A mixture of N-[(3-{8-bromo-3-[(trifluoromethyl)sulfanyl]indolizin-2-yl}-1,2,4-thiadiazol-5-yl)methyl]cyclopropanecarboxamide (80 mg, 0.17 mmol, 1 equiv), (3 S,4R)-3-fluoro-1-methylpiperidin-4-amine dihydrochloride (68.8 mg, 0.34 mmol, 2 equiv), Pd-PEPPSI-IHeptCl 3-chloropyridine (81.6 mg, 0.08 mmol, 0.5 equiv) and Cs₂CO₃ (327.6 mg, 1.01 mmol, 6 equiv) in dioxane (1 mL) was stirred for 2 h at 100 °C under nitrogen atmosphere. The resulting mixture was filtered, the filter cake was washed with acetonitrile. The filtrate was concentrated under reduced pressure. The residue was purified by Prep-TLC (CH₂Cl₂/(7 M NH₃ in MeOH=20/1) and Prep-HPLC (Column: XBridge Prep OBD C18 Column, 30*150 mm, 5 μm; Mobile Phase A: Water (10 mmol/L NH₄HCO₃+0.05% NH₃—H₂O), Mobile Phase B: ACN; Flow rate: 60 mL/min mL/min; Gradient: 36% B to 61% B in 8 min; Wave Length: 220 nm nm; RT1 (min): 7.4) to afford N-{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}cyclopropanecarboxamide (3.4 mg, 3.84%) as a light yellow solid. LC-MS: (M+H)⁺ found 529.15. ¹H NMR (400 MHz, DMSO-d₆) δ 9.17 (t, J=5.8 Hz, 1H), 8.02 (d, J=6.9 Hz, 1H), 7.84 (s, 1H), 6.82 (t, J=7.2 Hz, 1H), 6.19 (t, J=8.9 Hz, 2H), 5.02-4.69 (m, 3H), 3.63 (d, J=29.8 Hz, 1H), 3.05 (t, J=11.3 Hz, 1H), 2.83 (s, 1H), 2.20 (s, 4H), 2.10 (d, J=8.3 Hz, 2H), 1.69 m, 2H), 0.77 (d, J=5.2 Hz, 4H).

### Example 55—Preparation of N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]cyclopropanecarboxamide

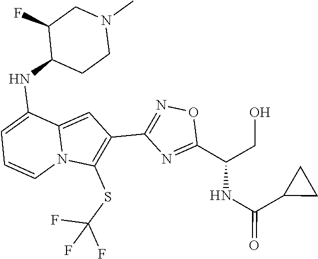 [SMILES: CN1CC[C@@H](Nc2cccn3c(SC(F)(F)F)c(-c4noc([C@H](CO)NC(=O)C5CC5)n4)cc23)[C@@H](F)C1]

Step 1: A mixture of 8-bromo-3-[(trifluoromethyl)sulfanyl]indolizine-2-carbonitrile (1 g, 3.11 mmol, 1 equiv), (3S,4R)-3-fluoro-1-methylpiperidin-4-amine dihydrochloride (958.1 mg, 4.67 mmol, 1.5 equiv), Cs₂CO₃ (5.07 g, 15.57 mmol, 5 equiv) and Pd-PEPPSI-IHeptCl 3-chloropyridine (303.3 mg, 0.31 mmol, 0.1 equiv) in 1,4-dioxane (10 mL) was stirred for 4 h at 100 °C under nitrogen atmosphere. The resulting mixture was filtered, the filter cake was washed with acetonitrile. The filtrate was concentrated under reduced pressure. The residue was purified by silica gel column chromatography, eluted with CH₂C2/MeOH (15:1) to afford 8-{[(3S,4R)-3-fluoro-1-methylpiperidin-4-yl]amino}-3-[(trifluoromethyl)sulfanyl]indolizine-2-carbonitrile (1.2 g, 103.48%) as a yellow solid. LC-MS: (M+H)⁺ found 373.2.

Step 2: A solution of 8-{[(3S,4R)-3-fluoro-1-methylpiperidin-4-yl]amino}-3-[(trifluoromethyl)sulfanyl]indolizine-2-carbonitrile (1.2 g, 3.22 mmol, 1 equiv), TEA (1.63 g, 16.11 mmol, 5 equiv) and NH₂OH HCl (0.67 g, 9.67 mmol, 3 equiv) in EtOH (10 mL) was stirred for 3 h at 80 °C under nitrogen atmosphere. The solution was diluted with EtOAc (50 mL) and washed with brine (3*50 mL). The organic layer was dried over anhydrous Na₂SO₄. After filtration, the filtrate was concentrated under reduced pressure. The crude product (1. Ig) was used in the next step directly without further purification. LC-MS: (M+H)⁺ found 406.3.

Step 3: A solution of (Z)-8-{[(3S,4R)-3-fluoro-1-methylpiperidin-4-yl]amino}-N′-hydroxy-3-[(trifluoromethyl) sulfanyl]indolizine-2-carboximidamide (300 mg, 0.74 mmol, 1 equiv), (2S)-2-[(tert-butoxycarbonyl)amino]-3-hydroxypropanoic acid (182.2 mg, 0.89 mmol, 1.2 equiv), HOBT (200 mg, 1.48 mmol, 2 equiv), EDCI (283.7 mg, 1.48 mmol, 2 equiv) and DIEA (956.4 mg, 7.40 mmol, 10 equiv) in DMF (6 mL) was stirred for 3 h at room temperature under nitrogen atmosphere. The resulting solution was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 15:1) to afford tert-butyl N-[(1S)-1-[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]-2-hydroxyethyl]carbamate (190 mg, 44.69%) as a yellow solid. LC-MS: (M+H)⁺ found 575.2.

Step 4: To a stirred solution of tert-butyl N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]carbamate (190 mg, 0.33 mmol, 1 equiv) in DCM (5 mL) were added HCl (gas) in 1,4-dioxane (0.5 mL) dropwise at room temperature. The resulting mixture was stirred for 0.5 h at room temperature, then basified to pH 8 with sat·NaHCO₃. The resulting mixture was extracted with CH₂Cl₂. The combined organic layers were dried over anhydrous Na₂SO₄. After filtration, the filtrate was concentrated under reduced pressure. The crude product (170 mg) was used in the next step directly without further purification. LC-MS: (M+H)⁺ found 409.1.

Step 5: A solution of (2S)-2-amino-2-[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]ethanol (170 mg, 0.36 mmol, 1 equiv), cyclopropanecarboxylic acid (37.0 mg, 0.43 mmol, 1.2 equiv), PyBOP (223.7 mg, 0.43 mmol, 1.2 equiv) and DIEA (231.5 mg, 1.79 mmol, 5 equiv) in DMF (2 mL) was stirred for 1 h at room temperature. The resulting solution was extracted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/7 M NH₃ in MeOH 15:1) (Column: CHIRALPAKIC-34.6*50 mm, 3.0 um; Mobile Phase A: Hex(0.2% DEA):(EtOH:DCM=1:1)=60:40; Gradient: isocratic; Injection Volume: 5.0 L mL) to afford N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]cyclopropanecarboxamide (17.2 mg, 8.85%) as a light yellow solid. LC-MS: (M+H)⁺ found 543.15. ¹H NMR (400 MHz, DMSO-d₆) δ 8.96 (d, J=7.2 Hz, 1H), 8.08 (d, J=6.9 Hz, 1H), 7.77 (s, 1H), 6.90 (t, J=7.2 Hz, 1H), 6.52 (d, J=7.8 Hz, 1H), 6.25 (d, J=7.7 Hz, 1H), 5.38 (s, 1H), 5.34-5.02 (m, 2H), 4.09-3.66 (m, 4H), 3.47 (s, 2H), 3.12 (s, 1H), 2.80 (s, 3H), 2.39-2.18 (m, 1H), 1.99 (d, J=13.9 Hz, 1H), 1.85-1.70 (m, 1H), 0.72 (m, 4H).

### Example 53—Preparation of N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]cyclopropanecarboxamide

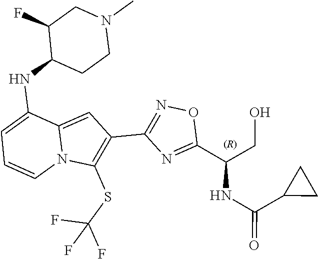

Step 1: A solution of (Z)-8-{[(3S,4R)-3-fluoro-1-methylpiperidin-4-yl]amino}-N′-hydroxy-3-[(trifluoromethyl) sulfanyl]indolizine-2-carboximidamide (400 mg, 0.99 mmol, 1 equiv), HOBT (266.6 mg, 1.97 mmol, 2 equiv), EDCI (378.3 mg, 1.97 mmol, 2 equiv) and DIEA (1.28 g, 9.87 mmol, 10 equiv) in DMF (5 mL) was stirred for 3 h at room temperature, then stirred overnight at 80 °C. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 15:1) to afford tert-butyl N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]carbamate (188 mg, 33.16%) as a light yellow solid. LC-MS: (M+H)~found 575.2.

Step 2: To a stirred solution of tert-butyl N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]carbamate (188 mg, 0.33 mmol, 1 equiv) in DCM (5 mL) was added HCl (gas) in 1,4-dioxane (0.5 mL) dropwise at room temperature. The resulting mixture was stirred for 0.5 h at room temperature, then basified to pH 8 with sat·NaHCO₃. The resulting mixture was extracted with CH₂Cl₂. The combined organic layers were dried over anhydrous Na₂SO₄. After filtration, the filtrate was concentrated under reduced pressure. The residue (140 mg) was used in the next step directly without further purification. LC-MS: (M+H)⁺ found 409.1.

Step 3: A solution of (2R)-2-amino-2-[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]ethanol (140 mg, 0.30 mmol, 1 equiv), cyclopropanecarboxylic acid (27.9 mg, 0.33 mmol, 1.10 equiv), PyBOP (184.3 mg, 0.35 mmol, 1.2 equiv) and DIEA (190.7 mg, 1.48 mmol, 5 equiv) in DMF (2 mL) was stirred for 1 h at room temperature. The resulting solution was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/7 M NH₃ in MeOH 15:1) and Prep-HPLC (Column: CHIRALPAKIC-34.6*50 mm, 3.0 um; Mobile Phase A: Hex(0.2% DEA):(EtOH:DCM=1:1)=60:40; Gradient: isocratic; Injection Volume: 5.0 L mL) to afford N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]-2-hydroxyethyl]cyclopropanecarboxamide (37 mg, 23.11%) as a light yellow solid. LC-MS: (M+H)⁻ found 543.30. ¹H NMR (400 MHz, DMSO-d₆) δ 8.94 (d, J=7.5 Hz, 1H), 8.03 (d, J=6.9 Hz, 1H), 7.79 (s, 1H), 6.86 (t, J=7.2 Hz, 1H), 6.32 (d, J=8.1 Hz, 1H), 6.21 (d, J=7.6 Hz, 1H), 5.35 (t, J=5.7 Hz, 1H), 5.24 (m 1H), 4.88 (d, J=49.2 Hz, 1H), 3.99-3.78 (m, 2H), 3.64 (d, J=29.0 Hz, 1H), 3.06 (s, 1H), 2.85 (s, 1H), 2.21 (s, 4H), 2.11 (d, J=9.1 Hz, 2H), 1.74 (m 2H), 0.72 (m 4H).

### Example 51—Preparation of N-[(1R*)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide

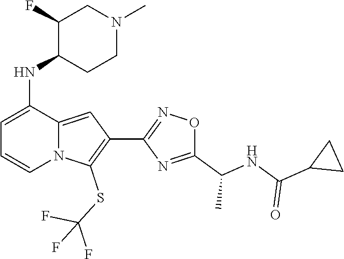 [SMILES: C[C@@H](NC(=O)C1CC1)c1nc(-c2cc3c(N[C@@H]4CCN(C)C[C@@H]4F)cccn3c2SC(F)(F)F)no1]

Step 1: To a stirred mixture of (Z)-8-bromo-N′-hydroxy-3-[(trifluoromethyl)sulfanyl]indolizine-2-carboximidamide (1 g, 2.82 mmol, 1 equiv), (2R)-2-[(tert-butoxycarbonyl)amino]propanoic acid (801.4 mg, 4.24 mmol, 1.5 equiv) and DIEA (3.65 g, 28.240 mmol, 10 equiv) in DMF (1 mL) were added HOBT (763.1 mg, 5.65 mmol, 2 equiv) and EDCI (1.08 g, 5.65 mmol, 2 equiv) at room temperature. The resulting mixture was stirred for 3 h at room temperature and then overnight at 80 °C. The resulting mixture was diluted with EA (100 mL) and washed with brine (3*100 mL). The organic layer was dried over anhydrous Na₂SO₄.

After filtration, the filtrate was concentrated under reduced pressure. The residue was purified by silica gel column chromatography, eluted with PE/EA (6:1) to afford tert-butyl N-[(1R)-1-(3-{8-bromo-3-[(trifluoromethyl) sulfanyl]indolizin-2-yl}-1,2,4-oxadiazol-5-yl)ethyl]carbamate (350 mg, 24.43%, 88 ee %) as a white solid. LC-MS: (M+H)⁺ found 507.1.

Step 2: A mixture of tert-butyl N-[(1R)-1-(3-{8-bromo-3-[(trifluoromethyl)sulfanyl]indolizin-2-yl}-1,2,4-oxadiazol-5-yl)ethyl]carbamate (460 mg, 0.91 mmol, 1 equiv), (3S,4R)-3-fluoro-1-methylpiperidin-4-amine dihydrochloride (279 mg, 1.36 mmol, 1.50 equiv), BINAP (282.3 mg, 0.45 mmol, 0.50 equiv), Cs₂CO₃ (1.48 g, 4.54 mmol, 5 equiv) and RAC-BINAP-PD-G3 (90 mg, 0.09 mmol, 0.10 equiv) in dioxane (4 mL) was stirred for 4 h at 100 °C under nitrogen atmosphere. The resulting mixture was filtered, the filter cake was washed with ACN. The filtrate was concentrated under reduced pressure. The residue was purified by reversed-phase flash chromatography (column, C18 silica gel; mobile phase, MeOH in water, 0% to 100% gradient in 20 min; detector, UV 254 nm) and Prep-TLC (CH₂Cl₂/MeOH 20:1) to afford tert-butyl N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]carbamate (120 mg, 23.69%, 45 ee %) as a grey solid. LC-MS: (M+H)⁺ found 559.2.

Step 3: To a stirred solution of tert-butyl N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]carbamate (120 mg, 0.21 mmol, 1 equiv) in DCM (2 mL) were added TFA (0.4 mL) at room temperature. The resulting solution was stirred for 0.5 h at room temperature, then basified to pH 8 with sat·NaHCO₃. The resulting mixture was extracted with DCM (3*50 mL). The combined organic layers were dried over anhydrous Na₂SO₄. After filtration, the filtrate was concentrated under reduced pressure to afford 2-{5-[(1R)-1-aminoethyl]-1,2,4-oxadiazol-3-yl}-N-[(3S,4R)-3-fluoro-1-methylpiperidin-4-yl]-3-[(trifluoromethyl)sulfanyl]indolizin-8-amine (120 mg, crude) as a yellow oil. LC-MS: (M+H)⁺ found 459.2.

Step 4: A solution of 2-{5-[(1R)-1-aminoethyl]-1,2,4-oxadiazol-3-yl}-N-[(3S,4R)-3-fluoro-1-methylpiperidin-4-yl]-3-[(trifluoromethyl)sulfanyl]indolizin-8-amine (120 mg, 0.26 mmol, 1 equiv), cyclopropanecarboxylic acid (27.0 mg, 0.31 mmol, 1.2 equiv), DIEA (169.1 mg, 1.31 mmol, 5.00 equiv) and PyBOP (163.4 mg, 0.31 mmol, 1.20 equiv) in DMF (2 mL) was stirred for 1 h at room temperature. The resulting solution was purified by reversed-phase flash chromatography (column, C18 silica gel, mobile phase, MeCN in water, 0% to 100% gradient in 20 min; detector, UV 254 nm) and Prep-TLC (CH₂Cl₂/7 M NH₃ in MeOH 20:1) to afford N-[(1R)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide (42 mg, 30.48%, 45 ee %) as a off-white solid. LC-MS: (M+H)⁺ found 527.2.

Step 5: The crude product (42 mg) was purified by Prep-HPLC (Column: CHIRALPAKIA3; Mobile Phase A: Hex(0.2% DEA):(EtOH:DCM=1:1)=80:20; Flow rate: 1 mL/min mL/min; Gradient: isocratic; Injection Volume: 3 L mL) to afford N-[(1R*)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide (18.7 mg, 44.52%, 100 ee %) as a white solid. LC-MS: (M+H)⁺ found 527.15. ¹H NMR (400 MHz, DMSO-d₆) δ 8.95 (d, J=7.3 Hz, 1H), 8.03 (d, J=6.8 Hz, 1H), 7.78 (s, 1H), 6.86 (t, J=7.2 Hz, 1H), 6.33 (d, J=8.1 Hz, 1H), 6.21 (d, J=7.7 Hz, 1H), 5.27 (m, 1H), 4.89 (d, J=49.2 Hz, 1H), 3.83-3.57 (m, 1H), 3.11 (s, 1H), 2.89 (d, J=10.4 Hz, 1H), 2.49-1.98 (m, 6H), 1.75-1.69 (m, 1H), 1.69-1.44 (m, 4H), 0.84-0.57 (m, 4H).

### Example 52—Preparation of N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide

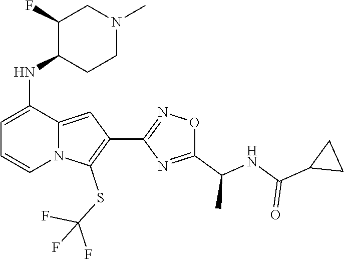 [SMILES: C[C@H](NC(=O)C1CC1)c1nc(-c2cc3c(N[C@@H]4CCN(C)C[C@@H]4F)cccn3c2SC(F)(F)F)no1]

Step 1: A solution of (Z)-8-bromo-N′-hydroxy-3-[(trifluoromethyl)sulfanyl]indolizine-2-carboximidamide (1 g, 2.82 mmol, 1 equiv), (2S)-2-[(tert-butoxycarbonyl)amino]propanoic acid (0.64 g, 3.39 mmol, 1.2 equiv), HOBT (0.76 g, 5.65 mmol, 2 equiv), EDCI (1.08 g, 5.65 mmol, 2 equiv) and DIEA (3.65 g, 28.24 mmol, 10 equiv) in DMF (10 mL) was stirred for 1 h at room temperature. Then the solution was heated at 80 °C for 5 h. The reaction was diluted with EA (100 mL) and washed with brine (3*100 mL). The organic layer was dried over anhydrous Na₂SO₄. After filtration, the filtrate was concentrated under reduced pressure. The residue was purified by silica gel column chromatography, eluted with PE/EA (6:1) to afford tert-butyl N-[(1S)-1-(3-{8-bromo-3-[(trifluoromethyl)sulfanyl]indolizin-2-yl}-1,2,4-oxadiazol-5-yl)ethyl]carbamate (1.152 g, 80.42%, 89.4 ee %) as a white solid. LC-MS: (M+H)⁺ found 507.1.

Step 2: A solution of tert-butyl N-[(1S)-1-(3-{8-bromo-3-[(trifluoromethyl)sulfanyl]indolizin-2-yl}-1,2,4-oxadiazol-5-yl)ethyl]carbamate (1 g, 1.97 mmol, 1 equiv), (3S,4R)-3-fluoro-1-methylpiperidin-4-amine dihydrochloride (0.61 g, 2.96 mmol, 1.5 equiv), RAC-BINAP-PD-G3 (0.39 g, 0.39 mmol, 0.2 equiv), BINAP (0.61 g, 0.99 mmol, 0.5 equiv) and Cs₂CO₃ (3.21 g, 9.86 mmol, 5 equiv) in dioxane (10 mL) was stirred for 1 h at 100 °C under nitrogen atmosphere. The mixture was allowed to cool down to room temperature. The resulting mixture was filtered, the filter cake was washed with DCM. The filtrate was washed with brine (50 mL), then dried over anhydrous Na₂SO₄. After filtration, the filtrate was concentrated under reduced pressure. The residue was purified by reversed-phase flash chromatography (column, C18 silica gel; mobile phase, MeCN in water, 0% to 100% gradient in 50 min; detector, UV 254 nm). This resulted in tert-butyl N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]carbamate (600 mg, 54.49%) as a yellow solid. LC-MS: (M+H)~found 559.2.

Step 3: A solution of tert-butyl N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]carbamate (300 mg, 0.54 mmol, 1 equiv) and TFA (0.6 mL, 8.08 mmol, 15.04 equiv) in DCM (3 mL) was stirred for 1 h at room temperature. The mixture was basified to pH 7 with saturated NaHCO₃, and then extracted with DCM (3*50 mL). The combined organic layers were dried over anhydrous Na₂SO₄. After filtration, the filtrate was concentrated under reduced pressure. The residue was purified by silica gel column chromatography, eluted with CH₂Cl₂/7 M NH₃ in MeOH (25:1) to afford 2-{5-[(1 S)-1-aminoethyl]-1,2,4-oxadiazol-3-yl}-N-[(3 S,4R)-3-fluoro-1-methylpiperidin-4-yl]-3-[(trifluoromethyl)sulfanyl]indolizin-8-amine (68 mg, 27.62%) as a yellow solid. LC-MS: (M+H)y found 459.2.

Step 4: A solution of 2-{5-[(1S)-1-aminoethyl]-1,2,4-oxadiazol-3-yl}-N-[(3S,4R)-3-fluoro-1-methylpiperidin-4-yl]-3-[(trifluoromethyl)sulfanyl]indolizin-8-amine (55 mg, 0.12 mmol, 1 equiv), cyclopropanecarboxylic acid (12.4 mg, 0.14 mmol, 1.2 equiv), PyBOP (81.2 mg, 0.16 mmol, 1.3 equiv) and DIEA (77.5 mg, 0.60 mmol, 5 equiv) in DMF (1 mL) was stirred for 1 h at room temperature. The resulting mixture was diluted with EA (50 mL) and washed with brine (3*50 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃+0.05% NH₁₁H₂O), Mobile Phase B: ACN; Flow rate: 60 mL/min mL/min; Gradient: 38% B to 63% B in 8 min; Wave Length: 220 nm nm; RT1 (min): 6.85) to afford N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide (26 mg, 40.72%, 8 ee %) as a white solid. LC-MS: (M+H)~found 527.15.

Step 5: The crude product (26 mg) was purified by CHIRAL-HPLC (Column: CHIRALPAKIA3; Mobile Phase A: Hex(0.2% DEA):(EtOH:DCM=1:1)=80:20; Flow rate: 1 mL/min mL/min; Gradient: isocratic; Injection Volume: 3 L mL) to afford N-[(1S)-1-[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]ethyl]cyclopropanecarboxamide (8.3 mg, 31.92%, 100 ee %) as a light brown solid. LC-MS: (M+H)⁺ found 527.15. ¹H NMR (400 MHz, DMSO-d₆) δ 8.95 (d, J=7.3 Hz, 1H), 8.03 (d, J=6.8 Hz, 1H), 7.78 (s, 1H), 6.86 (t, J=7.2 Hz, 1H), 6.41-6.29 (m, 1H), 6.21 (d, J=7.6 Hz, 1H), 5.27 (m, 1H), 4.90 (d, J=49.2 Hz, 1H), 3.66 (d, J=28.5 Hz, 1H), 3.13 (s, 1H), 2.90 (d, J=10.2 Hz, 1H), 2.47-1.93 (m, 6H), 1.73 (d, J=10.8 Hz, 1H), 1.64 (m, 1H), 1.57 (d, J=7.1 Hz, 3H), 0.79-0.62 (m, 4H).

### Example 54—Preparation of 6-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)-N-methylpyridine-3-carboxamide

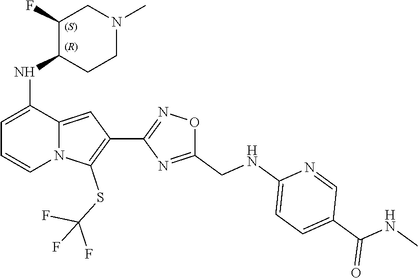

Step 1: A mixture of ethyl 8-bromoindolizine-2-carboxylate (2 g, 7.46 mmol, 1 equiv), 2-[(trifluoromethyl)sulfanyl]isoindole-1,3-dione (2.21 g, 8.95 mmol, 1.20 equiv), NaCl (87.19 mg, 1.49 mmol, 0.2 equiv) in DMF (10 mL) was stirred for 4 h at 90 °C under nitrogen atmosphere. The resulting mixture was diluted with EtOAc (100 mL), washed with brine, and dried over anhydrous Na₂SO₄. After filtration, the filtrate was concentrated under reduced pressure. The residue was purified by silica gel column chromatography, eluted with PE/EA (100:1) to afford ethyl 8-bromo-3-[(trifluoromethyl)sulfanyl]indolizine-2-carboxylate (1.8 g, 65.54%) as a light yellow solid. LC-MS: (M+H)⁺ found 368.1.

Step 2: A solution of ethyl 8-bromo-3-[(trifluoromethyl)sulfanyl]indolizine-2-carboxylate (1.8 g, 4.89 mmol, 1 equiv) and caustic soda (1 g, 24.45 mmol, 5 equiv) in MeOH (7 mL)/THF (7 mL)/H₂O (6 mL) was stirred for 1 h at room temperature. The resulting mixture was concentrated under reduced pressure. The mixture was acidified to pH 2 with HCl (3N). The precipitated solids were collected by filtration and dried under vacuum to afford 8-bromo-3-[(trifluoromethyl)sulfanyl]indolizine-2-carboxylic acid (1.8 g) as a light yellow solid. LC-MS: (M+H)⁺ found 340.1.

Step 3: To a stirred solution of 8-bromo-3-[(trifluoromethyl)sulfanyl]indolizine-2-carboxylic acid (1 g, 2.94 mmol, 1 equiv), Et₃N (0.89 g, 8.82 mmol, 3 equiv), and NH₄Cl (1.57 g, 29.40 mmol, 10 equiv) in DMF (10 mL) was added HATU (1.68 g, 4.41 mmol, 1.5 equiv) at 0 °C. The reaction mixture was stirred for 1 h at room temperature. The resulting mixture was diluted with ethyl acetate (100 mL), then washed with 3*100 mL of brine. The organic phase was dried over anhydrous sodium sulfate and concentrated under vacuum. The residue was washed with MeOH (3 mL) and filtered to give 8-bromo-3-[(trifluoromethyl)sulfanyl]indolizine-2-carboxamide (850 mg, 85.25%) as a white solid. LC-MS: (M+H)⁺ found 339.2.

Step 4: A mixture of 8-bromo-3-[(trifluoromethyl)sulfanyl]indolizine-2-carboxamide (1 g, 2.95 mmol, 1 equiv) and POCl₃ (0.99 g, 6.49 mmol, 2.2 equiv) in toluene (10 mL) was stirred at 110 °C for 1 h. The resulting mixture was cooled to room temperature and concentrated under vacuum. The residue was purified by silica gel column chromatography, eluted with PE/EA (10:1) to afford 8-bromo-3-[(trifluoromethyl)sulfanyl]indolizine-2-carbonitrile (0.8 g, 84.49%) as a white solid. LC-MS: (M+H)⁺ found 321.1.

Step 5: A mixture of 8-bromo-3-[(trifluoromethyl)sulfanyl]indolizine-2-carbonitrile (1 g, 3.11 mmol, 1 equiv), NH₂OH—HCl (770 mg, 4.67 mmol, 1.5 equiv) and Et₃N (0.95 g, 9.34 mmol, 3 equiv) in EtOH (10 mL) was stirred for 1 h at 80 °C. The resulting mixture was concentrated under vacuum. The residue was purified by silica gel column chromatography, eluted with PE/EA (5:1) to afford (Z)-8-bromo-N′-hydroxy-3-[(trifluoromethyl)sulfanyl]indolizine-2-carboximidamide (700 mg, 63.47%) as a white solid. LC-MS: (M+H)⁺ found 354.2.

Step 6: To a stirred solution of (Z)-8-bromo-N′-hydroxy-3-[(trifluoromethyl)sulfanyl]indolizine-2-carboximidamide (2 g, 5.65 mmol, 1 equiv), DIEA (7.30 g, 56.47 mmol, 10 equiv), and [(tert-butoxycarbonyl)amino]acetic acid (1.19 g, 6.77 mmol, 1.2 equiv) in DMF (20 mL) were added HOBT (1.53 g, 11.29 mmol, 2 equiv) and EDCI (2.17 g, 11.29 mmol, 2 equiv) at 0 °C. The reaction mixture was stirred at room temperature for 1 h and then 80 °C for 16 h. The mixture was purified by reversed-phase flash chromatography directly (column, C18 silica gel; mobile phase, MeCN in water (0.10% FA), 0% to 100% gradient in 20 min; detector, UV 254 nm) to give tert-butyl N-[(3-{8-bromo-3-[(trifluoromethyl)sulfanyl]indolizin-2-yl}-1,2,4-oxadiazol-5-yl)methyl]carbamate (1.8 g, 64.61%) as a white solid. LC-MS: (M+H)⁺ found 493.3.

Step 7: A mixture of tert-butyl N-[(3-{8-bromo-3-[(trifluoromethyl)sulfanyl]indolizin-2-yl}-1,2,4-oxadiazol-5-yl)methyl]carbamate vanadium (500 mg, 0.92 mmol, 1 equiv), (3S,4R)-3-fluoro-1-methylpiperidin-4-amine dihydrochloride (283 mg, 1.38 mmol, 1.5 equiv), RAC-BINAP-PD-G3 (183 mg, 0.18 mmol, 0.2 equiv), BINAP (228 mg, 0.37 mmol, 0.4 equiv), and Cs₂CO₃ (1.5 g, 4.59 mmol, 5 equiv) in dioxane (5 mL) were added was stirred for 6 h at 100 °C under nitrogen atmosphere. The resulting mixture was concentrated under reduced pressure. The residue was purified by silica gel column chromatography, eluted with CH₂Cl₂/MeOH (10:1) to afford tert-butyl N-{[3-(8-{[(3S)-3-fluoro-1-methylpiperidin-4-yl]amino}-3-[(trifluoromethyl) sulfanyl]indolizin-2-yl)-1,2,4-oxadiazol-5-yl]methyl}carbamate (300 mg, 59.96%) as a light brown solid. LC-MS: (M+H)⁺ found 545.2.

Step 8: A solution of tert-butyl N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}carbamate (280 mg, 0.51 mmol, 1 equiv) and 4M HCl (gas) in 1,4-dioxane (0.6 mL) in DCM (3 mL) was stirred for 0.5 h at room temperature. The resulting mixture was concentrated under reduced pressure to afford crude product 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (280 mg, crude) as a brown solid. LC-MS: (M+H)⁺ found 445.2.

Step 9: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (80 mg, 0.18 mmol, 1 equiv), TEA (54.6 mg, 0.54 mmol, 3 equiv) and methyl 6-fluoropyridine-3-carboxylate (41.9 mg, 0.27 mmol, 1.5 equiv) in NMP (2 mL) was stirred overnight at 90 °C. The resulting mixture was diluted with water (30 mL) and extracted with EtOAc (3*30 mL). The combined organic layers were concentrated under reduced pressure. The residue was purified by reversed-phase flash chromatography (column, C18 silica gel; mobile phase, MeCN in Water (10 mmol/L NH₄HCO₃), 0% to 100% gradient in 30 min; detector, UV 254 nm) to afford methyl 6-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)pyridine-3-carboxylate (40 mg, 35.28%) as a yellow solid. LC-MS: (M+H)⁺ found 580.2.

Step 10: A solution of methyl 6-({[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}amino)pyridine-3-carboxylate (40 mg, 0.07 mmol, 1 equiv) and NaOH (13.8 mg, 0.35 mmol, 5 equiv) in THE (0.2 mL)/MeOH (0.4 mL)/H₂O (0.4 mL) was stirred for 1 h at room temperature. The resulting solution was diluted with water (10 mL) and acidified to pH 5 with 1M HCl, then extracted with EtOAc (3*10 mL). The organic layer was dried over anhydrous Na₂SO₄. After filtration, the filtrate was concentrated under reduced pressure to afford 6-({[3-(8-{[(3 S,4R)-3-fluoro-1-methylpiperidin-4-yl]amino}-3-[(trifluoromethyl)sulfanyl]indolizin-2-yl)-1,2,4-oxadiazol-5-yl]methyl}amino)pyridine-3-carboxylic acid (35 mg) as a white solid. LC-MS: (M+H)⁺ found 566.0.

Step 11: A solution of 6-({[3-(8-{[(3S,4R)-3-fluoro-1-methylpiperidin-4-yl](methyl)amino}-3-[(trifluoromethyl) sulfanyl]indolizin-2-yl)-1,2,4-oxadiazol-5-yl]methyl}amino)pyridine-3-carboxylic acid (40 mg, 0.07 mmol, 1 equiv), methanamine, hydrochloride (14.0 mg, 0.21 mmol, 3 equiv), HATU (31.5 mg, 0.08 mmol, 1.2 equiv) and TEA (34.9 mg, 0.35 mmol, 5 equiv) in DMF (1 mL) was stirred for 1 h at room temperature. The resulting mixture was diluted with water (10 mL) and extracted with CH₂Cl (2*10 mL). The combined organic layers were concentrated under reduced pressure. The residue was purified by Prep-HPLC (Column: XBridge Prep OBD C18 Column, 30*150 mm, 5 μm; Mobile Phase A: Water (10 mmol/L NH₄HCO₃)+0.05% NH₃H₂O, Mobile Phase B: ACN; Flow rate: 60 mL/min mL/min; Gradient: 27% B to 54% B in 7 min; Wave Length: 254 nm/220 nm nm; RT1 (min): 7.77) to afford 6-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)-N-methylpyridine-3-carboxamide (6.3 mg, 15.45%) as a white solid. LC-MS: (M+H)⁺ found 579.35. ¹H NMR (400 MHz, DMSO-d₆) δ 8.44 (d, J=2.3 Hz, 1H), 8.15 (d, J=4.7 Hz, 1H), 8.01 (d, J=6.9 Hz, 1H), 7.94-7.83 (m, 2H), 7.74 (s, 1H), 6.84 (t, J=7.2 Hz, 1H), 6.68 (d, J=8.7 Hz, 1H), 6.21 (dd, J=16.2, 7.9 Hz, 2H), 4.97-4.72 (m, 3H), 3.62 (d, J=28.9 Hz, 1H), 3.04 (t, J=11.3 Hz, 1H), 2.84 (dd, J=14.9, 6.6 Hz, 1H), 2.72 (d, J=4.5 Hz, 3H), 2.19 (s, 4H), 2.13-2.00 (m, 2H), 1.68 (d, J=10.4 Hz, 1H).

### Example 50—Preparation of 2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)-N-methylpyrimidine-5-carboxamide

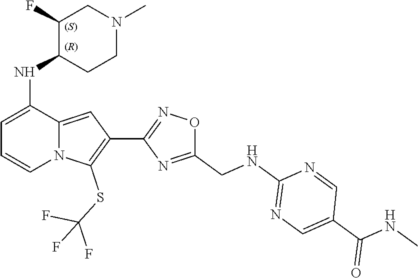

Step 1: A mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (130 mg, 0.29 mmol, 1 equiv), methyl 2-chloropyrimidine-5-carboxylate (55.5 mg, 0.32 mmol, 1.1 equiv), and KF (34.0 mg, 0.58 mmol, 2 equiv) in DMSO (1 mL) stirred for 40 min at room temperature. The resulting mixture was diluted with water (50 mL) and extracted with EtOAc (2*50 mL). The combined organic layers were dried over anhydrous Na₂SO₄. After filtration, the filtrate was concentrated under reduced pressure. The residue was purified by Prep-TLC (CH₂Cl₂/MeOH=10:1) to afford methyl 2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)pyrimidine-5-carboxylate (90 mg, 53.00%) as a yellow solid. LC-MS: (M+H)⁺ found 581.2.

Step 2: To a stirred solution of methyl 2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)pyrimidine-5-carboxylate (40 mg, 0.07 mmol, 1 equiv) in THE (1 mL)/H₂O (0.25 mL) was added LiOH (10 mg, 0.08 mmol, 1.2 equiv) at room temperature. The resulting mixture was stirred for 1 h at room temperature, then acidified to pH 5 with HCl (aq.). The resulting solution was extracted with DCM (3*20 mL). The combined organic layers were dried over anhydrous Na₂SO₄. After filtration, the filtrate was concentrated under reduced pressure to afford 2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)pyrimidine-5-carboxylic acid (23 mg, 58.92%) as a brown oil. LC-MS: (M+H)⁺ found 567.1.

Step 3: A solution of 2-({[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}amino)pyrimidine-5-carboxylic acid (50 mg, 0.09 mmol, 1 equiv), methanamine hydrochloride (7.8 mg, 0.11 mmol, 1.3 equiv), DIEA (57.0 mg, 0.44 mmol, 5 equiv), and HATU (43.6 mg, 0.11 mmol, 1.3 equiv) in DMF (1 mL) was stirred for 1 h at room temperature. The resulting solution purified by Prep-HPLC (Column: XBridge Shield RP18 OBD Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 30% B to 50% B in 7 min; Wave Length: 254 nm/220 nm nm; RT1 (min): 6.7; Number Of Runs: 2) to afford 2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}amino)-N-methylpyrimidine-5-carboxamide (4.9 mg, 9.48%) as a white solid. LC-MS: (M+H)⁺ found 580.35. ¹H NMR (400 MHz, DMSO-d₆) δ 8.72 (d, J=29.1 Hz, 2H), 8.51 (t, J=6.0 Hz, 1H), 8.33 (m, 1H), 8.01 (d, J=6.9 Hz, 1H), 7.74 (s, 1H), 6.84 (t, J=7.2 Hz, 1H), 6.23 (dd, J=26.3, 7.9 Hz, 2H), 4.98-4.74 (m, 3H), 3.62 (d, J=28.8 Hz, 1H), 3.04 (t, J=11.1 Hz, 1H), 2.83 (d, J=8.3 Hz, 1H), 2.74 (d, J=4.5 Hz, 3H), 2.39-2.26 (m, 1H), 2.19 (s, 3H), 2.10-2.01 (m, 2H), 1.68 (d, J=9.5 Hz, 1H).

### Example 2—Preparation of 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}pyrazole-4-carboxamide

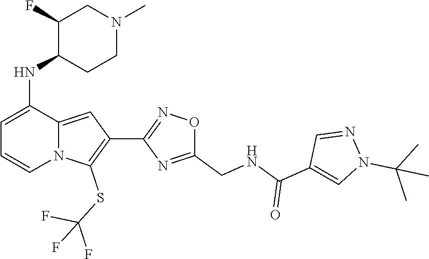 [SMILES: CN1CC[C@@H](Nc2cccn3c(SC(F)(F)F)c(-c4noc(CNC(=O)c5cnn(C(C)(C)C)c5)n4)cc23)[C@@H](F)C1]

Step 1: To a stirred solution of 1-tert-butylpyrazole-4-carboxylic acid (29 mg, 0.17 mmol, 1.5 equiv), DIEA (0.20 mL, 1.12 mmol, 10 equiv), and 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv) in DMF (1 mL) was added HATU (65 mg, 0.17 mmol, 1.5 equiv) at 0 °C. The resulting solution was stirred for 1 h at room temperature, then diluted with EtOAc (20 mL) and washed with brine (2*30 mL). The organic layer was dried over anhydrous Na₂SO₄. After filtration, the filtrate was concentrated under reduced pressure. The residue was purified by silica gel column chromatography (CH₂Cl₂/MeOH=10:1) and Prep-HPLC (Column: XBridge Prep OBD C18 Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 36% B to 61% B in 8 min; Wave Length: 220 nm nm; RT1 (min): 7.62) to afford 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}pyrazole-4-carboxamide (25.8 mg, 38.45%) as a light yellow solid. LC-MS: (M+H)~found 595.20. ¹H NMR (400 MHz, DMSO-d₆) δ 8.98 (t, J=5.7 Hz, 1H), 8.34 (s, 1H), 8.02 (d, J=6.8 Hz, 1H), 7.93 (s, 1H), 7.76 (s, 1H), 6.85 (t, J=7.2 Hz, 1H), 6.28 (d, J=8.1 Hz, 1H), 6.20 (d, J=7.7 Hz, 1H), 4.96-4.75 (m, 3H), 3.62 (d, J=28.4 Hz, 1H), 3.04 (t, J=11.3 Hz, 1H), 2.83 (d, J=8.1 Hz, 1H), 2.19 (s, 4H), 2.07 (t, J=10.9 Hz, 2H), 1.68 (d, J=10.3 Hz, 1H), 1.54 (s, 9H).

### Example 7—Preparation of 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}pyrrole-3-carboxamide

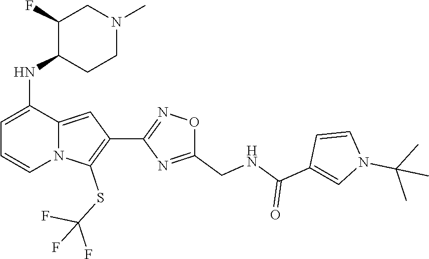 [SMILES: CN1CC[C@@H](Nc2cccn3c(SC(F)(F)F)c(-c4noc(CNC(=O)c5ccn(C(C)(C)C)c5)n4)cc23)[C@@H](F)C1]

Step 1: To a stirred solution of 1-tert-butylpyrrole-3-carboxylic acid (29 mg, 0.17 mmol, 1.5 equiv), 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine (50 mg, 0.11 mmol, 1 equiv) and DIEA (0.20 mL, 1.12 mmol, 10 equiv) in DMF (1 mL) was added HATU (64.2 mg, 0.17 mmol, 1.5 equiv) at 0 °C. The resulting solution was stirred for 1 h at room temperature. The resulting solution was diluted with EtOAc (20 mL) and washed with brine (2*30 mL). The organic layer was dried over anhydrous Na₂SO₄. After filtration, the filtrate was concentrated under reduced pressure. The residue was purified by silica gel column chromatography (CH₂Cl₂/MeOH=10:1) and Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 42% B to 62% B in 7 min; Wave Length: 254 nm/220 nm nm; RT1 (min): 6.67; Number Of Runs: 2) to afford 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}pyrrole-3-carboxamide (19.7 mg, 29.26%) as a light yellow solid. LC-MS: (M+H)⁺ found 594.25. ¹H NMR (400 MHz, DMSO-d₆) δ 8.66 (t, J=5.7 Hz, 1H), 8.02 (d, J=6.8 Hz, 1H), 7.76 (s, 1H), 7.55 (t, J=2.1 Hz, 1H), 6.99 (t, J=2.7 Hz, 1H), 6.85 (t, J=7.2 Hz, 1H), 6.50 (dd, J=3.0, 1.8 Hz, 1H), 6.24 (dd, J=30.8, 7.9 Hz, 2H), 4.98-4.66 (m, 3H), 3.62 (d, J=28.7 Hz, 1H), 3.12-2.97 (m, 1H), 2.87-2.78 (m, 1H), 2.19 (s, 4H), 2.13-2.01 (m, 2H), 1.73-1.64 (m, 1H), 1.49 (s, 9H).

### Example 27—Preparation of N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1,2-thiazole-5-carboxamide

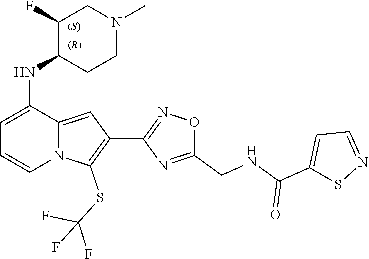

Step 1: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 1,2-thiazole-5-carboxylic acid (29.1 mg, 0.22 mmol, 2 equiv) and DIEA (145.4 mg, 1.12 mmol, 10 equiv) in DMF (1 mL) was added HATU (51.3 mg, 0.13 mmol, 1.2 equiv) at room temperature. The resulting solution was stirred for 1 h at room temperature. The resulting solution was diluted with EtOAc (30 mL) and washed with brine (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, Spm; Mobile Phase A: Water (10 mmol/L NH₄HCO₃)+0.05% NH₃H₂O, Mobile Phase B: ACN; Flow rate: 60 mL/min mL/min; Gradient: 35% B to 60% B in 7 min; Wave Length: 254 nm/220 nm nm; RT1 (min): 7.48) to afford N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1,2-thiazole-5-carboxamide (17.7 mg, 28.26%) as a white solid. LC-MS: (M+H)⁺ found 556.30. ¹H NMR (400 MHz, DMSO-d₆) δ 9.87 (t, J=5.7 Hz, 1H), 8.72 (d, J=1.8 Hz, 1H), 8.07-7.94 (m, 2H), 7.77 (d, J=0.8 Hz, 1H), 6.85 (t, J=7.2 Hz, 1H), 6.24 (dd, J=29.7, 7.9 Hz, 2H), 4.95-4.72 (m, 3H), 3.63 (d, J=29.2 Hz, 1H), 3.04 (t, J=11.2 Hz, 1H), 2.83 (d, J=8.0 Hz, 1H), 2.19 (s, 4H), 2.14-1.94 (m, 2H), 1.68 (d, J=10.5 Hz, 1H).

### Example 30—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-methylthiophene-2-carboxamide

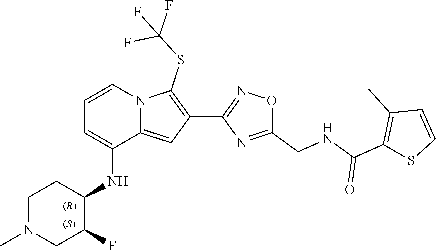

Step 1: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 3-methylthiophene-2-carboxylic acid (32.0 mg, 0.22 mmol, 2 equiv) and DIEA (145.4 mg, 1.12 mmol, 10 equiv) in DMF (1 mL) was added HATU (51.3 mg, 0.13 mmol, 1.2 equiv) at room temperature. The resulting solution was stirred for 1 h at room temperature. The resulting solution was diluted with EtOAc (30 mL) and washed with brine (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃)+0.05% NH₃—H₂O, Mobile Phase B: ACN; Flow rate: 60 mL/min mL/min; Gradient: 41% B to 66% B in 7 min; Wave Length: 254 nm/220 nm nm; RT1 (min): 7.5) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-methylthiophene-2-carboxamide (17.5 mg, 27.30%) as a white solid. LC-MS: (M+H)y found 569.30. ¹H NMR (400 MHz, DMSO-d₆) δ 8.80 (t, J=5.6 Hz, 1H), 8.02 (d, J=6.8 Hz, 1H), 7.77 (d, J=0.8 Hz, 1H), 7.66 (d, J=5.0 Hz, 1H), 7.01 (d, J=5.0 Hz, 1H), 6.85 (t, J=7.2 Hz, 1H), 6.27 (d, J=8.1 Hz, 1H), 6.21 (d, J=7.6 Hz, 1H), 4.92-4.78 (m, 3H), 3.63 (d, J=28.0 Hz, 1H), 3.13-2.98 (m, 1H), 2.83 (d, J=7.6 Hz, 1H), 2.46 (s, 3H), 2.34-2.19 (m, 4H), 2.07 (t, J=10.7 Hz, 2H), 1.69 (d, J=9.2 Hz, 1H).

### Example 13—Preparation of 2-tert-butyl-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1,3-oxazole-5-carboxamide

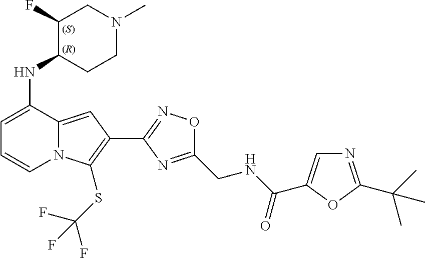

Step 1: A mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 2-tert-butyl-1,3-oxazole-5-carboxylic acid (22.8 mg, 0.13 mmol, 1.2 equiv), HATU (64.1 mg, 0.17 mmol, 1.5 equiv) and DIEA (145.4 mg, 1.12 mmol, 10 equiv) in DMF (1.5 mL) was stirred for 1 h at room temperature. The resulting mixture was diluted with EtOAc (50 mL) and washed with 3*50 mL of brine. The organic layer was dried over anhydrous Na₂SO₄. After filtration, the filtrate was concentrated under reduced pressure. The residue was purified by Prep-TLC (CH₂Cl₂/MeOH 20:1) and Prep-HPLC (Column: XBridge Prep OBD C18 Column, 30*150 mm, 5 μm; Mobile Phase A: Water(10 mmol/L NH₄HCO₃+0.05% NH₃·H₂O), Mobile Phase B: ACN; Flow rate: 60 mL/min mL/min; Gradient: 40% B to 65% B in 8 min; Wave Length: 220 nm nm; RT1 (min): 7.28) to afford 2-tert-butyl-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1,3-oxazole-5-carboxamide (11 mg, 16.14%) as a off-white solid. LC-MS: (M+H)⁺ found 596.15. ¹H NMR (400 MHz, DMSO-d₆) δ 9.41 (t, J=5.7 Hz, 1H), 8.02 (d, J=6.8 Hz, 1H), 7.83-7.68 (m, 2H), 6.85 (t, J=7.2 Hz, 1H), 6.27 (d, J=8.1 Hz, 1H), 6.20 (d, J=7.7 Hz, 1H), 4.97-4.76 (m, 3H), 3.62 (d, J=28.8 Hz, 1H), 3.04 (t, J=11.2 Hz, 1H), 2.83 (d, J=8.1 Hz, 1H), 2.19 (s, 4H), 2.06 (t, J=11.1 Hz, 2H), 1.68 (d, J=9.9 Hz, 1H), 1.37 (s, 9H).

### Example 14—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-methyl-4-oxo-3H-thieno[2,3-d]pyrimidine-6-carboxamide

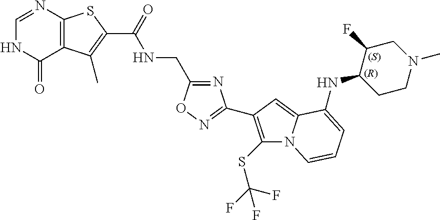

Step 1: To a stirred solution of 5-methyl-4-oxo-3H-thieno[2,3-d]pyrimidine-6-carboxylic acid (47.3 mg, 0.23 mmol, 1 equiv), HATU (102.6 mg, 0.27 mmol, 1.2 equiv), DIEA (116.3 mg, 0.90 mmol, 4 equiv) in DMF (2 mL) was added 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.23 mmol, 1 equiv) at room temperature. The resulting solution was stirred for 1 h at room temperature. The resulting mixture was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃)+0.05% NH₃—H₂O, Mobile Phase B: ACN; Flow rate: 60 mL/min mL/min; Gradient: 28% B to 53% B in 7 min; Wave Length: 254 nm/220 nm nm; RT1 (min): 7.82) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-methyl-4-oxo-3H-thieno[2,3-d]pyrimidine-6-carboxamide (13.6 mg, 9.36%) as a yellow solid. LC-MS: (M+H)⁺ found 637.10. ¹H NMR (400 MHz, DMSO-d₆) δ 12.56 (s, 1H), 9.06 (t, J=5.6 Hz, 1H), 8.18 (s, 1H), 8.03 (d, J=6.9 Hz, 1H), 7.77 (d, J=0.8 Hz, 1H), 6.85 (t, J=7.2 Hz, 1H), 6.23 (dd, J=17.0, 7.9 Hz, 2H), 4.98-4.74 (m, 3H), 3.63 (d, J=27.8 Hz, 1H), 3.04 (t, J=11.2 Hz, 1H), 2.78 (s, 4H), 2.19 (s, 4H), 2.07 (t, J=11.2 Hz, 2H), 1.69 (d, J=9.8 Hz, 1H).

### Example 16—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-oxo-1,3-dihydroindole-6-carboxamide

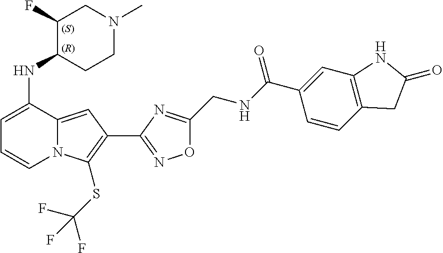

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (80 mg, 0.18 mmol, 1 equiv), 2-oxo-1,3-dihydroindole-6-carboxylic acid (31.9 mg, 0.18 mmol, 1 equiv), DIEA (93.1 mg, 0.72 mmol, 4 equiv), and HATU (88.9 mg, 0.23 mmol, 1.3 equiv) in DMF (4 mL) was stirred for 1 h at room temperature. The resulting solution was diluted with EtOAc (50 mL) and washed with briner (2*50 mL). The organic layer was dried over anhydrous Na₂SO₄. After filtration, the filtrate was concentrated under reduced pressure. The residue was purified by Prep-TLC (CH₂Cl₂/MeOH=8:1) and Prep-HPLC (Column: XBridge Shield RP18 OBD Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 30% B to 55% B in 7 min; Wave Length: 254 nm/220 nm nm; RT1 (min): 6.47; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-oxo-1,3-dihydroindole-6-carboxamide (11.7 mg, 10.47%) as a light yellow solid. LC-MS: (M+H)⁺ found 604.10. ¹H NMR (400 MHz, DMSO-d₆) δ 10.61 (s, 1H), 9.38 (t, J=5.6 Hz, 1H), 8.02 (d, J=6.8 Hz, 1H), 7.76 (d, J=0.9 Hz, 1H), 7.53 (dd, J=7.8, 1.6 Hz, 1H), 7.40-7.30 (m, 2H), 6.85 (t, J=7.2 Hz, 1H), 6.26 (d, J=8.0 Hz, 1H), 6.20 (d, J=7.6 Hz, 1H), 4.95-4.76 (m, 3H), 3.56 (s, 3H), 3.11-2.98 (m, 1H), 2.82 (d, J=8.2 Hz, 1H), 2.41-1.96 (m, 6H). 1.68 (d, J=10.1 Hz, 1H).

### Example 17—Preparation of N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-2-oxo-1,3-dihydroindole-7-carboxamide

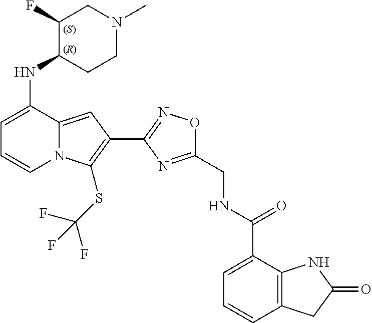

Step 1: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (120 mg, 0.27 mmol, 1 equiv), DIEA (0.24 mL, 1.35 mmol, 5 equiv), and 2-oxo-1,3-dihydroindole-7-carboxylic acid (52.6 mg, 0.30 mmol, 1.1 equiv) in DMF (2 mL) was added HATU (154.0 mg, 0.41 mmol, 1.5 equiv) at 0 °C. The resulting solution was stirred for 1 h at room temperature. The resulting solution was purified by Prep-HPLC (Column: XBridge Prep OBD C18 Column, 30*150 mm, 5 μm; Mobile Phase A: Water (10 mmol/L NH₄HCO₃)+0.05% NH₃—H₂O, Mobile Phase B: ACN; Flow rate: 60 mL/min mL/min; Gradient: 34% B to 59% B in 7 min; Wave Length: 254 nm/220 nm nm; RT1 (min): 7.57) to afford N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-2-oxo-1,3-dihydroindole-7-carboxamide (23.7 mg, 14.24%) as a white solid. LC-MS: (M+H)⁺ found 604.15. ¹H NMR (400 MHz, DMSO-d₆) δ 9.90 (s, 1H), 9.48 (t, J=5.6 Hz, 1H), 8.02 (d, J=6.8 Hz, 1H), 7.75 (dd, J=7.3, 1.0 Hz, 2H), 7.44-7.38 (m, 1H), 7.08 (dd, J=8.1, 7.3 Hz, 1H), 6.85 (t, J=7.2 Hz, 1H), 6.22 (dd, J=15.3, 7.8 Hz, 2H), 4.95-4.77 (m, 3H), 3.55 (s, 3H), 3.04 (t, J=11.2 Hz, 1H), 2.82 (d, J=8.5 Hz, 1H), 2.33-1.95 (m, 6H), 1.68 (d, J=10.5 Hz, 1H).

### Example 18—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-oxo-1,3-dihydroindole-5-carboxamide

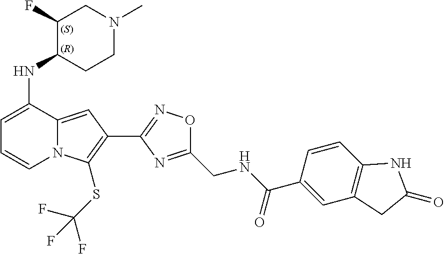

Step 1: To a stirred solution of 2-oxo-1,3-dihydroindole-5-carboxylic acid (35.9 mg, 0.20 mmol, 1 equiv), DIEA (130.9 mg, 1.01 mmol, 5 equiv) and 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (90 mg, 0.20 mmol, 1 equiv) in DMF (1.5 mL) was added PyBOP (115.9 mg, 0.22 mmol, 1.1 equiv) at 0 °C. The mixture was stirred at 25 °C for 30 min. The resulting solution was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 30% B to 53% B in 7 min; Wave Length: 254 nm/220 nm nm; RT1 (min): 6.77; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-oxo-1,3-dihydroindole-5-carboxamide (20.4 mg, 16.36%) as an off-white solid. LC-MS: (M+H)⁺ found 604.35. ¹H NMR (400 MHz, DMSO-d₆) 10.67 (s, 1H), 9.23 (t, J=5.6 Hz, 1H), 8.02 (d, J=6.8 Hz, 1H), 7.85-7.73 (m, 3H), 6.93-6.81 (m, 2H), 6.23 (dd, J=22.5, 7.8 Hz, 2H), 4.96-4.71 (m, 3H), 3.56 (s, 3H), 3.04 (t, J=11.1 Hz, 1H), 2.83 (d, J=8.7 Hz, 1H), 2.35-2.19 (m, 4H), 2.14-1.99 (m, 2H), 1.68 (d, J=11.0 Hz, 1H).

### Example 19—Preparation of 3-cyano-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1H-indole-5-carboxamide

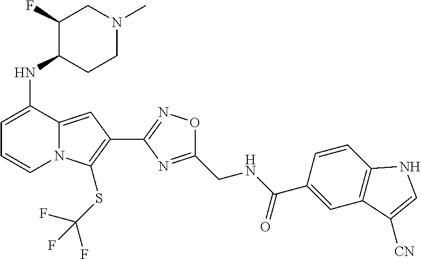 [SMILES: CN1CC[C@@H](Nc2cccn3c(SC(F)(F)F)c(-c4noc(CNC(=O)c5ccc6[nH]cc(C#N)c6c5)n4)cc23)[C@@H](F)C1]

Step 1: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.23 mmol, 1 equiv), DIEA (290.8 mg, 2.25 mmol, 10 equiv) and 3-cyano-1H-indole-5-carboxylic acid (20.9 mg, 0.11 mmol, 0.5 equiv) in DMF (1 mL) was added PyBOP (175.6 mg, 0.34 mmol, 1.5 equiv) at 0 °C. The resulting solution was stirred for 1 h at room temperature. The resulting solution was purified by Prep-HPLC (Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 35% B to 60% B in 8 min; Wave Length: 220 nm nm; RT1 (min): 7.78) to afford 3-cyano-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1H-indole-5-carboxamide (27.2 mg, 19.73%) as an off-white solid. LC-MS: (M+H)⁺ found 613.40. ¹H NMR (400 MHz, DMSO-d₆) δ 12.47 (s, 1H), 9.51 (t, J=5.6 Hz, 1H), 8.41-8.33 (m, 2H), 8.02 (d, J=6.8 Hz, 1H), 7.88 (dd, J=8.7, 1.7 Hz, 1H), 7.77 (d, J=0.9 Hz, 1H), 7.65 (d, J=8.6 Hz, 1H), 6.85 (t, J=7.2 Hz, 1H), 6.23 (dd, J=23.3, 7.9 Hz, 2H), 4.94-4.77 (m, 3H), 3.61 (d, J=28.5 Hz, 1H), 3.03 (t, J=11.3 Hz, 1H), 2.82 (d, J=8.4 Hz, 1H), 2.18 (s, 4H), 2.05 (t, J=11.3 Hz, 2H), 1.66 (s, 1H).

### Example 20—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(morpholin-4-yl)-1,3-thiazole-4-carboxamide

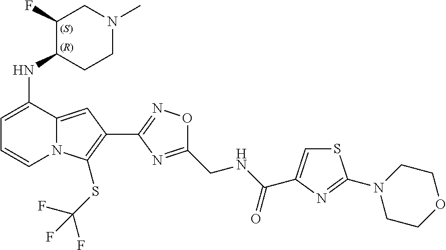

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.23 mmol, 1 equiv), 2-(morpholin-4-yl)-1,3-thiazole-4-carboxylic acid (33.7 mg, 0.16 mmol, 0.7 equiv), HATU (85.5 mg, 0.23 mmol, 1 equiv) and DIEA (145.4 mg, 1.13 mmol, 5 equiv) in DMF (2 mL) was stirred for 1 h at room temperature. The resulting solution was diluted with EtOAc (20 mL) and washed with brine (3*20 mL). The organic layer was dried over anhydrous Na₂SO₄. After filtration, the filtrate was concentrated under reduced pressure. The residue was purified by Prep-TLC (CH₂Cl₂/MeOH 10:1) and Prep-HPLC (Column: XBridge Prep OBD C18 Column, 19*250 mm, 5 μm; Mobile Phase A: Water (10 mmol/L NH₄HCO₃+0.05% NH₃·H₂O), Mobile Phase B: MEOH; Flow rate: 25 mL/min mL/min; Gradient: 57% B to 82% B in 10 min; Wave Length: 254 nm/220 nm nm; RT1 (min): 9.77; Number Of Runs: 4) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(morpholin-4-yl)-1,3-thiazole-4-carboxamide (18.8 mg, 12.72%) as a off-white solid. LC-MS: (M+H)⁺ found 641.15. ¹H NMR (400 MHz, DMSO-d₆) δ 8.94 (t, J=6.0 Hz, 1H), 8.02 (d, J=6.9 Hz, 1H), 7.76 (d, J=0.9 Hz, 1H), 7.55 (s, 1H), 6.85 (t, J=7.2 Hz, 1H), 6.23 (dd, J=28.4, 7.9 Hz, 2H), 4.96-4.74 (m, 3H), 3.74 (t, J=4.9 Hz, 4H), 3.62 (d, J=29.4 Hz, 1H), 3.48 (t, J=4.9 Hz, 4H), 3.04 (t, J=11.1 Hz, 1H), 2.83 (d, J=8.3 Hz, 1H), 2.19 (s, 4H), 2.07 (t, J=11.2 Hz, 2H), 1.68 (d, J=10.9 Hz, 1H).

### Example 32—Preparation of 3-fluoro-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}thiophene-2-carboxamide

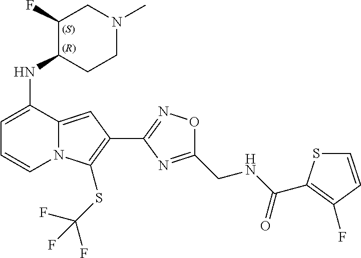

Step 1: A mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1 equiv), 3-fluorothiophene-2-carboxylic acid (23.7 mg, 0.16 mmol, 1.2 equiv), DIEA (87.2 mg, 0.67 mmol, 5 equiv) and HATU (77 mg, 020 mmol, 1.5 equiv) in DMF (1.5 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 20:1) and Prep-HPLC (Column: XBridge Shield RP18 OBD Column, 30*150 mm, 5 μm; Mobile Phase A: Water(10 mmol/L NH₄HCO₃+0.05% NH₃H₂O), Mobile Phase B: ACN; Flow rate: 60 mL/min mL/min; Gradient: 40% B to 65% B in 7 min; Wave Length: 254 nm/220 nm nm; RT1 (min): 6.4; Number Of Runs: 2) to afford 3-fluoro-N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}thiophene-2-carboxamide (18.4 mg, 23.52%) as a off-white solid. LC-MS: (M+H)⁺ found 573.10. ¹H NMR (400 MHz, DMSO-d₆) δ 8.75 (s, 1H), 8.02 (d, J=6.9 Hz, 1H), 7.87 (dd, J=5.5, 4.0 Hz, 1H), 7.77 (d, J=0.9 Hz, 1H), 7.16 (d, J=5.5 Hz, 1H), 6.85 (t, J=7.2 Hz, 1H), 6.28 (d, J=8.1 Hz, 1H), 6.20 (d, J=7.7 Hz, 1H), 4.95-4.74 (m, 3H), 3.62 (d, J=29.1 Hz, 1H), 3.04 (t, J=10.7 Hz, 1H), 2.82 (s, 1H), 2.19 (s, 4H), 2.13-2.00 (m, 2H), 1.68 (d, J=10.0 Hz, 1H).

### Example 43—Preparation of 2-(4-cyanophenoxy)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}acetamide

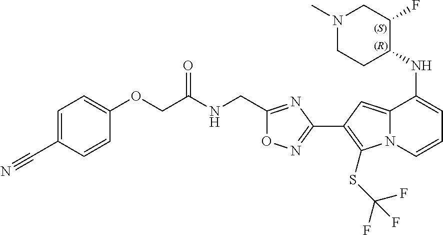

Step 1: A mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1 equiv), P-cyanophenoxyacetic acid (28.7 mg, 0.16 mmol, 1.2 equiv), HATU (77.0 mg, 0.20 mmol, 1.5 equiv) and DIEA (87.2 mg, 0.67 mmol, 5 equiv) in DMF (1.5 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 20:1) and Prep-HPLC (Column: XBridge Prep OBD C18 Column, 30*150 mm, 5 μm; Mobile Phase A: Water(10 mmol/L NH₄HCO₃)+0.05%₀NH₃—H₂O, Mobile Phase B: ACN; Flow rate: 60 mL/min mL/min; Gradient: 37% B to 62% B in 7 min; Wave Length: 254 nm/220 nm nm; RT1 (min): 7.85) to afford 2-(4-cyanophenoxy)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}acetamide (17 mg, 20.59%) as a yellow solid. LC-MS: (M+H)⁺ found 604.10. ¹H NMR (400 MHz, DMSO-d₆) δ 9.10 (t, J=5.8 Hz, 1H), 8.02 (d, J=6.8 Hz, 1H), 7.88-7.71 (m, 3H), 7.25-7.09 (m, 2H), 6.86 (t, J=7.2 Hz, 1H), 6.26 (d, J=8.1 Hz, 1H), 6.21 (d, J=7.7 Hz, 1H), 4.87 (d, J=49.5 Hz, 1H), 4.80-4.71 (m, 4H), 3.63 (d, J=28.4 Hz, 1H), 3.05 (t, J=11.4 Hz, 1H), 2.82 (s, 1H), 2.20 (s, 4H), 2.07 (t, J=10.2 Hz, 2H), 1.70 (d, J=9.5 Hz, 1H).

### Example 23—Preparation of N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1-methyl-3-oxo-2,4-dihydroquinoxaline-6-carboxamide

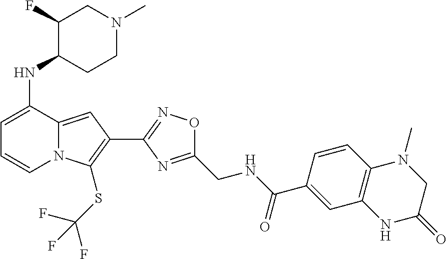 [SMILES: CN1CC[C@@H](Nc2cccn3c(SC(F)(F)F)c(-c4noc(CNC(=O)c5ccc6c(c5)NC(=O)CN6C)n4)cc23)[C@@H](F)C1]

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.225 mmol, 1 equiv) in DMF (1 mL) was treated with DIEA (203.6 mg, 1.58 mmol, 7 equiv) and PyBOP (175.6 mg, 0.34 mmol, 1.5 equiv) for 2 min at 0 °C followed by the addition of 1-methyl-3-oxo-2,4-dihydroquinoxaline-6-carboxylic acid (46.4 mg, 0.23 mmol, 1 equiv) at room temperature. The resulting solution was stirred for 1 h at room temperature. The resulting solution was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water(10 mmol/L NH₄HCO₃+0.05% NH₃·H₂O), Mobile Phase B: ACN; Flow rate: 60 mL/min mL/min; Gradient: 40% B to 60% B in 8 min; Wave Length: 220 nm nm; RT1 (min): 7.70) to afford N-{[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}-1-methyl-3-oxo-2,4-dihydroquinoxaline-6-carboxamide (29.8 mg, 20.94%) as an off-white solid. LC-MS: (M+H)⁺ found 633.15. ¹H NMR (400 MHz, DMSO-d₆) δ 10.60 (s, 1H), 9.10 (t, J=5.6 Hz, 1H), 8.02 (d, J=6.9 Hz, 1H), 7.76 (d, J=0.9 Hz, 1H), 7.54 (dd, J=8.5, 2.1 Hz, 1H), 7.35 (d, J=2.0 Hz, 1H), 6.85 (t, J=7.2 Hz, 1H), 6.75 (d, J=8.5 Hz, 1H), 6.26 (d, J=8.1 Hz, 1H), 6.20 (d, J=7.6 Hz, 1H), 4.78 (d, J=5.6 Hz, 3H), 3.79 (s, 2H), 3.62 (d, J=30.2 Hz, 1H), 3.04 (t, J=10.3 Hz, 1H), 2.86 (s, 4H), 2.19 (s, 4H), 2.14-2.01 (m, 2H), 1.68 (d, J=11.0 Hz, 1H).

### Example 31—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-(1-methylcyclopropyl)prop-2-ynamide

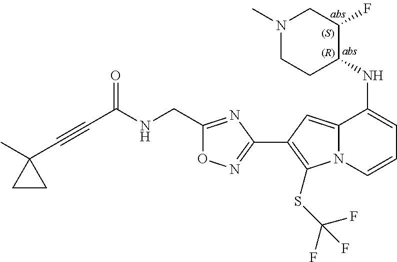

Step 1: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (90 mg, 0.20 mmol, 1 equiv), 3-(1-methylcyclopropyl) prop-2-ynoic acid (25.1 mg, 0.20 mmol, 1 equiv) and DIEA (0.18 mL, 1.01 mmol, 5 equiv) in DMF (1 mL) was added PyBOP (137.0 mg, 0.26 mmol, 1.3 equiv) at 0 °C. The resulting solution was stirred for 1 h at room temperature. The resulting mixture was diluted with water (30 mL) and extracted with DCM (3*30 mL). The combined organic layers were dried over anhydrous Na₂SO₄. After filtration, the filtrate was concentrated under reduced pressure. The residue was purified by Prep-TLC (CH₂Cl₂/MeOH=10:1) and Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 44% B to 62% B in 7 min; Wave Length: 254 nm/220 nm nm; RT1 (min): 6.42; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-(1-methylcyclopropyl)prop-2-ynamide (29 mg, 25.80%) as a light yellow solid. LCMS: (M+H)~found 551.40. ¹H NMR (400 MHz, DMSO-d₆) δ 9.32 (t, J=5.9 Hz, 1H), 8.02 (d, J=6.9 Hz, 1H), 7.77 (s, 1H), 6.86 (t, J=7.2 Hz, 1H), 6.25 (dd, J=34.0, 7.9 Hz, 2H), 4.87 (d, J=48.8 Hz, 1H), 4.65 (d, J=5.8 Hz, 2H), 3.63 (d, J=29.3 Hz, 1H), 3.10-2.99 (m, 1H), 2.83 (s, 1H), 2.30-2.20 (m, 4H), 2.14-2.03 (m, 2H), 1.70 (d, J=9.0 Hz, 1H), 1.28 (s, 3H), 0.98 (m, 2H), 0.80 (m, 2H).

### Example 33—Preparation of 3-amino-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide

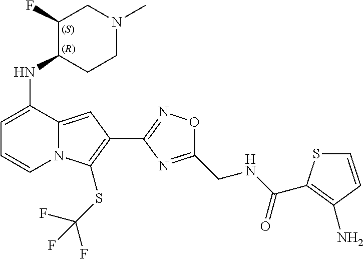

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (40 mg, 0.09 mmol, 1 equiv), 3-aminothiophene-2-carboxylic acid (12.9 mg, 0.09 mmol, 1 equiv), HATU (34.2 mg, 0.09 mmol, 1 equiv) and DIEA (34.9 mg, 0.27 mmol, 3 equiv) in DMF (1 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/7M NH₃ in MeOH 20:1) and Prep-HPLC (Column: XBridge Prep OBD C18 Column, 19*250 mm, 5 μm; Mobile Phase A: Water (10 mmol/L NH₄HCO₃+0.05% NH₃H₂O), Mobile Phase B: MEOH; Flow rate: 25 mL/min mL/min; Gradient: 53% B to 77% B in 10 min; Wave Length: 254 nm/220 nm nm; RT1 (min): 9.78; Number Of Runs: 5) to afford 3-amino-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide (3.1 mg, 6.05%) as a white solid. LC-MS: (M+H)⁺ found 570.10. ¹H NMR (400 MHz, DMSO-d₆) δ 8.34 (t, J=5.5 Hz, 1H), 8.02 (d, J=6.9 Hz, 1H), 7.76 (s, 1H), 7.46 (d, J=5.3 Hz, 1H), 6.85 (t, J=7.2 Hz, 1H), 6.61 (d, J=5.3 Hz, 1H), 6.51 (s, 2H), 6.28 (d, J=8.2 Hz, 1H), 6.20 (d, J=7.6 Hz, 1H), 4.86 (d, J=49.2 Hz, 1H), 4.69 (d, J=5.5 Hz, 2H), 3.62 (d, J=28.5 Hz, 1H), 3.04 (t, J=11.1 Hz, 1H), 2.82 (s, 1H), 2.19 (s, 4H), 2.07 (t, J=9.8 Hz, 2H), 1.69 (s, 1H).

### Example 34—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-6-methoxyquinoline-2-carboxamide

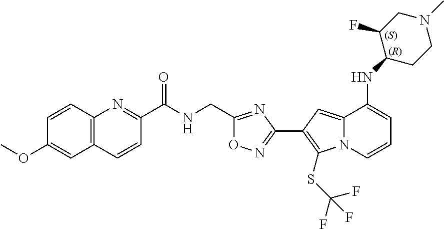

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 6-methoxyquinoline-2-carboxylic acid (22.9 mg, 0.11 mmol, 1 equiv), PyBOP (70.3 mg, 0.13 mmol, 1.2 equiv) and DIEA (72.7 mg, 0.56 mmol, 5.00 equiv) in DMF (1 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 15:1) and Prep-HPLC (Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 45% B to 70% B in 8 min, Wave Length: 220 nm nm; RT1 (min): 8.17) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-6-methoxyquinoline-2-carboxamide (10.9 mg, 15.39%) as a white solid. LC-MS: (M+H)⁺ found 630.20. ¹H NMR (400 MHz, DMSO-d₆) δ 9.69 (t, J=6.1 Hz, 1H), 8.47 (d, J=8.5 Hz, 1H), 8.14 (d, J=8.5 Hz, 1H), 8.06 (d, J=9.1 Hz, 1H), 8.01 (d, J=6.8 Hz, 1H), 7.75 (s, 1H), 7.55-7.51 (m, 2H), 6.84 (t, J=7.2 Hz, 1H), 6.21 (m 2H), 5.10-4.58 (m, 3H), 3.95 (s, 3H), 3.61 (d, J=29.3 Hz, 1H), 3.03 (t, J=11.2 Hz, 1H), 2.81 (d, J=9.7 Hz, 1H), 2.18 (s, 4H), 2.11-1.90 (m, 2H), 1.66 (d, J=11.8 Hz, 1H).

### Example 37—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methoxythiophene-2-carboxamide

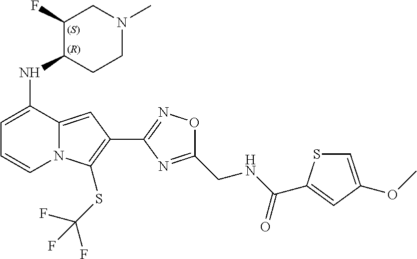

Step 1: To a stirred solution of 4-methoxythiophene-2-carboxylic acid (32.0 mg, 0.20 mmol, 1 equiv) in DMF (1.5 mL) was added PyBOP (158.1 mg, 0.30 mmol, 1.5 equiv), DIEA (130.9 mg, 1.01 mmol, 5 equiv) and 2-[5-(aminomethyl)-1,2,4-oxadiazol-3-yl]-N-[(3S,4R)-3-fluoro-1-methylpiperidin-4-yl]-3-[(trifluoro-methyl) sulfanyl]indolizin-8-amine (90 mg, 0.20 mmol, 1 equiv) at 0 °C. The reaction solution was stirred at r.t. for 1 h. The resulting mixture was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 42% B to 62% B in 7 min; Wave Length: 254 nm/220 nm nm; RT1 (min): 6.57; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methoxythiophene-2-carboxamide (43.3 mg, 36.54%) as an off-white solid. LC-MS: (M+H)⁺ found 585.30. ¹H NMR (400 MHz, DMSO-d₆) 9.39 (t, J=5.7 Hz, 1H), 8.02 (d, J=6.9 Hz, 1H), 7.76 (d, J=0.9 Hz, 1H), 7.55 (d, J=1.7 Hz, 1H), 6.98-6.81 (m, 2H), 6.23 (dd, J=23.1, 7.9 Hz, 2H), 4.96-4.77 (m, 3H), 3.78 (s, 3H), 3.63 (d, J=28.4 Hz, 1H), 3.04 (t, J=11.1 Hz, 1H), 2.82 (d, J=7.9 Hz, 1H), 2.19 (s, 4H), 2.07 (t, J=10.8 Hz, 2H), 1.68 (d, J=9.7 Hz, 1H).

### Example 38—Preparation of 4-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide

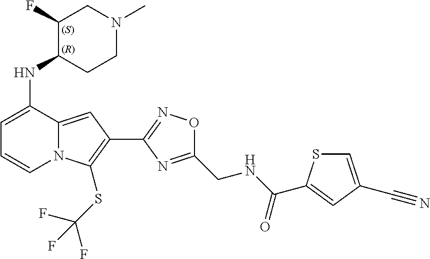

Step 1: To a stirred solution of 4-cyanothiophene-2-carboxylic acid (33 mg, 0.22 mmol, 1.2 equiv), 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine (80 mg, 0.18 mmol, 1 equiv), and DIEA (0.16 mL, 0.90 mmol, 5 equiv) in DMF (1 mL) was added PyBOP (112 mg, 0.22 mmol, 1.2 equiv) at 0 °C. The resulting solution was stirred for 1 h at room temperature, then diluted with EtOAc (20 mL) and washed with brine (3*20 mL). The organic layer was dried over anhydrous Na₂SO₄. After filtration, the filtrate was concentrated under reduced pressure. The residue was purified by silica gel column chromatography (CH₂Cl₂/MeOH=10:1) and Prep-HPLC (Column: XBridge Prep OBD C18 Column, 19*250 mm, 5 m; Mobile Phase A: Water (10 mmol/L NH₄HCO₃)+0.05% NH₃—H₂O, Mobile Phase B: ACN; Flow rate: 60 mL/min mL/min; Gradient: 38% B to 63% B in 7 min; Wave Length: 254 nm/220 nm nm; RT1 (min): 7.7) to afford 4-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide (36.2 mg, 34.70%) as a light yellow solid. LC-MS: (M+H)⁺ found 580.00. ¹H NMR (400 MHz, DMSO-d₆) δ 9.70 (t, J=5.7 Hz, 1H), 8.80 (d, J=1.3 Hz, 1H), 8.18 (d, J=1.3 Hz, 1H), 8.02 (d, J=6.9 Hz, 1H), 7.77 (d, J=0.8 Hz, 1H), 6.85 (t, J=7.2 Hz, 1H), 6.27 (d, J=8.1 Hz, 1H), 6.20 (d, J=7.7 Hz, 1H), 4.97-4.78 (m, 3H), 3.63 (d, J=28.9 Hz, 1H), 3.04 (t, J=11.0 Hz, 1H), 2.92-2.78 (m, 1H), 2.19 (s, 4H), 2.12-1.97 (m, 2H), 1.68 (d, J=9.5 Hz, 1H).

### Example 39—Preparation of 5-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide

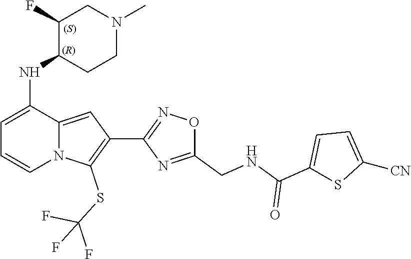

Step 1: To a stirred solution of 5-cyanothiophene-2-carboxylic acid (31.0 mg, 0.20 mmol, 1 equiv) in DMF (1.5 mL) was added PyBOP (126.5 mg, 0.24 mmol, 1.2 equiv), DIEA (130.9 mg, 1.01 mmol, 5 equiv) and 2-[5-(aminomethyl)-1,2,4-oxadiazol-3-yl]-N-[(3S,4R)-3-fluoro-1-methylpiperidin-4-yl]-3-[(trifluoro-methyl) sulfanyl]indolizin-8-amine (90 mg, 0.20 mmol, 1 equiv) at 0 °C. The reaction solution was stirred at r.t. for 1 h. The resulting solution was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 42% B to 62% B in 7 min; Wave Length: 254 nm/220 nm nm; RT1 (min): 6.73; Number Of Runs: 2) to afford 5-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide (37.5 mg, 31.63%) as a yellow solid. LC-MS: (M+H)⁺ found 580.30. ¹H NMR (400 MHz, DMSO-d₆) 9.85 (t, J=5.6 Hz, 1H), 8.07-7.99 (m, 2H), 7.95 (d, J=4.1 Hz, 1H), 7.76 (d, J=0.8 Hz, 1H), 6.85 (t, J=7.2 Hz, 1H), 6.23 (dd, J=23.5, 7.9 Hz, 2H), 4.94-4.73 (m, 3H), 3.62 (d, J=28.1 Hz, 1H), 3.04 (t, J=11.0 Hz, 1H), 2.83 (d, J=8.4 Hz, 1H), 2.19 (s, 4H), 2.07 (t, J=11.6 Hz, 2H), 1.68 (d, J=10.3 Hz, 1H).

### Example 41—Preparation of 3-(difluoromethoxy)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide

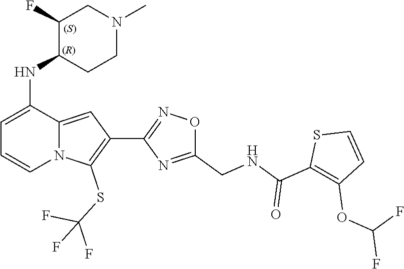

Step 1: To a stirred solution of 3-(difluoromethoxy)thiophene-2-carboxylic acid (48.1 mg, 0.25 mmol, 1.1 equiv), PyBOP (140.5 mg, 0.27 mmol, 1.2 equiv) and DIEA (0.20 mL, 1.13 mmol, 5 equiv) in DMF (1 mL) was added 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine (100 mg, 0.23 mmol, 1 equiv) at 0 °C. The resulting solution was stirred for 1 h at room temperature. The reaction was quenched by the addition of water (30 mL) and extracted with EtOAc (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃+0.05% NH₃—H₂O), Mobile Phase B: ACN; Flow rate: 60 mL/min mL/min; Gradient: 42% B to 67% B in 8 min; Wave Length: 220 nm nm; RT1 (min): 7.62) to afford 3-(difluoromethoxy)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}thiophene-2-carboxamide (32.4 mg, 23.18%) as a white solid. LC-MS: (M+H)y found 621.00. ¹H NMR (400 MHz, DMSO-d₆) δ 8.52 (t, J=5.8 Hz, 1H), 8.02 (d, J=6.9 Hz, 1H), 7.90 (d, J=5.5 Hz, 1H), 7.76 (d, J=0.9 Hz, 1H), 7.54-7.16 (m, 2H), 6.85 (t, J=7.3 Hz, 1H), 6.27 (d, J=8.1 Hz, 1H), 6.20 (d, J=7.6 Hz, 1H), 4.94-4.74 (m, 3H), 3.63 (d, J=29.1 Hz, 1H), 3.04 (t, J=11.2 Hz, 1H), 2.83 (d, J=7.5 Hz, 1H), 2.19 (s, 4H), 2.15-1.99 (m, 2H), 1.68 (d, J=10.4 Hz, 1H).

### Example 44—Preparation of tert-butyl N-{1-[2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}carbamoyl)eth-1-yn-1-yl]cyclopropyl}carbamate

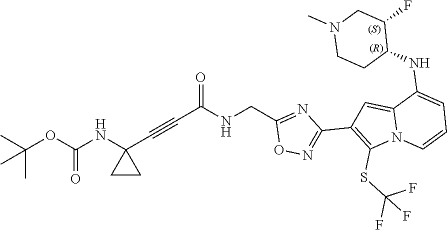

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (80 mg, 0.18 mmol, 1 equiv), 3-{1-[(tert-butoxycarbonyl) amino]cyclopropyl}prop-2-ynoic acid (40.5 mg, 0.18 mmol, 1 equiv), PyBOP (121.8 mg, 0.23 mmol, 1.3 equiv) and DIEA (116.3 mg, 0.90 mmol, 5 equiv) in DMF (1 mL) was stirred for 1 h at r.t. The resulting mixture was diluted with water (30 mL) and extracted with EtOAc (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃+0.05% NH₃—H₂O), Mobile Phase B: ACN; Flow rate: 60 mL/min mL/min; Gradient: 41% B to 66% B in 8 min; Wave Length: 220 nm nm; RT1 (min): 7.68) to afford tert-butyl N-{1-[2-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}carbamoyl)eth-1-yn-1-yl]cyclopropyl}carbamate (14.1 mg, 11.77%) as a white solid. LC-MS: (M+H)⁺ found 652.30. ¹H NMR (400 MHz, DMSO-d₆) δ 9.35 (s, 1H), 8.02 (d, J=6.8 Hz, 1H), 7.76 (d, J=18.5 Hz, 2H), 6.85 (t, J=7.2 Hz, 1H), 6.31 (d, J=7.9 Hz, 1H), 6.20 (d, J=7.6 Hz, 1H), 4.97-4.76 (m, 1H), 4.66 (d, J=5.8 Hz, 2H), 3.63 (d, J=28.8 Hz, 1H), 3.05 (t, J=10.9 Hz, 1H), 2.84 (d, J=7.0 Hz, 1H), 2.20 (s, 4H), 2.08 (t, J=9.4 Hz, 2H), 1.70 (s, 1H), 1.39 (s, 9H), 1.24-1.16 (m, 2H), 1.14-1.06 (m, 2H).

### Example 56—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-(2H-pyrazol-3-yl)thiophene-2-carboxamide

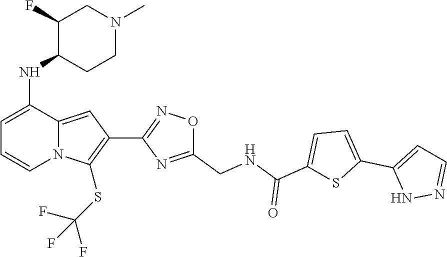 [SMILES: CN1CC[C@@H](Nc2cccn3c(SC(F)(F)F)c(-c4noc(CNC(=O)c5ccc(-c6ccn[nH]6)s5)n4)cc23)[C@@H](F)C1]

Step 1: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.23 mmol, 1 equiv) in DMF (1.2 mL) was treated with DIEA (203.6 mg, 1.58 mmol, 7 equiv) and PyBOP (175.6 mg, 0.34 mmol, 1.5 equiv) for 2 min at 0 °C followed by the addition of 5-(2H-pyrazol-3-yl)thiophene-2-carboxylic acid (30.6 mg, 0.16 mmol, 0.7 equiv). The resulting solution was stirred for 1 h at room temperature. The resulting mixture was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 34% B to 58% B in 7 min; Wave Length: 254 nm/220 nm nm; RT1 (min): 7.77) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-(2H-pyrazol-3-yl)thiophene-2-carboxamide (40.5 mg, 29.00%) as an off-white solid. LC-MS: (M+H)⁺ found 621.10. ¹H NMR (400 MHz, DMSO-d₆) δ 13.03 (s, 1H), 9.40 (t, J=5.7 Hz, 1H), 8.02 (d, J=6.9 Hz, 1H), 7.85-7.75 (m, 3H), 7.45 (d, J=3.9 Hz, 1H), 6.85 (t, J=7.2 Hz, 1H), 6.71 (t, J=2.1 Hz, 1H), 6.24 (dd, J=29.1, 7.9 Hz, 2H), 4.96-4.75 (m, 3H), 3.62 (d, J=28.7 Hz, 1H), 3.04 (t, J=11.0 Hz, 1H), 2.82 (d, J=7.6 Hz, 1H), 2.19 (s, 4H), 2.13-1.98 (m, 2H), 1.68 (d, J=10.7 Hz, 1H).

### Example 59—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}acetamide

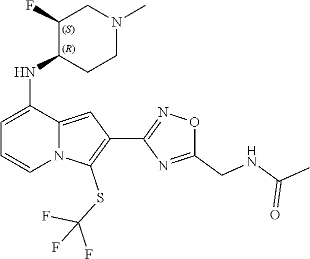

Step 1: To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (70 mg, 0.16 mmol, 1 equiv) in DCM (0.7 mL) were added TEA (0.04 mL, 0.31 mmol, 2 equiv) and acetic anhydride (48.2 mg, 0.47 mmol, 3 equiv) dropwise at 0 °C. The resulting mixture was stirred for 1 h at 0 °C. The resulting solution was diluted with EA (30 mL) and washed with sat·NaHCO₃ (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 28% B to 53% B in 8 min; Wave Length: 220 nm nm; RT1 (min): 7.5) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}acetamide (29.6 mg, 38.21%) as a light yellow solid. LCMS: (M+H)⁺ found 487.15. ¹H NMR (400 MHz, DMSO-d₆) δ 8.78 (t, J=5.7 Hz, 1H), 8.02 (d, J=6.9 Hz, 1H), 7.78 (s, 1H), 6.85 (t, J=7.2 Hz, 1H), 6.24 (dd, J=28.5, 7.8 Hz, 2H), 4.87 (d, J=49.7 Hz, 1H), 4.62 (d, J=5.7 Hz, 2H), 3.63 (d, J=29.2 Hz, 1H), 3.05 (t, J=11.2 Hz, 1H), 2.84 (d, J=7.1 Hz, 1H), 2.20 (s, 4H), 2.09 (d, J=8.6 Hz, 2H), 1.93 (s, 3H), 1.70 (d, J=8.7 Hz, 1H).

### Example 10—Preparation of 3,3-difluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclobutane-1-carboxamide

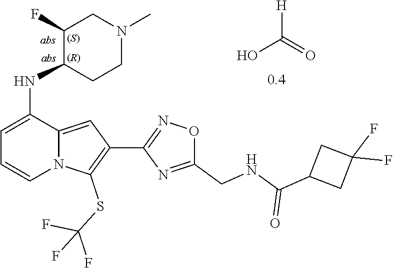

Step 1: To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50.0 mg, 0.10 mmol, 1.00 equiv) in DMF (2.00 mL) were added PyBOP (81.1 mg, 0.15 mmol, 1.50 equiv), DIEA (134.4 mg, 1.04 mmol, 10.00 equiv) and 3,3-difluorocyclobutane-1-carboxylic acid (17.0 mg, 0.12 mmol, 1.20 equiv). The resulting solution was stirred for 1 h at room temperature. The resulted solution was purified by reverse flash chromatography (Mobile Phase A: Water, Mobile Phase B: ACN; Flow rate: 80 mL/min; Gradient: 0% B to 100% B in 30 min; 254/220 nm) and Prep-HPLC (Column: Xselect CSH C18 OBD Column 30*150 mm 5 m; Mobile Phase A: Water (0.1% FA), Mobile Phase B: ACN; Flow rate: 60 mL/min; Gradient: 20% B to 35% B in 7 min; Wave Length: 254 nm/220 nm; RT(min): 6.3). This resulted in 3,3-difluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclobutane-1-carboxamide (FA salt; 17.5 mg, 29.4%) as a light yellow solid. LC-MS: (M+H)⁺ found 563.10. ¹H NMR (400 MHz, DMSO-d₆) δ 8.98 (t, J=5.7 Hz, 1H), 8.02 (d, J=6.8 Hz, 1H), 7.77 (s, 1H), 6.85 (t, J=7.2 Hz, 1H), 6.26 (d, J=8.1 Hz, 1H), 6.21 (d, J=7.6 Hz, 1H), 4.87 (d, J=48.0 Hz, 1H), 4.68 (d, J=5.7 Hz, 2H), 3.75-3.58 (m, 1H), 3.08-2.96 (m, 2H), 2.84 (s, 1H), 2.81-2.70 (m, 4H), 2.21 (s, 4H), 2.13-2.03 (m, 2H), 1.70 (d, J=11.0 Hz, 1H).

### Example 15—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3,3-dimethyl-2-oxo-1H-indole-5-carboxamide

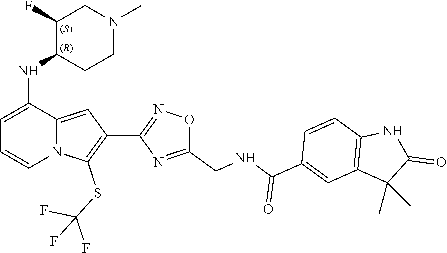

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 3,3-dimethyl-2-oxo-1H-indole-5-carboxylic acid (23.1 mg, 0.11 mmol, 1 equiv), PyBOP (70.3 mg, 0.13 mmol, 1.2 equiv), and DIEA (87.2 mg, 0.67 mmol, 6 equiv) in DMF (2 mL) was stirred for 1 h at room temperature. The resulting solution was diluted with EtOAc (50 mL) and washed with brine (2*50 mL). The organic layer was dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 30% B to 55% B in 7 min; Wave Length: 254 nm/220 nm nm; RT1 (min): 6.47; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3,3-dimethyl-2-oxo-1H-indole-5-carboxamide (41.9 mg, 58.73%) as a white solid. LC-MS: (M+H)⁺ found 632.35. ¹H NMR (400 MHz, DMSO-d₆) δ 10.66 (s, 1H), 9.25 (t, J=5.6 Hz, 1H), 8.02 (d, J=6.8 Hz, 1H), 7.88 (d, J=1.8 Hz, 1H), 7.84-7.72 (m, 2H), 6.94 (d, J=8.1 Hz, 1H), 6.85 (t, J=7.2 Hz, 1H), 6.26 (d, J=8.0 Hz, 1H), 6.20 (d, J=7.7 Hz, 1H), 4.98-4.71 (m, 3H), 3.62 (d, J=27.7 Hz, 1H), 3.04 (t, J=11.2 Hz, 1H), 2.82 (d, J=8.4 Hz, 1H), 2.19 (s, 4H), 2.13-2.03 (m, 2H), 1.69 (s, 1H), 1.29 (s, 6H).

### Example 21—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-methoxy-1H-indazole-3-carboxamide

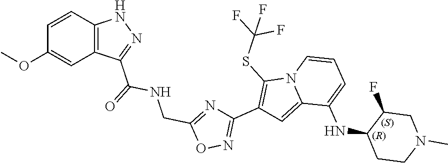

To a solution of 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine hydrochloride (50.0 mg, 0.10 mmol, 1.00 equiv) in DMF (1.00 mL) were added 5-methoxy-1H-indazole-3-carboxylic acid (21.6 mg, 0.11 mmol, 1.00 equiv), DIEA (145.4 mg, 1.12 mmol, 10.00 equiv) and PyBOP (87.8 mg, 0.17 mmol, 1.50 equiv). The resulting solution was stirred for 1 h at room temperature. The resulted solution was purified by reverse flash chromatography (Mobile Phase A: Water (0.1% FA), Mobile Phase B: ACN; Flow rate: 60 mL/min; Gradient: 0% B to 100% B in 30 min; 254/220 nm) and Prep-HPLC (XBridge Prep OBD C18 Column, 19*250 mm, 5 m, Mobile Phase A: Water (10 mmol/L NH₄HCO₃+0.05% NH₃H₂O), Mobile Phase B: MeOH; Flow rate: 25 mL/min; Gradient: 58% B to 83% B in 10 min; Wave Length: 254 nm/220 nm; RT(min): 9.73; Number Of Runs: 3). This resulted in N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-5-methoxy-1H-indazole-3-carboxamide (17.7 mg, 24.54%) as a white solid. LC-MS: (M+H)⁺ found 619.35. ¹H NMR (400 MHz, DMSO-d₆) δ 13.64 (s, 1H), 9.20 (t, J=5.9 Hz, 1H), 8.02 (d, J=6.8 Hz, 1H), 7.78-7.73 (m, 1H), 7.59-7.51 (m, 2H), 7.08 (dd, J=9.0, 2.5 Hz, 1H), 6.85 (t, J=7.2 Hz, 1H), 6.26 (d, J=8.1 Hz, 1H), 6.19 (d, J=7.6 Hz, 1H), 5.10-4.46 (m, 3H), 3.79 (s, 3H), 3.40 (d, J=11.2 Hz, 1H), 3.01 (d, J=11.2 Hz, 1H), 2.81 (d, J=8.6 Hz, 1H), 2.38-2.18 (m, 4H), 2.17-1.85 (m, 2H), 1.66 (d, J=10.4 Hz, 1H).

### Example 22—Preparation of tert-butyl N-[1-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}carbamoyl)cyclopropyl]carbamate

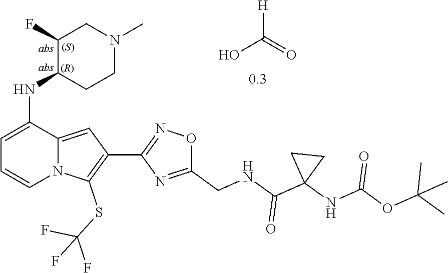

To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50.0 mg, 0.10 mmol, 1.00 equiv) in DMF (2.00 mL) were added PyBOP (81.1 mg, 0.15 mmol, 1.50 equiv), DIEA (134.4 mg, 1.0 mmol, 10.00 equiv) and 1-[(tert-butoxycarbonyl)amino]cyclopropane-1-carboxylic acid (25.1 mg, 0.12 mmol, 1.20 equiv). The resulting solution was stirred for 1 h at room temperature. The resulted solution was purified by reverse flash chromatography (Mobile Phase A: Water, Mobile Phase B: ACN; Flow rate: 80 mL/min; Gradient: 0% B to 100% B in 30 min; 254/220 nm) and Prep-HPLC (Column: Xselect CSH C18 OBD Column 30*150 mm 5 m; Mobile Phase A: Water (0.1% FA), Mobile Phase B: ACN; Flow rate: 60 mL/min; Gradient: 22% B to 35% B in 7 min; Wave Length: 254 nm/220 nm; RT(min): 6.9). This resulted in tert-butyl N-[1-({[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}carbamoyl)cyclopropyl]carbamate (FA salt; 14.2 mg, 21.1%) as a light yellow solid. LC-MS: (M+H)⁺ found 628.15. ¹H NMR (400 MHz, DMSO-d₆) δ 8.82-8.50 (m, 1H), 8.03 (d, J=6.8 Hz, 1H), 7.77 (s, 1H), 7.49-7.19 (m, 1H), 6.86 (t, J=7.2 Hz, 1H), 6.35-6.13 (m, 2H), 4.88 (d, 0.1=49.6 Hz, 1H), 4.64 (d, 0.1=5.7 Hz, 2H), 3.75 (s, 1H), 3.06 (t, 0.1=10.8 Hz, 1H), 2.85 (d, J=9.2 Hz, 1H), 2.38-2.20 (m, 4H), 2.19-2.00 (m, 2H), 1.75-1.67 (m, 1H), 1.39 (d, J=10.0 Hz, 9H), 1.27 (m, 2H), 0.94 (m, 2H).

### Example 24—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-hydroxycyclopropane-1-carboxamide

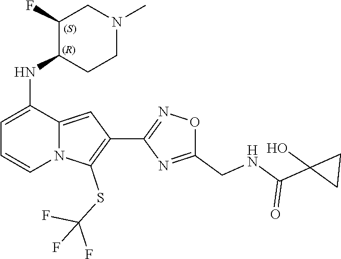

To a solution of 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine hydrochloride (50.0 mg, 0.10 mmol, 1.00 equiv) in DMF (2.00 mL) were added 1-hydroxycyclopropane-1-carboxylic acid (10.6 mg, 0.10 mmol, 1.00 equiv), PyBOP (81.2 mg, 0.15 mmol, 1.50 equiv) and DIEA (134.4 mg, 1.04 mmol, 10.00 equiv). The resulting solution was stirred for 1 h at room temperature. The resulted solution was purified by reverse flash chromatography (Mobile Phase A: Water (0.1% FA), Mobile Phase B: ACN; Flow rate: 60 mL/min; Gradient: 0% B to 100% B in 30 min; 254/220 nm) and Prep-HPLC (XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min; Gradient: 30% B to 55% B in 7 min; Wave Length: 254 nm/220 nm; RT(min): 7.23). This resulted in N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-hydroxycyclopropane-1-carboxamide (22.3 mg, 40.26%) as a white solid. LC-MS: (M+H)⁺ found 529.15. ¹H NMR (400 MHz, DMSO-d₆) δ 8.78 (t, J=5.9 Hz, 1H), 8.02 (d, J=6.9 Hz, 1H), 7.78 (s, 1H), 6.85 (t, J=7.2 Hz, 1H), 6.39 (s, 1H), 6.27 (d, J=8.1 Hz, 1H), 6.21 (d, J=7.6 Hz, 1H), 4.87 (d, J=49.4 Hz, 1H), 4.68 (d, J=5.9 Hz, 2H), 3.63 (d, J=28.1 Hz, 1H), 3.05 (t, J=11.0 Hz, 1H), 2.83 (s, 1H), 2.35-2.19 (m, 4H), 2.18-1.98 (m, 2H), 1.80-1.62 (m, 1H), 1.11-1.00 (m, 2H), 0.91-0.88 (m, 2H).

### Example 25—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}oxetane-3-carboxamide

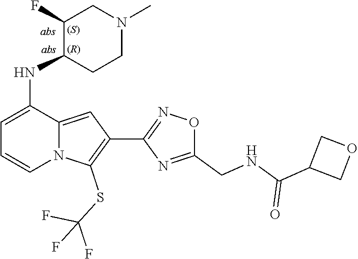

To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (40 mg, 0.09 mmol, 1 equiv), DIEA (0.05 mL, 0.27 mmol, 3 equiv) and oxetane-3-carboxylic acid (9.2 mg, 0.09 mmol, 1 equiv) in DMF (1 mL) was added PyBOP (60.9 mg, 0.12 mmol, 1.3 equiv) at 0 °C. The resulting solution was stirred for 1 h at room temperature. The resulting solution was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃)+0.05% NH₃—H₂O, Mobile Phase B: ACN; Flow rate: 60 mL/min mL/min; Gradient: 27% B to 54% B in 7 min; Wave Length: 254 nm/220 nm nm; RT1 (min): 7.27) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}oxetane-3-carboxamide (8.2 mg, 16.98%) as a light yellow solid. LCMS: (M+H)⁺ found 529.15. ¹H NMR (400 MHz, DMSO-d₆) δ 8.85 (t, J=5.7 Hz, 1H), 8.02 (d, J=6.8 Hz, 1H), 7.77 (d, J 0.9 Hz, 1H), 6.85 (t, J=7.2 Hz, 1H), 6.24 (dd, J=21.2, 7.8 Hz, 2H), 4.87 (d, J=49.5 Hz, 1H), 4.73-4.56 (m, 6H), 3.86 (tt, J=8.5, 6.6 Hz, 1H), 3.63 (d, J=28.7 Hz, 1H), 3.06 (dt, J=12.2, 6.5 Hz, 1H), 2.84 (d, J=7.9 Hz, 1H), 2.30-2.20 (m, 4H), 2.14-2.00 (m, 2H), 1.70 (d, J=9.7 Hz, 1H).

### Example 26—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-methoxycyclopropane-1-carboxamide

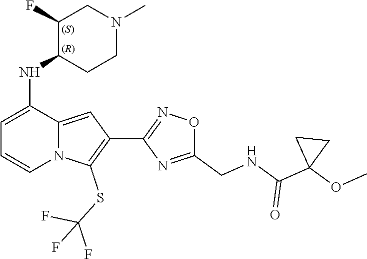

To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv) in DMF (1.5 mL) was added 1-methoxycyclopropane-1-carboxylic acid (19.6 mg, 0.17 mmol, 1.5 equiv), DIEA (72.7 mg, 0.56 mmol, 5 equiv) and PyBOP (87.8 mg, 0.17 mmol, 1.5 equiv) at 0 °C.

The reaction solution was stirred at room temperature for 1 h. The resulting mixture was diluted with water (50 mL) and extracted with DCM (3*50 mL). The combined organic layers were dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 37% B to 60% B in 7 min; Wave Length: 254 nm/220 nm nm; RT1 (min): 6.67; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-methoxycyclopropane-1-carboxamide (24.9 mg, 40.47%) as an off-white solid. LC-MS: (M+H)⁺ found 543.15. ¹H NMR (400 MHz, DMSO-d₆) 8.94 (t, J=5.9 Hz, 1H), 8.02 (d, J=6.8 Hz, 1H), 7.77 (d, J=0.8 Hz, 1H), 6.85 (t, J=7.2 Hz, 1H), 6.23 (dd, J=16.0, 7.9 Hz, 2H), 4.87 (d, J=49.4 Hz, 1H), 4.70 (d, J=5.9 Hz, 2H), 3.63 (d, J=28.3 Hz, 1H), 3.34 (s, 3H), 3.05 (t, J=11.1 Hz, 1H), 2.84 (d, J=8.0 Hz, 1H), 2.20 (s, 4H), 2.08 (t, J=10.9 Hz, 2H), 1.70 (d, J=9.5 Hz, 1H), 1.08 (m, 4H).

### Example 28—Preparation of 1-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclopropane-1-carboxamide

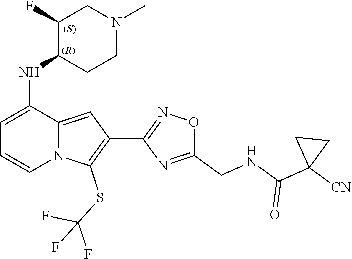

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv) in DMF (1.5 mL) was treated with DIEA (72.7 mg, 0.56 mmol, 5 equiv) and PyBOP (87.8 mg, 0.17 mmol, 1.5 equiv) for 2 min at 0 °C followed by the addition of 1-cyanocyclopropane-1-carboxylic acid (12.5 mg, 0.11 mmol, 1 equiv) at room temperature. The resulting mixture was stirred for 1 h at room temperature. The resulting solution was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 35% B to 60% B in 8 min; Wave Length: 220 nm nm; RT1 (min): 7.78) to afford 1-cyano-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}cyclopropane-1-carboxamide (18.4 mg, 30.43%) as an off-white solid. LC-MS: (M+H)⁺ found 538.35. ¹H NMR (400 MHz, DMSO-d₆) δ 9.10 (t, J=5.6 Hz, 1H), 8.02 (d, J=6.8 Hz, 1H), 7.78 (s, 1H), 6.86 (t, J=7.2 Hz, 1H), 6.25 (dd, J=30.1, 7.9 Hz, 2H), 4.87 (d, J=49.5 Hz, 1H), 4.68 (d, J=5.5 Hz, 2H), 3.63 (d, J=30.6 Hz, 1H), 3.05 (t, J=11.2 Hz, 1H), 2.84 (d, J=6.7 Hz, 1H), 2.30 (d, J=13.1 Hz, 1H), 2.20 (s, 3H), 2.14-2.01 (m, 2H), 1.68 (m, 3H), 1.56 (m, 2H).

### Example 35—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-6-methoxy-1-methylindole-2-carboxamide

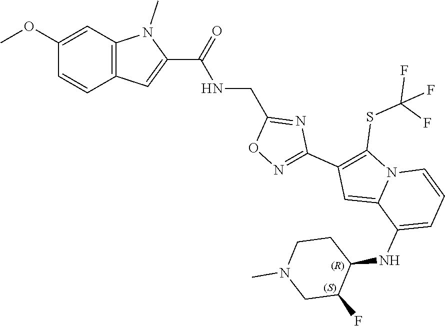

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1 equiv), 6-methoxy-1-methylindole-2-carboxylic acid (30.5 mg, 0.15 mmol, 1.1 equiv), PyBOP (84.3 mg, 0.16 mmol, 1.2 equiv) and DIEA (87.2 mg, 0.68 mmol, 5 equiv) in DMF (2 mL) was stirred for 1 h at room temperature. The resulting mixture was diluted with EtOAc (15 mL) and washed with brine (4*10 mL). The organic layer was dried over anhydrous Na₂SO₄. After filtration, the filtrate was concentrated under reduced pressure. The residue was purified by Prep-TLC (CH₂Cl₂/MeOH=20:1) and Prep-HPLC (Column: XBridge Prep OBD C18 Column, 30*150 mm, 5 μm; Mobile Phase A: Water(10 mmol/L NH₄HCO₃+0.05% NH₃H₂O), Mobile Phase B: ACN; Flow rate: 60 mL/min mL/min; Gradient: 45% B to 70% B in 8 min; Wave Length: 220 nm nm; RT1 (min): 7.8) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-6-methoxy-1-methylindole-2-carboxamide (23.7 mg, 27.65%) as a off-white solid. LC-MS: (M+H)yfound 632.15. ¹H NMR (400 MHz, DMSO-d₆) δ 9.26 (t, J=5.7 Hz, 1H), 8.02 (d, J=6.8 Hz, 1H), 7.78 (s, 1H), 7.55 (d, J=8.7 Hz, 1H), 7.20 (s, 1H), 7.04 (d, J=2.2 Hz, 1H), 6.85 (t, J=7.2 Hz, 1H), 6.77 (dd, J=8.7, 2.2 Hz, 1H), 6.26 (d, J=8.1 Hz, 1H), 6.20 (d, J=7.7 Hz, 1H), 4.97-4.74 (m, 3H), 3.98 (s, 3H), 3.84 (s, 3H), 3.62 (d, J=28.4 Hz, 1H), 3.04 (t, J=11.5 Hz, 1H), 2.81 (s, 1H), 2.19 (s, 4H), 2.06 (t, J=11.4 Hz, 2H), 1.68 (d, J=10.3 Hz, 1H).

### Example 36—Preparation of 4-fluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}oxane-4-carboxamide

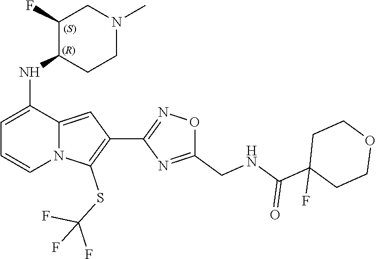

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (70 mg, 0.16 mmol, 1 equiv), PyBOP (98.4 mg, 0.19 mmol, 1.2 equiv), DIEA (0.14 mL, 0.79 mmol, 5 equiv) and 4-fluorooxane-4-carboxylic acid (25.7 mg, 0.17 mmol, 1.1 equiv) in DMF (0.7 mL) was stirred for 0.5 h at room temperature. The solution was diluted with water (30 mL) and extracted with EtOAc (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃)+0.05% NH₃—H₂O, Mobile Phase B: ACN; Flow rate: 60 mL/min mL/min; Gradient: 35% B to 60% B in 7 min; Wave Length: 254 nm/220 nm nm; RT1 (min): 7.2) to afford 4-fluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}oxane-4-carboxamide (17.7 mg, 19.34%) as a white solid. LC-MS: (M+H)⁺ found 575.15. ¹H NMR (400 MHz, DMSO-d₆) δ 9.09 (td, J=5.8, 2.4 Hz, 1H), 8.02 (d, J=6.8 Hz, 1H), 7.77 (s, 1H), 6.85 (t, J=7.2 Hz, 1H), 6.23 (dd, J=14.8, 7.9 Hz, 2H), 4.87 (d, J=49.4 Hz, 1H), 4.69 (d, J=5.7 Hz, 2H), 3.83 (m, 2H), 3.59 (td, J=11.8, 2.3 Hz, 3H), 3.05 (t, J=11.0 Hz, 1H), 2.84 (d, J=10.1 Hz, 1H), 2.20 (s, 4H), 2.18-1.97 (m, 4H), 1.83-1.64 (m, 3H).

### Example 40—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,3-benzothiazole-6-carboxamide

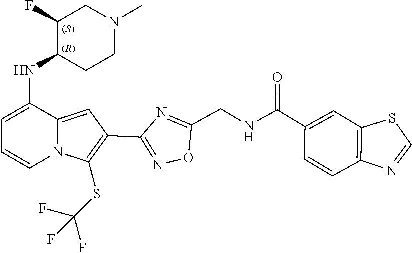

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 1,3-benzothiazole-6-carboxylic acid (22.2 mg, 0.12 mmol, 1.1 equiv), PyBOP (70.3 mg, 0.13 mmol, 1.2 equiv) and DIEA (72.7 mg, 0.56 mmol, 5 equiv) in DMF (1 mL) was stirred for 1 h at room temperature. The resulting solution was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 15:1) and Prep-HPLC (Column: XBridge Shield RP18 OBD Column, 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 37% B to 60% B in 7 min; Wave Length: 254 nm/220 nm nm; RT1 (min): 6.67; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,3-benzothiazole-6-carboxamide (18.0 mg, 26.42%) as a light yellow solid. LC-MS: (M+H)⁺ found 606.05. ¹H NMR (400 MHz, DMSO-d₆) δ 9.58 (d, J=7.8 Hz, 2H), 8.76 (d, J=1.7 Hz, 1H), 8.21 (d, J=8.5 Hz, 1H), 8.09 (m, 1H), 8.02 (d, J=6.9 Hz, 1H), 7.77 (s, 1H), 6.85 (t, J=7.2 Hz, 1H), 6.26 (d, J=8.0 Hz, 1H), 6.20 (d, J=7.6 Hz, 1H), 4.98-4.73 (m, 3H), 3.62 (d, J=28.2 Hz, 1H), 3.05 (d, J=11.2 Hz, 1H), 2.82 (d, J=8.8 Hz, 1H), 2.19 (s, 4H), 2.05 (t, J=11.4 Hz, 2H), 1.67 (d, J=11.0 Hz, 1H).

### Example 42—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methanesulfonylbenzamide

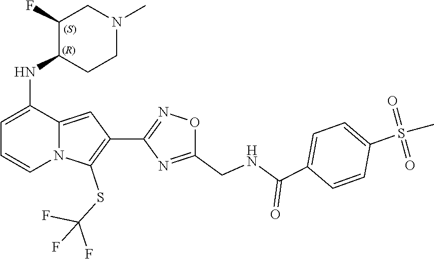

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1 equiv), P-methylsulfonylbenzoic acid (29.7 mg, 0.15 mmol, 1.1 equiv), PyBOP (84.3 mg, 0.16 mmol, 1.2 equiv) and DIEA (87.2 mg, 0.68 mmol, 5 equiv) in DMF (2 mL) was stirred for 1 h at room temperature. The resulting solution was diluted with EtOAc (15 mL) and washed with brine (4*10 mL). The organic layer was dried over anhydrous Na₂SO₄. After filtration, the filtrate was concentrated under reduced pressure. The residue was purified by Prep-TLC (CH₂Cl₂/MeOH=20:1) and Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 34% B to 59% B in 8 min; Wave Length: 220 nm nm; RT1 (min): 7.6) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methanesulfonylbenzamide (21.6 mg, 25.41%) as a off-white solid. LC-MS: (M+H)⁺ found 627.05. ¹H NMR (400 MHz, DMSO-d₆) δ 9.72 (t, J=5.6 Hz, 1H), 8.21-8.13 (m, 2H), 8.13-8.06 (m, 2H), 8.02 (d, J=6.9 Hz, 1H), 7.76 (d, J=0.8 Hz, 1H), 6.85 (t, J 7.2 Hz, 1H), 6.27 (d, J=8.1 Hz, 1H), 6.20 (d, J=7.7 Hz, 1H), 4.97-4.72 (m, 3H), 3.71-3.54 (m, 1H), 3.29 (s, 3H), 3.04 (t, J=11.4 Hz, 1H), 2.82 (d, J=7.5 Hz, 1H), 2.19 (s, 4H), 2.06 (t, J=11.2 Hz, 2H), 1.68 (d, J=10.3 Hz, 1H).

### Example 45—Preparation of tert-butyl N-[3-({[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}carbamoyl)bicyclo[1.1.1]pentan-1-yl]carbamate

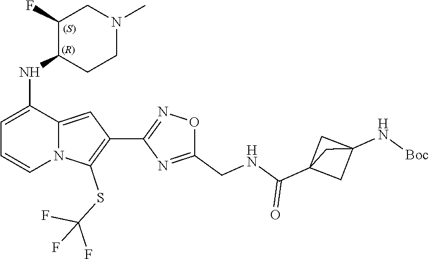

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (80 mg, 0.18 mmol, 1 equiv), 3-[(tert-butoxycarbonyl) amino]bicyclo[1.1.1]pentane-1-carboxylic acid (40.9 mg, 0.18 mmol, 1 equiv), PyBOP (112.4 mg, 0.22 mmol, 1.2 equiv) and DIEA (116.3 mg, 0.90 mmol, 5 equiv) in DMF (1 mL) was stirred for 1 h at room temperature. The resulting solution was diluted with water (30 mL) and extracted with DCM (3*30 mL). The combined organic layers were dried over anhydrous Na₂SO₄. After filtration, the filtrate was concentrated under reduced pressure. The residue was purified by Prep-HPLC (Column: XBridge Prep OBD C18 Column, 19*250 mm, 5 μm; Mobile Phase A: Water (10 mmol/L NH₄HCO₃+0.05% NH₃·H₂O), Mobile Phase B: MEOH; Flow rate: 25 mL/min mL/min; Gradient: 60% B to 83% B in 10 min, Wave Length: 254 nm/220 nm nm; RT1 (min): 9.27) to afford tert-butyl N-[3-({[3-(8-{[(3S,4R)-3-fluoro-1-methylpiperidin-4-yl]amino}-3-[(trifluoromethyl) sulfanyl]indolizin-2-yl)-1,2,4-oxadiazol-5-yl]methyl}carbamoyl)bicyclo[1.1.1]pentan-1-yl]carbamate (16.5 mg, 13.69%) as a white solid. LC-MS: (M+H)⁺ found 654.45. ¹H NMR (400 MHz, DMSO-d₆) δ 8.69 (t, J=5.8 Hz, 1H), 8.02 (d, J=6.9 Hz, 1H), 7.77 (s, 1H), 7.60 (s, 1H), 6.85 (t, J=7.2 Hz, 1H), 6.24 (dd, J=19.7, 7.8 Hz, 2H), 4.87 (d, J=49.4 Hz, 1H), 4.61 (d, J=5.7 Hz, 2H), 3.63 (d, J=29.8 Hz, 1H), 3.05 (t, J=11.2 Hz, 1H), 2.83 (s, 1H), 2.20 (s, 4H), 2.11 (s, 8H), 1.70 (d, J=9.3 Hz, 1H), 1.38 (s, 9H).

### Example 46—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-(4-fluorophenyl)cyclopropane-1-carboxamide

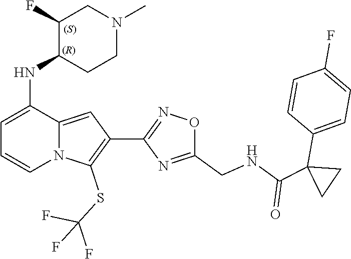

To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (90 mg, 0.20 mmol, 1 equiv), PyBOP (126.5 mg, 0.24 mmol, 1.2 equiv) and DIEA (130.9 mg, 1.01 mmol, 5 equiv) in DMF (1.0 mL) was added 1-(4-fluorophenyl)cyclopropane-1-carboxylic acid (40.1 mg, 0.22 mmol, 1.1 equiv) at 0 °C. The resulting solution was stirred for 0.5 h at room temperature. The resulting solution was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃)+0.05% NH₃·H₂O, Mobile Phase B: ACN; Flow rate: 60 mL/min mL/min, Gradient: 47% B to 74% B in 7 min; Wave Length: 254 nm/220 nm nm; RT1 (min): 7.02) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-(4-fluorophenyl)cyclopropane-1-carboxamide (40.4 mg, 32.46%) as a white solid. LC-MS: (M+H)~found 607.15. ¹H NMR (400 MHz, DMSO-d₆) δ 8.02 (d, J=6.8 Hz, 1H), 7.76 (d, J=0.9 Hz, 1H), 7.67 (t, J=5.7 Hz, 1H), 7.48-7.41 (m, 2H), 7.25-7.17 (m, 2H), 6.85 (t, J=7.2 Hz, 1H), 6.23 (dd, J=13.6, 7.9 Hz, 2H), 4.87 (d, J=49.5 Hz, 1H), 4.54 (d, J=5.7 Hz, 2H), 3.64 (d, J=28.8 Hz, 1H), 3.05 (t, J=11.1 Hz, 1H), 2.88-2.78 (m, 1H), 2.20 (s, 4H), 2.08 (t, J=11.1 Hz, 2H), 1.70 (d, J=9.7 Hz, 1H), 1.40 (m, 2H), 1.05 (m, 2H).

### Example 47—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-(trifluoromethyl)cyclopropane-1-carboxamide

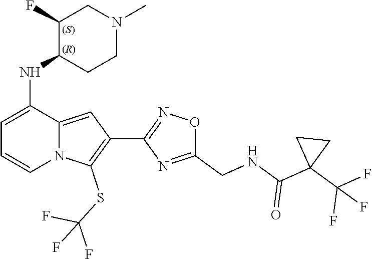

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (70 mg, 0.16 mmol, 1 equiv), 1-(trifluoromethyl)cyclopropane-1-carboxylic acid (24.3 mg, 0.16 mmol, 1 equiv), PyBOP (98.4 mg, 0.19 mmol, 1.2 equiv) and DIEA (101.8 mg, 0.79 mmol, 5 equiv) in DMF (1 mL) was stirred for 1 h at room temperature. The resulting solution was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃)+0.05% NH₃—H₂O, Mobile Phase B: ACN; Flow rate: 60 mL/min mL/min; Gradient: 44% B to 68% B in 7 min; Wave Length: 254 nm/220 nm nm; RT1 (min): 7.25) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1-(trifluoromethyl)cyclopropane-1-carboxamide (35.5 mg, 38.67%) as a white solid. LC-MS: (M+H)⁺ found 581.30. ¹H NMR (400 MHz, DMSO-d₆) δ 8.65 (t, J=5.5 Hz, 1H), 8.02 (d, J=6.8 Hz, 1H), 7.77 (d, J=0.9 Hz, 1H), 6.85 (t, J=7.2 Hz, 1H), 6.23 (dd, J=18.6, 7.8 Hz, 2H), 4.87 (d, J=49.4 Hz, 1H), 4.66 (d, J=5.6 Hz, 2H), 3.64 (d, J=28.9 Hz, 1H), 3.05 (t, J=11.2 Hz, 1H), 2.84 (d, J=7.6 Hz, 1H), 2.20 (s, 4H), 2.08 (t, J=10.8 Hz, 2H), 1.70 (d, J=9.8 Hz, 1H), 1.38 (d, J=7.5 Hz, 2H), 1.36-1.27 (m, 2H).

### Example 49—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-hydroxy-2-methylpropanamide

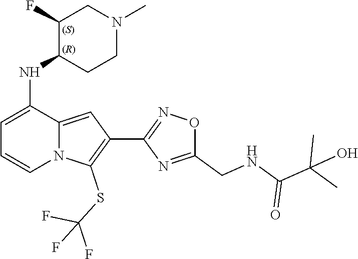

To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (80 mg, 0.18 mmol, 1 equiv) in anhydrous DMF (1 mL) was added acetonate (28.1 mg, 0.27 mmol, 1.5 equiv), PyBOP (140.5 mg, 0.27 mmol, 1.5 equiv) and DIEA (116.3 mg, 0.90 mmol, 5 equiv) at 0 °C. The reaction solution was stirred at room temperature for 1 h at room temperature. The resulting mixture was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD Cis Column, 30*150 mm, 5 m; Mobile Phase A: Water (10 mmol/L NH₄HCO₃)+0.05% NH₃—H₂O, Mobile Phase B: ACN; Flow rate: 60 mL/min mL/min; Gradient: 31% B to 56% B in 7 min; Wave Length: 254 nm/220 nm nm; RT1 (min): 7.18) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-hydroxy-2-methylpropanamide (42.1 mg, 42.67%) as a yellow solid. LC-MS: (M+H)~found 531.40. ¹H NMR (400 MHz, DMSO-d₆) 8.55 (t, J=5.9 Hz, 1H), 8.02 (d, J=6.8 Hz, 1H), 7.76 (s, 1H), 6.85 (t, J=7.2 Hz, 1H), 6.22 (t, J=8.8 Hz, 2H), 5.53 (s, 1H), 4.87 (d, J=49.5 Hz, 1H), 4.63 (d, J=5.9 Hz, 2H), 3.63 (d, J=28.3 Hz, 1H), 3.05 (t, J=11.1 Hz, 1H), 2.83 (d, J=7.8 Hz, 1H), 2.20 (s, 4H), 2.08 (t, J=11.0 Hz, 2H), 1.70 (d, J=9.6 Hz, 1H), 1.30 (s, 6H).

### Example 57—Preparation of N₁—{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-N4-methylbenzene-1,4-dicarboxamide

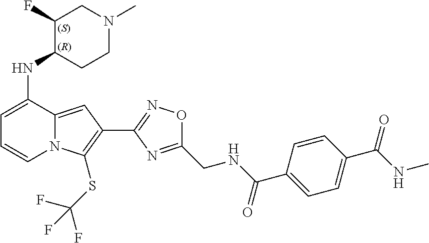

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 4-(methylcarbamoyl)benzoic acid (22.2 mg, 0.12 mmol, 1.1 equiv), PyBOP (70.3 mg, 0.13 mmol, 1.2 equiv) and DIEA (72.7 mg, 0.56 mmol, 5 equiv) in DMF (1 mL) was stirred for 1 h at room temperature. The resulting solution was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (CH₂Cl₂/MeOH 15:1) and Prep-HPLC (Column: XBridge Prep OBD C18 Column, 30*150 mm, 5 μm; Mobile Phase A: Water (10 mmol/L NH₄HCO₃)+0.05% NH₃—H₂O, Mobile Phase B: ACN; Flow rate: 60 mL/min mL/min; Gradient: 31% B to 56% B in 7 min; Wave Length: 254 nm/220 nm nm; RT1 (min): 7.23) to afford N₁—{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-N4-methylbenzene-1,4-dicarboxamide (15.7 mg, 23.04%) as a white solid. LC-MS: (M+H)⁺ found 606.15. ¹H NMR (400 MHz, DMSO-d₆) δ 9.54 (t, J=5.6 Hz, 1H), 8.60 (d, J=4.7 Hz, 1H), 8.16-7.83 (m, 5H), 7.77 (s, 1H), 6.85 (t, J=7.2 Hz, 1H), 6.26 (d, J 8.1 Hz, 1H), 6.20 (d, J 7.7 Hz, 1H), 5.00-4.71 (m, 3H), 3.62 (d, J=29.4 Hz, 1H), 3.04 (t, J=11.4 Hz, 1H), 2.81 (d, J=4.5 Hz, 4H), 2.19 (s, 4H), 2.14-1.97 (m, 2H), 1.68 (d, J=9.9 Hz, 1H).

### Example 58—Preparation of N₁—{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-N3-methylbenzene-1,3-dicarboxamide

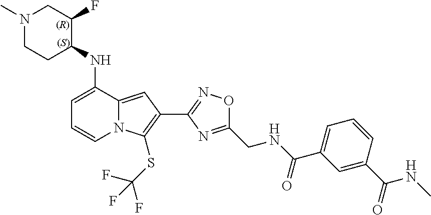

Step 1: A mixture of isophthalic acid, methyl ester (1 g, 5.55 mmol, 1 equiv), HATU (3.2 g, 8.32 mmol, 1.5 equiv) CH₃NH₂·HCl (0.5 g, 6.66 mmol, 1.2 equiv) and DIEA (2.9 g, 22.20 mmol, 4 equiv) in DMF (5 mL) was stirred for 1 h at room temperature. The resulting solution was purified by reversed-phase flash chromatography (column, C18 silica gel; mobile phase, MeCN in water (0.1% FA), 0 to 100% gradient in 20 min, detector, UV 254 nm) to afford methyl 3-(methylcarbamoyl)benzoate (1 g, 93.25%) as a off-white solid. LC-MS: (M+H)⁺ found 193.2.

Step 2: A mixture of methyl 3-(methylcarbamoyl)benzoate (1 g, 5.18 mmol, 1 equiv) and NaOH (2.1 g, 51.76 mmol, 10 equiv) in H₂O (10 mL)/MeOH (20 mL) was stirred for 2 h at room temperature. The resulting mixture was diluted with water (50 mL) and acidified to pH 2 with 1M HCl. The aqueous layer was extracted with EtOAc (3*50 mL). The combined organic layers were dried over anhydrous Na₂SO₄. After filtration, the filtrate was concentrated under reduced pressure to afford 3-(methylcarbamoyl)benzoic acid (820 mg, 88.42%) as a off-white solid. LC-MS: (M+H)⁺ found 179.1.

Step 3: A mixture of 3-(methylcarbamoyl)benzoic acid (24.2 mg, 0.13 mmol, 1.2 equiv), 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1.00 equiv), PyBOP (87.8 mg, 0.17 mmol, 1.5 equiv) and DIEA (72.7 mg, 0.56 mmol, 5 equiv) in DMF (2 mL) was stirred for 1 h at room temperature. The resulting solution was purified by Prep-TLC (CH₂Cl₂/MeOH 10:1) and Prep-HPLC (Column: XBridge Shield RP18 OBD Column, 30*150 mm, 5 m; Mobile Phase A: Water (10 mmol/L NH₄HCO₃+0.05% NH₃·H₂O), Mobile Phase B: ACN; Flow rate: 60 mL/min mL/min; Gradient: 30% B to 55% B in 7 min; Wave Length: 254 nm/220 nm nm; RT1 (min): 6.75; Number Of Runs: 2) to afford N₁—{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-N3-methylbenzene-1,3-dicarboxamide (15.6 mg, 22.90%) as a yellow solid. LC-MS: (M+H)⁺ found 606.15. ¹H NMR (400 MHz, DMSO-d₆) δ 9.55 (t, J=5.6 Hz, 1H), 8.59 (d, J=4.8 Hz, 1H), 8.39 (d, J=1.9 Hz, 1H), 8.12-7.92 (m, 3H), 7.76 (s, 1H), 7.61 (t, J=7.7 Hz, 1H), 6.85 (t, J=7.2 Hz, 1H), 6.26 (d, J=8.1 Hz, 1H), 6.20 (d, J=7.6 Hz, 1H), 4.96-4.73 (m, 3H), 3.62 (d, J=28.7 Hz, 1H), 3.04 (t, J=11.1 Hz, 1H), 2.80 (d, J=4.5 Hz, 4H), 2.19 (s, 4H), 2.06 (t, J=11.5 Hz, 2H), 1.68 (d, J=10.4 Hz, 1H).

### Example 60—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-methylpropanamide

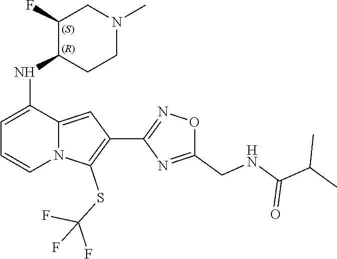

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (100 mg, 0.23 mmol, 1 equiv) in DMF (1.5 mL) was treated with DIEA (145.4 mg, 1.13 mmol, 5 equiv) and PyBOP (175.6 mg, 0.34 mmol, 1.5 equiv) for 2 min at 0 °C followed by the addition of isobutyric acid (19.8 mg, 0.23 mmol, 1 equiv). The resulting solution was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 μm; Mobile Phase A: Water (10 mmol/L NH₄HCO₃+0.05% NH₃—H₂O), Mobile Phase B: ACN; Flow rate: 60 mL/min mL/min; Gradient: 35% B to 60% B in 8 min; Wave Length: 220 nm nm; RT1 (min): 7.78) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-methylpropanamide (28.3 mg, 24.45%) as an off-white solid. LC-MS: (M+H)⁺ found 515.35. ¹H NMR (400 MHz, DMSO-d₆) δ 8.67 (t, J=5.7 Hz, 1H), 8.02 (d, J=6.8 Hz, 1H), 7.77 (d, J=0.9 Hz, 1H), 6.85 (t, J=7.2 Hz, 1H), 6.23 (dd, J=16.8, 7.8 Hz, 2H), 4.87 (d, J=49.5 Hz, 1H), 4.62 (d, J=5.7 Hz, 2H), 3.63 (d, J=29.3 Hz, 1H), 3.05 (t, J=11.3 Hz, 1H), 2.83 (s, 1H), 2.50-2.42 (m, 1H), 2.20 (s, 4H), 2.16-2.00 (m, 2H), 1.70 (d, J=10.4 Hz, 1H), 1.06 (d, J=6.8 Hz, 6H).

### Example 61—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-methoxy-2-methylpropanamide

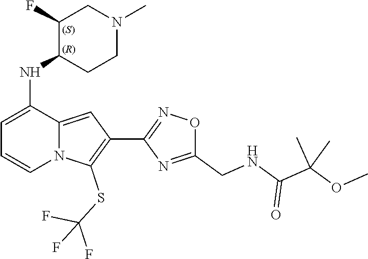

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 2-methoxy-2-methylpropanoic acid (13.3 mg, 0.11 mmol, 1 equiv), pybop (76.1 mg, 0.15 mmol, 1.3 equiv) and DIEA (0.06 mL, 0.34 mmol, 3 equiv) in DMF (1 mL) was stirred for 1 at room temperature. The resulting solution was diluted with water (30 mL) and extracted with EtOAc (3*30 mL). The organic layer was dried over anhydrous Na₂SO₄. After filtration, the filtrate was concentrated under reduced pressure. The residue was purified by Prep-HPLC (Column: XBridge Shield RP18 OBD Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 37% B to 60% B in 7 min; Wave Length: 254 nm/220 nm nm; RT1 (min): 6.13; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-methoxy-2-methylpropanamide (18.1 mg, 29.49%) as a light yellow solid. LCMS: (M+H)⁺ found 545.20. ¹H NMR (400 MHz, DMSO-d₆) δ 8.68 (t, J=5.9 Hz, 1H), 8.02 (d, J=6.8 Hz, 1H), 7.76 (d, J=0.8 Hz, 1H), 6.85 (t, J=7.2 Hz, 1H), 6.23 (dd, J=12.2, 7.9 Hz, 2H), 4.87 (d, J=49.4 Hz, 1H), 4.64 (d, J=5.9 Hz, 2H), 3.64 (d, J=28.1 Hz, 1H), 3.23 (s, 3H), 3.05 (t, J=11.1 Hz, 1H), 2.84 (d, J=7.7 Hz, 1H), 2.30 (d, J=13.2 Hz, 1H), 2.20 (s, 3H), 2.13-2.01 (m, 2H), 1.70 (d, J=9.7 Hz, 1H), 1.31 (s, 6H).

### Example 63—Preparation of (3R)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxybutanamide

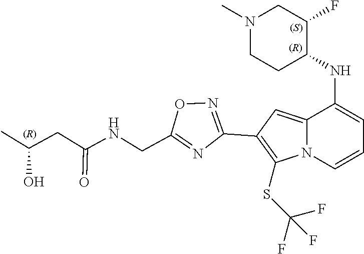

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1 equiv), (−)-3-hydroxybutyric acid (15.5 mg, 0.15 mmol, 1.1 equiv), PyBOP (84.3 mg, 0.16 mmol, 1.2 equiv) and DIEA (87.2 mg, 0.68 mmol, 5 equiv) in DMF (1 mL) was stirred for 30 min at room temperature. The resulting mixture was diluted with EtOAc (10 mL) and washed with brine (4*10 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃)+0.05% NH₃—H₂O, Mobile Phase B: ACN; Flow rate: 60 mL/min mL/min; Gradient: 26% B to 53% B in 7 min; Wave Length: 254 nm/220 nm nm; RT1 (min): 7.37) to afford (3R)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxybutanamide (16.5 mg, 22.65%) as a light yellow solid. LC-MS: (M+H)⁺ found 531.15. ¹H NMR (400 MHz, DMSO-d₆) δ 8.71 (t, J=5.7 Hz, 1H), 8.02 (d, J=6.9 Hz, 1H), 7.79-7.73 (m, 1H), 6.85 (t, J=7.2 Hz, 1H), 6.22 (dd, J=11.2, 7.9 Hz, 2H), 4.87 (d, J=49.4 Hz, 1H), 4.75-4.55 (m, 3H), 4.00 (dt, J=12.0, 6.2 Hz, 1H), 3.63 (d, J=28.8 Hz, 1H), 3.05 (s, 1H), 2.83 (d, J=8.0 Hz, 1H), 2.41-2.28 (m, 1H), 2.22 (d, J=19.4 Hz, 5H), 2.16-1.99 (m, 2H), 1.70 (d, J=9.9 Hz, 1H), 1.10 (d, J=6.1 Hz, 3H).

### Example 64—Preparation of (3S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxybutanamide

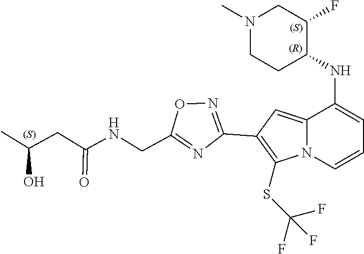

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), (S)-3-hydroxybutanoic acid (14.1 mg, 0.13 mmol, 1.2 equiv), PyBOP (70.3 mg, 0.13 mmol, 1.2 equiv) and DIEA (72.7 mg, 0.56 mmol, 5 equiv) in DMF (1 mL) was stirred for 1 h at room temperature under. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃)+0.05% NH₃H₂O, Mobile Phase B: ACN; Flow rate: 60 mL/min mL/min; Gradient: 26% B to 50% B in 7 min; Wave Length: 254 nm/220 nm nm; RT1 (min): 7.75) to afford (3S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxybutanamide (19.6 mg, 32.84%) as a light yellow solid. LC-MS: (M+H)⁺ found 531.15. ¹H NMR (400 MHz, DMSO-d₆) δ 8.71 (t, J=5.7 Hz, 1H), 8.02 (d, J=6.9 Hz, 1H), 7.76 (s, 1H), 6.85 (t, J=7.2 Hz, 1H), 6.22 (m, 2H), 4.86 (d, J=49.3 Hz, 1H), 4.76-4.55 (m, 3H), 4.00 (m, 1H), 3.63 (d, J=28.9 Hz, 1H), 3.05 (t, J=11.2 Hz, 1H), 2.83 (d, J=8.1 Hz, 1H), 2.34 (m, 1H), 2.22 (d, J=19.5 Hz, 5H), 2.08 (t, J=10.9 Hz, 2H), 1.70 (d, J=9.6 Hz, 1H), 1.10 (d, J=6.1 Hz, 3H).

### Example 65—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxy-3-methylbutanamide

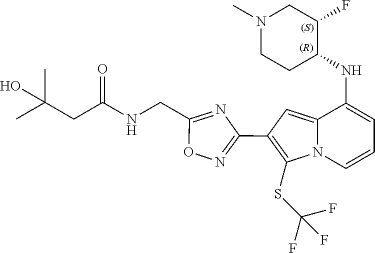

To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 3-hydroxyisovaleric acid (14.6 mg, 0.12 mmol, 1.1 equiv) and DIEA (72.7 mg, 0.56 mmol, 5 equiv) in DMF (1 mL) was added PyBOP (70.3 mg, 0.13 mmol, 1.2 equiv) at room temperature.

The resulting mixture was stirred for 1 h at room temperature. The resulting solution was diluted with EtOAc (30 mL) and washed with brine (3*30 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, Spm; Mobile Phase A: Water (10 mmol/L NH₄HCO₃+0.05% NH₃H₂O), Mobile Phase B: ACN; Flow rate: 60 mL/min mL/min; Gradient: 31% B to 56% B in 8 min; Wave Length: 220 nm nm; RT1 (min): 6.98) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxy-3-methylbutanamide (9.9 mg, 15.79%) as a white solid. LC-MS: (M+H)⁺ found 545.15. ¹H NMR (400 MHz, DMSO-d₆) δ 8.71 (t, J=5.7 Hz, 1H), 8.02 (d, J=6.9 Hz, 1H), 7.76 (s, 1H), 6.85 (t, J=7.2 Hz, 1H), 6.22 (t, J=9.2 Hz, 2H), 4.87 (d, J=49.5 Hz, 1H), 4.74 (s, 1H), 4.66 (d, J=5.7 Hz, 2H), 3.64 (d, J=29.7 Hz, 1H), 3.06 (t, J=11.3 Hz, 1H), 2.84 (d, J=8.5 Hz, 1H), 2.32 (s, 2H), 2.20 (s, 3H), 2.08 (t, J=11.3 Hz, 2H), 1.70 (d, J=10.5 Hz, 1H), 1.20 (s, 6H).

### Example 8—Preparation of (1r,3r)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxycyclobutane-1-carboxamide

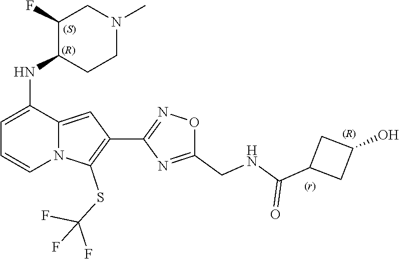

To a solution of 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine hydrochloride (50.0 mg, 0.10 mmol, 1.00 equiv) in DMF (1.00 mL) were added trans-3-hydroxycyclobutanecarboxylic acid (12.8 mg, 0.01 mmol, 1.00 equiv), PyBOP (75.4 mg, 0.15 mmol, 1.50 equiv) and DIEA (124.9 mg, 0.97 mmol, 10.00 equiv). The resulting solution was stirred for 1 h at room temperature. The resulted solution was purified by reverse flash chromatography (Mobile Phase A: Water (0.1% FA), Mobile Phase B: ACN; Flow rate: 60 mL/min; Gradient: 0% B to 100% B in 30 min; 254/220 nm) and Prep-HPLC (XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min; Gradient: 27% B to 52% B in 8 min; Wave Length: 220 nm; RT(min): 7.17). This resulted in (1r,3r)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxycyclobutane-1-carboxamide (20.2 mg, 38.10%) as a white solid. LC-MS: (M+H)⁺ found 543.10. ¹H NMR (400 MHz, DMSO-d₆) δ 8.64 (t, J=5.8 Hz, 1H), 8.03 (d, J=6.9 Hz, 1H), 7.76 (s, 1H), 6.86 (t, J=7.2 Hz, 1H), 6.28-6.10 (m, 2H), 5.06 (d, J=6.2 Hz, 1H), 4.92 (d, J=49.0 Hz, 1H), 4.63 (d, J=5.7 Hz, 2H), 4.26 (t, J=6.8 Hz, 1H), 3.68 (d, J=29.2 Hz, 1H), 3.28 (s, 1H), 2.98-2.87 (m, 2H), 2.42-2.20 (m, 6H), 2.19-1.95 (m, 4H), 1.90-1.60 (m, 1H).

### Example 9—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(5-methoxy-1H-indazol-3-yl)acetamide

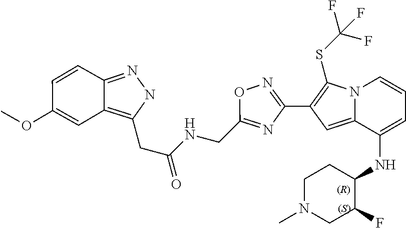

To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine hydrochloride (50.0 mg, 0.10 mmol, 1.00 equiv) in DMF (2.0 mL) were added PyBOP (81.2 mg, 0.15 mmol, 1.50 equiv), DIEA (134.4 mg, 1.04 mmol, 10.00 equiv) and (5-methoxy-1H-indazol-3-yl)acetic acid (25.7 mg, 0.12 mmol, 1.20 equiv). The resulting solution was stirred for 1 h at room temperature. The resulted solution was purified by reverse flash chromatography (Mobile Phase A: Water, Mobile Phase B: ACN; Flow rate: 80 mL/min; Gradient: 0% B to 100% B in 30 min; 254/220 nm) and Prep-HPLC (Column: XBridge Prep OBD C18 Column, 30*150 mm, 5 μm; Mobile Phase A: Water (10 mmol/L NH₄HCO₃+0.05% NH₃·H₂O), Mobile Phase B: ACN; Flow rate: 60 mL/min; Gradient: 33% B to 60% B in 7 min; Wave Length: 254 nm/220 nm; RT1 (min): 7.68). This resulted in N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(5-methoxy-1H-indazol-3-yl)acetamide (28.5 mg, 43.0%) as an off-white solid. LC-MS: (M+H)⁺ found 633.20. ¹H NMR (400 MHz, DMSO-d₆) δ 12.70 (s, 1H), 9.02 (t, J=5.8 Hz, 1H), 8.03 (d, J=6.9 Hz, 1H), 7.75 (d, J=0.9 Hz, 1H), 7.38 (d, J=9.0 Hz, 1H), 7.19 (d, J=2.3 Hz, 1H), 6.98 (dd, J=9.0, 2.4 Hz, 1H), 6.86 (t, J=7.2 Hz, 1H), 6.26-6.21 (m, 2H), 4.87 (d, J=49.6 Hz, 1H), 4.69 (d, J=5.7 Hz, 2H), 3.88 (s, 2H), 3.76 (s, 3H), 3.75-3.56 (m, 1H), 3.05 (t, J=11.1 Hz, 1H), 2.84 (d, J=7.4 Hz, 1H), 2.35-2.18 (m, 4H), 2.08 (t, J=9.7 Hz, 2H), 1.71 (d, J=9.4 Hz, 1H).

### Example 11—Preparation of (2S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,4-dioxane-2-carboxamide

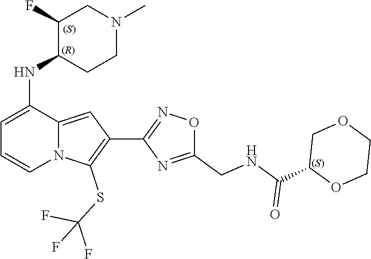

To a solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine hydrochloride (50.0 mg, 0.10 mmol, 1.00 equiv) in DMF (1.00 mL) were added (2S)-1,4-dioxane-2-carboxylic acid (13.7 mg, 0.10 mmol, 1.00 equiv), PyBOP (81.2 mg, 0.16 mmol, 1.50 equiv) and DIEA (134.4 mg, 1.04 mmol, 10.00 equiv). The resulting solution was stirred for 1 h at room temperature. The resulted solution was purified by reverse flash chromatography (Mobile Phase A: Water (0.1% FA), Mobile Phase B: ACN; Flow rate: 60 mL/min; Gradient: 0% B to 100% B in 30 min; 254/220 nm) and Prep-HPLC (XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min; Gradient: 31% B to 57% B in 7 min; Wave Length: 254 nm/220 nm; RT(min): 7.52). This resulted in (2S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,4-dioxane-2-carboxamide (11.6 mg, 19.94%) as a white solid. LC-MS: (M+H)⁺ found 559.10. ¹H NMR (400 MHz, DMSO-d₆) δ 8.74 (t, 0.1=5.9 Hz, 1H), 8.02 (d, J=6.8 Hz, 1H), 7.77 (s, 1H), 6.85 (t, J=7.2 Hz, 1H), 6.35-6.10 (m, 2H), 4.87 (d, J=49.5 Hz, 1H), 4.73-4.58 (m, 2H), 4.16 (dd, J=9.4, 3.1 Hz, 1H), 3.89 (td, J=11.1, 10.7, 2.9 Hz, 2H), 3.76-3.65 (m, 3H), 3.62-3.42 (m, 2H), 3.05 (t, J=11.2 Hz, 1H), 2.84 (d, J=7.9 Hz, 1H), 2.40-2.18 (m, 4H), 2.17-2.02 (m, 2H), 1.70 (d, J=9.4 Hz, 1H).

### Example 12—Preparation of 2-(dimethylamino)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,3-oxazole-5-carboxamide

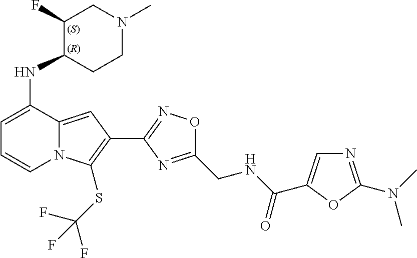

Step 1: A mixture of ethyl 2-amino-1,3-oxazole-5-carboxylate (400 mg, 2.56 mmol, 1 equiv), dimethylamine hydrochloride (626.7 mL, 7.69 mmol, 3 equiv) and K2CO₃ (1.77 g, 12.81 mmol, 5 equiv) in ACN (10 mL) was stirred for 2 h at room temperature. The resulting mixture was diluted with water (50 mL) and extracted with DCM (3*50 mL). The combined organic layers were dried over anhydrous Na₂SO₄. After filtration, the filtrate was concentrated under reduced pressure. This resulted in ethyl 2-(dimethylamino)-1,3-oxazole-5-carboxylate (400 mg, 84.77%) as a light yellow oil. LC-MS: (M+H)⁺ found 185.1.

Step 2: To a stirred mixture of ethyl 2-(dimethylamino)-1,3-oxazole-5-carboxylate (500 mg, 2.72 mmol, 1 equiv) in THF (2 mL)/H₂O (2 mL) was added NaOH (108.6 mg, 2.72 mmol, 1 equiv) at 0 °C. The resulting mixture was stirred for 2 h at room temperature, then acidified to pH 3 with 1 M HCl. The resulting solution was extracted with DCM (3*50 mL). The combined organic layers were dried over anhydrous Na₂SO₄. After filtration, the filtrate was concentrated under reduced pressure to afford 2-(dimethylamino)-1,3-oxazole-5-carboxylic acid (400 mg, 94.37%) as an off-white solid. LC-MS: (M+H)⁺ found 157.1.

Step 3: A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (60 mg, 0.14 mmol, 1.00 equiv), DIEA (122.1 mg, 0.95 mmol, 7 equiv), 2-(dimethylamino)-1,3-oxazole-5-carboxylic acid (42.2 mg, 0.27 mmol, 2 equiv) and PyBOP (105.4 mg, 0.20 mmol, 1.5 equiv) in DMF (2 mL) was stirred for 1 h at room temperature. The resulting mixture was diluted with EtOAc (20 mL) and washed with briner (3*20 mL). The organic layer was dried over anhydrous Na₂SO₄. After filtration, the filtrate was concentrated under reduced pressure. The residue was purified by Prep-HPLC (Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 35% B to 60% B in 8 min; Wave Length: 220 nm nm; RT1 (min): 7.78) to afford 2-(dimethylamino)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,3-oxazole-5-carboxamide (26.6 mg, 33.82%) as a off-white solid. LC-MS: (M+H)⁺ found 583.20. ¹H NMR (400 MHz, DMSO-d₆) δ 8.96 (t, J=5.8 Hz, 1H), 8.02 (d, J=6.8 Hz, 1H), 7.79-7.74 (m, 1H), 7.53 (s, 1H), 6.85 (t, J=7.2 Hz, 1H), 6.28 (d, J=8.1 Hz, 1H), 6.20 (d, J=7.7 Hz, 1H), 4.98-4.68 (m, 3H), 3.62 (d, J=29.0 Hz, 1H), 3.07 (s, 7H), 2.83 (d, J=7.5 Hz, 1H), 2.19 (s, 4H), 2.07 (t, J=10.2 Hz, 2H), 1.68 (d, J=9.2 Hz, 1H).

### Example 29—Preparation of (2R)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,4-dioxane-2-carboxamide

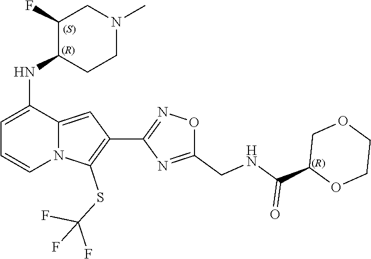

To a solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine hydrochloride (50.0 mg, 0.10 mmol, 1.00 equiv) in DMF (1.00 mL) were added (2S)-1,4-dioxane-2-carboxylic acid (13.7 mg, 0.10 mmol, 1.00 equiv), PyBOP (81.2 mg, 0.16 mmol, 1.50 equiv) and DIEA (134.4 mg, 1.04 mmol, 10.00 equiv). The resulting solution was stirred for 1 h at room temperature. The resulted solution was purified by reverse flash chromatography (Mobile Phase A: Water (0.1% FA), Mobile Phase B: ACN; Flow rate: 60 mL/min; Gradient: 0% B to 100% B in 30 min; 254/220 nm) and Prep-HPLC (XBridge Prep OBD C18 Column, 30*150 mm, 5 m; Mobile Phase A: Water (10 mmol/L NH₄HCO₃+0.05% NH₃—H₂O), Mobile Phase B: ACN; Flow rate: 60 mL/min; Gradient: 31% B to 57% B in 7 min; Wave Length: 254 nm/220 nm; RT(min): 7.52). This resulted in (2S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-1,4-dioxane-2-carboxamide (11.6 mg, 19.94%) as a white solid. LC-MS: (M+H)⁺ found 559.10. ¹H NMR (400 MHz, DMSO-d₆) δ 8.74 (t, J=5.9 Hz, 1H), 8.02 (d, J=6.8 Hz, 1H), 7.77 (s, 1H), 6.85 (t, J=7.2 Hz, 1H), 6.35-6.10 (m, 2H), 4.87 (d, J=49.5 Hz, 1H), 4.73-4.58 (m, 2H), 4.16 (dd, J=9.4, 3.1 Hz, 1H), 3.89 (td, J=11.1, 10.7, 2.9 Hz, 2H), 3.76-3.65 (m, 3H), 3.62-3.42 (m, 2H), 3.05 (t, J=11.2 Hz, 1H), 2.84 (d, J=7.9 Hz, 1H), 2.40-2.18 (m, 4H), 2.17-2.02 (m, 2H), 1.70 (d, J=9.4 Hz, 1H).

### Example 29—Preparation of 3-fluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}bicyclo[1.1.1]pentane-1-carboxamide

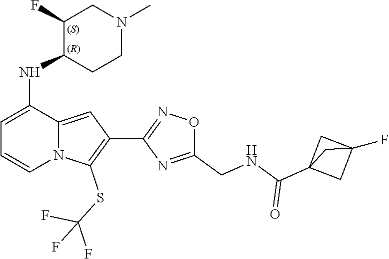

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), 3-fluorobicyclo[1.1.1]pentane-1-carboxylic acid (17.6 mg, 0.13 mmol, 1.2 eq), DIEA (72.7 mg, 0.56 mmol, 5 equiv) and PyBOP (70.3 mg, 0.13 mmol, 1.2 equiv) in DMF (1.5 mL) was stirred for 1 h at room temperature. The resulting solution was diluted with EtOAc (30 mL) and washed with brine (3*30 mL). The organic layer was dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 37% B to 60% B in 7 min; Wave Length: 254 nm/220 nm nm; RT1 (min): 6.52; Number Of Runs: 2) to afford 3-fluoro-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}bicyclo[1.1.1]pentane-1-carboxamide (19.6 mg, 31.02%) as a white solid. LC-MS: (M+H)⁺ found 557.15. ¹H NMR (400 MHz, DMSO-d₆) δ 8.90 (t, J=5.7 Hz, 1H), 8.02 (d, J=6.9 Hz, 1H), 7.78 (s, 1H), 6.86 (t, J=7.2 Hz, 1H), 6.24 (m, 2H), 4.87 (d, J=49.3 Hz, 1H), 4.65 (d, J=5.7 Hz, 2H), 3.64 (d, J=29.0 Hz, 1H), 3.05 (t, J=11.2 Hz, 1H), 2.84 (d, J=8.1 Hz, 1H), 2.33 (d, J=2.6 Hz, 7H), 2.20 (s, 3H), 2.14-2.00 (m, 2H), 1.70 (d, J=9.7 Hz, 1H).

### Example 62—Preparation of (1s,3s)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxycyclobutane-1-carboxamide

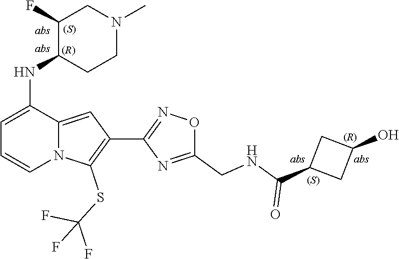

To a stirred mixture of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine hydrochloride (50.0 mg, 0.10 mmol, 1.00 equiv) in DMF (2.00 mL) were added PyBOP (81.2 mg, 0.15 mmol, 1.50 equiv), DIEA (134.4 mg, 1.04 mmol, 10.00 equiv) and (1s,3s)-3-hydroxycyclobutane-1-carboxylic acid (14.5 mg, 0.12 mmol, 1.20 equiv). The resulting solution was stirred for 1 h at room temperature. The resulted solution was purified by reverse flash chromatography (Mobile Phase A: Water, Mobile Phase B: ACN; Flow rate: 80 mL/min; Gradient: 0% B to 100% B in 30 min; 254/220 nm) and prep-HPLC (Column: XBridge Prep OBD C18 Column, 30*150 mm, 5 μm; Mobile Phase A: Water (10 mmol/L NH₄HCO₃+0.05% NH₃—H₂O), Mobile Phase B: ACN; Flow rate: 60 mL/min; Gradient: 25% B to 52% B in 7 min; Wave Length: 254 nm/220 nm; RT(min): 7.63). This resulted in (1s,3s)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-3-hydroxycyclobutane-1-carboxamide (14.9 mg, 25.9%) as an off-white solid. LC-MS: (M+H)⁺ found 543.15. ¹H NMR (400 MHz, DMSO-d₆) δ 8.66 (t, J=5.7 Hz, 1H), 8.02 (d, J=6.8 Hz, 1H), 7.77 (s, 1H), 6.86 (t, J=7.2 Hz, 1H), 6.32-6.16 (m, 2H), 5.15 (d, J=6.9 Hz, 1H), 4.87 (d, J=49.6 Hz, 1H), 4.62 (d, J=5.7 Hz, 2H), 3.95 (m, 1H), 3.76-3.57 (m, 1H), 3.05 (t, J=10.8 Hz, 1H), 2.84 (d, J=7.7 Hz, 1H), 2.47-2.02 (m, 9H), 1.97 (m, 2H), 1.70 (d, J=10.0 Hz, 1H).

### Example 66—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(morpholin-4-yl)-1,3-oxazole-5-carboxamide

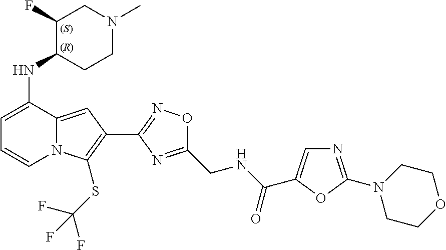

Step 1: A mixture of ethyl 2-chloro-1,3-oxazole-5-carboxylate (500 mg, 2.85 mmol, 1 equiv), morpholine (496.2 mg, 5.69 mmol, 2 equiv) and K₂CO₃ (1.18 g, 8.54 mmol, 3 equiv) in MeCN (8 mL) was stirred at 80 °C for 2 h. The resulting solution was stirred for 1 h at room temperature. The resulting mixture was diluted with water (30 mL) and washed with brine (3*30 mL). The organic layer was dried over anhydrous Na₂SO₄. After filtration, the filtrate was concentrated under reduced pressure to afford ethyl 2-(morpholin-4-yl)-1,3-oxazole-5-carboxylate (600 mg, 93.13%) as a yellow oil. LC-MS: (M+H)⁺ found 227.2.

Step 2: To a stirred solution of ethyl 2-(morpholin-4-yl)-1,3-oxazole-5-carboxylate (590 mg, 2.61 mmol, 1 equiv) in THF (4 mL)/water (4 mL) was added NaOH (208.6 mg, 5.22 mmol, 2 equiv). The reaction mixture was stirred at room temperature for 1 h. The resulting mixture concentrated under reduced pressure and then acidified to pH 6 with 1 M HCl. The precipitated solids were collected by filtration to afford 2-(morpholin-4-yl)-1,3-oxazole-5-carboxylic acid (500 mg, 96.74%) as an off-white solid. LC-MS: (M+H)⁺ found 199.0.

Step 3: To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (55 mg, 0.12 mmol, 1 equiv) in DMF (1.5 mL) was added 2-(morpholin-4-yl)-1,3-oxazole-5-carboxylic acid (49.1 mg, 0.25 mmol, 2 equiv), PyBOP (96.6 mg, 0.19 mmol, 1.5 equiv) and DIEA (80.0 mg, 0.62 mmol, 5 equiv) at 0 °C. The resulting solution was stirred for 1 h at room temperature. The resulting solution was diluted with EA (30 mL) and washed with brine (3*30 mL). The organic layer was dried over anhydrous Na₂SO₄. After filtration, the filtrate was concentrated under reduced pressure.

The residue was purified by Prep-HPLC (Column: XBridge Prep OBD C₁₈ Column, 30*150 mm, 5 μm; Mobile Phase A: Water (10 mmol/L NH₄HCO₃)+0.05% NH₃·H₂O, Mobile Phase B: ACN; Flow rate: 60 mL/min mL/min; Gradient: 30% B to 55% B in 7 min; Wave Length: 254 nm/220 nm nm; RT1 (min): 7.53) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-2-(morpholin-4-yl)-1,3-oxazole-5-carboxamide (24.5 mg, 31.19%) as an off-white solid. LC-MS: (M+H)~found 625.35. ¹H NMR (400 MHz, DMSO-d₆) 9.03 (t, J=5.8 Hz, 1H), 8.02 (d, J=6.9 Hz, 1H), 7.76 (s, 1H), 7.57 (s, 1H), 6.85 (t, J=7.2 Hz, 1H), 6.27 (d, J=8.1 Hz, 1H), 6.20 (d, J=7.6 Hz, 1H), 4.94-4.75 (m, 3H), 3.61 (dt, J=66.7, 4.7 Hz, 9H), 3.05 (t, J=11.3 Hz, 1H), 2.82 (s, 1H), 2.20 (s, 4H), 2.07 (t, J=10.8 Hz, 2H), 1.69 (d, J=9.9 Hz, 1H).

### Example 67—Preparation of N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}morpholine-4-carboxamide

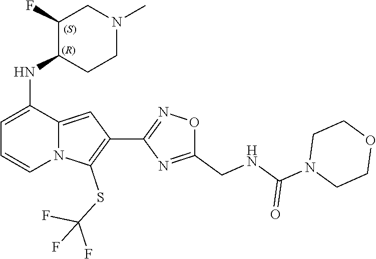

Step 1: To a stirred mixture of triphosgene (120.2 mg, 0.40 mmol, 1.2 equiv) in DCM (2 mL) was added 2-[5-(aminomethyl)-1,2,4-oxadiazol-3-yl]-N-[(3S,4R)-3-fluoro-1-methylpiperidin-4-yl]-3-[(trifluoromethyl) sulfanyl]indolizin-8-amine (150 mg, 0.34 mmol, 1 equiv) and DIEA (174.5 mg, 1.35 mmol, 4 equiv) dropwise at 0 °C. Then the resulting mixture was concentrated under reduced pressure. This resulted in N-[(3S,4R)-3-fluoro-1-methylpiperidin-4-yl]-2-[5-(isocyanatomethyl)-1,2,4-oxadiazol-3-yl]-3-[(trifluoromethyl)sulfanyl]indolizin-8-amine (100 mg, 62.98%) as a yellow solid. LC-MS: (M+H)⁺ found 471.1.

Step 2: A solution of N-[(3S,4R)-3-fluoro-1-methylpiperidin-4-yl]-2-[5-(isocyanatomethyl)-1,2,4-oxadiazol-3-yl]-3-[(trifluoromethyl)sulfanyl]indolizin-8-amine (50 mg, 0.11 mmol, 1 equiv) in DCM (1 mL) was treated with morpholine (18.5 mg, 0.21 mmol, 2 equiv) for 30 min at 0 °C. The resulting mixture was concentrated under reduced pressure. The residue was purified by Prep-HPLC (Column: XBridge Shield RP18 OBD Column, 30*150 mm, 5 μm; Mobile Phase A: Water (10 mmol/L NH₄HCO₃+0.05% NH₃—H₂O), Mobile Phase B: ACN; Flow rate: 60 mL/min mL/min; Gradient: 30% B to 50% B in 7 min; Wave Length: 254 nm/220 nm nm; RT1 (min): 6.53; Number Of Runs: 2) to afford N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}morpholine-4-carboxamide (19.7 mg, 33.24%) as an off-white solid. LC-MS: (M+H)⁺ found 558.25. ¹H NMR (400 MHz, DMSO-d₆) δ 8.02 (d, J=6.8 Hz, 1H), 7.78 (d, J=0.9 Hz, 1H), 7.52 (t, J=5.5 Hz, 1H), 6.85 (t, J=7.2 Hz, 1H), 6.24 (dd, J=29.1, 7.8 Hz, 2H), 4.87 (d, J=49.5 Hz, 1H), 4.58 (d, J=5.4 Hz, 2H), 3.75-3.53 (m, 5H), 3.32 (s, 4H), 3.05 (t, J=11.2 Hz, 1H), 2.83 (d, J=7.0 Hz, 1H), 2.39-2.14 (m, 4H), 2.08 (t, J=9.3 Hz, 2H), 1.69 (d, J=7.9 Hz, 1H).

### Example 68—Preparation of (3R)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methylmorpholine-3-carboxamide

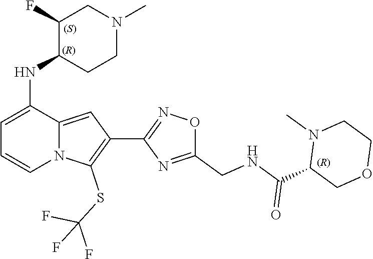

A solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), (3R)-4-methylmorpholine-3-carboxylic acid hydrochloride (24.5 mg, 0.13 mmol, 1.2 equiv), DIEA (43.6 mg, 0.34 mmol, 3 equiv), and PyBOP (76.1 mg, 0.15 mmol, 1.3 equiv) in DMF (1 mL) was stirred for 1 h at room temperature. The resulting mixture was diluted with EtOAc (50 mL) and washed with brien (2*50 mL). The organic layer was dried over anhydrous Na₂SO₄. After filtration, the filtrate was concentrated under reduced pressure. The residue was purified by Prep-HPLC (Column: XBridge Shield RP18 OBD Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient: 30% B to 55% B in 7 min; Wave Length: 254 nm/220 nm nm; RT1 (min): 6.27; Number Of Runs: 2) to afford (3R)-N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methylmorpholine-3-carboxamide (38.7 mg, 60.06%) as a white solid. LC-MS: (M+H)⁺ found 572.30. ¹H NMR (400 MHz, DMSO-d₆) δ 8.80 (t, J=5.9 Hz, 1H), 8.02 (d, I=6.8 Hz, 1H), 7.75 (d, J=0.9 Hz, 1H), 6.85 (t, J=7.2 Hz, 1H), 6.22 (t, J=7.8 Hz, 2H), 4.87 (d, J=49.4 Hz, 1H), 4.75-4.53 (m, 2H), 3.79 (dt, J=10.8, 3.5 Hz, 2H), 3.63 (d, J=28.7 Hz, 1H), 3.52 (td, J=11.4, 2.4 Hz, 1H), 3.44-3.38 (m, 1H), 3.10-3.01 (m, 1H), 2.88-2.66 (m, 3H), 2.25 (s, 3H), 2.19 (d, J=4.7 Hz, 4H), 2.09 (d, J=8.8 Hz, 3H), 1.70 (d, J=9.5 Hz, 1H).

### Example 69—Preparation of (3S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methylmorpholine-3-carboxamide

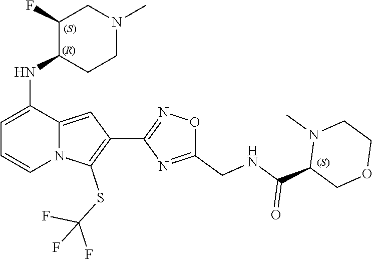

To a stirred solution of 2-[5-(aminomethyl)-1,2,4-oxadiazol-3-yl]-N-[(3S,4R)-3-fluoro-1-methylpiperidin-4-yl]-3-[(trifluoromethyl)sulfanyl]indolizin-8-amine (50 mg, 0.11 mmol, 1 equiv), (3S)-4-methyl-3-morpholinecarboxylic acid hydrochloride (25 mg, 0.13 mmol, 1.2 equiv) and DIEA (0.10 mL, 0.57 mmol, 5.10 equiv) in DMF (1 mL) was added PyBOP (71 mg, 0.13 mmol, 1.2 equiv) at 0 °C. The resulting solution was stirred for 1 h at room temperature. The resulting solution was diluted with EtOAc (30 mL) and washed with brine (2*30 mL). The organic layer was dried over anhydrous Na₂SO₄. After filtration, the filtrate was concentrated under reduced pressure. The residue was purified by Prep-HPLC (Column: XBridge Shield RP18 OBD Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 30% B to 55% B in 7 min; Wave Length: 254 nm/220 nm nm; RT1 (min): 6.27) to afford (3S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methylmorpholine-3-carboxamide (33.9 mg, 52.35%) as a off-white solid. LC-MS: (M+H)⁺ found 572.25. ¹H NMR (400 MHz, DMSO-d₆) δ 8.80 (t, J=5.9 Hz, 1H), 8.02 (d, J=6.9 Hz, 1H), 7.76 (d, J=0.9 Hz, 1H), 6.85 (t, J=7.2 Hz, 1H), 6.22 (t, J=8.5 Hz, 2H), 4.87 (d, J=49.4 Hz, 1H), 4.73-4.56 (m, 2H), 3.79 (dt, J=11.3, 3.5 Hz, 2H), 3.72-3.45 (m, 2H), 3.39 (dd, 0.1=11.1, 10.1 Hz, 1H), 3.05 (t, J=11.4 Hz, 1H), 2.89-2.61 (m, 3H), 2.36-2.14 (m, 8H), 2.13-2.01 (m, 2H), 1.70 (d, J=9.9 Hz, 1H).

### Example 5—Preparation of (3S)—N-{[3-(8-{[(3S,4R)-3-fluoro-1-methylpiperidin-4-yl]amino}-3-[(trifluoromethyl)sulfanyl]indolizin-2-yl)-1,2,4-oxadiazol-5-yl]methyl}-4-methylmorpholine-3-carboxamide

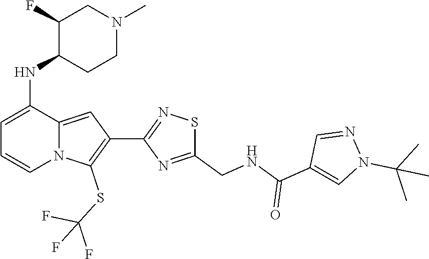 [SMILES: CN1CC[C@@H](Nc2cccn3c(SC(F)(F)F)c(-c4nsc(CNC(=O)c5cnn(C(C)(C)C)c5)n4)cc23)[C@@H](F)C1]

Step 1: A mixture of 1-(3-{8-bromo-3-[(trifluoromethyl)sulfanyl]indolizin-2-yl}-1,2,4-thiadiazol-5-yl)methanamine (100 mg, 0.24 mmol, 1 equiv), 1-tert-butylpyrazole-4-carboxylic acid (49.3 mg, 0.29 mmol, 1.2 equiv), HATU (92.9 mg, 0.24 mmol, 1 equiv) and DIEA (94.8 mg, 0.73 mmol, 3 equiv) in DMF (1 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (PE/EA 2:1) to afford N-[(3-{8-bromo-3-[(trifluoromethyl)sulfanyl]indolizin-2-yl}-1,2,4-thiadiazol-5-yl)methyl]-1-tert-butylpyrazole-4-carboxamide (60 mg, 43.89%) as a light yellow solid. LC-MS: (M+H)⁺ found 559.3.

Step 2: A mixture of N-[(3-{8-bromo-3-[(trifluoromethyl)sulfanyl]indolizin-2-yl}-1,2,4-thiadiazol-5-yl)methyl]-1-tert-butylpyrazole-4-carboxamide (60 mg, 0.11 mmol, 1 equiv), Pd-PEPPSI-IHeptCl 3-chloropyridine (52.2 mg, 0.05 mmol, 0.5 equiv) and Cs₂CO₃ (209.7 mg, 0.64 mmol, 6 equiv) in dioxane (1 mL) was stirred for 2 h at 100 °C under nitrogen atmosphere. The resulting mixture was filtered and the filter cake was washed with MeCN. The filtrate was concentrated under reduced pressure. The residue was purified by Prep-TLC (CH₂Cl₂/7 M NH₃ in MeOH=20:1) and Prep-HPLC (Column: XBridge Shield RP18 OBD Column, 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 40% B to 63% B in 7 min; Wave Length: 254 nm/220 nm nm; RT1 (min): 6.47; Number Of Runs: 1) to afford 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}pyrazole-4-carboxamide (8.6 mg, 13.13%) as a light yellow solid. to afford 1-tert-butyl-N—{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}pyrazole-4-carboxamide (8.6 mg, 13.13%) as a light yellow solid. LC-MS: (M+H)⁺ found 611.15. ¹H NMR (400 MHz, DMSO-d₆) δ 9.16 (t, J=5.8 Hz, 1H), 8.36 (s, 1H), 8.03 (d, J=6.9 Hz, 1H), 7.95 (s, 1H), 7.85 (s, 1H), 6.82 (t, J=7.2 Hz, 1H), 6.20 m, 2H), 5.00-4.78 (m, 3H), 3.82-3.52 (m, 1H), 3.08 (s, 1H), 2.86 (s, 1H), 2.23 (s, 4H), 2.19-2.03 (m, 2H), 1.72 (d, J=11.6 Hz, 1H), 1.54 (s, 9H).

### Example 6—Preparation of 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}pyrrole-3-carboxamide

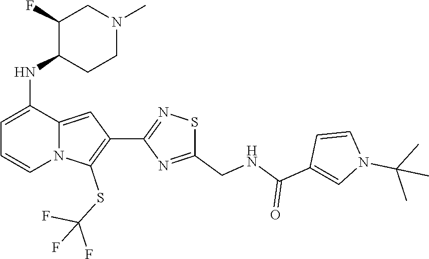 [SMILES: CN1CC[C@@H](Nc2cccn3c(SC(F)(F)F)c(-c4nsc(CNC(=O)c5ccn(C(C)(C)C)c5)n4)cc23)[C@@H](F)C1]

Step 1: A mixture of 1-(3-{8-bromo-3-[(trifluoromethyl)sulfanyl]indolizin-2-yl}-1,2,4-thiadiazol-5-yl)methanamine (100 mg, 0.244 mmol, 1 equiv), 1-tert-butylpyrrole-3-carboxylic acid (49.0 mg, 0.29 mmol, 1.20 equiv), HATU (92.91 mg, 0.24 mmol, 1.00 equiv) and DIEA (94.8 mg, 0.73 mmol, 3.00 equiv) in DMF (2 mL) was stirred for 1 h at room temperature. The resulting mixture was diluted with EtOAc (50 mL) and washed with brine (3*50 mL). The organic layer was dried over anhydrous Na₂SO₄. After filtration, the filtrate was concentrated under reduced pressure. The residue was purified by Prep-TLC (PE/EA 2:1) to afford N-[(3-{8-bromo-3-[(trifluoromethyl)sulfanyl]indolizin-2-yl}-1,2,4-thiadiazol-5-yl)methyl]-1-tert-butylpyrazole-4-carboxamide (40 mg, 29.26%) as a light yellow solid. LC-MS: (M+H)⁺ found 558.3.

Step 2: A mixture of N-[(3-{8-bromo-3-[(trifluoromethyl)sulfanyl]indolizin-2-yl}-1,2,4-thiadiazol-5-yl)methyl]-1-tert-butylpyrrole-3-carboxamide (40 mg, 0.07 mmol, 1 equiv), Pd-PEPPSI-IHeptCl 3-chloropyridine (34.9 mg, 0.04 mmol, 0.5 equiv) and Cs₂CO₃ (140.0 mg, 0.43 mmol, 6 equiv) in dioxane (1 mL) was stirred for 2 h at 100 °C under nitrogen atmosphere. The resulting mixture was filtered and the filter cake was washed with MeCN. The filtrate was concentrated under reduced pressure. The residue was purified by Prep-TLC (CH₂Cl₂/7M NH₃ in MeOH 20:1) and Prep-HPLC (Column: Waters Xbridge C18 OBD Column 30*150 mm 5 μm, Mobile Phase A: Water(10 mmol/L NH₄HCO₃+0.5% NH₃—H₂O), Mobile Phase B: ACN; Flow rate: 60 mL/min mL/min; Gradient: 43% B to 70% B in 7 min; Wave Length: 254/220 nm; RT1 (min): 6.28) to afford 1-tert-butyl-N-{[3-(8-{[(3S,4R)-3-fluoro-1-methylpiperidin-4-yl]amino}-3-[(trifluoromethyl)sulfanyl]indolizin-2-yl)-1,2,4-thiadiazol-5-yl]methyl}pyrrole-3-carboxamide (1.5 mg, 3.43%) as a light yellow solid. LC-MS: (M+H)⁺ found 610.20. ¹H NMR (400 MHz, DMSO-d₆) δ 8.86 (d, J=5.6 Hz, 1H), 8.03 (d, J=6.8 Hz, 1H), 7.84 (s, 1H), 7.57 (s, 1H), 7.01 (d, J=2.8 Hz, 1H), 6.82 (t, J=7.1 Hz, 1H), 6.52 (d, J=2.7 Hz, 1H), 6.19 (t, J=8.3 Hz, 2H), 5.05-4.69 (m, 3H), 3.72 (m, 1H), 3.06 (s, 1H), 2.85 (s, 1H), 2.21 (s, 4H), 2.09 (d, J=10.5 Hz, 2H), 1.72 (s, 1H), 1.50 (s, 9H).

### Assays

### Surface Plasmon Resonance (SPR) Assay Protocol

SPR experiments are performed on a Biacore 8K instrument. Biotinylated recombinant p53 Y220C mutant protein (amino acid residues 94-293) is immobilized onto a streptavidin sensor chip (Sensor Chip SA), by flowing the protein solution through the sensor chip at typically 10 μg/mL concentration, 5 μL/min flow rate for 70 seconds. Compounds are 2-fold, 7-point serial diluted; the top concentration varies depending on the potency. Compound binding affinities are measured in the multi-cycle kinetics mode, at 30 μL/min flow rate with 60 seconds association time and 120 seconds dissociation time. The running buffer contains 50 mM Tris, pH 7.5, 100 mM NaCl, 1 mM DTT, 0.01% Brij35, 0.05% Tween-20 and 1% DMSO. The assay temperature is maintained at 16 or 20 °C and data are fit into the 1:1 binding model using the Biacore Insight Evaluation software.

### Thermal Shift Assay Protocol

5 μM recombinant p53 Y220C mutant protein (amino acid residues 94-293) is incubated with 100 μM compound for 3 hours at 20 °C in buffer containing 20 mM HEPES, pH7.4, 100 mM NaCl, 0.01% Pluronic F-127, and 1% DMSO in the presence of 400-fold diluted Sypro Orange (ThermoFisher Scientific catalog number 4461146), on a 384-well PCR plate (Applied Biosystems catalog number 4483285). The volume is 5 μL/well. The sealed plate is then loaded onto a QuantStudio 7 Flex instrument for melting temperature (T_{M}) measurement. The temperature is increased from 30 °C to 50 °C, at a rate of 0.03 °C/second. Data are fit into a Boltzmann two-state model to determine the T_{M}.

### Luciferase Reporter Assay Protocol

Luciferase reporter (Luc) driven by p53 response element (LTV-p53-Luc (SKU #: LTLR007); G&P Biosciences) and inducible p53 Y220C construct (Tet-One inducible expression system; Takara) were stably expressed in NCIH1299 cells by lentiviral transduction. The cells were then used for p53 reporter assay. 5,000 cells were seeded in each well of 384 plate and cultured in 0.5 ug/mL doxycycline (D3072; Sigma) containing medium for 24 hours before compound treatment. After 6 hour of compound treatment, equal volume of One-Glo reagent (E6110; Promega) was added to each well and the plate was incubated for 5 minutes with shaking at room temperature. The luciferase activity was immediately measured with PheraStar microplate reader. Luciferase activity was stimulated by 10 uM of a tool compound as positive control and AC₅₀ was calculated.

### Cell Proliferation Assay

Inducible p53 Y220C and R273H constructs (Tet-One inducible expression system; Takara) were stably expressed in NCIH1299 cells by lentiviral transduction. NUGC3, A549, BxPC3, T3M4, HuH7, HCC2935, MFE296, NCIH1299, NCIH1299-inducible p53 Y220C, and NCIH1299-inducible p53 R273H cell lines were used for cell proliferation assay. 250-500 cells were seeded in each well of 384 plate and then compounds were dispensed into each well using Echo or Tecan. After 5 days of incubation, equal volume of CellTiter-Glo reagent (G7570; Promega) was added into each well and the plate was incubated at room temperature for 10 minutes with shaking. The luminescent signal was measured by PheraStar microplate reader and IC₅₀ was calculated.

Table A shows results of the SPR p53 Y220C Kd_kinetic (nM), TSA p53 Y220C dTM (deg C), Luc p53 Y220C AC₅₀ (nM), CTG H1299 p53 Y220C IC₅₀ (nM), CTG H1299 p53 R273H IC₅₀ (nM), and CTG NUGC3 p53 Y220C IC₅₀ (nM) assays. Activity categories are as follows:
SPR p53 Y220C Kd_kinetic (nM): D≥1000≥C≥100>B≥10>A
TSA p53 Y220C dTM (deg C, 100 μM): A≥10>B≥5>C≥1>D
TSA p53Y220C dTM (deg C, 10 μM): A≥10>B≥5>C≥1>D
CTG NUGC3 p53 Y22C IC₅₀ (nM): D≥3000>C≥1500>B≥750>A
CTG H1299 p53 Y220C IC₅₀ (nM): D≥3000>C≥1500>B≥750>A
CTG H1299 p53 R273H IC₅₀ (nM): A≥10000>B≥8000>C≥5000>D
Luc p53 Y220C AC₅₀ (nM): D≥3000>C≥1500>B≥750>A

TABLE A

| | SPR P53 | | TSA P53 | | | | |
|---|---|---|---|---|---|---|---|
| | Y220C | TSA P53 | Y220C dTM | Luc P53 | CTG NUGC3 | CTG H1299 | CTG H1299 |
| Example | Kd_kinetic | Y220C dTM | (deg C, | Y220C | P53 Y220C | P53 Y220C | P53 R273H |
| No. | (nM) | (deg C) | 10 uM) | AC50 (nM) | IC50 (nM) | IC50 (nM) | IC50 (nM) |
| 1 | B | B | B | D | D | D | A |
| 2 | A | | | A | A | A | |
| 3 | A | | | A | A | A | |
| 4 | B | | | D | D | D | |
| 5 | C | | | D | D | D | |
| 6 | D | | | D | D | D | |
| 7 | A | | | A | A | A | |
| 8 | A | | | C | C | B | |
| 9 | C | | | D | C | C | |
| 10 | B | | | C | D | D | |
| 11 | A | | | B | B | B | |
| 12 | A | | | A | A | A | |
| 13 | A | | | A | A | A | |
| 14 | B | | | C | B | B | |
| 15 | A | | | A | A | A | |
| 16 | A | | | B | A | B | |
| 17 | A | | | A | A | B | |
| 18 | A | | | B | A | B | |
| 19 | B | | | B | A | A | |
| 20 | A | | | A | A | A | |
| 21 | C | | | C | C | C | |
| 22 | B | | | D | C | D | |
| 23 | A | | | A | A | A | |
| 24 | A | | | A | B | B | |
| 25 | A | | | C | C | C | |
| 26 | B | | | C | C | C | |
| 27 | A | | | A | A | B | |
| 28 | A | | | A | A | A | |
| 29 | A | | | B | B | B | |
| 30 | B | | | B | C | C | |
| 31 | A | | | A | A | A | |
| 32 | A | | | A | A | A | |
| 33 | A | | | A | A | A | |
| 34 | B | | | D | C | C | |
| 35 | B | | | D | C | C | |
| 36 | B | | | C | C | C | |
| 37 | A | | | A | A | A | |
| 38 | B | | | B | A | A | |
| 39 | B | | | B | A | A | |
| 40 | B | | | A | A | A | |
| 41 | B | | | B | B | B | |
| 42 | A | | | A | A | A | |
| 43 | B | | | B | B | B | |
| 44 | A | | | A | B | A | |
| 45 | B | | | D | C | D | |
| 46 | C | | | D | D | D | |
| 47 | B | | | D | D | D | |
| 48 | B | | | D | D | D | |
| 49 | B | | | C | D | C | |
| 50 | A | | | A | B | | |
| 51 | B | | | D | D | D | |
| 52 | B | | | D | D | D | |
| 53 | B | | | D | D | | |
| 54 | A | | | A | A | A | |
| 55 | B | | | D | D | | |
| 56 | B | | | B | A | A | |
| 57 | A | | | A | A | A | |
| 58 | A | | | A | A | A | |
| 59 | A | | | B | B | A | |
| 60 | B | | | C | C | C | |
| 61 | B | | | D | D | D | |
| 62 | A | | | C | C | B | |
| 63 | A | | | C | C | D | |
| 64 | B | | | D | D | D | |
| 65 | B | | | D | D | D | |
| 66 | A | | | A | A | A | |
| 67 | A | | | B | C | B | |
| 68 | B | | | D | D | | |
| 69 | B | | | D | D | | |
| 70 | A | | | A | A | | |
| 71 | A | | | A | A | | |
| 72 | A | | | A | A | | |
| 73 | A | | | A | B | | |
| 74 | A | | | B | C | | |
| 75 | A | | | B | B | | |
| 76 | A | | | A | B | | |
| 77 | A | | | A | A | | |
| 78 | A | | | A | A | | |
| 79 | A | | | A | A | | |
| 80 | A | | | C | C | | |
| 81 | A | | | A | B | | |
| 82 | A | | | A | A | | |
| 83 | A | | | B | B | | |
| 84 | B | | | D | D | | |
| 85 | A | | | A | A | | |
| 86 | A | | | A | | | |
| 87 | A | | | B | | | |
| 88 | B | | | A | | | |
| 89 | A | | | A | A | | |
| 90 | A | | | A | | | |
| 91 | A | | | A | | | |
| 92 | A | | | A | | | |
| 93 | A | | | A | A | | |
| 94 | A | | | A | A | | |
| 95 | A | | | A | | | |
| 96 | A | | | A | | | |
| 97 | A | | | A | | | |
| 98 | A | | | B | B | | |
| 99 | | | | | | | |
| 100 | | | | | | | |
| 101 | | | | | | | |
| 102 | | | | | | | |
| 103 | | | | | | | |
| 104 | | | | | | | |
| 105 | | | | | | | |
| 106 | | | | | | | |
| 107 | | | | | | | |
| 108 | | | | | | | |
| 109 | | | | | | | |
| 110 | | | | | | | |

What is claimed is:

1. A compound of Formula (I) 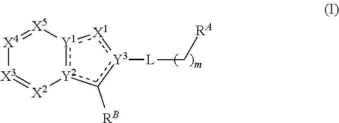
or a pharmaceutically acceptable salt thereof, wherein:
X¹ is CR¹ or N;
R¹ is hydrogen, halogen, cyano, —OR⁴, —NR⁴R⁵, —C(═O)R⁴, —OC(═O)R⁴, —C(═O)OR⁴, —C(═O)NR⁴R⁵, —SR⁴, —S(═O)R⁴, —S(O₂)R⁴, —NR⁴C(═O)R⁵, —R⁴C(1═O(RP, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
each of X², X³, X⁴, and X⁵ are CH, N, CR² or CR³, wherein two or more of X², X³, X⁴, and X⁵ are independently CH, CR², or CR³;
each of Y¹, Y², and Y³ are C or N, wherein one of Y¹, Y², and Y³ is N;
R^{A} is hydrogen, —OR⁶, —NR⁶R⁷, —C(═O)R⁶, —R⁶C(═O)R⁷, —OC(═O)R⁶, —OC(═O)NR⁶, —C(═O)OR⁶, —NR⁶C(═O)OR⁷, —C(═O)NR⁶R⁷, —SR⁶, —S(═O)R⁶, —S(O₂)R⁶, —S(O₂)NR⁶, —NR⁶S(O₂)R⁷, —NR⁶C(═O)R⁷, —NR⁶C(═O)NR⁷, —SiR⁶R⁷R⁸, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl;
R^{B} is halogen, cyano, hydroxyl, —NR⁸R⁹, —OR⁸, —C(═O)NR⁸R⁹, —C(═O)R⁸, —C(═O)OR⁸, —NR⁸C(═O)OR⁹, —OC(═O)R⁸, —OC(═O)NR⁸, —C(═O)NR⁸R⁹, —NR⁸C(═O)R⁹, —NR⁸C(═O)NR⁹, —SR⁸, —S(═O)R⁸, —S(O₂)R⁸, —S(O₂)NR⁸, —NR⁸S(O₂)R⁹, —R⁸C(═O)R⁹, —NR⁸C(═O)R⁹, —NR⁸C(═O)NR⁹, optionally substituted C1-C6 alkyl, C1-C6 haloalkyl, optionally substituted C2-C6 alkenyl, optionally substituted C2-C6 alkynyl, optionally substituted C3-C10 cycloalkyl, optionally substituted phenyl, optionally substituted 3-12 membered heterocyclyl, or optionally substituted 5-10 membered heteroaryl;
each R² is 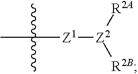
Z¹ is a bond, —C═O—, —S(O₂)-optionally substituted C1-C6 alkylene, optionally substituted C2-C6 alkenylene, optionally substituted C2-C6 alkynylene, or an optionally substituted C3-C4 cycloalkylene;
Z² is CR^{2C}, N, O, or a bond;
wherein when Z² is O, R^{2B} is absent; when Z¹ is a bond and Z² is a bond, R^{2B} is absent and R^{2A} is directly connected to Formula (I) via Z¹;
R^{2A} and R^{2B} are independently hydrogen, —C(═O)R¹⁰, —C(═O)OR¹⁰, —C(═O)NR¹⁰R¹¹, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl; or
R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl; or Z² is 0 and R^{2B} is absent;
R²C is hydrogen, halogen, or C1-C6 alkyl;
each R³ is independently halogen, cyano, —NR¹²R¹³, —OR¹², —C(═O)NR¹²R¹³, —C(═O)R¹², —C(═O)OR¹², —OC(═O)R¹², —NR¹²(C═O)NR¹²R¹³, —SR¹², —S(═O)R¹², —S(O₂)R¹², —S(O₂)NR¹²R¹³, —NR¹²S(O₂)NR¹³R¹⁴, —R¹²C(═O)R¹³, —NR¹²C(═O)R¹³, optionally substituted C1-C6 alkyl, optionally substituted C2-C6 alkenyl, optionally substituted C2-C6 alkynyl, optionally substituted C3-C6 cycloalkyl, optionally substituted phenyl, optionally substituted 4-6 membered heterocyclyl, or optionally substituted 5-6 membered heteroaryl;
L is an optionally substituted 4-6 membered heterocyclylene or an optionally substituted 5-6 membered heteroarylene;
m is 0, 1, or 2; and
each R⁴, R⁵, R⁶, R⁷, R⁸, R⁹, R¹⁰, R¹¹, R¹², R¹³, and R¹⁴ are independently hydrogen, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, or optionally substituted 5-10 membered heteroaryl.

2. The compound of claim 1, wherein the compound of Formula (I), or a pharmaceutically acceptable salt thereof, is selected from the group consisting of: 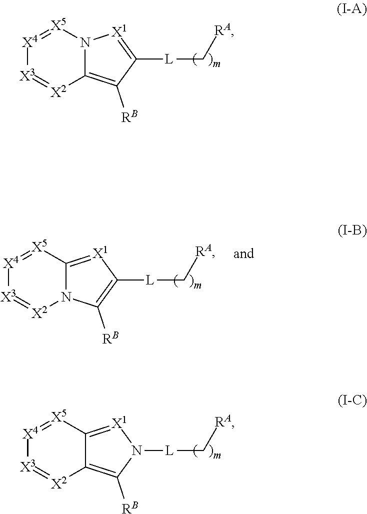
or a pharmaceutically acceptable salt of any of the foregoing.

3. The compound of claim 1 or 2, wherein one of X², X³, X⁴, and X⁵ is N.

4. The compound of claim 1 or 2, wherein two of X², X³, X⁴, and X⁵ are N.

5. The compound of any one of claims 1-4, wherein X¹ is CR¹.

6. The compound of any one of claims 1-5, wherein R¹ is hydrogen.

7. The compound of any one of claims 1-5, wherein R¹ is halogen.

8. The compound of any one of claims 1-5, wherein R¹ is cyano.

9. The compound of any one of claims 1-5, wherein R¹ is —OR⁴.

10. The compound of any one of claims 1-5, wherein R¹ is —NR⁴R⁵.

11. The compound of any one of claims 1-5, wherein R¹ is —C(═O)R⁴.

12. The compound of any one of claims 1-5, wherein R¹ is —OC(═O)R⁴.

13. The compound of any one of claims 1-5, wherein R¹ is —C(═O)OR⁴.

14. The compound of any one of claims 1-5, wherein R¹ is —C(═O)NR⁴R⁵.

15. The compound of any one of claims 1-5, wherein R¹ is —SR⁴.

16. The compound of any one of claims 1-5, wherein R¹ is —S(═O)R⁴.

17. The compound of any one of claims 1-5, wherein R¹ is —S(O₂)R⁴.

18. The compound of any one of claims 1-5, wherein R¹ is —NR⁴C(═O)R⁵.

19. The compound of any one of claims 1-5, wherein R¹ is —R⁴C(═O)R⁵.

20. The compound of any one of claims 1-5, wherein R¹ is optionally substituted C1-C6 alkyl.

21. The compound of any one of claims 1-5, wherein R¹ is optionally substituted C2-C6 alkenyl.

22. The compound of any one of claims 1-5, wherein R¹ is optionally substituted C2-C6 alkynyl.

23. The compound of any one of claims 1-5, wherein R¹ is optionally substituted C3-C10 cycloalkyl.

24. The compound of any one of claims 1-5, wherein R¹ is optionally substituted phenyl.

25. The compound of any one of claims 1-5, wherein R¹ is optionally substituted 4-12 membered heterocyclyl.

26. The compound of any one of claims 1-5, wherein R¹ is optionally substituted 5-10 membered heteroaryl.

27. The compound of any one of claims 1-4, wherein X¹ is N.

28. The compound of any one of claims 1-27, wherein R^{A} is hydrogen.

29. The compound of any one of claims 1-27, wherein R^{A} is —OR⁶.

30. The compound of any one of claims 1-27, wherein R^{A} is —NR⁶R⁷.

31. The compound of any one of claims 1-27, wherein R^{A} is —C(═O)R⁶.

32. The compound of any one of claims 1-27, wherein R^{A} is —R⁶C(═O)R⁷.

33. The compound of any one of claims 1-27, wherein R^{A} is —OC(═O)R⁶.

34. The compound of any one of claims 1-27, wherein R^{A} is —OC(═O)NR⁶.

35. The compound of any one of claims 1-27, wherein R^{A} is —C(═O)OR⁶.

36. The compound of any one of claims 1-27, wherein R^{A} is —NR⁶C(═O)OR⁷.

37. The compound of any one of claims 1-27, wherein R^{A} is —C(═O)NR⁶R⁷.

38. The compound of any one of claims 1-27, wherein R^{A} is —SR⁶.

39. The compound of any one of claims 1-27, wherein R^{A} is —S(═O)R⁶.

40. The compound of any one of claims 1-27, wherein R^{A} is —S(O₂)R⁶.

41. The compound of any one of claims 1-27, wherein R^{A} is —S(O₂)NR⁶.

42. The compound of any one of claims 1-27, wherein R^{A} is —NR⁶S(O₂)R⁷.

43. The compound of any one of claims 1-27, wherein R^{A} is —NR⁶C(═O)R⁷.

44. The compound of any one of claims 1-27, wherein R^{A} is —NR⁶C(═O)NR⁷.

45. The compound of any one of claims 1-27, wherein R^{A} is —SiR⁶R⁷R⁸.

46. The compound of any one of claims 1-27, wherein R^{A} is optionally substituted C1-C6 alkyl.

47. The compound of any one of claims 1-27, wherein R^{A} is optionally substituted C2-C6 alkenyl.

48. The compound of any one of claims 1-27, wherein R^{A} is optionally substituted C2-C6 alkynyl.

49. The compound of any one of claims 1-27, wherein R^{A} is optionally substituted C3-C10 cycloalkyl.

50. The compound of any one of claims 1-27, wherein R^{A} is optionally substituted phenyl.

51. The compound of any one of claims 1-27, wherein R^{A} is optionally substituted 4-12 membered heterocyclyl.

52. The compound of any one of claims 1-27, wherein R^{A} is optionally substituted 5-10 membered heteroaryl.

53. The compound of any one of claims 1-52, wherein R^{B} is halogen.

54. The compound of any one of claims 1-52, wherein R^{B} is cyano.

55. The compound of any one of claims 1-52, wherein R^{B} is hydroxyl.

56. The compound of any one of claims 1-52, wherein R^{B} is —NR⁸R⁹.

57. The compound of any one of claims 1-52, wherein R^{B} is —OR⁸.

58. The compound of any one of claims 1-52, wherein R^{B} is —C(═O)NR⁸R⁹.

59. The compound of any one of claims 1-52, wherein R^{B} is —C(═O)R⁸.

60. The compound of any one of claims 1-52, wherein R^{B} is —C(═O)OR⁸.

61. The compound of any one of claims 1-52, wherein R^{B} is —NR⁸C(═O)OR⁹

62. The compound of any one of claims 1-52, wherein R^{B} is —OC(═O)R⁸.

63. The compound of any one of claims 1-52, wherein R^{B} is —OC(═O)NR⁸.

64. The compound of any one of claims 1-52, wherein R^{B} is —C(═O)NR⁸R⁹.

65. The compound of any one of claims 1-52, wherein R^{B} is —NR⁸C(═O)R⁹.

66. The compound of any one of claims 1-52, wherein R^{B} is —NR⁸C(═O)NR⁹.

67. The compound of any one of claims 1-52, wherein R^{B} is —SR⁸.

68. The compound of any one of claims 1-52, wherein R^{B} is —S(═O)R⁸.

69. The compound of any one of claims 1-52, wherein R^{B} is —S(O₂)R⁸.

70. The compound of any one of claims 1-52, wherein R^{B} is —S(O₂)NR⁸.

71. The compound of any one of claims 1-52, wherein R^{B} is —NR⁸S(O₂)R⁹.

72. The compound of any one of claims 1-52, wherein R^{B} is —R⁸C(═O)R⁹.

73. The compound of any one of claims 1-52, wherein R^{B} is —NR⁸C(═O)R⁹.

74. The compound of any one of claims 1-52, wherein R^{B} is —NR⁸C(═O)NR⁹.

75. The compound of any one of claims 1-52, wherein R^{B} is optionally substituted C1-C6 alkyl.

76. The compound of any one of claims 1-52, wherein R^{B} is C1-C6 haloalkyl.

77. The compound of any one of claims 1-52, wherein R^{B} is optionally substituted C2-C6 alkenyl.

78. The compound of any one of claims 1-52, wherein R^{B} is optionally substituted C2-C6 alkynyl.

79. The compound of any one of claims 1-52, wherein R^{B} is optionally substituted C3-C10 cycloalkyl.

80. The compound of any one of claims 1-52, wherein R^{B} is optionally substituted phenyl.

81. The compound of any one of claims 1-52, wherein R^{B} is optionally substituted 3-12 membered heterocyclyl.

82. The compound of any one of claims 1-52, wherein R^{B} is optionally substituted 5-10 membered heteroaryl.

83. The compound of any one of claims 1-82, wherein each R² is

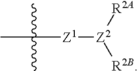

84. The compound of any one of claims 1-83, wherein one of X², X³, X⁴, and X⁵ is CR² and the remaining X², X³, X⁴, and X⁵ are CH, N, or CR³.

85. The compound of any one of claims 1-84, wherein Z¹ is a bond.

86. The compound of any one of claims 1-84, wherein Z¹ is —C═O—.

87. The compound of any one of claims 1-84, wherein Z¹ is —S(O₂)—.

88. The compound of any one of claims 1-84, wherein Z¹ is optionally substituted C1-C6 alkylene.

89. The compound of any one of claims 1-84, wherein Z¹ is optionally substituted C2-C6 alkenylene.

90. The compound of any one of claims 1-84, wherein Z¹ is optionally substituted C2-C6 alkynylene.

91. The compound of any one of claims 1-84, wherein Z¹ is an optionally substituted C3-C4 cycloalkylene.

92. The compound of any one of claims 1-91, wherein Z² is N.

93. The compound of any one of claims 1-91, wherein Z² is O and R²¹ is absent.

94. The compound of any one of claims 1-91, wherein Z² is a bond.

95. The compound of any one of claims 1-91, wherein Z² is CR²C.

96. The compound of any one of claims 1-91 or 95, wherein R^{2C} is hydrogen.

97. The compound of any one of claims 1-91 or 95, wherein R^{2C} is halogen.

98. The compound of any one of claims 1-91 or 95, wherein R²C is C1-C6 alkyl.

99. The compound of any one of claims 1-84, wherein Z¹ is a bond and Z² is a bond, R^{2B} is absent and R^{2A} is directly connected to Formula (I) via Z¹.

100. The compound of any one of claims 1-99, wherein R^{2A} is hydrogen.

101. The compound of any one of claims 1-99, wherein R^{2A} is —C(═O)R¹⁰.

102. The compound of any one of claims 1-99, wherein R^{2A} is —C(═O)OR¹⁰.

103. The compound of any one of claims 1-99, wherein R^{2A} is —C(═O)NR¹⁰R¹¹.

104. The compound of any one of claims 1-99, wherein R^{2A} is —S(═O)R¹⁰.

105. The compound of any one of claims 1-99, wherein R^{2A} is —S(O₂)R¹⁰.

106. The compound of any one of claims 1-99, wherein R^{2A} is optionally substituted C1-C6 alkyl.

107. The compound of any one of claims 1-99, wherein R^{2A} is optionally substituted C2-C6 alkenyl.

108. The compound of any one of claims 1-99, wherein R^{2A} is optionally substituted C2-C6 alkynyl.

109. The compound of any one of claims 1-99, wherein R^{2A} is optionally substituted C3-C10 cycloalkyl.

110. The compound of any one of claims 1-99, wherein R^{2A} is optionally substituted phenyl.

111. The compound of any one of claims 1-99, wherein R^{2A} is optionally substituted 4-12 membered heterocyclyl.

112. The compound of any one of claims 1-99, wherein R^{2A} is optionally substituted 5-10 membered heteroaryl.

113. The compound of any one of claims 1-98 and 100-112, wherein R^{2B} is hydrogen.

114. The compound of any one of claims 1-98 and 100-112, wherein R^{2B} is —C(═O)R¹⁰.

115. The compound of any one of claims 1-98 and 100-112, wherein R^{2B} is —C(═O)OR¹⁰.

116. The compound of any one of claims 1-98 and 100-112, wherein R^{2B} is —C(═O)NR¹⁰R¹¹.

117. The compound of any one of claims 1-98 and 100-112, wherein R^{2B} is —S(═O)R¹⁰.

118. The compound of any one of claims 1-98 and 100-112, wherein R^{2B} is —S(O₂)R¹⁰.

119. The compound of any one of claims 1-98 and 100-112, wherein R^{2B} is optionally substituted C1-C6 alkyl.

120. The compound of any one of claims 1-98 and 100-112, wherein R^{2B} is optionally substituted C2-C6 alkenyl.

121. The compound of any one of claims 1-98 and 100-112, wherein R^{2B} is optionally substituted C2-C6 alkynyl.

122. The compound of any one of claims 1-98 and 100-112, wherein R^{2B} is optionally substituted C3-C10 cycloalkyl.

123. The compound of any one of claims 1-98 and 100-112, wherein R^{2B} is optionally substituted phenyl.

124. The compound of any one of claims 1-98 and 100-112, wherein R^{2B} is optionally substituted 4-12 membered heterocyclyl.

125. The compound of any one of claims 1-98 and 100-112, wherein R^{2B} is optionally substituted 5-10 membered heteroaryl.

126. The compound of any one of claims 1-98, wherein one of R^{2A} and R^{2B} is hydrogen, C1-C6 alkyl, or C3-C10 cycloalkyl, and the other of R^{2A} and R^{2B} is hydrogen, —C(═O)R¹⁰, —C(═O)OR¹⁰, —C(═O)NR¹⁰R¹¹, —S(═O)R¹⁰, —S(O₂)R¹⁰, optionally substituted C1-C6 alkyl, optionally substituted C2-C6 alkenyl, optionally substituted C2-C6 alkynyl, optionally substituted C3-C10 cycloalkyl, optionally substituted phenyl, optionally substituted 4-12 membered heterocyclyl, optionally substituted 5-10 membered heteroaryl.

127. The compound of any one of claims 1-98, wherein R^{2A} and R^{2B} together with the atom to which they are attached together form an optionally substituted 4-10 membered cycloalkyl, an optionally substituted phenyl, an optionally substituted 5-10 membered heteroaryl, or an optionally substituted 4-12 membered heterocyclyl.

128. The compound of any one of claims 1-127, wherein one of X², X³, X⁴, and X⁵ is CR², one of X², X³, X⁴, and X⁵ is CR³, and the remaining X², X³, X⁴, and X⁵ are CH or N.

129. The compound of any one of claims 1-2 or 5-127, wherein one of X², X³, X⁴, and X⁵ is CR², one of X², X³, X⁴, and X⁵ is CR³, and the remaining X², X³, X⁴, and X⁵ are CH.

130. The compound of any one of claims 1-129, wherein R³ is halogen.

131. The compound of any one of claims 1-129, wherein R³ is cyano.

132. The compound of any one of claims 1-129, wherein R³ is —NR¹²R¹³.

133. The compound of any one of claims 1-129, wherein R³ is —OR¹².

134. The compound of any one of claims 1-129, wherein R³ is —C(═O)NR¹²R¹³.

135. The compound of any one of claims 1-129, wherein R³ is —C(═O)R¹².

136. The compound of any one of claims 1-129, wherein R³ is —C(═O)OR¹².

137. The compound of any one of claims 1-129, wherein R³ is —OC(═O)R¹².

138. The compound of any one of claims 1-129, wherein R³ is —NR¹²(C═O)NR¹²R¹³.

139. The compound of any one of claims 1-129, wherein R³ is —SR¹².

140. The compound of any one of claims 1-129, wherein R³ is —S(═O)R¹².

141. The compound of any one of claims 1-129, wherein R³ is —S(O₂)R¹².

142. The compound of any one of claims 1-129, wherein R³ is —S(O₂)NR¹²R¹³.

143. The compound of any one of claims 1-129, wherein R³ is —NR¹²S(O₂)NR¹³R¹⁴.

144. The compound of any one of claims 1-129, wherein R³ is —R¹²C(═O)R¹³.

145. The compound of any one of claims 1-129, wherein R³ is —NR¹²C(═O)R¹³.

146. The compound of any one of claims 1-129, wherein R³ is optionally substituted C1-C6 alkyl.

147. The compound of any one of claims 1-129, wherein R³ is optionally substituted C2-C6 alkenyl.

148. The compound of any one of claims 1-129, wherein R³ is optionally substituted C2-C6 alkynyl.

149. The compound of any one of claims 1-129, wherein R³ is optionally substituted C3-C6 cycloalkyl.

150. The compound of any one of claims 1-129, wherein R³ is optionally substituted phenyl.

151. The compound of any one of claims 1-129, wherein R³ is optionally substituted 4-6 membered heterocyclyl.

152. The compound of any one of claims 1-129, wherein R³ is optionally substituted 5-6 membered heteroaryl.

153. The compound of any one of claims 1-152, wherein m is 0.

154. The compound of any one of claims 1-152, wherein m is 1.

155. The compound of any one of claims 1-152, wherein m is 2.

156. The compound of any one of claims 1-155, wherein L is an optionally substituted 5-6 membered heteroarylene.

157. The compound of any one of claims 1-156, wherein L is a 5-6 membered heteroarylene.

158. The compound of any one of claims 1-155, wherein L is an optionally substituted 4-6 membered heterocyclylene.

159. The compound of any one of claims 1-155 or 158, wherein L is a 4-6 membered heterocyclylene.

160. A compound of Formula (I), or a pharmaceutically acceptable salt thereof, selected from the compounds described in List 1, or a pharmaceutically acceptable salt of any of the foregoing.

161. A pharmaceutical composition comprising a compound of any one of claims 1-160, or a pharmaceutically acceptable salt thereof.

162. A method of treating cancer in a subject in need thereof, comprising administering to the subject a therapeutically effective amount of a compound of any one of claims 1-160, or a pharmaceutically acceptable salt thereof, or the pharmaceutical composition of claim 161.

163. A method of treating cancer in a subject previously identified as having one or more p53 mutations, comprising administering to the subject a therapeutically effective amount of a compound of any one of claims 1-160, or a pharmaceutically acceptable salt thereof, or the pharmaceutical composition of claim 161.

164. A method of treating cancer in a subject in need thereof, comprising:
(a) determining that the subject has one or more p53 mutations, and
(b) administering to the subject a therapeutically effective amount of a compound of any one of claims 1-160, or a pharmaceutically acceptable salt thereof, or the pharmaceutical composition of claim 161.


SEQUENCE LISTING (1 sequence)

SEQ ID NO: 1 - protein, 393 aa, Homo sapiens [sequence omitted]